(19) United States
(12) Patent Application Publication
(10) Pub. No.: US 2023/0125075 A1
(43) Pub. Date: Apr. 27, 2023

(54) Composition Containing Paper Mulberry Extracts

(71) Applicant: LG Household & Health Care Ltd., Seoul (KR)

(72) Inventors: Sang Gwon Seo, Seoul (KR); Dong Jun Park, Seoul (KR); Ho Song Cho, Seoul (KR); Gil Nam Kim, Seoul (KR); Jae Hee Kim, Seoul (KR); Jun Hyeong Park, Seoul (KR); Mu Hyun Jin, Seoul (KR)

(73) Assignee: LG Household & Health Care Ltd., Seoul (KR)

(21) Appl. No.: 17/790,306

(22) PCT Filed: Jan. 4, 2021
(86) PCT No.: PCT/KR2021/000034

§ 371 Date: Dec. 27, 2022

(30) Foreign Application Priority Data
Jan. 2, 2020 (KR) 10-2020-0000283

(51) Int. Cl. A61K 36/185; A61P 7/00; A61P 3/06; A61P 3/04; A61P 37/04; A61P 5/24; A61P 37/08; A61P 3/10; A61P 25/00; A61P 1/16; A61K 36/344; A61K 36/28; A23L 33/105; A23C 9/152; A21D 2/36; A23L 2/52; A23L 2/02

(52) U.S. Cl. CPC A61K 36/185; A61P 7/00; A61P 3/06; A61P 3/04; A61P 37/04; A61P 5/24; A61P 37/08; A61P 3/10; A61P 25/00; A61P 1/16; A61K 36/344; A61K 36/28; A23L 33/105; A23C 9/152; A21D 2/36; A23L 2/52; A23L 2/02; A61K 2236/331; A61K 2236/333

(57) ABSTRACT

The present invention relates to a composition including plant extracts. The composition according to the present invention has a blood circulation improvement effect by restoring nitric oxide (NO) reduced by oxLDL, has a cholesterol improvement effect by inhibiting cholesterol synthesis, has an anti-obesity effect by promoting breakdown of triglycerides, has an immune function improvement effect by increasing interferon gamma, has a female menopause improvement effect by activating estrogen signaling, has an anti-allergic effect by inhibiting IgE that causes allergies, has a blood glucose control effect by promoting intracellular glucose uptake, has a cognitive function improvement effect by inhibiting beta-secretase activity, has a liver health improvement effect by restoring liver cell viability decreased by alcohol (ethanol) treatment, and has an andropause improvement effect by activating androgen signaling. Due to these characteristics, the composition may be used as a pharmaceutical, food, or health functional food composition.

### CROSS-REFERENCE TO RELATED APPLICATIONS

This application is a National Stage Entry of PCT International Application No. PCT/KR2021/000034, which was filed on Jan. 4, 2021, and claims priority to Korean Patent Application No. 10-2020-0000283, filed on Jan. 2, 2020.

### TECHNICAL FIELD

The present invention relates to a composition containing plant extracts, and more particularly, to a composition having a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and an andropause improvement effect.

### BACKGROUND ART

Recently, thrombosis-related diseases, which account for about 39% of the causes of death worldwide, are also increasing in Korea due to westernization of diet, excessive stress, and aging of the population.

As thrombolytic enzymes currently in use, streptokinase, urokinase, tissue plasminogen activator (t-PA), and the like are known. These enzymes mainly dissolve thrombus through an indirect mechanism that activates plasminogen into plasmin in vivo. The above plasminogen activators have problems such as allergies, fever, local bleeding, short half-life, and high cost, so these activators are limitedly used for therapeutics for thrombosis. Streptokinase and urokinase are exogenous enzymatic substances that convert plasminogen to plasmin. In addition to side effects such as fever, allergies, local bleeding, and hypotension, these enzymes are very expensive and cannot be used to prevent thrombus formation in the body.

Accordingly, there is a need for research on substances having excellent safety and a blood circulation improvement effect.

In addition, cholesterol is present everywhere in our body, including brain, nerves, muscles, skin, liver, intestines, and heart, and is known as an important substance involved in various physiological and biochemical reactions. For example, cholesterol is used as a component of cell membranes in the body and is used as a raw material for bile acids involved in digestion of hormones, vitamin D, and lipids. However, cholesterol is known to be closely related to development of cardiovascular diseases, such as arteriosclerosis and type 2 diabetes, which are representative adult diseases, and the number of patients are rapidly increasing in recent years. Cholesterol is an essential substance in our body. However, when an excess of cholesterol is present, cholesterol accumulates in the vascular endothelial cells or intima to form plaque, which leads to vascular diseases such as hyperlipidemia. Hyperlipidemia causes secondary diseases such as arteriosclerosis, hypertension, obesity, and diabetes. As such, since the total amount of blood cholesterol shows a close correlation with occurrence of coronary artery disease, it is very important to maintain the homeostasis of blood cholesterol. The cholesterol level in the blood is maintained at a certain level through synthesis of cholesterol in the liver cells and reabsorption of cholesterol in the enterocytes. Hyperlipidemia refers to a condition that causes inflammation due to the presence of an excess of cholesterol or triglycerides in the blood. Hyperlipidemia does not show any specific symptoms, but is a risk factor for high blood pressure, arteriosclerosis, and stroke. When cholesterol is accumulated due to abnormalities in cholesterol regulation in the body, hypercholesterolemia, characterized by the elevated level of total cholesterol in the blood, develops. When cholesterol metabolism control is abnormal, in addition to total cholesterol, lipoprotein components such as LDL-cholesterol and HDL-cholesterol are quantitatively changed. LDL-cholesterol deposits on the arterial wall, injuring blood vessels and causing arteriosclerosis. On the other hand, HDL-cholesterol removes harmful LDL-cholesterol and suppresses occurrence of cardiovascular diseases such as arteriosclerosis, heart disease, and stroke. Accordingly, even when a total cholesterol level is normal, high LDL-cholesterol or low HDL-cholesterol may cause arteriosclerosis.

So far, studies have been conducted to lower blood cholesterol levels. Functional raw materials derived from natural products that have a cholesterol improvement effect include Changnyeong onion extracts, barley beta-glucan extracts, linseed, puer tea extracts, red rice, plant stanol ester, policosanol-sugar cane wax alcohol, spirulina, Unibex bamboo extracts, CJ hibiscus complex extracts, aloe complex extract powder, aloe extract powder, and the like. To treat patients with high cholesterol, in parallel with drug treatment, attempts to separate substances having a cholesterol improvement effect from natural products are continuously being conducted.

In addition, obesity is known as an important risk factor causing occurrence of diabetes, hypertension, hyperlipidemia, respiratory disease, brain disease, cancer, and the like. Accordingly, interest in treatment and prevention of obesity is increasing. Currently, medical expenses and mortality due to obesity and obesity-related diseases are rapidly increasing not only in Korea but also worldwide. Currently, it is estimated that there are about 2 billion obese people in the world, and in Korea, 1 in 3 people is classified as obese.

Various studies are being conducted to treat obesity, and most anti-obesity drugs developed to date are mainly related to the mechanism of suppressing appetite or suppressing fat absorption. Obesity drugs developed through the mechanism of suppressing appetite include Sibutramine, Phentermine, Phendimetrazine, Phentermine/Topiramate complex, Locaserin, and the like. Since the above therapeutic agents may cause side effects in the central nervous system or cardiovascular system, most of the therapeutic agents are prohibited or used very limitedly. In addition, Orlistat, a therapeutic agent for treating obesity with the mechanism of inhibiting fat absorption, is known to be relatively safe, and the drug can be taken for a long time. However, Orlistat has side effects such as diarrhea, steatorrhea, and fecal incontinence. In particular, when the main cause of obesity is excessive carbohydrate intake rather than fat intake, as in Koreans, Orlistat is known to be ineffective (Sangman Kim, STUDY ON ORLISTAT. Journal of the Korean Society for Obesity 1998; 7(4): 287-92.). Accordingly, since an anti-obesity agent having a weight control effect and is safe for long-term administration has not yet been developed, there is a need for a preventive or therapeutic agent for obesity through a novel mechanism other than mechanisms known so far.

In addition, immunostimulation is one of important therapeutic strategies to reinforce the body's defense mechanisms against various diseases such as cancer and inflammatory diseases. Immunostimulation increases the activity of immune cells and stimulates immune responses to strengthen the immune system. For example, macrophages play a major role in the immune response. By phagocytosis of macrophages, microorganisms and other pyrogenic particles are absorbed into macrophages, and cytokines such as tumor necrosis factor-α (TNF-α), interleukin-1β (IL-1β), and interleukin-12 (IL-12) and cytotoxic and inflammatory substances such as nitric oxide (NO) are secreted to stimulate the immune response (Wolf et al., 1994; Lee and Hong, 2011; Murray and Wynn, 2011). Accordingly, increasing macrophage activity may be one means for enhancing immunity.

Natural products have been used in treatment of various diseases in Asian countries. In particular, natural products have been used as tonics for immunostimulation, health maintenance, and longevity. Components having various structures contained in natural products, such as polysaccharides, β-glucans, saponins, and curcuminoids, have been proposed as immune enhancers in clinical and drug applications (Ragupathi et al., 2008; Won et al., 2011; Sun et al., 2005).

In addition, menopause in women is a phenomenon in which menstruation stops due to loss of function of the ovaries, which occurs about 50 years after birth. Menopause is a physiological change that accompanies loss of fertility and is not a pathological phenomenon, but a genetically determined phenomenon. Currently, the average lifespan of Korean women is 81.2 years (2011: Statistics Korea), and the average age of menopause for Korean women as stipulated by the Korean Society of Obstetrics and Gynecology is 50 years old. These statistics show that women live in a state of depletion of female hormones for more than one third of their lives.

When a woman enters menopause, changes occur throughout the body, including the vascular system, musculoskeletal system, genitourinary system, and cranial nerve system, due to imbalance and decrease in secretion of female hormones. That is, menopause is accompanied by various diseases including vasomotor symptoms and psychological symptoms, such as hot flashes, night sweats, sleep disturbance, fatigue, depression, anxiety, difficulty in concentration, and memory impairment; dyspareunia and frequency of urination due to urogenital atrophy; loss of skin elasticity due to collagen reduction; sagging of the breast; cardiovascular and musculoskeletal symptoms; and dementia. Although menopausal symptoms differ from person to person, it has been reported that women's quality of life decreases in proportion to the frequency, severity, and duration of menopausal symptoms. In addition, as aging progresses, menopausal symptoms may develop into chronic diseases.

Hormonal therapy, drug therapy, exercise therapy, and diet may be applied to treat menopausal symptoms. However, hormonal therapy, which is mainly used medically, may increase the risk of developing breast cancer. In addition, when female hormone treatment is performed for a long period of time, uterine cancer, thrombosis/vascular diseases, gallbladder disease, and hypertension may be induced. Accordingly, in recent years, to replace drug therapy such as estrogen therapy, research on phytoestrogen, which is known to have a function similar to estrogen, has been intensively conducted.

In addition, allergy is a type of hypersensitivity reaction caused by immune imbalance and is a reaction to a specific antigen called an allergen. Specifically, allergy is a reaction induced by binding of an antigen-specific IgE antibody to mast cells or basophiles. When a large amount of IgE antibody specific for an allergen is produced, these antibodies bind to Fc receptors on the surface of mast cells or basophils. When allergens bind with the antibody-Fc receptor complex, these cells are activated and secrete various physiologically active substances such as cytokines, prostaglandins, leukotriene, histamine, bradykinin, and tryptase, and changes such vasodilatation, increased vascular permeability, smooth muscle contraction, and inflammatory responses are induced. These responses may occur locally or systemically, depending on the type and amount of mediators produced by these cells. Allergy-related diseases include allergic atopic dermatitis, systemic allergy, allergic rhinitis, asthma, food allergy, and skin allergy. The population with allergic diseases was 0.9 per 100,000 in 1979, but increased to 1.4 in 1998, showing an increase rate of about 55.6%. As a result, the importance of alleviating and preventing allergic diseases has been highlighted.

Therapeutic agents used for treatment of allergies may be broadly divided into steroidal anti-inflammatory drugs or nonsteroidal anti-inflammatory drugs and antihistamine drugs or anti-leukotriene drugs. Most of the former drugs have strong immunosuppressive action, so these drugs can have a temporary sedative effect within a short period of time. However, since there are mild side effects such as nausea and serious side effects such as growth inhibition and osteoporosis, problems may occur when taking the former drugs for a long time. The latter drugs may show temporary relief, but the drugs have side effects such as drowsiness and dizziness, which limits use thereof. Accordingly, there is a need for substances derived from natural extracts having few side effects in the body and high anti-allergic activity.

In addition, diabetes mellitus is a disease characterized by chronic hyperglycemia caused by insufficient insulin action and accompanied by various characteristic metabolic abnormalities. Since insulin is mainly involved in carbohydrate metabolism, diabetes is basically caused by abnormalities in carbohydrate metabolism. However, since diabetes affects all nutrient metabolism in the body, diabetes is a holistic metabolic disease. In particular, at present, diabetes is considered the most important chronic disease, and the incidence of diabetes is particularly high in developed countries. Diabetes is diagnosed based on a plasma glucose concentration in venous blood. In diabetes screening, two independent tests are performed on different days. When the plasma glucose concentration on an empty stomach (usually fasting for 8 to 12 hours before the test) is 126 mg/dL or higher in both tests, the subject is diagnosed with diabetes.

The diabetic population in Korea was 1.5% in the 1970s and 8 to 10% in the 1990s, showing an increasing trend. Accordingly, the incidence rate of chronic diabetic complications is also rapidly increasing.

Diabetes is largely divided into insulin-dependent diabetes mellitus and non-insulin-dependent diabetes mellitus. Insulin-dependent diabetes mellitus occurs mainly in puberty and requires insulin injection therapy. Non-insulin-dependent diabetes mellitus, which accounts for 84% of diabetic patients, mainly develops slowly in adults, so non-insulin-dependent diabetes mellitus is mainly treated with diet and exercise prescription.

Treatment of diabetes requires administration of a blood glucose control drug, and insulin is widely used as a conventional blood glucose control agent. As oral preparations, sulfonyl urea-based drugs and biguanide-based drugs are mainly used.

However, since these drugs are expensive and have to be administered for a lifetime, burden on the patient is considerable in terms of cost. In addition, since these drugs have various side effects such as allergies, myelosuppression, and hypoglycemia, patients with liver or kidney disorders, or patients with diseases such as hypotension, myocardial infarction, or hypoxia, or the elderly require caution when using these drugs.

Therefore, in consideration of the specificity of the disease treatment that requires drug administration for a lifetime, there is an urgent need for development of health functional foods and natural medicines without side effects, if possible.

In addition, Alzheimer's disease, a type of senile dementia, causes cognitive impairment such as loss of memory and learning ability. According to research results, the cognitive dysfunction is caused by damage to acetylcholinergic nerve cells at the base of the brain. Based on these results, drugs that can enhance function of acetylcholinergic nerve cells have been developed, such as agonists for muscarine acetylcholine receptors, acetylcholine production promoters, and acetylcholinesterase inhibitors. Although tacrine has been developed as an acetylcholinesterase inhibitor, tacrine has side effects such as gastrointestinal disorders. Accordingly, the need to search for active materials from natural plant materials is emerging.

In addition, it is reported that the number of modern people with fatty liver disease is increasing due to drinking habits and Western-style eating habits that prefer fatty foods. Fatty liver refers to an unhealthy condition in which the fat content in the liver exceeds 5%, and it is known that fatty liver is caused by various complex causes. Fatty liver is largely divided into alcoholic fatty liver caused by excessive drinking and nonalcoholic fatty liver associated with other causes. Thereamong, alcoholic steatohepatitis (ASH), a fatty liver caused by alcohol, is the second leading cause of death among adults in their 40s and 50s in developed countries after cancer. In Korea, which has the highest alcohol consumption in developed countries, the incidence rate of fatty liver was reported to be 32% in 2009 for adults. Unlike alcoholic fatty liver, which is directly related to alcohol, non-alcoholic steatohepatitis (NASH) is a disease in which triglycerides are accumulated in the liver regardless of alcohol. NASH is important because NASH is clinically recognized as a progressive liver disease and is recognized as a preceding disease causing cirrhosis or liver cancer. In particular, NASH is recognized as a risk factor for major adult diseases such as obesity, insulin resistance diabetes, and hyperlipidemia, and there is a need to actively prevent and treat the disease (Jonathan et al., Metabolism. 65: 1096-1108, 2016).

In addition, andropause (PADAM; partial androgen deficiency in aging male) is a process in which men generally experience decline in physical and mental aspects according to aging of the body. In andropause, masculine characteristics, overall life vitality, or mood gradually decrease due to a decrease in secretion of male hormones. Menopausal symptoms include irritability, emotional anxiety, depression, dizziness, hot flash, sweating, sleep disturbance, decreased vitality, memory loss, decreased work performance, decreased libido, skin aging, decreased bone density, and increased visceral fat.

Menopause begins to occur around the age of 50 in men as in women, and the incidence of menopause is proportional to age. As people age, adrenal gland function, sperm fertility, and Leydig cell function reduce, and serum testosterone decreases, leading to andropause symptoms. Besides environmental factors, the main cause of andropause is the decline of the male hormone-driven endocrine system. Accordingly, conventionally, a treatment method of supplementing male hormones has been the main treatment method. Male hormone replacement therapy may be performed using oral drugs, liniments, patch-type formulations, injections, or the like.

As a hormone associated with andropause, testosterone is the main circulating androgen in men. Testosterone is mainly secreted by Leydig cells of the testes and is mainly involved in development and maintenance of secondary sexual characteristics in men. Circulating testosterone in the body may be converted to dihydrotestosterone (DHT) by 5α-reductase. In addition, testosterone may be converted to estradiol by a complex of aromatase enzymes found in the liver, fat, and testes. Aromatization of testosterone to estradiol may lead to andropause symptoms such as the gynecomastia symptom. Increasing testosterone and decreasing levels of dihydrotestosterone and estradiol may help relieve symptoms of andropause.

Conversion of testosterone to DHT is important in many respects. For example, DHT binds SHBG with a much higher affinity than testosterone. In addition, in many tissues, the activity of testosterone determines conversion into DHT, which is bound to cytosolic receptor proteins. The steroid receptor complex is translocated to the nucleus and cellular changes related to transcription and androgen action are initiated.

98% of testosterone binds to proteins and circulates with the blood. In men, approximately 40% of testosterone binds to high affinity sex hormone binding globulin (SHBG), and the remaining 60% binds weakly to albumin. Accordingly, testosterone may be measured through clinical trials. Testosterone present in the blood without being bound to proteins is called “free testosterone”, and “total testosterone” or “testosterone” includes free testosterone and protein-bound testosterone.

SHBG plays a role in regulating action of testosterone by binding to testosterone. When SHBG binds to testosterone, testosterone loses hormonal activity thereof, and when SHBG is separated from testosterone, testosterone recovers hormonal activity. 40 to 80% of testosterone binds to SHBG and exists in a biologically inactive state. As a result of examining men in their 40s and 70s, the amount of SHBG was found to increase. Increase in SHBG in the blood with aging further decreases bioavailable testosterone, which may exacerbate andropause symptoms.

Male hormone (androgen) drug supplement therapy is a treatment method for andropause complex syndrome. However, such drug supplement therapy may exhibit liver toxicity, may increase LDL by affecting blood lipid metabolism, and may reduce HDL and cause cardiovascular disease. In addition, administration of male hormones may adversely affect liver and lipid status, cardiovascular and prostate diseases, and sleep and behavioral disorders. There is also a problem that requires regular examination for these side effects. It has also been reported that testosterone therapy cannot be given to asymptomatic patients or patients with overt prostate cancer.

In recent years, to overcome the problems of such drug therapy, development of a therapeutic agent for andropause complex syndrome containing natural herbal medicines as a main ingredient is being attempted. However, the related art using such herbal materials as main components is mainly limited to studies related to sexual function, which is a fragmentary symptom of andropause complex syndrome. Also, the above technology has nothing to do with fundamentally improving the symptoms of andropause complex syndrome, such as osteoporosis, decreased muscle strength, and decreased motivation. So far, development of agents for treatment of andropause complex syndrome is insufficient.

### DISCLOSURE

### Technical Problem

The present inventors confirmed that 111 plant extracts had a blood circulation improvement effect by restoring nitric oxide (NO) reduced by oxLDL, had a cholesterol improvement effect by inhibiting cholesterol synthesis, had an anti-obesity effect by promoting breakdown of triglycerides, had an immune function improvement effect by increasing interferon gamma, had a female menopause improvement effect by activating estrogen signaling, had an anti-allergic effect by inhibiting IgE that causes allergies, had a blood glucose control effect by promoting intracellular glucose uptake, had a cognitive function improvement effect by inhibiting beta-secretase activity, had a liver health improvement effect by restoring liver cell viability decreased by alcohol (ethanol) treatment, and had an andropause improvement effect by activating androgen signaling. Based on these results, the present inventors conducted further studies to complete the present invention.

Therefore, the present invention has been made in view of the above problems, and it is one object of the present invention to provide a composition for improving blood circulation, improving cholesterol, suppressing obesity, improving immune function, improving female menopause, suppressing allergies, controlling blood glucose, improving cognitive function, improving liver health, and improving andropause including, as active ingredients, one or more plant extracts selected from the group consisting of Island thistle (scientific name: Cirsium nipponicum (Maxim.) Makino), Deodeok (scientific name: Codonopsis lanceolata), Ulleung goldenrod (scientific name: Solidago virgaurea), goat's beard (scientific name: Aruncus dioicus), Ulleungdo aster (scientific name: Aster glehnii), Myeongyi (scientific name: Allium victorialis or Allium ulleungense), Aging chive (scientific name: Allium senescens), Silvery mountain ash (scientific name: Sorbus commixta), Ulleungdo thyme (scientific name: Thymus quinquecostatus var. japonica HARA), Wild chervil (scientific name: Anthriscus sylvestris), Giant Knotweed (scientific name: Fallopia sachalinensis), Mono maple (scientific name: Acer pictum), Ulleungdo ladyfern (scientific name: Athyrium acutipinnulum), Wild wasabi (scientific name: Wasabia japonica), Yellow Mongolian snakegourd (scientific name: Trichosanthes kirilowii), Pungdo solomon's seal (scientific name: Polygonatum odoratum), Songak (scientific name: Hedera rhombea), Ulleungdo sweet violet (scientific name: Viola woosanensis), Korean bellflower (scientific name: Campanula takesimana), Ulleungdo turk's-cap lily (scientific name: Lilium hansonii), Ulleungdo spike speedwell (scientific name: Pseudolysimachion insulare), Coastal moss-like stonecrop (scientific name: Sedum oryzifolium), Water chickweed (Giant chickweed) (scientific name: Stellaria aquatica), Heartleaf Houttuynia (scientific name: Houttuynia cordata Thunb.), Ulleungdo liverleaf (scientific name: Hepatica maxima (Nakai) Nakai), Chinese buttercup (scientific name: Ranunculus quelpaertensis (H. Lev.) Nakai), Tricuspidate falsenettle (scientific name: Boehmeria tricuspis (Hance) Makino), Long-calyx pink (scientific name: Dianthus longicalyx Miq.), Curled dock (scientific name: Rumex crispus L.), Erect St. Johnswort (scientific name: Hypericum erectum Thunb.), Five-leaf gynostemma (scientific name: Gynostemmapentaphylla (Thunb.) Makino), Bird's egg cucumber (scientific name: Melothria japonica (Thunb.) Maxim.), Ulleungdo rockcress (scientific name: Arabis takesimana Nakai), Hedge Mustard (scientific name: Sisymbrium officinale (L.) Scop.), Fauriei rosebay (scientific name: Rhododendron fauriei), Asian prince's pine (scientific name: Chimaphila japonica Miq.), Marlberry (scientific name: Ardisia japonica (Thunb.) Blume), Spoon-leaf yellow loosestrife (scientific name: Lysimachia mauritiana Lam.), Climbing hydrangea (scientific name: Hydrangea petiolaris Siebold & Zucc.), Stringy stonecrop (scientific name: Sedum sarmentosum Bunge), Ulleungdo stonecrop (scientific name: Sedum takesimense Nakai), Foam flower (scientific name: Tiarella polyphylla D. Don), East Asian cinquefoil (scientific name: Potentilla chinensis Ser.), Indigobush Amorpha (scientific name: Amorpha fruticosa L.), Sericea lespedeza (scientific name: Lespedeza cuneata (Dum. Cours.) G. Don.), Alfalfa (scientific name: Medicago sativa L.), Amur vetch (scientific name: Vicia amurensis Oett.), Hairy purple loosestrife (scientific name: Lythrum salicaria L.), South enchanter's nightshade (scientific name: Circaea mollis Siebold & Zucc.), Long-seed willowherb (scientific name: Epilobium pyrricholophum Franch. & Say.), Evening Primrose (scientific name: Oenothera biennis L.), Stolon golden saxifrage (scientific name: Chrysosplenium flagelliferum F. Schmidt), Serrate-petal rockfoil (scientific name: Saxifraga fortunei var. incisolobata (Engl. & Irmsch.) Nakai), Ulleungdo deadnettle (scientific name: Lamium takesimense NAKAI.), Island ninebark (scientific name: Physocarpus insularis (Nakai) Nakai), Island corydalis (scientific name: Corydalis ilistipes Nakai), Ulleungdo raspberry (scientific name: Rubus takesimensis Nakai), Korea Dystaenia (scientific name: Dystaenia takeshimana (Nakai) Kitag), Ulleungdo violet (scientific name: Viola takeshimana Nakai), Spindle Tree (scientific name: Euonymus japonicus Thunb.), Crimson grapevine (scientific name: Vitis coignetiae Pulliat ex Planch.), Aralia continentalis (scientific name: Aralia cordata var. continentalis (Kitag.) Y. C. Chu), Japanese Angelica (scientific name: Aralia elata (Miq.) Seem.), Glossy-leaf paper plant (scientific name: Fatsia japonica (Thunb.) Decne. & Planch.), Three-leaf clematis (scientific name: Clematis apiifolia DC.), Lyre-leaf nightshade (scientific name: Solanum lyratum Thunb.), Ivy morning glory (scientific name: Calystegia hederacea Wall.), Beach morning glory (scientific name: Calystegia soldanella (L.) Roem. & Schult.), East Asian beautyberry (scientific name: Callicarpa japonica Thunb.), Korean mint (scientific name: Agastache rugosa (Fisch. & C. A. Mey.) Kuntze), Small-flower Asian calamint (scientific name: Clinopodium chinense var. parviflorum (Kudo) H. Hara), Henbit deadnettle (scientific name: Lamium amplexicaule L.), Oriental motherwort (scientific name: Leonurus japonicus Houtt.), Long-stalk low meadow-rue (scientific name: Thalictrum kemense Fr.), Korean spice viburnum (scientific name: Viburnum carlesii Hemsl.), Asian greater celandine (scientific name: Chelidonium majus var. asiaticum (H. Hara) Ohwi), Seashore spatulate aster (scientific name: Aster spathulifolius Maxim.), Leopard plant (scientific name: Farfugium japonicum (L.) Kitam.), Oriental yellowhead (scientific name: Inula britannica var. japonica (Thunb.) Franch. & Say.), Giant butterbur (scientific name: Petasites japonicus (Siebold & Zucc.) Maxim.), Ciliated-fruit sedge (scientific name: Carex blepharicarpa Franch.), Short-stem sedge (scientific name: Carex breviculmis R. Br.), Purple maiden silvergrass (scientific name: Miscanthus sinensis var. purpurascens (Andersson) Rendle), Foxtail fountaingrass (scientific name: Pennisetum alopecuroides (L.) Spreng.), Arrow bamboo (scientific name: Pseudosasa japonica (Siebold & Zucc. ex Steud.) Makino ex Nakai), Kuril bamboo (scientific name: Sasa kurilensis (Rupr.) Makino & Shibata), Shrubby sophora (scientific name: Sophora flavescens Aiton), Orange Daylily (scientific name: Hemerocallis fulva (L.) L.), Tiger lily (scientific name: Lilium lancifolium Thunb.), Big blue lilyturf (scientific name: Liriope platyphylla F. T. Wang & T. Tang), False lily of the valley (scientific name: Maianthemum dilatatum (A. W. Wood) A. Nelson & J. F. Macbr.), Japanese Cedar (scientific name: Cryptomeria japonica (Thunb. ex L. f.) D. Don), Thunberg's bay-tree (scientific name: Machilus thunbergii Siebold & Zucc.), Ulleungdo white pine (scientific name: Pinus parviflora Siebold & Zucc.), Ulleungdo hemlock (scientific name: Tsuga sieboldii Carriere), Sericeous newlitsea (scientific name: Neolitsea sericea (Blume) Koidz.), Macropodous daphniphyllum (scientific name: Daphniphyllum macropodum Miq.), Scabrous aphananthe (scientific name: Aphananthe aspera (Thunb.) Planch.), Caudate-leaf hackberry (scientific name: Celtis jessoensis Koidz.), Manchurian Elm (scientific name: Ulmus laciniata (Trautv.) Mayr), Montane alder (scientific name: Alnus maximowiczii Callier), Ulleungdo linden (scientific name: Tilia insularis Nakai), Fragrant snowbell (scientific name: Styrax obassia Siebold & Zucc.), Ulleungdo flowering cherry (scientific name: Prunus takesimensis Nakai), Spotted laurel (scientific name: Aucuba japonica Thunb.), Ulleungdo maple (scientific name: Acer takesimense Nakai), Ulleungdo amur corktree (scientific name: Phellodendron insulare Nakai), Alianthus-like prickly-ash (scientific name: Zanthoxylum ailanthoides Siebold & Zucc.), Wax-leaf privet (scientific name: Ligustrum japonicum Thunb.), Ulleungdo honeysuckle (scientific name: Lonicera insularis Nakai), and Mountain hydrangea (scientific name: Hydrangea serrata).

### Technical Solution

In accordance with one aspect of the present invention, provided is a composition for improving blood circulation, improving cholesterol, suppressing obesity, improving immune function, improving female menopause, suppressing allergies, controlling blood glucose, improving cognitive function, improving liver health, and improving andropause including, as active ingredients, one or more plant extracts selected from the group consisting of Island thistle (scientific name: Cirsium nipponicum (Maxim.) Makino), Deodeok (scientific name: Codonopsis lanceolata), Ulleung goldenrod (scientific name: Solidago virgaurea), goat's beard (scientific name: Aruncus dioicus), Ulleungdo aster (scientific name: Aster glehnii), Myeongyi (scientific name: Allium victorialis or Allium ulleungense), Aging chive (scientific name: Allium senescens), Silvery mountain ash (scientific name: Sorbus commixta), Ulleungdo thyme (scientific name: Thymus quinquecostatus var. japonica HARA), Wild chervil (scientific name: Anthriscus sylvestris), Giant Knotweed (scientific name: Fallopia sachalinensis), Mono maple (scientific name: Acer pictum), Ulleungdo ladyfern (scientific name: Athyrium acutipinnulum), Wild wasabi (scientific name: Wasabia japonica), Yellow Mongolian snakegourd (scientific name: Trichosanthes kirilowii), Pungdo solomon's seal (scientific name: Polygonatum odoratum), Songak (scientific name: Hedera rhombea), Ulleungdo sweet violet (scientific name: Viola woosanensis), Korean bellflower (scientific name: Campanula takesimana), Ulleungdo turk's-cap lily (scientific name: Lilium hansonii), Ulleungdo spike speedwell (scientific name: Pseudolysimachion insulare), Coastal moss-like stonecrop (scientific name: Sedum oryzifolium), Water chickweed (Giant chickweed) (scientific name: Stellaria aquatica), Heartleaf Houttuynia (scientific name: Houttuynia cordata Thunb.), Ulleungdo liverleaf (scientific name: Hepatica maxima (Nakai) Nakai), Chinese buttercup (scientific name: Ranunculus quelpaertensis (H. Lev.) Nakai), Tricuspidate falsenettle (scientific name: Boehmeria tricuspis (Hance) Makino), Long-calyx pink (scientific name: Dianthus longicalyx Miq.), Curled dock (scientific name: Rumex crispus L.), Erect St. Johnswort (scientific name: Hypericum erectum Thunb.), Five-leaf gynostemma (scientific name: Gynostemma pentaphylla (Thunb.) Makino), Bird's egg cucumber (scientific name: Melothria japonica (Thunb.) Maxim.), Ulleungdo rockcress (scientific name: Arabis takesimana Nakai), Hedge Mustard (scientific name: Sisymbrium officinale (L.) Scop.), Fauriei rosebay (scientific name: Rhododendron fauriei), Asian prince's pine (scientific name: Chimaphila japonica Miq.), Marlberry (scientific name: Ardisia japonica (Thunb.) Blume), Spoon-leaf yellow loosestrife (scientific name: Lysimachia mauritiana Lam.), Climbing hydrangea (scientific name: Hydrangea petiolaris Siebold & Zucc.), Stringy stonecrop (scientific name: Sedum sarmentosum Bunge), Ulleungdo stonecrop (scientific name: Sedum takesimense Nakai), Foam flower (scientific name: Tiarella polyphylla D. Don), East Asian cinquefoil (scientific name: Potentilla chinensis Ser.), Indigobush Amorpha (scientific name: Amorpha fruticosa L.), Sericea lespedeza (scientific name: Lespedeza cuneata (Dum. Cours.) G. Don.), Alfalfa (scientific name: Medicago sativa L.), Amur vetch (scientific name: Vicia amurensis Oett.), Hairy purple loosestrife (scientific name: Lythrum salicaria L.), South enchanter's nightshade (scientific name: Circaea mollis Siebold & Zucc.), Long-seed willowherb (scientific name: Epilobium pyrricholophum Franch. & Say.), Evening Primrose (scientific name: Oenothera biennis L.), Stolon golden saxifrage (scientific name: Chrysosplenium flagelliferum F. Schmidt), Serrate-petal rockfoil (scientific name: Saxifraga fortunei var. incisolobata (Engl. & Irmsch.) Nakai), Ulleungdo deadnettle (scientific name: Lamium takesimense NAKAI.), Island ninebark (scientific name: Physocarpus insularis (Nakai) Nakai), Island corydalis (scientific name: Corydalis ilistipes Nakai), Ulleungdo raspberry (scientific name: Rubus takesimensis Nakai), Korea Dystaenia (scientific name: Dystaenia takeshimana (Nakai) Kitag), Ulleungdo violet (scientific name: Viola takeshimana Nakai), Spindle Tree (scientific name: Euonymus japonicus Thunb.), Crimson grapevine (scientific name: Vitis coignetiae Pulliat ex Planch.), Aralia continentalis (scientific name: Aralia cordata var. continentalis (Kitag.) Y. C. Chu), Japanese Angelica (scientific name: Aralia elata (Miq.) Seem.), Glossy-leaf paper plant (scientific name: Fatsia japonica (Thunb.) Decne. & Planch.), Three-leaf clematis (scientific name: Clematis apiifolia DC.), Lyre-leaf nightshade (scientific name: Solanum lyratum Thunb.), Ivy morning glory (scientific name: Calystegia hederacea Wall.), Beach morning glory (scientific name: Calystegia soldanella (L.) Roem. & Schult.), East Asian beautyberry (scientific name: Callicarpa japonica Thunb.), Korean mint (scientific name: Agastache rugosa (Fisch. & C. A. Mey.) Kuntze), Small-flower Asian calamint (scientific name: Clinopodium chinense var. parviflorum (Kudo) H. Hara), Henbit deadnettle (scientific name: Lamium amplexicaule L.), Oriental motherwort (scientific name: Leonurus japonicus Houtt.), Long-stalk low meadow-rue (scientific name: Thalictrum kemense Fr.), Korean spice viburnum (scientific name: Viburnum carlesii Hemsl.), Asian greater celandine (scientific name: Chelidonium majus var. asiaticum (H. Hara) Ohwi), Seashore spatulate aster (scientific name: Aster spathulifolius Maxim.), Leopard plant (scientific name: Farfugium japonicum (L.) Kitam.), Oriental yellowhead (scientific name: Inula britannica var. japonica (Thunb.) Franch. & Say.), Giant butterbur (scientific name: Petasites japonicus (Siebold & Zucc.) Maxim.), Ciliated-fruit sedge (scientific name: Carex blepharicarpa Franch.), Short-stem sedge (scientific name: Carex breviculmis R. Br.), Purple maiden silvergrass (scientific name: Miscanthus sinensis var. purpurascens (Andersson) Rendle), Foxtail fountaingrass (scientific name: Pennisetum alopecuroides (L.) Spreng.), Arrow bamboo (scientific name: Pseudosasa japonica (Siebold & Zucc. ex Steud.) Makino ex Nakai), Kuril bamboo (scientific name: Sasa kurilensis (Rupr.) Makino & Shibata), Shrubby sophora (scientific name: Sophora flavescens Aiton), Orange Daylily (scientific name: Hemerocallis fulva (L.) L.), Tiger lily (scientific name: Lilium lancifolium Thunb.), Big blue lilyturf (scientific name: Liriope platyphylla F. T. Wang & T. Tang), False lily of the valley (scientific name: Maianthemum dilatatum (A. W. Wood) A. Nelson & J. F. Macbr.), Japanese Cedar (scientific name: Cryptomeria japonica (Thunb. ex L. f.) D. Don), Thunberg's bay-tree (scientific name: Machilus thunbergii Siebold & Zucc.), Ulleungdo white pine (scientific name: Pinus parviflora Siebold & Zucc.), Ulleungdo hemlock (scientific name: Tsuga sieboldii Carriere), Sericeous newlitsea (scientific name: Neolitsea sericea (Blume) Koidz.), Macropodous daphniphyllum (scientific name: Daphniphyllum macropodum Miq.), Scabrous aphananthe (scientific name: Aphananthe aspera (Thunb.) Planch.), Caudate-leaf hackberry (scientific name: Celtis jessoensis Koidz.), Manchurian Elm (scientific name: Ulmus laciniata (Trautv.) Mayr), Montane alder (scientific name: Alnus maximowiczii Callier), Ulleungdo linden (scientific name: Tilia insularis Nakai), Fragrant snowbell (scientific name: Styrax obassia Siebold & Zucc.), Ulleungdo flowering cherry (scientific name: Prunus takesimensis Nakai), Spotted laurel (scientific name: Aucuba japonica Thunb.), Ulleungdo maple (scientific name: Acer takesimense Nakai), Ulleungdo amur corktree (scientific name: Phellodendron insulare Nakai), Alianthus-like prickly-ash (scientific name: Zanthoxylum ailanthoides Siebold & Zucc.), Wax-leaf privet (scientific name: Ligustrum japonicum Thunb.), Ulleungdo honeysuckle (scientific name: Lonicera insularis Nakai), and Mountain hydrangea (scientific name: Hydrangea serrata).

### Advantageous Effects

A composition including plant extracts according to the present invention has a blood circulation improvement effect by restoring nitric oxide (NO) reduced by oxLDL, has a cholesterol improvement effect by inhibiting cholesterol synthesis, has an anti-obesity effect by promoting breakdown of triglycerides, has an immune function improvement effect by increasing interferon gamma, has a female menopause improvement effect by activating estrogen signaling, has an anti-allergic effect by inhibiting IgE that causes allergies, has a blood glucose control effect by promoting intracellular glucose uptake, has a cognitive function improvement effect by inhibiting beta-secretase activity, has a liver health improvement effect by restoring liver cell viability decreased by alcohol (ethanol) treatment, and has an andropause improvement effect by activating androgen signaling. Due to these characteristics, the composition can be used as a pharmaceutical or food composition.

### BEST MODE

Hereinafter, the configuration of the present invention will be described in detail.

The present invention relates to a composition including, as active ingredients, one or more plant extracts selected from the group consisting of Island thistle (scientific name: Cirsium nipponicum (Maxim.) Makino), Deodeok (scientific name: Codonopsis lanceolata), Ulleung goldenrod (scientific name: Solidago virgaurea), goat's beard (scientific name: Aruncus dioicus), Ulleungdo aster (scientific name: Aster glehnii), Myeongyi (scientific name: Allium victorialis or Allium ulleungense), Aging chive (scientific name: Allium senescens), Silvery mountain ash (scientific name: Sorbus commixta), Ulleungdo thyme (scientific name: Thymus quinquecostatus var. japonica HARA), Wild chervil (scientific name: Anthriscus sylvestris), Giant Knotweed (scientific name: Fallopia sachalinensis), Mono maple (scientific name: Acer pictum), Ulleungdo ladyfern (scientific name: Athyrium acutipinnulum), Wild wasabi (scientific name: Wasabia japonica), Yellow Mongolian snakegourd (scientific name: Trichosanthes kirilowii), Pungdo solomon's seal (scientific name: Polygonatum odoratum), Songak (scientific name: Hedera rhombea), Ulleungdo sweet violet (scientific name: Viola woosanensis), Korean bellflower (scientific name: Campanula takesimana), Ulleungdo turk's-cap lily (scientific name: Lilium hansonii), Ulleungdo spike speedwell (scientific name: Pseudolysimachion insulare), Coastal moss-like stonecrop (scientific name: Sedum oryzifolium), Water chickweed (Giant chickweed) (scientific name: Stellaria aquatica), Heartleaf Houttuynia (scientific name: Houttuynia cordata Thunb.), Ulleungdo liverleaf (scientific name: Hepatica maxima (Nakai) Nakai), Chinese buttercup (scientific name: Ranunculus quelpaertensis (H. Lev.) Nakai), Tricuspidate falsenettle (scientific name: Boehmeria tricuspis (Hance) Makino), Long-calyx pink (scientific name: Dianthus longicalyx Miq.), Curled dock (scientific name: Rumex crispus L.), Erect St. Johnswort (scientific name: Hypericum erectum Thunb.), Five-leaf gynostemma (scientific name: Gynostemmapentaphylla (Thunb.) Makino), Bird's egg cucumber (scientific name: Melothria japonica (Thunb.) Maxim.), Ulleungdo rockcress (scientific name: Arabis takesimana Nakai), Hedge Mustard (scientific name: Sisymbrium officinale (L.) Scop.), Fauriei rosebay (scientific name: Rhododendron fauriei), Asian prince's pine (scientific name: Chimaphila japonica Miq.), Marlberry (scientific name: Ardisia japonica (Thunb.) Blume), Spoon-leaf yellow loosestrife (scientific name: Lysimachia mauritiana Lam.), Climbing hydrangea (scientific name: Hydrangea petiolaris Siebold & Zucc.), Stringy stonecrop (scientific name: Sedum sarmentosum Bunge), Ulleungdo stonecrop (scientific name: Sedum takesimense Nakai), Foam flower (scientific name: Tiarella polyphylla D. Don), East Asian cinquefoil (scientific name: Potentilla chinensis Ser.), Indigobush Amorpha (scientific name: Amorpha fruticosa L.), Sericea lespedeza (scientific name: Lespedeza cuneata (Dum. Cours.) G. Don.), Alfalfa (scientific name: Medicago sativa L.), Amur vetch (scientific name: Vicia amurensis Oett.), Hairy purple loosestrife (scientific name: Lythrum salicaria L.), South enchanter's nightshade (scientific name: Circaea mollis Siebold & Zucc.), Long-seed willowherb (scientific name: Epilobium pyrricholophum Franch. & Say.), Evening Primrose (scientific name: Oenothera biennis L.), Stolon golden saxifrage (scientific name: Chrysosplenium flagelliferum F. Schmidt), Serrate-petal rockfoil (scientific name: Saxifraga fortunei var. incisolobata (Engl. & Irmsch.) Nakai), Ulleungdo deadnettle (scientific name: Lamium takesimense NAKAI.), Island ninebark (scientific name: Physocarpus insularis (Nakai) Nakai), Island corydalis (scientific name: Corydalis ilistipes Nakai), Ulleungdo raspberry (scientific name: Rubus takesimensis Nakai), Korea Dystaenia (scientific name: Dystaenia takeshimana (Nakai) Kitag), Ulleungdo violet (scientific name: Viola takeshimana Nakai), Spindle Tree (scientific name: Euonymus japonicus Thunb.), Crimson grapevine (scientific name: Vitis coignetiae Pulliat ex Planch.), Aralia continentalis (scientific name: Aralia cordata var. continentalis (Kitag.) Y. C. Chu), Japanese Angelica (scientific name: Aralia elata (Miq.) Seem.), Glossy-leaf paper plant (scientific name: Fatsia japonica (Thunb.) Decne. & Planch.), Three-leaf clematis (scientific name: Clematis apiifolia DC.), Lyre-leaf nightshade (scientific name: Solanum lyratum Thunb.), Ivy morning glory (scientific name: Calystegia hederacea Wall.), Beach morning glory (scientific name: Calystegia soldanella (L.) Roem. & Schult.), East Asian beautyberry (scientific name: Callicarpa japonica Thunb.), Korean mint (scientific name: Agastache rugosa (Fisch. & C. A. Mey.) Kuntze), Small-flower Asian calamint (scientific name: Clinopodium chinense var. parviflorum (Kudo) H. Hara), Henbit deadnettle (scientific name: Lamium amplexicaule L.), Oriental motherwort (scientific name: Leonurus japonicus Houtt.), Long-stalk low meadow-rue (scientific name: Thalictrum kemense Fr.), Korean spice viburnum (scientific name: Viburnum carlesii Hemsl.), Asian greater celandine (scientific name: Chelidonium majus var. asiaticum (H. Hara) Ohwi), Seashore spatulate aster (scientific name: Aster spathulifolius Maxim.), Leopard plant (scientific name: Farfugium japonicum (L.) Kitam.), Oriental yellowhead (scientific name: Inula britannica var. japonica (Thunb.) Franch. & Say.), Giant butterbur (scientific name: Petasites japonicus (Siebold & Zucc.) Maxim.), Ciliated-fruit sedge (scientific name: Carex blepharicarpa Franch.), Short-stem sedge (scientific name: Carex breviculmis R. Br.), Purple maiden silvergrass (scientific name: Miscanthus sinensis var. purpurascens (Andersson) Rendle), Foxtail fountaingrass (scientific name: Pennisetum alopecuroides (L.) Spreng.), Arrow bamboo (scientific name: Pseudosasa japonica (Siebold & Zucc. ex Steud.) Makino ex Nakai), Kuril bamboo (scientific name: Sasa kurilensis (Rupr.) Makino & Shibata), Shrubby sophora (scientific name: Sophora flavescens Aiton), Orange Daylily (scientific name: Hemerocallis fulva (L.) L.), Tiger lily (scientific name: Lilium lancifolium Thunb.), Big blue lilyturf (scientific name: Liriope platyphylla F. T. Wang & T. Tang), False lily of the valley (scientific name: Maianthemum dilatatum (A. W. Wood) A. Nelson & J. F. Macbr.), Japanese Cedar (scientific name: Cryptomeria japonica (Thunb. ex L. f.) D. Don), Thunberg's bay-tree (scientific name: Machilus thunbergii Siebold & Zucc.), Ulleungdo white pine (scientific name: Pinus parviflora Siebold & Zucc.), Ulleungdo hemlock (scientific name: Tsuga sieboldii Carriere), Sericeous newlitsea (scientific name: Neolitsea sericea (Blume) Koidz.), Macropodous daphniphyllum (scientific name: Daphniphyllum macropodum Miq.), Scabrous aphananthe (scientific name: Aphananthe aspera (Thunb.) Planch.), Caudate-leaf hackberry (scientific name: Celtis jessoensis Koidz.), Manchurian Elm (scientific name: Ulmus laciniata (Trautv.) Mayr), Montane alder (scientific name: Alnus maximowiczii Callier), Ulleungdo linden (scientific name: Tilia insularis Nakai), Fragrant snowbell (scientific name: Styrax obassia Siebold & Zucc.), Ulleungdo flowering cherry (scientific name: Prunus takesimensis Nakai), Spotted laurel (scientific name: Aucuba japonica Thunb.), Ulleungdo maple (scientific name: Acer takesimense Nakai), Ulleungdo amur corktree (scientific name: Phellodendron insulare Nakai), Alianthus-like prickly-ash (scientific name: Zanthoxylum ailanthoides Siebold & Zucc.), Wax-leaf privet (scientific name: Ligustrum japonicum Thunb.), Ulleungdo honeysuckle (scientific name: Lonicera insularis Nakai), and Mountain hydrangea (scientific name: Hydrangea serrata).

The composition including plant extracts as active ingredients according to the present invention may be used as a composition for improving blood circulation, improving cholesterol, suppressing obesity, improving immune function, improving female menopause, suppressing allergies, controlling blood glucose, improving cognitive function, improving liver health, and improving andropause.

According to an embodiment of the present invention, since the composition including plant extracts as active ingredients according to the present invention exhibits a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and an andropause improvement effect at a low concentration, the composition may be used as an active ingredient for a food or pharmaceutical composition for improving blood circulation, improving cholesterol, suppressing obesity, improving immune function, improving female menopause, suppressing allergies, controlling blood glucose, improving cognitive function, improving liver health, and improving andropause.

In addition, the present invention provides use of the plant extracts to prepare a food or pharmaceutical composition for improving blood circulation, improving cholesterol, suppressing obesity, improving immune function, improving female menopause, suppressing allergies, controlling blood glucose, improving cognitive function, improving liver health, and/or improving andropause and a method of improving blood circulation, improving cholesterol, suppressing obesity, improving immune function, improving female menopause, suppressing allergies, controlling blood glucose, improving cognitive function, improving liver health, and/or improving andropause including a step of administering the plant extracts.

In the present invention, the type of plant is not limited, and a cultivated plant may be used, or a commercially available plant may be used, and the source of the plant is not limited.

The plant extracts of the present invention may be extracted according to a method known in the art, and the method is not particularly limited. Alternatively, commercially available extracts may be used.

Regardless of regions in which plants are grown, the plant extracts according to the present invention may have a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and/or an andropause improvement effect. Particularly, plant extracts collected from Ulleungdo may have a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and/or an andropause improvement effect.

The plant extracts may be obtained using any part of a plant, and there is no limitation on extraction sites. Obtaining the plant extracts is not limited by the shape of a plant, and a process of obtaining the extracts includes a process of drying the plant. For example, the plant may be the whole, roots, stems, leaves, fruits, flowers, shoots, branches, bark, sap, bulbils, and/or seeds of the above-described plant.

In the present invention, the term “extracts” include extracts and all formulations that may be formed using extracts, such as extracts obtained by extracting the above-described plants, a diluted or concentrated liquid of the extracts, a product obtained by drying the extracts, a substance prepared by adjusting or purifying the extracts, a fermented product of the extracts, or a mixture thereof. In addition, the extracts include juice obtained by filtrating the product obtained after directly pressing or pulverizing the plants. The plant may be extracted as it is or may be extracted by oriental medicine processing. The “oriental medicine processing ( [symbol] )” refers to a pharmaceutical technology that changes the original properties of medicines by processing the medicines based on oriental medicine theory. For example, the “oriental medicine processing” includes the “cho ( [symbol] )” method of roasting medicinal materials, the “ja ( [symbol] )” method in which a liquid auxiliary material is permeated into medicinal materials by roasting the medicinal materials with a certain amount of the liquid auxiliary material, and the “steam ( [symbol] )” method in which liquid auxiliary materials according to the oriental medicine processing regulations for each medicinal material are added into an appropriate container, and the mixture is mixed, heated, and dried to an appropriate degree.

In the present invention, an extraction method is not particularly limited, and extraction may be performed according to a method commonly used in the art. Non-limiting examples of the extraction method include a solvent extraction method, a hot water extraction method, an ultrasonic extraction method, a filtration method, a reflux extraction method, and the like, and the methods may be performed alone, or two or more methods may be used in combination.

In the present invention, the type of extraction solvent used for extraction is not particularly limited, and any solvent known in the art may be used. In the present invention, the extracts may be obtained by performing extraction using water, a low-grade alcohol having 1 to 6 carbon atoms, or a mixture thereof. In addition, non-limiting examples of the extraction solvent include water; a low-grade alcohol having 1 to 6 carbon atoms, such as methanol, ethanol, propyl alcohol, and butyl alcohol; a polyhydric alcohol such as glycerin, butylene glycol, and propylene glycol; a hydrocarbon-based solvent such as methyl acetate, ethyl acetate, acetone, benzene, hexane, diethyl ether, and dichloromethane; or a mixture thereof. Specifically, as the extraction solvent, water, a low-grade alcohol, 1,3-butylene glycol, and ethyl acetate may be used alone, or two or more thereof may be used in combination. In this case, when two or more solvents are mixed and used, the mixing ratio between the solvents is not particularly limited.

In the present invention, extraction may be performed at an extraction temperature of 10 to 80° C., specifically 15 to 50° C. for an extraction time of 2 hours to 3 days, specifically 12 hours to 18 days using a solvent of 1 to 100 times by weight, specifically 1 to 50 times by weight, more specifically 2 to 20 times by weight based on the weight of the dry matter of the plants. The extraction method may include a process of obtaining a liquid crude extract by performing extraction 1 to 5 times consecutively for the dried material and the crushed material.

In the present invention, to remove solid particles suspended in the extracts, the solid particles may be filtered out from the extracts by filtration (e.g., using nylon or filter paper). In addition, the extracts may be used after performing filtration using freeze filtration or the like, or the filtered extracts may be used after drying using freeze drying, hot air drying, spray drying, or the like.

The liquid crude extract may be separated from the dried lysate of a plant by a method such as reduced pressure filtration and then subjected to a process of concentration or drying. For example, the liquid crude extract may be a concentrated solution obtained by performing reduced pressure concentration at 20 to 100° C., preferably 30 to 70° C. using a vacuum rotary concentrator, and a powdered extract may be obtained by drying the liquid extract. When necessary, the concentrated or powdered extract may be used by dissolving the extract in water, alcohol, dimethyl sulfoxide (DMSO), or a mixed solvent thereof.

In the present invention, the active ingredient may be a fraction of a plant extract.

In the present invention, the term “fraction” refers to a specific component or a group consisting of specific components separated from a mixture including various components by performing fractionation.

In the present invention, the fractionation method for obtaining the fraction is not particularly limited, and fractionation may be performed according to a method commonly used in the art. Non-limiting examples of the fractionation method include a method of obtaining a fraction from extracts by treating a predetermined solvent to plant extracts obtained by extracting a plant.

In the present invention, the kind of solvent used to obtain the fraction is not particularly limited, and any solvent known in the art may be used. Non-limiting examples of the fractionation solvent include polar solvents such as water and alcohols; and non-polar solvents, such as hexane, ethyl acetate, chloroform, dichloromethane, and butanol. These solvents may be used alone or two or more thereof may be used in combination. When an alcohol is used as the fractionation solvent, an alcohol having 1 to 6 carbon atoms may be used.

In the present invention, the term “blood circulation improvement effect” refers to improving blood circulation through vasodilation or by inhibiting platelet aggregation.

In the present invention, the term “cholesterol improvement effect” refers to inhibiting cholesterol synthesis.

In the present invention, the term “anti-obesity effect” refers to reducing body fat.

In the present invention, the term “immune function improvement effect” refers to suppressing immune hypersensitivity reaction or promoting immune function.

In the present invention, the term “menopause improvement effect” refers to improving the health of menopausal women or men.

In the present invention, the term “anti-allergic effect” refers to suppressing immune hypersensitivity reaction, improving nasal conditions deteriorated due to immune hypersensitivity reaction, or improving skin conditions deteriorated due to immune hypersensitivity reaction.

In the present invention, the term “cognitive function improvement effect” refers to improving cognitive function deteriorated due to aging.

In the present invention, the term “liver health improvement effect” refers to improving liver health or protecting the liver from alcohol damage.

In the present invention, the term “blood glucose control effect” refers to a glycemic control effect including suppressing an increase in the concentration of blood glucose after a meal by promoting absorption of sugar into cells.

In the present invention, the term “effective amount” or “including as an active ingredient” means an amount of extracts capable of exhibiting a blood circulation improvement effect, inhibiting cholesterol synthesis, exhibiting an anti-obesity effect, improving immune function, exhibiting a female menopause improvement effect, exhibiting an anti-allergic effect, exhibiting a blood glucose control effect, exhibiting a cognitive function improvement effect, exhibiting a liver health improvement effect, or exhibiting an andropause improvement effect. When the composition of the present invention includes an effective amount of the plant extracts, the composition may provide a blood circulation improvement effect, a cholesterol control effect, an anti-obesity effect, an immune function improvement effect, a female menopause improvement effect, an anti-allergic effect, a blood glucose control effect, a liver health improvement effect, and an andropause improvement effect.

In the food composition and the pharmaceutical composition according to the present invention, based on a total weight of the food composition or the pharmaceutical composition, plant extracts are preferably included in an amount of 0.0001 to 10 parts by weight.

The present invention provides a pharmaceutical composition for improving blood circulation, improving cholesterol, suppressing obesity, improving immune function, improving female menopause, suppressing allergies, controlling blood glucose, improving cognitive function, improving liver health, and/or improving andropause including plant extracts as active ingredients.

In the present invention, the term “pharmaceutical composition” may be used as a concept including the meaning of “quasi-drugs” or “drugs”.

The pharmaceutical composition may be prepared as a solution using an oil or aqueous medium as a solvent, may be prepared in the form of a suspension or emulsion, or may be prepared in the form of extracts, powder, granules, tablets, or capsules.

In addition, the composition may further include one or more active ingredients exhibiting the same or similar function. For example, the composition may include known ingredients for improving blood circulation, improving cholesterol, suppressing obesity, improving immune function, improving female menopause, suppressing allergies, controlling blood glucose, improving cognitive function, improving liver health, and improving andropause. When the composition includes additional ingredients for improving blood circulation, improving cholesterol, suppressing obesity, improving immune function, improving female menopause, suppressing allergies, controlling blood glucose, improving cognitive function, improving liver health, and improving andropause, the composition of the present invention may have a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and an andropause improvement effect. When adding the above ingredients, safety according to combined use, ease of formulation, and stability of active ingredients may be considered. In a specific example of the present invention, the composition may further include ingredients for improving blood circulation, improving cholesterol, suppressing obesity, improving immune function, improving female menopause, suppressing allergies, controlling blood glucose, improving cognitive function, improving liver health, and improving andropause known in the art. Based on a total weight of the composition, the additional ingredients may be included in an amount of 0.0001 to 10 parts by weight. In this case, the content range may be adjusted according to requirements such as safety and ease of formulation of the plant extracts.

In addition, the composition of the present invention may further include pharmaceutically acceptable carriers.

The pharmaceutically acceptable carrier may contain various ingredients such as buffers, sterile water for injection, normal saline or phosphate buffered saline, sucrose, histidine, salts, and polysorbates.

The composition of the present invention may be administered via an oral or parenteral route. The composition may be administered in the form of a general pharmaceutical preparation. For example, in clinical administration, the composition may be administered in various oral and parenteral formulations. When the composition is formulated, the composition may be prepared using general diluents or excipients such as fillers, extenders, binders, wetting agents, disintegrants, and surfactants.

Solid preparations for oral administration include tablets, pills, powders, granules, capsules, and the like. The solid preparation may be prepared by mixing one or more excipients selected from starch, calcium carbonate, sucrose or lactose, and gelatin with the pharmaceutical composition of the present invention.

In addition to simple excipients, lubricants such as magnesium stearate talc are also used. Liquid formulations for oral administration include suspensions, oral solutions, emulsions, syrups, and the like. In addition to water and liquid paraffin, which are commonly used simple diluents, various excipients, for example, wetting agents, sweetening agents, fragrances, preservatives, and the like may be included.

Formulations for parenteral administration include sterile aqueous solutions, non-aqueous solutions, suspensions, emulsions, freeze-dried preparations, and suppositories. The non-aqueous solutions and the suspensions may include propylene glycol, polyethylene glycol, vegetable oils such as olive oil, and injectable esters such as ethyl oleate. As the bases of the suppositories, witepsol, macrogol, tween 61, cacao butter, laurin butter, glycerogelatin, and the like may be used.

The effective amount of the plant extracts included in the composition of the present invention may vary depending on formulation of the composition, a method of applying the compound to the skin, and a length of time the compound stays on the skin. For example, when the composition is formulated into a pharmaceutical formulation, the composition may include a higher concentration of the plant extracts than when formulated as a cosmetic that is routinely applied to the skin. Accordingly, based on the amount of the plant extracts, a daily dose may be 0.1 to 100 mg/kg, preferably 30 to 80 mg/kg, more preferably 50 to 60 mg/kg, and the pharmaceutical formulation may be administered at a frequency of 1 to 6 administrations per day.

The composition of the present invention may be used alone or in combination with surgery, radiation therapy, hormone therapy, chemotherapy, and methods using biological response modifiers.

In addition, the composition for improving blood circulation, improving cholesterol, suppressing obesity, improving immune function, improving female menopause, suppressing allergies, controlling blood glucose, improving cognitive function, improving liver health, and/or improving andropause including the plant extracts of the present invention as active ingredients may be provided as a quasi-drug.

In the present invention, the “quasi-drug” includes the plant extracts as active ingredients. In addition, when necessary, the “quasi-drug” may include a pharmaceutically acceptable carrier, an excipient, or a diluent. As long as the effect of the present invention is not hindered, the pharmaceutically acceptable carrier, excipient, or diluent may be used without particular limitation. For example, a filler, an extender, a binder, a wetting agent, a disintegrant, a surfactant, a lubricant, a sweetening agent, a perfuming agent, or a preservative may be included, but the present invention is not limited thereto.

For example, the quasi-drug may be a disinfectant cleaner, a shower foam, an ointment, a wet tissue, a coating agent, and the like. The quasi-drug is preferably prepared as a semi-solid preparation such as an ointment for external use and a lotion, without being limited thereto. A method of formulating the quasi-drug, a method of using the same, and the dose and components thereof may be appropriately selected from conventional techniques known in the art.

In addition, the present invention provides a skin external preparation for improving blood circulation, improving cholesterol, suppressing obesity, improving immune function, improving female menopause, suppressing allergies, controlling blood glucose, improving cognitive function, improving liver health, and/or improving andropause including the plant extracts of the present invention as active ingredients.

When the plant extracts are used as a skin external preparation, the skin external preparation may further include fatty substances, organic solvents, solubilizers, thickening and gelling agents, emollients, antioxidants, suspending agents, stabilizers, foaming agents, fragrances, surfactants, water, ionic or non-ionic emulsifiers, fillers, sequestering agents and chelates agents, preservatives, vitamins, blockers, humectants, essential oils, dyes, pigments, hydrophilic or lipophilic active agents, lipid vesicles, or supplements commonly used for external preparations for skin in the field of dermatology. In addition, the above ingredients may be introduced in an amount generally used in the field of dermatology.

When the plant extracts are provided as a skin external preparation, the skin external preparation may be prepared in the form of an ointment, a patch, a gel, a cream, or a spray, without being limited thereto.

In addition, the present invention relates to a food composition for improving blood circulation, improving cholesterol, suppressing obesity, improving immune function, improving female menopause, suppressing allergies, controlling blood glucose, improving cognitive function, improving liver health, and improving andropause including the plant extracts.

When the plant extracts of the present invention are provided as a food composition, the composition may include food supplements in addition to active ingredients.

The food supplement means a component added to preserve food and is added to manufacture health functional food of each formulation. The food supplements may be appropriately selected and used by those skilled in the art. For example, the food supplements include various nutrients, vitamins, minerals (electrolytes), flavoring agents such as synthetic flavoring agents and natural flavoring agents, staining agents and fillers, pectic acid and salts thereof, alginic acid and salts thereof, organic acids, protective colloidal thickeners, pH regulators, stabilizers, preservatives, glycerin, alcohols, carbonating agent used in carbonated beverages, and the like, without being limited thereto.

In addition, the food composition may include a health functional food. In the present invention, the “health functional food” refers to a food group that allows the function of a food to be expressed to meet a specific purpose using a physical, biochemical, or bioengineering method, or a food designed and processed to sufficiently express body control functions related to biological defense rhythm control, disease prevention, and health recovery of a food composition. The health functional food has an active health maintenance or promotion effect compared to general food, and a health supplement food refers to a food intended for health supplement. In some cases, the terms functional food, health food, and dietary supplement are used interchangeably.

Specifically, the health functional food is a food prepared by adding the plant extracts to food materials such as beverages, teas, spices, gum, and confectionery, or a food prepared by encapsulating, powdering, or suspension of the plant extracts of the present invention. When the health functional food is ingested, a specific health effect may be obtained. In particular, unlike general drugs, since the health functional food uses food as a raw material, there is an advantage in that there are no side effects that may occur when taking drugs for a long time.

The food may include food scientifically acceptable food supplement additives, and may further include carriers, excipients, and diluents commonly used in manufacture of health functional foods.

Each of the above components included in the food composition according to the present invention may be included in the food composition of the present invention within a range that does not exceed the maximum amount prescribed in each country's food safety norms.

Since the composition is commonly used in food compositions, the composition may include additional ingredients capable of improving odor, taste, visual, and the like. For example, the composition may include vitamins A, C, D, E, B1, B2, B6, and B12, niacin, biotin, folate, panthotenic acid, and the like. In addition, the composition may include minerals such as zinc (Zn), iron (Fe), calcium (Ca), chromium (Cr), magnesium (Mg), manganese (Mn), copper (Cu), and chromium (Cr). In addition, the composition may include amino acids such as lysine, tryptophan, cysteine, and valine. The health functional food of the present invention may be prepared in various forms without any particular limitation, may include all foods in a conventional sense, and may be used interchangeably with terms known in the art, such as functional food.

In addition, according to selection of those skilled in the art, the health functional food of the present invention may be prepared by mixing other auxiliary ingredients that may be included in food and known additives. Examples of foods to which the health functional food of the present invention may be added include meat, sausage, bread, chocolate, candy, snacks, confectionery, pizza, ramen, other noodles, gums, dairy products including ice cream, various soups, beverages, tea, drinks, alcoholic beverages, and vitamin complexes. The health functional food may be added to juice, tea, jelly, and juice prepared by using the plant extracts of the present invention as a main component. In addition, the health functional food may be added to animal feed.

When the food formulation is a beverage, as in a conventional beverage, various flavoring agents or natural carbohydrates may be contained as additional ingredients. The natural carbohydrates include monosaccharides such as glucose and fructose, disaccharides such as maltose and sucrose, polysaccharides such as dextrin and cyclodextrin, and sugar alcohols such as xylitol, sorbitol, and erythritol. As the sweetener, natural sweeteners such as thaumatin and Stevia extracts, synthetic sweeteners such as saccharin and aspartame, and the like may be used. Based on 100 mL of the composition of the present invention, the natural carbohydrate is contained in an amount of about 0.01 to 0.04 g, preferably about 0.02 to 0.03 g.

Food formulations other than those described above may include various nutrients, vitamins, electrolytes, flavoring agents, coloring agents, pectic acid and salts thereof, alginic acid and salts thereof, organic acids, protective colloidal thickeners, pH adjusting agents, stabilizers, preservatives, glycerin, alcohols, carbonation agents used in carbonated beverages, and the like. Other food formulations may contain pulp for manufacture of natural fruit juices, fruit juice beverages, and vegetable beverages. These ingredients may be used independently or in combination. The proportion of these additives is not very important but is generally selected in the range of 0.01 to 0.1 parts by weight per 100 parts by weight of the composition of the present invention.

Since the health functional food of the present invention may be consumed daily, a blood circulation improvement effect, a cholesterol improvement effect, an anti-obesity effect, an immune function improvement effect, a female menopause improvement effect, an anti-allergic effect, a blood glucose control effect, a cognitive function improvement effect, a liver health improvement effect, and an andropause improvement effect may be obtained.

When the plant extracts are used as a food additive, the plant extracts may be suitably used according to a conventional method. For example, the plant extracts may be added alone or may be used in combination with other foods or food ingredients. The input amount of the active ingredients may be appropriately determined according to the purpose of use thereof (prevention, health, or therapeutic treatment). In general, in manufacture of food or beverage, the composition of the present invention is added in an amount of 15 parts by weight or less, preferably 10 parts by weight or less. However, for health and hygiene purposes or for long-term intake for health control, the composition may be added in an amount less than or equal to the above range. Since there is no problem in terms of safety, the active ingredient may be used in an amount exceeding the above range.

Hereinafter, embodiments of the present invention will be described in detail. However, the following examples are only illustrative of the present invention, and the content of the present invention is not limited to the following examples.

### Preparation Example 1: Preparation of Plant Extracts

A 70% aqueous ethanol solution was added to the whole plant, roots, stems, leaves, fruits, flowers, shoots, branches, bark, sap, bulbils, or seeds of each of plants 1 to 111. At this time, the 70% aqueous ethanol solution was added in a volume corresponding to 20 times the mass of each plant tissue. Then, extraction was performed at room temperature for 3 days, and then filtration under reduced pressure was performed. Then, the filtered extracts were concentrated and dried using a rotary evaporator (Buchi, Switzerland) to prepare plant extracts.

### Experimental Example 1: Blood Circulation Improvement Effect—Measuring Recovery of Reduction in Nitric Oxide Induced by oxLDL

To confirm the blood circulation improvement effect of the plant extracts, recovery of reduction in nitric oxide induced by oxidized low density lipoprotein (oxLDL) was measured. First, HUVEC cells were suspended in a Ham's F-12K (Kaighn's modification) culture medium, and the suspended cells were aliquoted into a culture plate. Then, plant extracts (samples) diluted at various concentrations were added to the culture plate containing the cells. At this time, 200 ppm of oxLDL was used as a positive control. The sample-treated cells were placed in a 5% CO₂ incubator set at 37° C. and cultured for 24 to 48 hours, and then the supernatant was taken and used as an analysis sample.

To analyze nitric oxide, a Griess reagent was prepared by mixing equal amounts of component A and component B. Then, 250 μl of the sample (culture medium) and 50 μl of the Griess reagent were added to a plate and mixed thoroughly. Next, to prepare a sodium nitrite solution, a nitrite standard solution was diluted with distilled water to a concentration between 1 and 100 mM (reference sample). Under light-blocked conditions, a mixture of the sample and the Griess reagent was incubated at room temperature for 30 minutes. Thereafter, the absorbance (OD) of the nitrite-containing sample with respect to the reference sample was measured at 548 nm. At this time, as a positive control, 0.1 μM of rosuvastatin was treated, and the positive control showed a 100% recovery result.

In this experiment, it was confirmed whether the level of nitric oxide decreased by oxLDL was recovered by the plant extracts. At this time, as a result of the experiment, the amount of nitric oxide was quantified. Nitric oxide plays a role in dilating blood vessels. Accordingly, an increase in secretion of nitric oxide indicates that blood circulation is improved.

The experimental results for each sample are shown in the tables below. When the absorbance value increases as the concentration of the sample increases, it indicates that the amount of nitric oxide (NO) generated by the sample increases. Accordingly, when the amount of nitric oxide decreased by oxLDL is restored by sample treatment, it indicates that the sample is involved in dilating blood vessels in vivo.

### Experimental Example 2. Lipid-Cholesterol Improvement Effect—Measurement of Cholesterol Synthesis in HepG2 Cells

To confirm the lipid-cholesterol improvement effect of the plant extracts, an appropriate number of HepG2 cells was seeded and cultured using a IMEM culture medium (containing 10% of FCS, 4.5 g/L of glucose, 4 mM glutamine, 10 μg/ml of insulin, 50 μg/ml of penicillin, 50 μg/ml of streptomycin, and 1% of non-essential amino acids). At this time, the culture medium was replaced every 2 days, and the cells were used for the experiment on the 12th day after seeding.

1 ml of a labeled acetate solution ([¹⁴C] sodium acetate) was added to the cells, and the cells were incubated in a CO₂ incubator. After 1 hour of incubation, the culture medium was discarded, the cells were washed with ice-cold PBS, 2 M NaOH was added to detach the cells, and the cells were incubated at 95° C. for 1 hour to induce saponification of lipids. Then, [³H] cholesterol (about 20,000 dpm/sample) was added as a recovery marker, and lipids were extracted with a chloroform-methanol (2:1 (v/v)) solution. Then, after separating lipides through thin layer chromatohrapy (TLC), a band corresponding to free cholesterol was scraped and placed in a scintillation vial for measurement. The results for the above experiment were expressed as the amount of acetate incorporated per protein (acetate (nmole) incorporated/protein (mg)). In this case, simvastatin and atorvastatin were used as positive controls, and IC₅₀ thereof were 31.8 nM and 70.4 nM, respectively.

In this experimental example, it was confirmed whether the plant extracts inhibit cholesterol synthesis. As a result of the experiment, the amount of cholesterol was quantified. A decrease in cholesterol level means that a cholesterol improvement effect is obtained.

The experimental results for each sample are shown in the tables below. The effect of the test substance on the intracellular cholesterol content was expressed as a relative ratio when the control group (treated with dimethyl sulfoxide (DMSO) only) was 100% or was expressed as the amount incorporated acetate ([¹⁴C]-acetate (nmole) incorporated/4 h/well). As the measured value increases, the amount of synthesized cholesterol increases. That is, a decrease in cholesterol synthesis indicates improvement in cholesterol.

### Experimental Example 3. Anti-Obesity Effect—Measurement of Glycerol Content in Culture Medium of 3T3-L1 Fibroblasts

To confirm the anti-obesity effect of the plant extracts, the content of glycerol in the culture medium of 3T3-L1 fibroblasts was measured.

First, 3T3-L1 fibroblasts were cultured using a DMEM culture medium (proliferation culture medium) containing 10% bovine serum to an appropriate cell number on a plate. Then, using a DMEM culture medium (differentiation culture medium) containing 10% FBS, the cells were cultured until the plate was filled with the cells, and then were further cultured for 2 to 3 days. The existing culture medium was replaced with a culture medium to induce cell differentiation, which contains insulin (5 μg/mL), dexamethasone (0.25 μM), and 3-isobutyl-1-methylxanthine (0.5 mM), and was followed by incubation for 2 to 3 days. Afterwards, the culture medium was replaced with a medium containing only insulin and the cells were incubated for 2 to 3 days, and then the culture medium was replaced again with a DMEM culture medium containing 10% FBS and the cells were again incubated for 6 to 8 days (incubated until a point at which fat accumulation is observed in the cells). The anti-obesity substance was added before or after differentiation induction according to the purpose of the experiment. The cell culture medium was centrifuged at 10,000 rpm for 10 minutes, and the supernatant was obtained using a Pasteur pipette. The obtained supernatant was transferred to a plate and heated at 65° C. for 8 min to inactivate enzymes released from the cells. Next, a glycerol probe was dissolved in 220 μl of DMSO, and a glycerol enzyme mixture (Cayman Chemical, USA; Item No. 10010962) was dissolved in an assay buffer. These solutions were shielded from light and stored at −20° C. until experiment was performed. A 1 mM glycerol standard solution was prepared by diluting 10 μl of a glycerol standard solution with 990 μl of an analysis buffer. Then, the 1 mM glycerol standard solution was thoroughly mixed, 0, 2, 4, 6, 8, and 10 μl of the 1 Mm glycerol standard solution were respectively added to each well, and the analysis buffer was added to the each well to adjust the total volume to 50 μl. 50 μl of the sample (culture medium in which enzymes are inactivated) was added to each well. A reaction mixture containing 46 μl of the analysis buffer, 2 μl of the glycerol probe, and 2 μl of the glycerol enzyme mixture was prepared. 50 μl of the reaction mixture was added to each well containing the standard solution and the sample and was thoroughly mixed. Then, each well was shielded from light and left at room temperature for 30 minutes. Then, absorbance was measured at 570 nm. At this time, a group treated with 0.5 ppm orlistat was used as a positive control group, and the positive control group showed an increase of 160% compared to the negative control group.

In this experiment, it was confirmed whether the plant extracts promote decomposition of triglycerides. As a result of the experiment, the amount of glycerol was quantified. Glycerol is a by-product of lipolysis. Accordingly, as the level of glycerol increases, an anti-obesity effect increases.

The experimental results for each sample are shown in the tables below. When lipolysis is enhanced by the test substance, the amount of triglycerides accumulated in adipocytes decreases and the amount of glycerol released increases. Accordingly, an increase in the amount of released glycerol means an increase in decomposition of triglycerides. Through this experiment, it can be confirmed that the test substance has an anti-obesity effect.

### Experimental Example 4. Immune Function Improvement Effect—Measurement of Interferon Gamma (IFN-γ) Produced in Splenocytes/Macrophages

To confirm the immune function improvement effect of the plant extracts, the amount of interferon gamma produced in splenocytes and macrophages was measured. First, THP-1 cells, which are macrophages, and NK-92 cells, which are natural killer cells, were suspended in an RPMI (Roswell Park Memorial Institute)-1640 culture medium (for THP-1 cells) and a MEMA (Minimum Essential Media Alpha) culture medium (for NK-92 cells), respectively. The suspended cells were seeded on culture plates (24 wells/96 wells). The plant extracts (sample) diluted to various concentrations were added to the cells. At this time, lipopolysaccharide (LPS) (0.1 to 1 μg/mL) was used as a positive control. The sample-treated cells were placed in a 5% CO₂ incubator set to 37° C. and incubated for 24 to 48 hours. Then, the supernatant was taken and used as an analysis sample.

The experiment was performed using an enzyme-linked immunosorbent assay (ELISA) method. After coating a 96-well plate with an IFN-γ primary antibody, 1% bovine serum albumin (BSA) was added to block the remaining space. Then, distilled water (blank), an IFN-γ standard solution, and each sample were added to the wells in an amount of 100 μl and incubated at room temperature for 2 hours. 100 μl of a biotin-bound IFN-γ secondary antibody was added to the wells and incubated at room temperature for 2 hours. 100 μl of avidin-bound horseradish peroxidase (HRP) was added and incubated at room temperature or 37° C. for 30 minutes. 100 μl of a TMB substrate solution was added and incubated at room temperature for 4 to 14 minutes. 50 μl of a stop solution (sulfuric acid) was added to stop the reaction. Absorbance at 450 nm wavelength was measured using an ELISA reader. The IFN-γ content of each sample was calculated based on the absorbance of the standard solution.

In this experiment, the effect of the plant extracts on production of interferon gamma was confirmed. As a result of the experiment, the amount of interferon gamma was quantified. As the level of interferon gamma increases, the degree of improvement in immune function increases.

The experimental results for each sample are shown in the tables below. The absorbance is proportional to the IFN-γ content. In addition, increase in IFN-γ production means that immunity is enhanced, such as promotion of differentiation of T lymphocytes, activation of macrophages, and promotion of production of IgG antibodies in B cells.

### Experimental Example 5. Female Menopause Improvement Effect—Analysis of Competitive Binding of Ligand to Estrogen Receptor

To confirm the female menopause improvement effect of the plant extracts, the degree of competitive binding of a ligand to an estrogen receptor (ER) was analyzed. First, 10 μl of [³H]-17β-estradiol (E2), 50 μl of rat uterine cytosol, and 230 μl of 50 mM Tris buffer (pH 7.4) were mixed. Then, in a drum roller, the mixture was reacted with 10 μl of the plant extracts diluted in various concentrations (incubated at 4° C. for 20 hours). After reaction, 750 μl of cold 60% hydroxylapatite (HAP) was added to the reaction mixture, and the reaction mixture was placed on ice for 20 minutes while vortexing the reaction mixture for 10 seconds at 5 minute intervals. Then, the reaction mixture was centrifuged at 600 g at 4° C. for 5 minutes to separate free ligands and bound ligands. At this time, the free ligands mean ER not bound to [³H]-E2 or the plant extracts, and the bound ligands mean ER bound to [³H]-E2 or the plant extracts. HAP was adsorbed to the bound ligands and used to separate the free and bound ligands. Then, the separated bound ligands (pellets) were washed three times with 2 ml of 50 mM Tris buffer, and 2 ml of ethanol cooled to 4° C. was added to the pellets. The ethanol-added pellets were placed on ice for 15 minutes while vortexing the pellets for 10 seconds at 5 minute intervals, and then were centrifuged at 600 g at 4° C. for 10 minutes. In this process, radiolabeled E2 ([³H]-E2) was extracted. That is, [³H]-E2 was dissolved in ethanol, and the remainder precipitated to form pellets. Then, the extracted [³H]-E2 present in the supernatant was poured into a vial containing 10 ml of a scintillation cocktail. Then, to determine whether the plant extracts prevented the radioactive isotope-labeled E2 ([³H]-E2) from binding to estrogen receptors, radioactivity present in the vial was measured using a β-counter. Based on these results, the binding ability of the plant extracts to estrogen receptors could be confirmed. Results for groups in which 0.03 nM, 0.1 nM, 0.33 nM, 1 nM, 10 nM, and 100 nM of unlabeled E2 (estradiol) were added instead of the plant extracts were used to construct a standard curve.

In this experiment, it was confirmed whether the plant extracts activate signaling related to the female hormone estrogen. The ability to bind to the estrogen receptor was quantified. As the degree of binding to estrogen increases, the degree of female menopause improvement increases.

Experimental results for each sample are shown in the tables below, and the concentration of estrogen receptor (ER) was calculated based on the measured radioactivity values.

### Experimental Example 6. Anti-Allergic Effect—IgE Secretion Measurement

To confirm the anti-allergic effect of the plant extracts, the secretion amount of immunoglobulin E was measured. First, U266 cells were treated with a test substance while culturing the U266 cells in an RPMI1640 culture medium containing 10% FBS for 3 days. After 3 days of treatment with the test substance, the cell culture medium was harvested. IgE capture antibody was added to the plate and left for 1 day. The plate was blocked with 2% BSA. The collected culture medium was added to the plate and mixed by shaking. IgE detection antibody was added to the plate and mixed by shaking. Streptavidin-HRP was added to the plate, and after blocking light, the mixture was shaken. A substrate was added to the plate, and after blocking light, the mixture was mixed by shaking until the color of the mixture changed to blue. After confirming that the color had changed, a 2 N sulfuric acid solution was added to the plate and mixed by shaking until the color changed to yellow. After measuring the absorbance at 450 nm and 570 nm, results were analyzed by subtracting the absorbance at 570 nm from the absorbance at 450 nm. At this time, as a positive control, 1 μM dexamethasone was used, and as a result, an inhibitory effect of 32% was observed. As a result of treatment with 0.1 μM hydrocortisone, an inhibitory effect of 35% was observed.

In this experiment, it was confirmed whether the plant extracts inhibit IgE that induces allergy. As a result of the experiment, the amount of IgE was quantified. As the level of IgE decreases, the anti-allergic effect increases.

Experimental results for each sample are shown in the tables below. Since IgE causes immune hypersensitivity, a decrease in IgE secretion indicates that allergy is alleviated.

### Experimental Example 7. Blood Glucose Control Effect—Glucose Uptake Measurement (2-NBDG Method)

To confirm the blood glucose control effect of the plant extracts, the glucose uptake capacity of cells treated with the plant extracts was measured. First, C2C12 cells were seeded on a 96-well culture plate at a cell density of 1×10⁴ cells/well. When a state of subconfluence was reached, which is within 24 hours, the experiment was started.

[Caution]

Experiments should be performed within 48 hours after inoculation of cells. Accurate data is difficult to obtain when a culture plate is full of cells due to prolonged incubation time or when the number of cells is too small.

When saturated, the culture medium was removed, and 100 μl of a fresh medium containing 10 μM 2-NBDG or 100 μl of a fresh medium not containing 10 μM 2-NBDG (control) was added to each well. At this time, a test substance to be evaluated was included in the culture medium. That is, the cells were treated with the test substance by changing the culture medium. After treatment for 1 hour, wells were washed 2 times with pre-cold PBS. Then, cells in each well were collected and resuspended in 200 μl of a pre-cold medium, and the nuclei of the cells were stained with 1 μg/mL of propidium iodine (PI).

[Caution]

After nuclear staining, the sample should be stored at 4° C. until measured by automated cell analysis (FACS). When conducting an experiment, it is recommended to start measurement within 30 minutes.

Measurement was performed with FACS for about 20 seconds. Event results of about 2,000 single cells were collected, and statistical analysis was performed on the event results. At this time, 1 mM metformin treatment was used as a positive control, and as a result, a 180% increase effect was observed.

In this experiment, it was determined whether the plant extracts promote intracellular glucose uptake. As a result of the experiment, the amount of glucose uptake into cells was quantified. That is, an increase in glucose uptake into cells means that the blood glucose control effect is improved.

The experimental results for each sample are shown in the tables below. As the value of measured fluorescence intensity increases, the glucose absorption rate increases. An increase in glucose absorption indicates a decrease in blood glucose levels. Thus, an increase in glucose intake indicates that diabetes symptoms are relieved.

### Experimental Example 8. Cognitive Function Improvement Effect—Measurement of β-Secretase Activity

To confirm the cognitive function improvement effect of the plant extracts, the activity of β-secretase treated with the plant extracts was measured. First, reagents and solvents required for the experiment were prepared:

1) β-secretase cleavage enzyme (BACE) analysis buffer (50 mM sodium acetate, pH 4.5)

2) BACE1 enzyme (1 unit/ml)

3) 40 μl of a BACE1 substrate (750 nM Rh-EVNLDAEFK-quencher)+3,960 μl of a BACE1 analysis buffer (use within 24 hours after preparation)

4) Stop solution (2.5 M sodium acetate)

10 μl of the analysis buffer, 10 μl of the BACE1 substrate, the β-secretase enzyme (1 units/ml), and 10 μl of the test substance were added to a black 96-microwell plate.

[Caution]

Various concentrations of test substances were prepared and used. At this time, the analysis buffer was used.

Fluorescence intensity was measured at Ex 545 nm/Ex 585 nm, and the mixture was reacted at room temperature for 1 hour.

[Caution]

The plate was wrapped with foil to block light.

10 μl of the stop solution was added to stop the reaction. Fluorescence intensity was measured at Ex 545/Em 585 nm wavelengths. The measured fluorescence intensity values were substituted into the following general formula.

Inhibition rate (%)=[1−{(S−S0)/(C−C0)}×100 [General formula]

C=Fluorescence intensity after 60 minutes of control treatment,

C0=Fluorescence intensity before control treatment (at 0 minutes of treatment),

S=Fluorescence intensity after 60 minutes of sample treatment, and

S0=Fluorescence intensity before sample treatment (at 0 minutes of treatment)

In this experiment, it was confirmed whether the plant extracts inhibit the activity of β-secretase. As a result of the experiment, the activity of β-secretase was quantified. As the activity of β-secretase is reduced, the cognitive function improvement effect increases.

The experimental results for each sample are shown in the tables below. β-secretase cleaves an APP amino acid terminus to produce amyloid-beta, which reduces cognitive function. Accordingly, a decrease in the activity of β-secretase indicate that cognitive function is improved.

### Experimental Example 9. Liver Health Improvement Effect—Measurement of Liver Cell Viability

To confirm the liver health improvement effect of the plant extracts, the viability of liver cells treated with the plant extracts was measured. First, HepG2 cells were cultured at 37° C. under 5% CO₂ atmosphere in a DMEM culture medium containing 10% FBS. Then, the cultured HepG2 cells were seeded on a 24-well plate at a cell density of 3×10⁵ cells/well, followed by incubation for 1 day. Then, various concentrations of test substances were added to HepG2 cells, followed by incubation for 24 hours.

[Caution]

When the cells are treated with the test substances, approximately 70 to 80% of the area of the well should be filled with the cells.

An MTT solution dissolved in PBS at a concentration of 5 mg/mL was diluted to 10% with the culture medium. The solution was dispensed in a volume of 0.5 to 1.0 mL in a 24-well plate and incubated for 4 hours in an incubator.

[Caution]

Since the MTT solution is light-sensitive, incubation should be performed with the plate wrapped in foil.

After 4 hours of incubation, the culture medium containing the MTT solution was removed, and 1 mL of DMSO was added and was shaken for 10 minutes. Then, absorbance was measured at 540 nm. At this time, as positive controls, the cells were treated with \textcircled{1} 100 mM ethanol and \textcircled{2} 100 mM ethanol+100 ppm Silymarin, respectively. In the case of \textcircled{1} 100 mM ethanol, liver cell viability was reduced by 31%. In the case of \textcircled{2} 100 mM ethanol+100 ppm Silymarin, liver cell viability was restored to 86%.

In this experiment, it was confirmed whether the plant extracts restore liver cell viability decreased by alcohol (ethanol) treatment. As a result of the experiment, liver cell viability was quantified. That is, as liver cell viability increases, the liver health improvement effect increases.

The experimental results for each sample are shown in the tables below. As the absorbance value increases, liver cell viability increases. Accordingly, the plant extracts may have a positive effect on improving liver health.

### Experimental Example 10. Andropause Improvement Effect—Measurement of Transcriptional Activity of Androgen Receptor

To confirm the andropause improvement effect of the plant extracts, the transcriptional activity of an androgen receptor was measured. Specifically, the experiment was performed using an LNCaP cell line into which an ARE reporter plasmid was inserted. First, cells were cultured at 37° C. under 5% CO₂ atmosphere in a DMEM culture medium containing 10% FBS. Then, the cultured cells were transferred to a plate, followed by incubation for 2 days. Then, test substances were added to the cells, followed by incubation for 1 day. Then, a substrate was added, and luciferase activity was measured.

In this experiment, it was confirmed whether the plant extracts activate the androgen signaling. As a result of the experiment, the transcriptional activity of the androgen receptor was quantified. That is, as the transcriptional activity of the androgen receptor increases, the andropause improvement effect is increased.

Experimental results for each sample are shown in the tables below. Since the androgen receptor plays an important role in male hormone signaling, the increased activity of this receptor means that the plant extracts may alleviate andropause symptoms.

### Example 1. Island thistle

TABLE 1

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 1 | Island thistle | | | | | | |
| 1-1 | Whole plant | 1 | 23.95 | 42.08 | 143.45 | 89.91 | 137.23 |
| | | 10 | 49.55 | 38.86 | 291.18 | 137.35 | 228.99 |
| | | 100 | 83.56 | 30.25 | 496.54 | 352.61 | 274.39 |
| 1-2 | Roots | 1 | 21.48 | 47.85 | 119.61 | 91.03 | 112.68 |
| | | 10 | 35.37 | 34.54 | 308.70 | 174.67 | 120.70 |
| | | 100 | 82.17 | 20.24 | 475.76 | 459.75 | 210.89 |
| 1-3 | Stems | 1 | 28.32 | 78.25 | 166.97 | 88.06 | 131.74 |
| | | 10 | 40.84 | 40.49 | 260.49 | 138.91 | 102.25 |
| | | 100 | 74.21 | 21.51 | 351.82 | 309.10 | 225.07 |
| 1-4 | Leaves | 1 | 23.04 | 74.95 | 158.86 | 106.64 | 109.74 |
| | | 10 | 31.16 | 37.24 | 254.55 | 194.52 | 108.31 |
| | | 100 | 83.85 | 21.04 | 492.50 | 485.15 | 307.42 |
| 1-5 | Fruits | 1 | 24.44 | 43.26 | 113.60 | 101.02 | 114.38 |
| | | 10 | 31.20 | 40.11 | 344.64 | 133.08 | 238.58 |
| | | 100 | 76.18 | 26.91 | 478.03 | 316.14 | 327.09 |
| 1-6 | Flowers | 1 | 22.87 | 58.84 | 185.58 | 107.39 | 122.39 |
| | | 10 | 46.29 | 37.59 | 284.99 | 128.82 | 125.02 |
| | | 100 | 85.61 | 23.76 | 407.95 | 350.37 | 211.17 |

TABLE 2

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 1 | Island thistle | | | | | | |
| 1-1 | Whole plant | 1 | 101.95 | 108.41 | 110.47 | 70.75 | 106.50 |
| | | 10 | 81.37 | 136.07 | 80.41 | 89.99 | 312.55 |
| | | 100 | 78.13 | 152.78 | 45.23 | 95.07 | 340.94 |
| 1-2 | Roots | 1 | 95.99 | 98.49 | 92.20 | 77.82 | 113.89 |
| | | 10 | 84.15 | 136.77 | 52.26 | 91.41 | 210.37 |
| | | 100 | 71.87 | 133.53 | 29.62 | 104.14 | 356.21 |
| 1-3 | Stems | 1 | 100.97 | 104.19 | 103.90 | 78.41 | 119.41 |
| | | 10 | 88.14 | 137.14 | 84.64 | 81.65 | 292.41 |
| | | 100 | 72.20 | 148.34 | 10.15 | 100.79 | 434.06 |
| 1-4 | Leaves | 1 | 101.59 | 111.90 | 98.01 | 71.27 | 113.07 |
| | | 10 | 89.75 | 121.43 | 73.81 | 91.85 | 183.46 |
| | | 100 | 80.00 | 141.29 | 40.74 | 110.24 | 399.21 |
| 1-5 | Fruits | 1 | 97.57 | 103.93 | 96.58 | 84.60 | 196.42 |
| | | 10 | 85.34 | 115.19 | 45.26 | 76.33 | 221.09 |
| | | 100 | 80.54 | 159.70 | 44.21 | 94.52 | 319.74 |
| 1-6 | Flowers | 1 | 103.34 | 106.00 | 88.71 | 71.31 | 198.54 |
| | | 10 | 82.97 | 107.77 | 80.78 | 87.25 | 179.66 |
| | | 100 | 80.41 | 130.10 | 8.91 | 108.34 | 332.78 |

### Example 2. Deodeok

TABLE 3

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 2 | Deodeok | | | | | | |
| 2-1 | Whole plant | 1 | 21.94 | 60.72 | 125.50 | 95.28 | 103.10 |
| | | 10 | 48.74 | 38.21 | 211.57 | 142.85 | 215.44 |
| | | 100 | 73.38 | 22.83 | 360.01 | 359.47 | 327.14 |
| 2-2 | Roots | 1 | 26.11 | 102.76 | 98.61 | 98.87 | 111.74 |
| | | 10 | 32.45 | 102.79 | 110.85 | 143.08 | 234.50 |
| | | 100 | 74.39 | 102.25 | 96.95 | 120.32 | 322.32 |
| 2-3 | Stems | 1 | 29.98 | 96.41 | 101.85 | 88.54 | 92.08 |
| | | 10 | 45.28 | 98.03 | 113.14 | 116.29 | 113.21 |
| | | 100 | 76.13 | 87.25 | 85.83 | 159.78 | 212.01 |
| 2-4 | Leaves | 1 | 22.95 | 100.75 | 144.06 | 81.03 | 141.51 |
| | | 10 | 33.90 | 96.28 | 130.33 | 119.31 | 238.48 |
| | | 100 | 76.80 | 108.11 | 186.37 | 120.85 | 265.13 |
| 2-5 | Fruits | 1 | 24.35 | 41.41 | 188.90 | 86.98 | 96.32 |
| | | 10 | 50.98 | 30.16 | 284.03 | 115.64 | 223.96 |
| | | 100 | 79.30 | 29.10 | 440.82 | 309.33 | 320.70 |
| 2-6 | Flowers | 1 | 24.95 | 89.60 | 145.63 | 103.29 | 119.82 |
| | | 10 | 46.85 | 40.75 | 286.68 | 168.40 | 125.24 |
| | | 100 | 74.19 | 27.26 | 421.43 | 388.83 | 326.15 |

TABLE 4

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 2 | Deodeok | | | | | | |
| 2-1 | Whole plant | 1 | 96.24 | 113.18 | 101.62 | 80.60 | 165.56 |
| | | 10 | 86.28 | 115.57 | 95.45 | 76.57 | 223.17 |
| | | 100 | 71.75 | 159.42 | 1.87 | 97.18 | 318.56 |
| 2-2 | Roots | 1 | 98.89 | 104.41 | 104.85 | 71.90 | 196.21 |
| | | 10 | 89.72 | 101.02 | 94.02 | 67.21 | 221.03 |
| | | 100 | 75.58 | 104.10 | 90.63 | 68.00 | 439.80 |
| 2-3 | Stems | 1 | 98.01 | 103.04 | 107.56 | 64.79 | 186.01 |
| | | 10 | 95.13 | 94.37 | 108.17 | 68.77 | 314.13 |
| | | 100 | 78.70 | 97.60 | 106.65 | 70.36 | 336.18 |
| 2-4 | Leaves | 1 | 96.14 | 81.15 | 90.14 | 75.43 | 146.51 |
| | | 10 | 91.32 | 99.26 | 100.66 | 64.20 | 203.86 |
| | | 100 | 78.12 | 88.48 | 108.32 | 66.38 | 379.63 |
| 2-5 | Fruits | 1 | 97.44 | 113.49 | 90.05 | 80.79 | 90.22 |
| | | 10 | 84.73 | 136.70 | 63.97 | 82.60 | 302.61 |
| | | 100 | 75.34 | 149.22 | 24.81 | 103.45 | 372.80 |
| 2-6 | Flowers | 1 | 101.04 | 100.68 | 108.88 | 68.11 | 171.95 |
| | | 10 | 80.79 | 108.80 | 74.60 | 76.04 | 202.31 |
| | | 100 | 72.27 | 157.22 | 30.20 | 105.52 | 421.03 |

### Example 3. Ulleung Goldenrod

TABLE 5

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 3 | Ulleung | | | | | | |
| | goldenrod | | | | | | |
| 3-1 | Whole plant | 1 | 24.06 | 90.57 | 145.77 | 82.75 | 99.5 |
| | | 10 | 48.35 | 40.73 | 201.26 | 122.13 | 228.29 |
| | | 100 | 74.07 | 21.96 | 452.89 | 454.42 | 303.88 |
| 3-2 | Roots | 1 | 20.70 | 59.94 | 151.44 | 110.91 | 85.87 |
| | | 10 | 49.20 | 33.09 | 242.81 | 162.22 | 129.21 |
| | | 100 | 75.65 | 25.32 | 458.71 | 489.78 | 281.14 |
| 3-3 | Stems | 1 | 24.19 | 74.97 | 157.55 | 83.59 | 110.46 |
| | | 10 | 30.18 | 34.09 | 279.22 | 139.27 | 233.75 |
| | | 100 | 73.07 | 22.10 | 426.85 | 427.92 | 276.61 |
| 3-4 | Leaves | 1 | 27.50 | 105.73 | 166.72 | 87.60 | 92.39 |
| | | 10 | 30.06 | 97.85 | 107.57 | 158.15 | 170.60 |
| | | 100 | 73.59 | 86.77 | 179.37 | 358.56 | 315.23 |
| 3-5 | Fruits | 1 | 23.23 | 57.22 | 159.27 | 105.19 | 91.59 |
| | | 10 | 34.60 | 35.98 | 274.84 | 122.18 | 190.96 |
| | | 100 | 84.84 | 20.49 | 414.24 | 484.68 | 214.71 |
| 3-6 | Flowers | 1 | 25.68 | 81.22 | 103.54 | 107.28 | 147.90 |
| | | 10 | 41.64 | 30.73 | 302.53 | 126.74 | 167.52 |
| | | 100 | 81.10 | 29.28 | 361.44 | 388.72 | 307.65 |

TABLE 6

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 3 | Ulleung | | | | | | |
| | goldenrod | | | | | | |
| 3-1 | Whole plant | 1 | 101.20 | 101.08 | 87.01 | 81.97 | 133.62 |
| | | 10 | 83.26 | 117.72 | 45.97 | 78.10 | 287.11 |
| | | 100 | 78.67 | 154.74 | 45.57 | 102.38 | 363.05 |
| 3-2 | Roots | 1 | 103.48 | 106.24 | 108.01 | 75.31 | 140.94 |
| | | 10 | 86.20 | 120.35 | 80.81 | 80.00 | 203.76 |
| | | 100 | 80.27 | 158.68 | 42.11 | 103.83 | 379.26 |
| 3-3 | Stems | 1 | 101.33 | 97.81 | 108.16 | 73.87 | 111.54 |
| | | 10 | 91.64 | 117.77 | 76.84 | 75.26 | 198.32 |
| | | 100 | 80.61 | 142.14 | 3.41 | 102.92 | 360.88 |
| 3-4 | Leaves | 1 | 99.15 | 108.00 | 93.41 | 65.40 | 151.49 |
| | | 10 | 94.50 | 140.83 | 58.40 | 93.66 | 330.99 |
| | | 100 | 78.73 | 156.29 | 42.41 | 105.45 | 390.25 |
| 3-5 | Fruits | 1 | 100.30 | 98.85 | 103.81 | 78.00 | 206.98 |
| | | 10 | 93.44 | 109.95 | 74.31 | 80.01 | 211.60 |
| | | 100 | 74.25 | 145.55 | 20.41 | 98.96 | 457.87 |
| 3-6 | Flowers | 1 | 103.43 | 99.31 | 89.23 | 75.56 | 182.33 |
| | | 10 | 91.71 | 139.44 | 75.72 | 91.34 | 235.96 |
| | | 100 | 77.53 | 147.61 | 2.67 | 96.55 | 395.17 |

### Example 4. Goat's Beard

TABLE 7

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 4 | Goat's beard | | | | | | |
| 4-1 | Whole plant | 1 | 22.55 | 80.30 | 107.66 | 85.35 | 105.67 |
| | | 10 | 46.06 | 35.65 | 200.43 | 183.44 | 152.81 |
| | | 100 | 75.67 | 21.12 | 387.82 | 395.44 | 265.84 |
| 4-2 | Roots | 1 | 23.86 | 70.73 | 166.12 | 92.95 | 145.29 |
| | | 10 | 45.40 | 30.85 | 264.85 | 178.84 | 219.18 |
| | | 100 | 81.55 | 21.99 | 490.19 | 374.44 | 202.42 |
| 4-3 | Stems | 1 | 28.54 | 52.11 | 110.55 | 95.27 | 101.13 |
| | | 10 | 38.23 | 33.48 | 313.23 | 125.24 | 160.08 |
| | | 100 | 80.67 | 30.93 | 388.61 | 428.10 | 345.44 |
| 4-4 | Leaves | 1 | 24.01 | 43.86 | 101.47 | 107.00 | 118.71 |
| | | 10 | 41.68 | 31.83 | 262.40 | 190.73 | 191.75 |
| | | 100 | 72.73 | 21.75 | 449.43 | 457.91 | 256.98 |
| 4-5 | Fruits | 1 | 20.83 | 72.67 | 151.34 | 86.76 | 121.41 |
| | | 10 | 36.04 | 31.00 | 291.16 | 171.69 | 134.64 |
| | | 100 | 73.87 | 27.23 | 473.10 | 313.60 | 342.70 |
| 4-6 | Flowers | 1 | 30.58 | 56.57 | 100.28 | 83.32 | 90.65 |
| | | 10 | 44.33 | 31.65 | 201.96 | 188.73 | 226.71 |
| | | 100 | 85.01 | 21.61 | 461.45 | 366.36 | 244.23 |
| 4-7 | Shoots | 1 | 20.20 | 43.16 | 127.34 | 93.74 | 109.68 |
| | | 10 | 25.82 | 31.88 | 144.80 | 148.04 | 232.89 |
| | | 100 | 25.42 | 29.49 | 193.10 | 407.67 | 273.76 |

TABLE 8

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 4 | Goat's beard | | | | | | |
| 4-1 | Whole plant | 1 | 98.22 | 112.03 | 95.48 | 68.40 | 161.09 |
| | | 10 | 91.06 | 117.55 | 57.38 | 85.44 | 321.55 |
| | | 100 | 78.56 | 159.28 | 37.60 | 96.52 | 493.59 |
| 4-2 | Roots | 1 | 98.58 | 114.83 | 96.59 | 77.83 | 124.99 |
| | | 10 | 83.80 | 123.39 | 74.52 | 75.66 | 217.63 |
| | | 100 | 78.32 | 157.96 | 26.57 | 91.43 | 483.37 |
| 4-3 | Stems | 1 | 98.09 | 108.44 | 102.31 | 65.77 | 103.10 |
| | | 10 | 81.90 | 132.69 | 77.19 | 90.31 | 178.69 |
| | | 100 | 76.10 | 133.25 | 50.07 | 93.74 | 422.94 |
| 4-4 | Leaves | 1 | 97.95 | 103.23 | 92.06 | 77.57 | 182.85 |
| | | 10 | 94.87 | 127.54 | 79.55 | 88.39 | 251.37 |
| | | 100 | 73.88 | 144.82 | 5.40 | 90.46 | 321.18 |
| 4-5 | Fruits | 1 | 101.90 | 107.14 | 98.17 | 76.72 | 154.17 |
| | | 10 | 82.82 | 118.04 | 56.94 | 90.33 | 208.59 |
| | | 100 | 80.11 | 134.61 | 24.26 | 107.45 | 442.09 |
| 4-6 | Flowers | 1 | 96.77 | 103.83 | 109.54 | 77.31 | 183.63 |
| | | 10 | 88.90 | 109.63 | 91.35 | 88.86 | 180.11 |
| | | 100 | 79.81 | 151.17 | 16.29 | 100.80 | 336.41 |
| 4-7 | Shoots | 1 | 96.71 | 109.66 | 36.64 | 71.03 | 172.16 |
| | | 10 | 85.38 | 112.40 | 17.34 | 71.34 | 284.27 |
| | | 100 | 79.44 | 136.40 | 20.88 | 71.20 | 453.64 |

### Example 5. Ulleungdo Aster

TABLE 9

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 5 | Ulleungdo | | | | | | |
| | aster | | | | | | |
| 5-1 | Whole plant | 1 | 22.45 | 47.89 | 196.70 | 100.82 | 80.14 |
| | | 10 | 46.98 | 31.64 | 219.46 | 122.07 | 209.68 |
| | | 100 | 70.77 | 26.35 | 484.35 | 380.04 | 245.24 |
| 5-2 | Roots | 1 | 25.28 | 62.12 | 156.17 | 104.81 | 117.49 |
| | | 10 | 45.66 | 32.78 | 312.85 | 160.87 | 191.23 |
| | | 100 | 77.61 | 25.48 | 456.60 | 473.94 | 284.77 |
| 5-3 | Stems | 1 | 20.67 | 89.29 | 168.78 | 106.49 | 154.88 |
| | | 10 | 36.92 | 40.23 | 220.90 | 136.22 | 228.72 |
| | | 100 | 84.39 | 25.78 | 353.76 | 301.41 | 332.39 |
| 5-4 | Leaves | 1 | 30.09 | 48.24 | 183.63 | 101.56 | 148.60 |
| | | 10 | 41.42 | 40.67 | 319.53 | 148.54 | 223.90 |
| | | 100 | 83.23 | 30.81 | 462.55 | 442.82 | 334.77 |
| 5-5 | Fruits | 1 | 22.65 | 48.47 | 118.37 | 101.75 | 85.09 |
| | | 10 | 47.61 | 32.22 | 217.49 | 194.07 | 139.94 |
| | | 100 | 73.82 | 28.45 | 433.75 | 454.05 | 325.84 |
| 5-6 | Flowers | 1 | 25.26 | 79.92 | 108.39 | 83.53 | 139.24 |
| | | 10 | 47.85 | 34.11 | 260.39 | 128.95 | 134.68 |
| | | 100 | 78.67 | 27.67 | 388.12 | 460.81 | 309.82 |
| 5-7 | Shoots | 1 | 20.17 | 95.98 | 101.34 | 107.18 | 122.45 |
| | | 10 | 38.74 | 102.07 | 76.48 | 182.57 | 111.55 |
| | | 100 | 80.33 | 103.37 | 133.30 | 410.41 | 257.59 |

TABLE 10

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 5 | Ulleungdo | | | | | | |
| | aster | | | | | | |
| 5-1 | Whole plant | 1 | 98.95 | 102.41 | 88.38 | 73.44 | 145.15 |
| | | 10 | 84.23 | 140.55 | 73.70 | 94.78 | 283.31 |
| | | 100 | 76.50 | 134.75 | 50.82 | 110.83 | 377.87 |
| 5-2 | Roots | 1 | 98.77 | 108.26 | 110.61 | 78.90 | 104.37 |
| | | 10 | 89.20 | 134.93 | 61.06 | 83.18 | 242.99 |
| | | 100 | 79.39 | 143.10 | 29.22 | 93.86 | 388.46 |
| 5-3 | Stems | 1 | 101.64 | 95.42 | 110.09 | 83.79 | 201.55 |
| | | 10 | 94.57 | 140.60 | 85.73 | 78.18 | 273.79 |
| | | 100 | 78.50 | 155.92 | 36.52 | 100.87 | 412.65 |
| 5-4 | Leaves | 1 | 98.77 | 105.26 | 110.30 | 66.03 | 199.39 |
| | | 10 | 94.07 | 124.32 | 84.33 | 77.75 | 333.71 |
| | | 100 | 72.13 | 160.02 | 17.41 | 91.47 | 435.07 |
| 5-5 | Fruits | 1 | 103.04 | 111.13 | 80.64 | 72.00 | 180.10 |
| | | 10 | 91.32 | 105.34 | 75.09 | 95.09 | 195.51 |
| | | 100 | 73.66 | 139.30 | 49.91 | 101.50 | 390.69 |
| 5-6 | Flowers | 1 | 95.71 | 110.01 | 106.26 | 65.88 | 197.03 |
| | | 10 | 84.73 | 113.51 | 64.89 | 77.06 | 221.85 |
| | | 100 | 79.01 | 155.15 | 21.64 | 93.66 | 406.54 |
| 5-7 | Shoots | 1 | 95.97 | 101.06 | 105.85 | 65.78 | 104.86 |
| | | 10 | 89.21 | 114.73 | 106.09 | 76.76 | 181.33 |
| | | 100 | 80.97 | 155.58 | 98.67 | 106.18 | 322.14 |

### Example 6. Myeongyi

1) Myeongyi (Allium victoriallis)

TABLE 11

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 6 | Myeongyi | | | | | | |
| 6-1 | Whole plant | 1 | 29.57 | 86.84 | 143.37 | 97.83 | 127.49 |
| | | 10 | 50.25 | 33.07 | 252.30 | 146.06 | 147.14 |
| | | 100 | 79.89 | 22.88 | 414.82 | 466.35 | 304.41 |
| 6-2 | Roots | 1 | 10.33 | 91.39 | 117.71 | 99.15 | 82.28 |
| | | 10 | 18.97 | 95.00 | 232.89 | 114.48 | 121.96 |
| | | 100 | 27.99 | 94.63 | 427.59 | 426.43 | 281.81 |
| 6-3 | Stems | 1 | 21.08 | 68.30 | 124.82 | 109.42 | 113.90 |
| | | 10 | 48.59 | 35.44 | 216.71 | 122.98 | 164.46 |
| | | 100 | 77.27 | 30.80 | 389.77 | 391.75 | 266.48 |
| 6-4 | Leaves | 1 | 20.94 | 113.73 | 172.82 | 94.87 | 131.29 |
| | | 10 | 34.10 | 94.03 | 292.90 | 133.64 | 158.66 |
| | | 100 | 25.70 | 96.74 | 444.31 | 467.04 | 307.69 |
| 6-5 | Fruits | 1 | 23.17 | 75.04 | 126.19 | 97.16 | 97.89 |
| | | 10 | 46.80 | 38.87 | 221.49 | 180.48 | 144.79 |
| | | 100 | 76.98 | 24.13 | 465.50 | 469.75 | 286.94 |
| 6-6 | Flowers | 1 | 20.11 | 61.41 | 103.63 | 97.93 | 99.31 |
| | | 10 | 43.49 | 38.23 | 235.16 | 190.37 | 222.24 |
| | | 100 | 72.94 | 21.12 | 449.83 | 337.20 | 233.11 |

TABLE 12

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 6 | Myeongyi | | | | | | |
| 6-1 | Whole plant | 1 | 96.24 | 97.45 | 109.15 | 78.39 | 178.39 |
| | | 10 | 83.05 | 136.29 | 75.60 | 86.67 | 261.27 |
| | | 100 | 78.73 | 133.75 | 4.64 | 108.54 | 320.29 |
| 6-2 | Roots | 1 | 99.72 | 98.12 | 89.70 | 79.84 | 143.86 |
| | | 10 | 88.53 | 126.86 | 89.74 | 75.44 | 312.87 |
| | | 100 | 79.58 | 148.17 | 18.61 | 79.04 | 423.96 |
| 6-3 | Stems | 1 | 99.78 | 103.14 | 89.44 | 84.00 | 93.63 |
| | | 10 | 83.18 | 112.36 | 56.71 | 95.58 | 271.12 |
| | | 100 | 70.16 | 158.05 | 37.50 | 102.27 | 451.01 |
| 6-4 | Leaves | 1 | 96.52 | 114.00 | 98.54 | 65.65 | 124.46 |
| | | 10 | 83.93 | 129.88 | 91.66 | 64.32 | 249.11 |
| | | 100 | 72.01 | 135.64 | 33.84 | 59.62 | 430.38 |
| 6-5 | Fruits | 1 | 103.99 | 97.89 | 93.90 | 72.79 | 155.93 |
| | | 10 | 90.35 | 122.37 | 73.27 | 78.25 | 257.87 |
| | | 100 | 72.50 | 142.35 | 38.68 | 105.29 | 426.04 |
| 6-6 | Flowers | 1 | 95.83 | 98.94 | 86.72 | 72.64 | 191.42 |
| | | 10 | 87.44 | 133.43 | 72.41 | 94.74 | 289.82 |
| | | 100 | 74.07 | 137.04 | 42.04 | 90.80 | 475.34 |

2) Myeongyi (Allium ulleungense)

TABLE 13

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 6 | Myeongyi | | | | | | |
| 6-21 | Whole plant | 1 | 31.33 | 82.74 | 160.11 | 102.17 | 133.78 |
| | | 10 | 55.77 | 30.11 | 300.31 | 155.81 | 153.98 |
| | | 100 | 86.17 | 15.78 | 455.55 | 480.25 | 333.13 |
| 6-22 | Roots | 1 | 25.44 | 90.11 | 150.19 | 105.51 | 90.18 |
| | | 10 | 33.85 | 85.14 | 282.22 | 133.78 | 141.81 |
| | | 100 | 40.17 | 81.16 | 450.91 | 444.41 | 303.18 |
| 6-23 | Stems | 1 | 30.11 | 65.37 | 150.17 | 114.43 | 119.91 |
| | | 10 | 55.87 | 30.81 | 267.99 | 137.11 | 182.21 |
| | | 100 | 83.18 | 22.79 | 420.81 | 405.19 | 283.97 |
| 6-24 | Leaves | 1 | 35.77 | 80.16 | 200.88 | 116.66 | 150.31 |
| | | 10 | 68.18 | 45.18 | 350.66 | 180.97 | 198.98 |
| | | 100 | 87.11 | 10.81 | 503.31 | 521.51 | 367.18 |
| 6-25 | Fruits | 1 | 28.11 | 70.18 | 166.16 | 108.87 | 102.22 |
| | | 10 | 50.10 | 33.77 | 233.41 | 190.34 | 167.18 |
| | | 100 | 80.17 | 20.82 | 493.31 | 487.11 | 308.17 |
| 6-26 | Flowers | 1 | 30.99 | 55.93 | 133.87 | 106.31 | 107.41 |
| | | 10 | 48.17 | 32.61 | 272.22 | 200.28 | 252.13 |
| | | 100 | 75.18 | 15.35 | 469.99 | 363.13 | 289.65 |

TABLE 14

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydro- |
| | | | 1 uM) | mM) | Inhibitor IV | ppm) | testosterone |
| | | | | | 15 nM) | | 100 nM) |
| 6 | Myeongyi | | | | | | |
| 6-21 | Whole plant | 1 | 95.11 | 101.11 | 99.99 | 80.11 | 191.33 |
| | | 10 | 80.16 | 140.99 | 70.61 | 88.30 | 280.34 |
| | | 100 | 72.22 | 152.27 | 3.38 | 110.31 | 350.99 |
| 6-22 | Roots | 1 | 98.91 | 103.30 | 88.98 | 80.35 | 160.31 |
| | | 10 | 85.13 | 138.18 | 88.91 | 82.18 | 333.33 |
| | | 100 | 75.33 | 155.61 | 15.61 | 89.18 | 447.71 |
| 6-23 | Stems | 1 | 99.01 | 104.10 | 88.14 | 85.11 | 100.01 |
| | | 10 | 81.81 | 126.91 | 55.61 | 99.99 | 270.15 |
| | | 100 | 65.33 | 161.10 | 35.76 | 105.31 | 475.55 |
| 6-24 | Leaves | 1 | 90.16 | 121.00 | 98.13 | 80.61 | 166.61 |
| | | 10 | 78.66 | 147.18 | 49.66 | 94.38 | 287.18 |
| | | 100 | 63.31 | 167.90 | 2.50 | 103.35 | 497.17 |
| 6-25 | Fruits | 1 | 98.89 | 102.23 | 94.10 | 80.31 | 160.93 |
| | | 10 | 87.61 | 125.61 | 70.33 | 98.35 | 268.17 |
| | | 100 | 68.16 | 150.53 | 35.18 | 108.18 | 443.95 |
| 6-26 | Flowers | 1 | 92.22 | 101.11 | 85.18 | 77.69 | 203.63 |
| | | 10 | 83.38 | 135.91 | 60.33 | 95.91 | 302.43 |
| | | 100 | 70.00 | 145.18 | 39.87 | 101.17 | 491.13 |

### Example 7. Aging Chive

TABLE 15

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 7 | Aging chive | | | | | | |
| 7-1 | Whole plant | 1 | 28.07 | 51.33 | 125.93 | 101.89 | 93.82 |
| | | 10 | 32.71 | 36.56 | 229.93 | 172.29 | 209.51 |
| | | 100 | 75.62 | 28.34 | 424.73 | 403.99 | 252.76 |
| 7-2 | Roots | 1 | 21.64 | 80.15 | 106.17 | 94.42 | 139.34 |
| | | 10 | 43.37 | 33.92 | 276.77 | 134.35 | 149.42 |
| | | 100 | 70.51 | 22.10 | 488.32 | 368.84 | 247.22 |
| 7-3 | Stems | 1 | 28.62 | 48.06 | 127.55 | 91.59 | 139.77 |
| | | 10 | 45.15 | 37.65 | 224.08 | 119.65 | 130.73 |
| | | 100 | 84.80 | 20.55 | 465.91 | 399.98 | 347.63 |
| 7-4 | Leaves | 1 | 24.88 | 99.88 | 95.93 | 86.01 | 106.58 |
| | | 10 | 46.54 | 116.37 | 122.42 | 176.95 | 156.09 |
| | | 100 | 79.79 | 94.26 | 97.08 | 453.84 | 235.02 |
| 7-5 | Fruits | 1 | 29.84 | 54.48 | 133.81 | 81.73 | 121.30 |
| | | 10 | 46.60 | 35.91 | 228.69 | 110.57 | 219.90 |
| | | 100 | 71.32 | 22.03 | 454.58 | 431.78 | 330.99 |
| 7-6 | Flowers | 1 | 26.90 | 76.02 | 167.99 | 98.29 | 145.86 |
| | | 10 | 36.24 | 33.89 | 238.07 | 145.13 | 138.72 |
| | | 100 | 89.93 | 23.14 | 492.46 | 493.39 | 225.08 |

TABLE 16

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 μM) | mM) | Inhibitor IV | ppm) | 100 nM) |
| | | | | | 15 nM) | | |
| 7 | Aging chive | | | | | | |
| 7-1 | Whole plant | 1 | 98.18 | 107.61 | 100.68 | 66.56 | 173.25 |
| | | 10 | 90.33 | 109.33 | 75.26 | 77.75 | 237.46 |
| | | 100 | 70.64 | 154.73 | 10.97 | 110.58 | 346.89 |
| 7-2 | Roots | 1 | 97.64 | 95.62 | 83.81 | 80.47 | 205.67 |
| | | 10 | 82.59 | 116.89 | 95.82 | 77.57 | 207.16 |
| | | 100 | 76.76 | 130.83 | 26.56 | 105.44 | 343.69 |
| 7-3 | Stems | 1 | 96.89 | 115.92 | 105.59 | 79.12 | 124.89 |
| | | 10 | 95.58 | 129.82 | 93.91 | 78.46 | 218.58 |
| | | 100 | 76.13 | 154.61 | 23.28 | 105.69 | 473.34 |
| 7-4 | Leaves | 1 | 101.07 | 100.03 | 95.87 | 70.03 | 94.95 |
| | | 10 | 94.76 | 118.64 | 58.06 | 75.85 | 219.46 |
| | | 100 | 73.34 | 142.20 | 4.69 | 96.76 | 395.37 |
| 7-5 | Fruits | 1 | 103.17 | 100.89 | 96.81 | 84.67 | 90.65 |
| | | 10 | 93.40 | 110.62 | 74.95 | 78.56 | 261.66 |
| | | 100 | 73.96 | 139.57 | 11.69 | 98.98 | 375.00 |
| 7-6 | Flowers | 1 | 99.24 | 100.83 | 87.62 | 81.91 | 186.21 |
| | | 10 | 89.59 | 112.66 | 85.14 | 90.08 | 252.37 |
| | | 100 | 79.78 | 136.17 | 40.43 | 109.33 | 434.49 |

### Example 8. Silvery Mountain Ash

TABLE 17

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 8 | Silvery | | | | | | |
| | mountain | | | | | | |
| | ash | | | | | | |
| 8-1 | Whole plant | 1 | 25.05 | 77.16 | 156.35 | 93.49 | 107.50 |
| | | 10 | 50.69 | 37.86 | 297.44 | 161.28 | 112.15 |
| | | 100 | 71.89 | 27.48 | 455.32 | 489.78 | 268.90 |
| 8-2 | Roots | 1 | 22.13 | 73.26 | 169.56 | 86.18 | 142.46 |
| | | 10 | 32.68 | 36.18 | 213.38 | 166.63 | 221.50 |
| | | 100 | 75.07 | 30.14 | 473.55 | 402.16 | 313.79 |
| 8-3 | Stems | 1 | 22.80 | 93.74 | 160.53 | 95.01 | 87.71 |
| | | 10 | 29.86 | 89.96 | 289.28 | 133.14 | 250.55 |
| | | 100 | 14.13 | 94.91 | 469.25 | 106.45 | 304.01 |
| 8-4 | Leaves | 1 | 28.71 | 72.06 | 113.34 | 98.41 | 128.40 |
| | | 10 | 45.38 | 35.81 | 208.12 | 119.44 | 108.77 |
| | | 100 | 79.62 | 28.79 | 498.80 | 308.21 | 201.30 |
| 8-5 | Fruits | 1 | 19.30 | 90.91 | 141.85 | 107.63 | 122.06 |
| | | 10 | 27.37 | 82.57 | 350.07 | 84.29 | 218.92 |
| | | 100 | 10.73 | 88.83 | 398.30 | 159.69 | 324.73 |
| 8-6 | Flowers | 1 | 23.00 | 40.65 | 134.64 | 100.21 | 137.00 |
| | | 10 | 38.49 | 35.76 | 252.85 | 140.86 | 206.56 |
| | | 100 | 82.78 | 23.12 | 440.17 | 370.17 | 226.00 |

TABLE 18

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 8 | Silvery | | | | | | |
| | mountain | | | | | | |
| | ash | | | | | | |
| 8-1 | Whole plant | 1 | 99.40 | 110.88 | 89.85 | 78.39 | 190.61 |
| | | 10 | 89.62 | 123.37 | 61.94 | 92.97 | 264.42 |
| | | 100 | 74.79 | 142.32 | 26.76 | 109.84 | 345.73 |
| 8-2 | Roots | 1 | 98.18 | 108.50 | 99.67 | 69.11 | 131.15 |
| | | 10 | 80.51 | 110.25 | 76.67 | 80.44 | 231.79 |
| | | 100 | 71.45 | 141.15 | 10.72 | 108.89 | 486.21 |
| 8-3 | Stems | 1 | 100.11 | 110.57 | 84.84 | 80.21 | 153.53 |
| | | 10 | 91.78 | 95.94 | 69.34 | 84.52 | 326.67 |
| | | 100 | 70.14 | 101.34 | 9.56 | 102.69 | 454.37 |
| 8-4 | Leaves | 1 | 98.94 | 107.39 | 107.46 | 78.19 | 91.14 |
| | | 10 | 84.76 | 130.06 | 87.49 | 93.06 | 210.76 |
| | | 100 | 80.86 | 152.86 | 8.11 | 93.31 | 448.02 |
| 8-5 | Fruits | 1 | 95.15 | 100.91 | 105.51 | 72.82 | 128.76 |
| | | 10 | 86.26 | 109.01 | 75.67 | 95.72 | 317.33 |
| | | 100 | 72.23 | 101.63 | 11.27 | 110.88 | 402.18 |
| 8-6 | Flowers | 1 | 98.19 | 102.05 | 100.90 | 85.65 | 134.92 |
| | | 10 | 89.58 | 136.47 | 82.80 | 94.00 | 212.97 |
| | | 100 | 71.85 | 159.22 | 0.65 | 92.35 | 449.08 |

### Example 9. Ulleungdo Thyme

TABLE 19

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 9 | Ulleungdo | | | | | | |
| | thyme | | | | | | |
| 9-1 | Whole plant | 1 | 25.36 | 57.96 | 144.543 | 93.44 | 133.99 |
| | | 10 | 49.96 | 34.36 | 215.04 | 119.92 | 221.59 |
| | | 100 | 83.95 | 21.10 | 482.50 | 443.04 | 279.18 |
| 9-2 | Roots | 1 | 22.69 | 69.11 | 177.17 | 104.32 | 89.48 |
| | | 10 | 38.05 | 36.38 | 288.41 | 154.45 | 240.85 |
| | | 100 | 78.54 | 26.39 | 479.93 | 428.79 | 223.96 |
| 9-3 | Stems | 1 | 28.24 | 84.59 | 179.55 | 84.38 | 117.44 |
| | | 10 | 30.15 | 33.86 | 296.27 | 110.50 | 204.00 |
| | | 100 | 77.97 | 25.97 | 352.88 | 430.61 | 316.92 |
| 9-4 | Leaves | 1 | 25.69 | 54.96 | 173.79 | 107.00 | 111.02 |
| | | 10 | 45.64 | 31.26 | 234.22 | 126.99 | 119.58 |
| | | 100 | 75.54 | 25.04 | 409.27 | 431.62 | 296.07 |
| 9-5 | Fruits | 1 | 21.54 | 63.91 | 183.64 | 95.44 | 110.19 |
| | | 10 | 43.79 | 38.23 | 328.99 | 121.66 | 152.47 |
| | | 100 | 83.63 | 21.43 | 360.30 | 331.86 | 290.23 |
| 9-6 | Flowers | 1 | 28.77 | 48.81 | 142.73 | 101.87 | 110.92 |
| | | 10 | 42.96 | 33.39 | 225.11 | 112.43 | 160.44 |
| | | 100 | 85.27 | 22.39 | 366.78 | 430.68 | 273.81 |

TABLE 20

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 9 | Ulleungdo | | | | | | |
| | thyme | | | | | | |
| 9-1 | Whole plant | 1 | 100.65 | 101.98 | 100.93 | 72.75 | 187.85 |
| | | 10 | 85.27 | 137.00 | 65.78 | 93.29 | 185.58 |
| | | 100 | 76.37 | 159.79 | 17.25 | 109.93 | 348.42 |
| 9-2 | Roots | 1 | 96.52 | 108.74 | 84.44 | 78.20 | 174.91 |
| | | 10 | 84.66 | 121.04 | 77.01 | 83.64 | 270.10 |
| | | 100 | 75.89 | 154.80 | 23.11 | 98.84 | 333.44 |
| 9-3 | Stems | 1 | 103.04 | 100.28 | 97.32 | 82.13 | 157.44 |
| | | 10 | 88.41 | 126.80 | 48.70 | 77.52 | 231.57 |
| | | 100 | 78.43 | 133.49 | 30.39 | 104.29 | 415.30 |
| 9-4 | Leaves | 1 | 98.29 | 107.16 | 108.38 | 82.92 | 102.11 |
| | | 10 | 90.36 | 133.01 | 52.29 | 95.96 | 278.68 |
| | | 100 | 75.75 | 160.61 | 50.73 | 90.47 | 344.70 |
| 9-5 | Fruits | 1 | 102.38 | 108.79 | 107.56 | 78.81 | 117.41 |
| | | 10 | 80.21 | 121.75 | 95.05 | 84.39 | 310.69 |
| | | 100 | 75.16 | 159.53 | 14.41 | 109.65 | 493.69 |
| 9-6 | Flowers | 1 | 100.39 | 100.58 | 89.92 | 67.66 | 198.44 |
| | | 10 | 92.46 | 139.71 | 90.04 | 91.06 | 338.71 |
| | | 100 | 74.84 | 143.02 | 1.25 | 98.84 | 489.15 |

### Example 10. Wild Chervil

TABLE 21

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 10 | Wild chervil | | | | | | |
| 10-1 | Whole plant | 1 | 25.34 | 88.88 | 126.44 | 95.38 | 137.36 |
| | | 10 | 33.88 | 35.55 | 330.67 | 181.17 | 231.59 |
| | | 100 | 73.30 | 29.03 | 389.16 | 360.01 | 329.74 |
| 10-2 | Roots | 1 | 20.65 | 62.92 | 172.21 | 92.92 | 120.62 |
| | | 10 | 50.16 | 30.10 | 313.40 | 132.24 | 186.96 |
| | | 100 | 84.21 | 20.92 | 431.79 | 410.49 | 215.30 |
| 10-3 | Stems | 1 | 26.13 | 73.53 | 150.34 | 82.82 | 136.86 |
| | | 10 | 41.01 | 36.28 | 227.75 | 141.36 | 218.00 |
| | | 100 | 78.42 | 25.50 | 408.49 | 311.23 | 241.55 |
| 10-4 | Leaves | 1 | 24.45 | 57.68 | 196.64 | 102.95 | 141.37 |
| | | 10 | 32.62 | 39.75 | 248.69 | 138.54 | 168.67 |
| | | 100 | 77.78 | 27.29 | 478.89 | 376.99 | 202.60 |
| 10-5 | Fruits | 1 | 25.51 | 42.95 | 156.21 | 81.80 | 119.18 |
| | | 10 | 35.52 | 34.56 | 262.16 | 137.06 | 209.84 |
| | | 100 | 85.96 | 23.03 | 482.26 | 467.67 | 261.88 |
| 10-6 | Flowers | 1 | 23.38 | 74.09 | 126.01 | 110.14 | 143.72 |
| | | 10 | 45.04 | 31.94 | 251.11 | 180.74 | 165.34 |
| | | 100 | 76.21 | 22.27 | 421.22 | 372.55 | 274.02 |

TABLE 22

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 10 | Wild chervil | | | | | | |
| 10-1 | Whole plant | 1 | 95.32 | 104.72 | 100.21 | 68.76 | 116.47 |
| | | 10 | 92.78 | 133.27 | 81.08 | 80.16 | 221.89 |
| | | 100 | 77.46 | 138.36 | 24.58 | 97.59 | 420.80 |
| 10-2 | Roots | 1 | 103.48 | 100.16 | 81.38 | 85.45 | 198.37 |
| | | 10 | 87.16 | 120.30 | 85.17 | 79.68 | 283.01 |
| | | 100 | 77.49 | 148.09 | 41.48 | 107.00 | 382.19 |
| 10-3 | Stems | 1 | 103.53 | 113.22 | 84.96 | 83.99 | 93.61 |
| | | 10 | 80.13 | 128.38 | 49.47 | 79.28 | 306.16 |
| | | 100 | 72.23 | 154.88 | 49.33 | 109.85 | 377.78 |
| 10-4 | Leaves | 1 | 97.07 | 103.41 | 99.67 | 83.50 | 169.07 |
| | | 10 | 97.91 | 92.45 | 56.08 | 91.70 | 270.95 |
| | | 100 | 99.46 | 87.59 | 9.76 | 104.04 | 478.85 |
| 10-5 | Fruits | 1 | 95.30 | 115.44 | 109.69 | 81.67 | 163.79 |
| | | 10 | 81.27 | 105.11 | 58.22 | 89.25 | 321.13 |
| | | 100 | 75.19 | 134.05 | 31.94 | 96.97 | 495.32 |
| 10-6 | Flowers | 1 | 99.27 | 110.00 | 107.75 | 77.95 | 178.42 |
| | | 10 | 95.60 | 137.84 | 52.10 | 90.87 | 268.32 |
| | | 100 | 78.67 | 149.39 | 4.41 | 96.89 | 338.16 |

### Example 11. Giant Knotweed

TABLE 23

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 11 | Giant | | | | | | |
| | Knotweed | | | | | | |
| 11-1 | Whole plant | 1 | 26.32 | 62.98 | 170.47 | 106.58 | 91.53 |
| | | 10 | 36.54 | 40.43 | 346.75 | 110.29 | 123.58 |
| | | 100 | 76.96 | 24.31 | 403.58 | 400.31 | 223.41 |
| 11-2 | Roots | 1 | 27.11 | 82.29 | 178.63 | 110.38 | 102.26 |
| | | 10 | 38.07 | 34.65 | 237.27 | 188.60 | 121.01 |
| | | 100 | 77.96 | 30.12 | 372.67 | 489.59 | 229.76 |
| 11 -3 | Stems | 1 | 26.99 | 81.92 | 126.42 | 99.67 | 142.46 |
| | | 10 | 42.11 | 32.45 | 232.40 | 189.49 | 194.51 |
| | | 100 | 71.68 | 23.42 | 448.88 | 321.31 | 333.82 |
| 11-4 | Leaves | 1 | 20.34 | 82.83 | 124.54 | 89.88 | 134.34 |
| | | 10 | 33.20 | 33.48 | 315.84 | 170.30 | 106.63 |
| | | 100 | 85.65 | 21.22 | 486.06 | 490.66 | 246.01 |
| 11-5 | Fruits | 1 | 22.31 | 75.20 | 186.74 | 81.98 | 112.29 |
| | | 10 | 32.75 | 40.24 | 343.80 | 128.58 | 121.48 |
| | | 100 | 79.02 | 24.30 | 447.56 | 484.34 | 258.57 |
| 11-6 | Flowers | 1 | 25.77 | 55.69 | 158.85 | 106.12 | 141.95 |
| | | 10 | 34.38 | 34.69 | 338.45 | 117.74 | 108.72 |
| | | 100 | 71.12 | 23.20 | 451.57 | 332.15 | 348.63 |

TABLE 24

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 11 | Giant | | | | | | |
| | Knotweed | | | | | | |
| 11-1 | Whole plant | 1 | 99.65 | 107.48 | 87.82 | 70.71 | 124.16 |
| | | 10 | 87.57 | 126.93 | 70.41 | 81.95 | 186.89 |
| | | 100 | 71.94 | 146.02 | 15.50 | 109.09 | 437.60 |
| 11-2 | Roots | 1 | 96.35 | 114.79 | 107.28 | 79.20 | 146.25 |
| | | 10 | 95.87 | 119.35 | 53.84 | 87.40 | 221.45 |
| | | 100 | 77.24 | 151.45 | 3.70 | 99.98 | 358.01 |
| 11-3 | Stems | 1 | 96.48 | 97.75 | 102.82 | 65.26 | 101.24 |
| | | 10 | 95.05 | 126.00 | 47.46 | 93.39 | 284.88 |
| | | 100 | 71.07 | 139.81 | 47.90 | 95.55 | 500.26 |
| 11-4 | Leaves | 1 | 96.80 | 102.16 | 91.62 | 67.71 | 128.34 |
| | | 10 | 87.76 | 126.14 | 85.10 | 92.58 | 309.63 |
| | | 100 | 76.24 | 157.73 | 42.01 | 91.37 | 477.16 |
| 11-5 | Fruits | 1 | 96.01 | 108.53 | 88.65 | 79.37 | 140.10 |
| | | 10 | 86.40 | 105.69 | 63.48 | 83.36 | 302.50 |
| | | 100 | 80.70 | 149.35 | 18.38 | 107.85 | 389.20 |
| 11-6 | Flowers | 1 | 103.80 | 112.63 | 80.64 | 74.13 | 132.42 |
| | | 10 | 85.36 | 125.06 | 76.57 | 76.38 | 267.97 |
| | | 100 | 72.87 | 144.50 | 4.68 | 106.33 | 468.28 |

### Example 12. Mono Maple

TABLE 25

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 12 | Mono maple | | | | | | |
| 12-1 | Whole plant | 1 | 23.00 | 87.97 | 152.59 | 105.47 | 99.79 |
| | | 10 | 46.50 | 31.93 | 202.16 | 115.08 | 138.47 |
| | | 100 | 76.14 | 20.97 | 433.95 | 402.77 | 228.50 |
| 12-2 | Roots | 1 | 21.73 | 67.96 | 161.26 | 86.48 | 100.81 |
| | | 10 | 49.42 | 37.90 | 281.96 | 162.86 | 146.23 |
| | | 100 | 77.35 | 23.52 | 412.76 | 372.43 | 250.68 |
| 12-3 | Stems | 1 | 21.81 | 41.43 | 163.21 | 101.54 | 132.54 |
| | | 10 | 38.55 | 34.69 | 318.16 | 193.59 | 219.27 |
| | | 100 | 78.15 | 20.17 | 423.15 | 381.91 | 235.32 |
| 12-4 | Leaves | 1 | 20.07 | 49.96 | 191.83 | 85.80 | 110.57 |
| | | 10 | 33.31 | 31.51 | 294.52 | 171.18 | 217.42 |
| | | 100 | 75.12 | 27.15 | 439.70 | 344.92 | 270.95 |
| 12-5 | Fruits | 1 | 29.77 | 56.78 | 115.91 | 100.77 | 120.79 |
| | | 10 | 39.89 | 40.96 | 341.71 | 173.22 | 238.31 |
| | | 100 | 73.35 | 29.97 | 419.95 | 337.38 | 200.71 |
| 12-6 | Flowers | 1 | 30.17 | 53.03 | 191.98 | 80.89 | 126.05 |
| | | 10 | 45.77 | 32.34 | 215.89 | 189.27 | 204.05 |
| | | 100 | 78.16 | 27.71 | 447.50 | 312.72 | 319.77 |
| 12-7 | Sap | 1 | 30.73 | 63.80 | 133.74 | 99.05 | 95.81 |
| | | 10 | 45.40 | 39.44 | 340.24 | 151.68 | 170.39 |
| | | 100 | 72.97 | 25.88 | 413.63 | 377.84 | 347.78 |

TABLE 26

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 12 | Mono maple | | | | | | |
| 12-1 | Whole plant | 1 | 98.72 | 106.40 | 94.07 | 85.57 | 109.16 |
| | | 10 | 87.69 | 111.66 | 45.24 | 83.36 | 184.85 |
| | | 100 | 74.38 | 130.72 | 42.34 | 94.50 | 470.34 |
| 12-2 | Roots | 1 | 95.51 | 113.25 | 108.49 | 72.47 | 170.57 |
| | | 10 | 91.47 | 117.00 | 75.31 | 94.68 | 206.06 |
| | | 100 | 80.68 | 154.40 | 44.72 | 110.86 | 309.08 |
| 12-3 | Stems | 1 | 96.14 | 101.42 | 107.86 | 71.61 | 184.18 |
| | | 10 | 83.81 | 128.03 | 76.12 | 85.93 | 237.67 |
| | | 100 | 78.20 | 153.68 | 5.39 | 103.80 | 468.32 |
| 12-4 | Leaves | 1 | 102.70 | 95.75 | 82.82 | 75.33 | 141.98 |
| | | 10 | 82.90 | 114.02 | 68.37 | 83.89 | 195.88 |
| | | 100 | 75.94 | 139.94 | 14.37 | 104.64 | 500.69 |
| 12-5 | Fruits | 1 | 95.88 | 96.72 | 93.73 | 69.31 | 155.40 |
| | | 10 | 81.06 | 105.00 | 90.32 | 76.11 | 252.40 |
| | | 100 | 80.66 | 149.10 | 17.93 | 108.31 | 325.39 |
| 12-6 | Flowers | 1 | 95.30 | 115.39 | 93.50 | 66.48 | 94.23 |
| | | 10 | 81.37 | 128.58 | 85.84 | 93.59 | 206.75 |
| | | 100 | 75.79 | 144.28 | 4.61 | 110.95 | 435.07 |
| 12-7 | Sap | 1 | 96.07 | 101.96 | 89.05 | 68.98 | 93.01 |
| | | 10 | 84.59 | 131.20 | 68.88 | 88.34 | 204.38 |
| | | 100 | 74.65 | 137.77 | 44.98 | 103.29 | 396.76 |

### Example 13. Ulleungdo Ladyfern

TABLE 27

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 13 | Ulleungdo | | | | | | |
| | ladyfern | | | | | | |
| 13-1 | Whole plant | 1 | 20.72 | 54.64 | 107.13 | 107.36 | 133.69 |
| | | 10 | 38.67 | 34.49 | 346.40 | 144.48 | 213.88 |
| | | 100 | 81.81 | 21.05 | 385.39 | 345.92 | 316.54 |
| 13-2 | Roots | 1 | 21.36 | 60.38 | 129.94 | 90.17 | 114.42 |
| | | 10 | 39.09 | 31.44 | 269.44 | 187.92 | 142.61 |
| | | 100 | 71.85 | 23.40 | 467.11 | 402.20 | 264.19 |
| 13-3 | Stems | 1 | 26.21 | 54.10 | 105.16 | 92.02 | 128.10 |
| | | 10 | 49.93 | 36.41 | 204.81 | 128.58 | 151.90 |
| | | 100 | 81.51 | 27.78 | 354.31 | 398.27 | 334.02 |
| 13-4 | Leaves | 1 | 23.87 | 66.72 | 149.55 | 90.40 | 87.05 |
| | | 10 | 47.61 | 36.82 | 221.58 | 198.39 | 211.63 |
| | | 100 | 71.03 | 24.59 | 411.13 | 411.66 | 334.55 |
| 13-5 | Fruits | 1 | 25.53 | 74.57 | 149.89 | 98.57 | 128.40 |
| | | 10 | 33.29 | 30.52 | 214.66 | 116.67 | 202.05 |
| | | 100 | 71.55 | 26.91 | 358.91 | 414.26 | 302.86 |
| 13-6 | Flowers | 1 | 30.94 | 79.15 | 114.06 | 82.23 | 142.54 |
| | | 10 | 41.82 | 32.85 | 334.32 | 167.36 | 225.86 |
| | | 100 | 75.61 | 30.83 | 466.12 | 429.03 | 298.44 |

TABLE 28

| | | | | Blood | Cognitive | | |
|---|---|---|---|---|---|---|---|
| | | | Anti- | glucose- | ability-β- | | Andropause- |
| | | | allergy-IgE | Glucose | secretase | | AR |
| | | | secretion | absorption | activity | Liver health- | transcriptional |
| | | Conc. | (% of | (% of | (% of | Cell viability | activity |
| No. | Sample | (ppm) | control) | control) | control) | (% of control) | (% of control) |
| | Negative | | — | — | — | 68.25 | — |
| | control | | | | | (Ethanol 100 | |
| | | | | | | nm) | |
| | Positive | | 67.27 | 183.77 | 50.00 | 88.28 | 753.39 |
| | control | | (Dexamethasone | (Metformin 1 | (β-Secretase | (Silymarin 100 | (Dihydrotestosterone |
| | | | 1 uM) | mM) | Inhibitor IV | ppm) | 100 nM) |
| | | | | | 15 nM) | | |
| 13 | Ulleungdo | | | | | | |
| | ladyfern | | | | | | |
| 13-1 | Whole plant | 1 | 103.87 | 108.11 | 86.14 | 71.56 | 159.42 |
| | | 10 | 93.23 | 118.86 | 88.46 | 90.77 | 298.38 |
| | | 100 | 70.82 | 155.00 | 32.16 | 100.44 | 409.26 |
| 13-2 | Roots | 1 | 100.02 | 112.60 | 107.94 | 78.90 | 159.36 |
| | | 10 | 91.05 | 123.26 | 62.29 | 77.61 | 198.12 |
| | | 100 | 77.22 | 141.61 | 32.40 | 105.50 | 434.35 |
| 13-3 | Stems | 1 | 99.85 | 97.14 | 80.20 | 65.97 | 129.59 |
| | | 10 | 93.47 | 130.89 | 82.80 | 77.87 | 335.73 |
| | | 100 | 79.50 | 130.03 | 9.34 | 90.17 | 386.82 |
| 13-4 | Leaves | 1 | 95.31 | 109.26 | 110.54 | 82.22 | 164.55 |
| | | 10 | 94.29 | 123.25 | 79.09 | 79.12 | 227.75 |
| | | 100 | 70.04 | 160.47 | 33.91 | 108.58 | 490.81 |
| 13-5 | Fruits | 1 | 97.78 | 111.39 | 109.23 | 82.31 | 138.34 |
| | | 10 | 86.22 | 110.78 | 75.06 | 90.23 | 335.98 |
| | | 100 | 73.90 | 137.50 | 35.06 | 99.79 | 400.44 |
| 13-6 | Flowers | 1 | 100.06 | 112.72 | 82.78 | 81.08 | 115.66 |
| | | 10 | 82.05 | 105.14 | 70.79 | 87.79 | 311.17 |
| | | 100 | 76.24 | 130.75 | 47.04 | 109.59 | 479.40 |

### Example 14. Wild Wasabi

TABLE 29

| | | | Blood | Lipid- | Anti-obesity- | | |
|---|---|---|---|---|---|---|---|
| | | | circulation- | cholesterol | Glycerol | | Menopause- |
| | | | NO Conc. | synthesis | Conc. | Immunity- | ER binding |
| | | Conc. | (% of | (% of | (% of | IFNg secretion | capacity |
| No. | Sample | (ppm) | control) | control) | control) | (% of control) | (% of control) |
| | Negative | | 21.32 | — | — | — | — |
| | control | | (oxLDL 200 | | | | |
| | | | ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 14 | Wild wasabi | | | | | | |
| 1.4-1 | Whole plant | 1 | 22.78 | 67.68 | 143.76 | 96.01 | 137.81 |
| | | 10 | 47.50 | 31.79 | 281.23 | 178.90 | 161.65 |
| | | 100 | 85.42 | 22.34 | 430.71 | 431.59 | 297.18 |
| 14-2 | Roots | 1 | 30.35 | 100.17 | 170.49 | 88.39 | 93.57 |
| | | 10 | 38.39 | 95.04 | 311.73 | 147.45 | 132.36 |
| | | 100 | 70.07 | 85.09 | 366.16 | 478.51 | 248.68 |
| 14-3 | Stems | 1 | 28.12 | 105.35 | 115.50 | 98.32 | 114.54 |
| | | 10 | 43.01 | 92.03 | 282.95 | 134.42 | 166.02 |
| | | 100 | 72.60 | 92.39 | 486.28 | 493.17 | 290.74 |
| 14.4 | Leaves | 1 | 20.81 | 90.88 | 109.26 | 84.62 | 142.88 |
| | | 10 | 49.49 | 87.26 | 229.87 | 111.17 | 143.15 |
| | | 100 | 83.07 | 81.82 | 409.54 | 447.98 | 230.90 |
| 14-5 | Fruits | 1 | 22.04 | 89.28 | 163.56 | 110.95 | 130.95 |
| | | 10 | 48.71 | 39.67 | 267.56 | 125.24 | 122.73 |
| | | 100 | 78.08 | 20.91 | 449.57 | 438.13 | 228.70 |
| 14-6 | Flowers | 1 | 26.00 | 72.46 | 158.83 | 96.21 | 121.65 |
| | | 10 | 35.80 | 32.91 | 212.13 | 113.37 | 106.58 |
| | | 100 | 70.22 | 22.08 | 452.66 | 404.31 | 227.14 |

TABLE 30

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 14 | Wild wasabi | | | | | | |
| 14-1 | Whole plant | 1 | 96.79 | 106.16 | 99.91 | 68.73 | 208.96 |
| | | 10 | 93.68 | 133.34 | 69.68 | 87.74 | 231.70 |
| | | 100 | 70.67 | 133.85 | 4.73 | 110.52 | 468.06 |
| 14-2 | Roots | 1 | 99.87 | 95.19 | 108.51 | 74.00 | 203.07 |
| | | 10 | 84.12 | 110.41 | 57.28 | 69.46 | 216.41 |
| | | 100 | 73.83 | 158.54 | 11.79 | 71.20 | 395.30 |
| 14-3 | Stems | 1 | 100.32 | 97.34 | 107.19 | 71.53 | 124.19 |
| | | 10 | 94.34 | 112.05 | 72.15 | 62.56 | 232.69 |
| | | 100 | 77.10 | 151.05 | 30.81 | 61.11 | 394.75 |
| 14-4 | Leaves | 1 | 99.54 | 108.31 | 84.11 | 74.30 | 94.13 |
| | | 10 | 94.25 | 131.66 | 56.62 | 80.39 | 277.57 |
| | | 100 | 80.79 | 138.58 | 19.42 | 83.21 | 443.06 |
| 14-5 | Fruits | 1 | 101.86 | 114.54 | 97.33 | 68.71 | 172.44 |
| | | 10 | 93.58 | 132.49 | 90.68 | 94.44 | 214.06 |
| | | 100 | 76.63 | 147.46 | 19.32 | 102.92 | 349.68 |
| 14-6 | Flowers | 1 | 102.03 | 97.52 | 97.77 | 70.63 | 210.59 |
| | | 10 | 83.01 | 111.62 | 58.53 | 79.69 | 209.35 |
| | | 100 | 70.65 | 158.92 | 32.93 | 91.16 | 483.29 |

### Example 15. Yellow Mongolian Snakegourd

TABLE 31

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 15 | Yellow | | | | | | |
| | Mongolian | | | | | | |
| | snakegourd | | | | | | |
| 15-1 | Whole plant | 1 | 30.38 | 56.89 | 168.56 | 94.87 | 117.69 |
| | | 10 | 39.98 | 32.70 | 219.87 | 110.33 | 223.10 |
| | | 100 | 85.60 | 22.75 | 466.90 | 333.89 | 306.01 |
| 15-2 | Roots | 1 | 24.68 | 84.79 | 141.57 | 91.44 | 116.63 |
| | | 10 | 32.05 | 39.35 | 332.07 | 159.10 | 217.82 |
| | | 100 | 78.60 | 22.63 | 460.99 | 480.51 | 305.48 |
| 15-3 | Stems | 1 | 23.03 | 54.28 | 117.16 | 81.50 | 86.06 |
| | | 10 | 42.46 | 38.17 | 319.03 | 138.60 | 100.34 |
| | | 100 | 76.25 | 22.91 | 419.34 | 398.32 | 320.67 |
| 15-4 | Leaves | 1 | 28.55 | 48.72 | 200.24 | 83.95 | 129.60 |
| | | 10 | 49.44 | 40.56 | 244.98 | 181.59 | 194.22 |
| | | 100 | 76.51 | 29.56 | 414.14 | 500.92 | 241.09 |
| 15-5 | Fruits | 1 | 24.66 | 75.70 | 129.46 | 86.84 | 127.27 |
| | | 10 | 35.20 | 31.14 | 227.74 | 122.00 | 229.62 |
| | | 100 | 75.56 | 27.20 | 425.17 | 475.16 | 282.57 |
| 15-6 | Flowers | 1 | 29.87 | 68.99 | 142.09 | 103.58 | 126.82 |
| | | 10 | 38.59 | 38.41 | 220.23 | 176.79 | 239.84 |
| | | 100 | 74.25 | 25.76 | 384.69 | 343.17 | 245.24 |

TABLE 32

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 15 | Yellow | | | | | | |
| | Mongolian | | | | | | |
| | snakegourd | | | | | | |
| 15-1 | Whole plant | 1 | 100.96 | 100.65 | 95.43 | 80.77 | 168.58 |
| | | 10 | 95.86 | 110.42 | 95.42 | 90.05 | 301.85 |
| | | 100 | 70.29 | 156.50 | 34.53 | 110.56 | 343.94 |
| 15-2 | Roots | 1 | 97.37 | 106.39 | 108.95 | 80.68 | 12.8.21 |
| | | 10 | 85.32 | 129.41 | 47.11 | 77.56 | 220.38 |
| | | 100 | 79.83 | 158.13 | 8.75 | 97.03 | 334.03 |
| 15-3 | Stems | 1 | 98.19 | 102.03 | 82.77 | 80.01 | 99.49 |
| | | 10 | 91.82 | 120.36 | 76.27 | 75.18 | 171.81 |
| | | 100 | 75.50 | 139.76 | 46.66 | 109.76 | 488.40 |
| 15-4 | Leaves | 1 | 102.64 | 102.98 | 83.72 | 81.87 | 95.26 |
| | | 10 | 90.30 | 106.44 | 66.92 | 89.34 | 199.50 |
| | | 100 | 70.91 | 134.96 | 25.68 | 104.04 | 441.94 |
| 15-5 | Fruits | 1 | 101.53 | 109.83 | 101.97 | 76.45 | 98.77 |
| | | 10 | 81.36 | 120.91 | 48.47 | 93.97 | 327.35 |
| | | 100 | 77.17 | 144.43 | 45.98 | 105.29 | 459.43 |
| 15-6 | Flowers | 1 | 100.28 | 105.05 | 106.99 | 85.91 | 108.15 |
| | | 10 | 80.04 | 133.84 | 67.01 | 82.73 | 224.06 |
| | | 100 | 79.04 | 140.45 | 354 | 109.15 | 357.36 |

### Example 16. Pungdo Solomon's Seal

TABLE 33

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 16 | Pungdo | | | | | | |
| | Solomon's seal | | | | | | |
| 16-1 | Whole plant | 1 | 28.63 | 63.69 | 169.75 | 96.48 | 124.15 |
| | | 10 | 36.26 | 36.88 | 321.82 | 184.94 | 141.47 |
| | | 100 | 84.91 | 30.88 | 427.40 | 425.44 | 292.48 |
| 16-2 | Roots | 1 | 27.19 | 72.35 | 115.00 | 96.11 | 112.58 |
| | | 10 | 43.64 | 35.70 | 340.33 | 139.96 | 137.26 |
| | | 100 | 71.57 | 29.48 | 369.43 | 343.48 | 241.04 |
| 16-3 | Stems | 1 | 24.18 | 74.38 | 125.82 | 109.06 | 82.46 |
| | | 10 | 33.86 | 30.00 | 311.86 | 185.11 | 161.50 |
| | | 100 | 79.54 | 30.11 | 453.65 | 329.90 | 328.58 |
| 16-4 | Leaves | 1 | 23.53 | 57.76 | 193.28 | 89.71 | 113.47 |
| | | 10 | 35.09 | 38.10 | 262.43 | 157.54 | 149.85 |
| | | 100 | 76.93 | 26.93 | 370.66 | 412.67 | 269.97 |
| 16-5 | Fruits | 1 | 30.80 | 82.42 | 113.62 | 106.26 | 84.83 |
| | | 10 | 38.57 | 38.91 | 272.29 | 111.91 | 104.76 |
| | | 100 | 75.43 | 28.77 | 481.70 | 423.54 | 287.93 |
| 16-6 | Flowers | 1 | 29.26 | 77.02 | 104.07 | 100.76 | 93.75 |
| | | 10 | 42.17 | 38.77 | 318.44 | 198.12 | 113.47 |
| | | 100 | 73.74 | 21.78 | 492.36 | 409.81 | 231.36 |

TABLE 34

| | | | | Blood | Cognitive | | |
|---|---|---|---|---|---|---|---|
| | | | Anti- | glucose- | ability-β- | | Andropanse-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | ( % of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 16 | Pungdo | | | | | | |
| | Solomon's seal | | | | | | |
| 16-1 | Whole plant | 1 | 96.92 | 110.84 | 101.94 | 71.17 | 119.45 |
| | | 10 | 92.29 | 118.24 | 74.79 | 82.71 | 332.58 |
| | | 100 | 80.97 | 147.18 | 47.26 | 91.15 | 452.40 |
| 16-2 | Roots | 1 | 99.77 | 111.55 | 96.50 | 80.21 | 101.79 |
| | | 10 | 80.41 | 133.86 | 70.12 | 75.52 | 211.23 |
| | | 100 | 72.21 | 132.34 | 5.64 | 96.73 | 363.29 |
| 16-3 | Stems | 1 | 97.33 | 113.21 | 100.56 | 65.77 | 112.29 |
| | | 10 | 86.17 | 110.67 | 80.87 | 76.60 | 194.71 |
| | | 100 | 74.05 | 135.31 | 9.53 | 93.55 | 404.36 |
| 16-4 | Leaves | 1 | 102.14 | 102.27 | 103.59 | 84.21 | 167.64 |
| | | 10 | 90.13 | 135.10 | 46.48 | 93.86 | 248.39 |
| | | 100 | 71.67 | 151.42 | 50.58 | 101.31 | 465.24 |
| 16-5 | Fruits | 1 | 97.62 | 112.89 | 104.19 | 82.11 | 110.81 |
| | | 10 | 88.68 | 137.29 | 64.19 | 89.76 | 200.45 |
| | | 100 | 70.41 | 149.52 | 29.10 | 98.43 | 354.12 |
| 16-6 | Flowers | 1 | 97.09 | 110.85 | 102.25 | 75.50 | 153.79 |
| | | 10 | 95.42 | 114.84 | 55.51 | 94.26 | 220.85 |
| | | 100 | 71.97 | 148.22 | 31.82 | 101.58 | 304.92 |

### Example 17. Songak

TABLE 35

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 17 | Songak | | | | | | |
| 17-1 | Whole plant | 1 | 29.17 | 80.67 | 178.33 | 110.08 | 123.31 |
| | | 10 | 32.55 | 35.59 | 216.70 | 198.60 | 145.57 |
| | | 100 | 73.42 | 20.85 | 406.87 | 327.83 | 220.41 |
| 17-2 | Roots | 1 | 23.82 | 49.89 | 114.36 | 89.26 | 116.11 |
| | | 10 | 50.94 | 32.24 | 292.51 | 135.77 | 151.03 |
| | | 100 | 80.93 | 26.64 | 398.00 | 306.19 | 309.41 |
| 17-3 | Stems | 1 | 22.62 | 64.62 | 183.36 | 108.70 | 150.32 |
| | | 10 | 31.85 | 36.27 | 342.99 | 143.92 | 182.91 |
| | | 100 | 73.29 | 25.30 | 416.44 | 364.04 | 216.15 |
| 17-4 | Leaves | 1 | 25.91 | 83.48 | 165.69 | 107.75 | 98.96 |
| | | 10 | 42.23 | 36.20 | 322.66 | 174.35 | 163.86 |
| | | 100 | 70.68 | 23.01 | 476.51 | 432.85 | 324.27 |
| 17-5 | Fruits | 1 | 27.91 | 90.57 | 188.11 | 108.13 | 126.21 |
| | | 10 | 41.88 | 39.44 | 274.49 | 192.15 | 246.10 |
| | | 100 | 80.75 | 29.50 | 422.74 | 318.08 | 271.97 |
| 17-6 | Flowers | 1 | 22.66 | 60.36 | 155.22 | 100.08 | 94.07 |
| | | 10 | 38.43 | 31.86 | 343.56 | 124.94 | 224.17 |
| | | 100 | 77.72 | 25.12 | 486.03 | 301.15 | 271.95 |

TABLE 36

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 17 | Songak | | | | | | |
| | Whole plant | 1 | 100.69 | 111.58 | 89.95 | 70.52 | 116.24 |
| | | 10 | 81.61 | 131.99 | 49.50 | 90.77 | 259.13 |
| | | 100 | 79.48 | 157.50 | 20.31 | 90.56 | 462.57 |
| 17-2 | Roots | 1 | 98.70 | 99.84 | 98.94 | 78.73 | 100.82 |
| | | 10 | 94.89 | 123.83 | 84.25 | 95.86 | 287.75 |
| | | 100 | 80.93 | 155.90 | 33.05 | 107.11 | 389.99 |
| 17-3 | Stems | 1 | 97.00 | 100.90 | 104.97 | 82.25 | 132.32 |
| | | 10 | 87.21 | 136.26 | 76.91 | 82.02 | 223.81 |
| | | 100 | 77.24 | 130.08 | 26.34 | 105.32 | 345.74 |
| 17-4 | Leaves | 1 | 100.03 | 109.26 | 85.12 | 78.77 | 173.86 |
| | | 10 | 81.90 | 112.04 | 80.19 | 83.59 | 171.51 |
| | | 100 | 70.47 | 142.14 | 40.97 | 101.93 | 379.72 |
| 17-5 | Fruits | 1 | 99.01 | 104.03 | 89.97 | 67.98 | 169.83 |
| | | 10 | 90.66 | 118.97 | 48.45 | 85.11 | 297.29 |
| | | 100 | 74.43 | 130.54 | 36.36 | 104.14 | 437.89 |
| 17-6 | Flowers | 1 | 99.21 | 110.46 | 104.40 | 69.38 | 92.04 |
| | | 10 | 89.97 | 107.71 | 65.04 | 95.62 | 302.77 |
| | | 100 | 70.50 | 158.60 | 0.97 | 92.49 | 497.93 |

### Example 18. Ulleungdo Sweet Violet

TABLE 37

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 18 | Ulleungdo | | | | | | |
| | sweet violet | | | | | | |
| 18-1 | Whole plant | 1 | 22.23 | 75.71 | 125.42 | 92.72 | 103.55 |
| | | 10 | 33.53 | 35.09 | 285.73 | 114.93 | 128.18 |
| | | 100 | 83.01 | 23.77 | 460.31 | 350.01 | 231.67 |
| 18-2 | Roots | 1 | 27.15 | 86.14 | 181.86 | 82.90 | 109.91 |
| | | 10 | 39.25 | 38.78 | 273.59 | 187.18 | 164.42 |
| | | 100 | 76.13 | 28.33 | 382.92 | 401.75 | 233.99 |
| 18-3 | Stems | 1 | 25.73 | 54.64 | 106.81 | 99.32 | 85.38 |
| | | 10 | 49.12 | 34.04 | 300.92 | 157.73 | 118.83 |
| | | 100 | 81.50 | 20.86 | 419.11 | 407.30 | 222.67 |
| 18-4 | Leaves | 1 | 24.29 | 70.44 | 158.94 | 89.98 | 121.06 |
| | | 10 | 31.50 | 40.02 | 328.85 | 194.90 | 146.50 |
| | | 100 | 79.99 | 20.12 | 465.74 | 474.24 | 226.47 |
| 18-5 | Fruits | 1 | 29.00 | 87.33 | 191.15 | 82.24 | 112.41 |
| | | 10 | 38.90 | 39.19 | 335.79 | 135.00 | 189.19 |
| | | 100 | 77.01 | 27.11 | 371.14 | 490.29 | 338.12 |
| 18-6 | Flowers | 1 | 29.10 | 65.95 | 191.82 | 96.97 | 85.67 |
| | | 10 | 36.15 | 37.42 | 274.95 | 152.31 | 196.72 |
| | | 100 | 75.39 | 24.69 | 482.84 | 349.57 | 216.85 |

TABLE 38

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Sily marin | (Dihydro- |
| | | | methasone | 1 mM) | Inhibitor IV | 100 ppm) | testosterone |
| | | | 1 uM) | | 15 nM) | | 100 nM) |
| 18 | Ulleungdo | | | | | | |
| | sweet violet | | | | | | |
| 18-1 | Whole plant | 1 | 96.05 | 112.56 | 92.23 | 72.47 | 147.97 |
| | | 10 | 84.38 | 130.84 | 79.68 | 82.79 | 278.16 |
| | | 100 | 71.07 | 159.16 | 20.36 | 109.13 | 374.86 |
| 18-2 | Roots | 1 | 101.18 | 103.31 | 81.18 | 83.58 | 187.09 |
| | | 10 | 92.96 | 124.66 | 69.56 | 84.24 | 225.75 |
| | | 100 | 73.63 | 148.85 | 2.00 | 96.03 | 480.65 |
| 18-3 | Stems | 1 | 100.26 | 108.83 | 106.71 | 75.26 | 193.63 |
| | | 10 | 84.49 | 123.68 | 48.88 | 86.88 | 189.25 |
| | | 100 | 70.62 | 136.44 | 3.44 | 102.49 | 478.49 |
| 18-4 | Leaves | 1 | 97.48 | 96.74 | 95.99 | 84.46 | 113.85 |
| | | 10 | 94.64 | 118.26 | 72.04 | 78.08 | 288.73 |
| | | 100 | 78.00 | 133.30 | 45.30 | 105.39 | 428.23 |
| 18-5 | Fruits | 1 | 103.86 | 106.64 | 100.81 | 80.95 | 150.48 |
| | | 10 | 89.14 | 105.01 | 81.59 | 85.83 | 211.75 |
| | | 100 | 80.47 | 138.09 | 45.77 | 99.09 | 446.59 |
| 18-6 | Flowers | 1 | 101.35 | 111.28 | 84.87 | 82.13 | 144.06 |
| | | 10 | 93.04 | 140.03 | 94.57 | 87.85 | 218.52 |
| | | 100 | 78.07 | 155.53 | 41.01 | 90.09 | 326.57 |

### Example 19. Korean Bellflower

TABLE 39

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 19 | Korean | | | | | | |
| | bellflower | | | | | | |
| 19-1 | Whole plant | 1 | 24.00 | 41.22 | 140.51 | 94.23 | 139.96 |
| | | 10 | 48.47 | 31.85 | 313.62 | 200.58 | 244.37 |
| | | 100 | 70.99 | 24.36 | 457.27 | 346.88 | 332.90 |
| 19-2 | Roots | 1 | 22.13 | 74.77 | 156.23 | 110.53 | 103.00 |
| | | 10 | 34.75 | 39.06 | 239.02 | 184.21 | 229.11 |
| | | 100 | 70.39 | 25.90 | 419.48 | 304.51 | 309.57 |
| 19-3 | Stems | 1 | 22.89 | 58.26 | 159.48 | 82.85 | 123.78 |
| | | 10 | 37.14 | 30.65 | 289.13 | 165.99 | 122.66 |
| | | 100 | 75.52 | 24.52 | 404.39 | 475.07 | 292.83 |
| 19-4 | Leaves | 1 | 21.23 | 78.50 | 113.49 | 84.22 | 113.29 |
| | | 10 | 46.29 | 34.14 | 345.44 | 143.64 | 107.83 |
| | | 100 | 78.14 | 23.37 | 465.19 | 459.79 | 291.88 |
| 19-5 | Fruits | 1 | 28.45 | 57.48 | 168.19 | 99.72 | 115.90 |
| | | 10 | 44.63 | 39.58 | 298.86 | 135.12 | 112.26 |
| | | 100 | 83.14 | 26.93 | 395.03 | 428.58 | 335.93 |
| 19-6 | Flowers | 1 | 30.25 | 45.76 | 100.21 | 98.95 | 101.84 |
| | | 10 | 41.28 | 33.86 | 293.93 | 193.53 | 220.35 |
| | | 100 | 75.91 | 29.31 | 365.66 | 339.62 | 221.04 |

TABLE 40

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 19 | Korean | | | | | | |
| | bellflower | | | | | | |
| 19-1 | Whole plant | 1 | 98.88 | 110.20 | 100.85 | 66.69 | 99.68 |
| | | 10 | 88.02 | 126.64 | 75.34 | 90.85 | 281.82 |
| | | 100 | 73.68 | 130.39 | 49.62 | 96.14 | 439.59 |
| 19-2 | Roots | 1 | 101.00 | 95.99 | 80.56 | 81.45 | 117.14 |
| | | 10 | 90.83 | 108.30 | 80.76 | 77.23 | 243.05 |
| | | 100 | 72.25 | 160.15 | 23.08 | 101.11 | 495.83 |
| 19-3 | Stems | 1 | 95.63 | 100.75 | 90.44 | 85.20 | 169.45 |
| | | 10 | 89.63 | 131.92 | 68.61 | 79.12 | 290.66 |
| | | 100 | 73.71 | 147.80 | 1.69 | 100.40 | 472.06 |
| 19-4 | Leaves | 1 | 97.13 | 114.21 | 103.06 | 76.02 | 141.59 |
| | | 10 | 86.00 | 111.89 | 51.06 | 82.82 | 236.64 |
| | | 100 | 79.28 | 149.87 | 27.31 | 98.94 | 474.97 |
| 19-5 | Fruits | 1 | 96.25 | 96.30 | 103.09 | 73.63 | 143.07 |
| | | 10 | 84.03 | 139.39 | 92.05 | 95.30 | 297.29 |
| | | 100 | 73.53 | 149.35 | 43.86 | 94.38 | 454.05 |
| 19-6 | Flowers | 1 | 98.81 | 112.06 | 101.44 | 81.27 | 201.53 |
| | | 10 | 84.60 | 114.14 | 54.43 | 79.45 | 224.18 |
| | | 100 | 74.81 | 149.69 | 5.19 | 101.43 | 436.46 |

### Example 20. Ulleungdo Turk's-Cap Lily

TABLE 41

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 20 | Ulleungdo | | | | | | |
| | turk's-cap lily | | | | | | |
| 20-1 | Whole plant | 1 | 20.29 | 52.58 | 183.31 | 107.96 | 129.71 |
| | | 10 | 31.74 | 31.68 | 224.70 | 134.47 | 132.14 |
| | | 100 | 81.64 | 25.37 | 429.50 | 339.46 | 339.60 |
| 20-2 | Roots | 1 | 23.28 | 49.11 | 126.15 | 103.09 | 108.12 |
| | | 10 | 48.43 | 40.61 | 241.14 | 168.04 | 189.37 |
| | | 100 | 75.94 | 22.59 | 470.58 | 457.46 | 280.03 |
| 20-3 | Stems | 1 | 23.76 | 54.00 | 191.15 | 97.84 | 97.62 |
| | | 10 | 45.76 | 39.90 | 244.34 | 157.90 | 126.37 |
| | | 100 | 78.38 | 23.77 | 491.32 | 351.95 | 331.08 |
| 20-4 | Leaves | 1 | 25.68 | 62.93 | 133.49 | 98.74 | 103.64 |
| | | 10 | 30.76 | 37.19 | 266.99 | 164.99 | 118.85 |
| | | 100 | 79.98 | 26.61 | 471.22 | 450.88 | 253.58 |
| 20-5 | Fruits | 1 | 25.27 | 86.24 | 107.13 | 96.24 | 95.42 |
| | | 10 | 43.66 | 39.32 | 308.62 | 138.34 | 106.55 |
| | | 100 | 70.83 | 26.90 | 458.43 | 429.67 | 211.01 |
| 20-6 | Flowers | 1 | 28.31 | 69.56 | 151.51 | 98.99 | 94.32 |
| | | 10 | 41.38 | 31.47 | 203.07 | 192.08 | 140.73 |
| | | 100 | 85.52 | 22.93 | 405.65 | 310.76 | 316.96 |

TABLE 42

| | | | | Blood | Cognitive | | |
|---|---|---|---|---|---|---|---|
| | | | Anti- | glucose- | ability-p- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 20 | Ulleungdo | | | | | | |
| | turk's-cap lily | | | | | | |
| 20-1 | Whole plant | 1 | 97.65 | 98.65 | 94.72 | 68.36 | 199.95 |
| | | 10 | 93.29 | 114.41 | 82.52 | 86.66 | 298.74 |
| | | 100 | 74.48 | 149.74 | 15.89 | 92.70 | 374.14 |
| 20-2 | Roots | 1 | 101.45 | 98.27 | 107.72 | 77.78 | 183.69 |
| | | 10 | 87.12 | 130.62 | 48.43 | 78.53 | 228.94 |
| | | 100 | 71.09 | 136.51 | 22.61 | 103.76 | 421.11 |
| 20-3 | Stems | 1 | 98.30 | 113.21 | 91.25 | 85.08 | 111.42 |
| | | 10 | 92.83 | 122.66 | 83.81 | 81.80 | 246.71 |
| | | 100 | 71.88 | 149.64 | 19.15 | 98.75 | 334.67 |
| 20-4 | Leaves | 1 | 98.01 | 115.78 | 91.60 | 74.05 | 188.14 |
| | | 10 | 95.25 | 125.96 | 88.57 | 86.32 | 228.23 |
| | | 100 | 74.11 | 146.47 | 48.04 | 110.90 | 392.31 |
| 20-5 | Fruits | 1 | 101.08 | 105.85 | 91.30 | 67.42 | 97.47 |
| | | 10 | 81.39 | 139.41 | 78.57 | 77.02 | 268.92 |
| | | 100 | 70.69 | 160.56 | 3.96 | 90.13 | 309.59 |
| 20-6 | Flowers | 1 | 101.86 | 110.69 | 104.44 | 84.44 | 173.09 |
| | | 10 | 87.48 | 134.50 | 49.92 | 79.77 | 240.95 |
| | | 100 | 73.80 | 160.89 | 38.36 | 98.55 | 392.02 |

### Example 21. Ulleungdo Spike Speedwell

TABLE 43

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 uM) | 10 μM) | | 1 ng/ml) |
| 21 | Ulleungdo | | | | | | |
| | spike speedwell | | | | | | |
| 21-1 | Whole plant | 1 | 24.22 | 87.04 | 159.01 | 82.93 | 83.94 |
| | | 10 | 32.03 | 30.10 | 205.62 | 122.37 | 201.16 |
| | | 100 | 81.96 | 29.15 | 452.63 | 451.29 | 323.72 |
| 21-2 | Roots | 1 | 20.98 | 76.81 | 182.07 | 100.15 | 103.15 |
| | | 10 | 31.54 | 33.91 | 310.30 | 148.70 | 235.68 |
| | | 100 | 84.41 | 28.75 | 393.03 | 372.90 | 243.45 |
| 21-3 | Stems | 1 | 25.67 | 65.99 | 103.07 | 102.43 | 86.34 |
| | | 10 | 45.74 | 30.84 | 327.57 | 113.43 | 183.44 |
| | | 100 | 80.34 | 29.77 | 381.62 | 481.18 | 209.69 |
| 21-4 | Leaves | 1 | 23.84 | 45.14 | 197.00 | 81.67 | 149.03 |
| | | 10 | 50.89 | 38.17 | 201.26 | 193.16 | 150.51 |
| | | 100 | 77.88 | 27.11 | 382.62 | 305.06 | 202.59 |
| 21-5 | Fruits | 1 | 25.82 | 69.73 | 117.70 | 82.23 | 98.36 |
| | | 10 | 46.34 | 39.17 | 282.20 | 148.28 | 242.88 |
| | | 100 | 78.60 | 24.04 | 456.52 | 422.39 | 281.27 |
| 21-6 | Flowers | 1 | 22.25 | 63.82 | 102.42 | 84.14 | 96.38 |
| | | 10 | 50.75 | 33.20 | 254.23 | 197.05 | 108.05 |
| | | 100 | 81.54 | 29.77 | 492.27 | 467.55 | 232.20 |

TABLE 44

| | | | | Blood | Cognitive | | |
|---|---|---|---|---|---|---|---|
| | | | Anti- | glucose- | ability-β- | | Andropanse-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 21 | Ulleungdo | | | | | | |
| | spike speedwell | | | | | | |
| 21-1 | Whole plant | 1 | 96.22 | 113.77 | 80.86 | 75.80 | 176.22 |
| | | 10 | 89.86 | 115.53 | 89.35 | 86.30 | 319.26 |
| | | 100 | 72.54 | 139.57 | 5.73 | 106.35 | 429.58 |
| 21-2 | Roots | 1 | 101.36 | 107.75 | 94.09 | 85.02 | 161.17 |
| | | 10 | 90.45 | 125.57 | 51.93 | 94.46 | 218.38 |
| | | 100 | 79.56 | 136.55 | 25.32 | 97.87 | 478.09 |
| 21-3 | Stems | 1 | 103.81 | 101.09 | 100.52 | 85.12 | 117.81 |
| | | 10 | 84.00 | 127.03 | 45.45 | 85.20 | 179.25 |
| | | 100 | 71.04 | 153.54 | 41.01 | 94.16 | 487.59 |
| 21-4 | Leaves | 1 | 95.62 | 113.62 | 97.57 | 82.27 | 143.18 |
| | | 10 | 90.10 | 117.35 | 71.97 | 84.21 | 262.15 |
| | | 100 | 77.70 | 141.50 | 12.49 | 102.07 | 345.74 |
| 21-5 | Fruits | 1 | 102.88 | 110.73 | 105.53 | 73.08 | 152.35 |
| | | 10 | 94.60 | 129.01 | 74.83 | 78.37 | 253.81 |
| | | 100 | 76.26 | 150.50 | 11.72 | 99.48 | 421.44 |
| 21-6 | Flowers | 1 | 97.52 | 111.74 | 86.55 | 66.45 | 106.88 |
| | | 10 | 85.68 | 116.63 | 63.57 | 86.53 | 204.32 |
| | | 100 | 72.27 | 146.20 | 30.41 | 96.13 | 404.06 |

### Example 22. Coastal Moss-Like Stonecrop

TABLE 45

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 22 | Coastal | | | | | | |
| | moss-like | | | | | | |
| | stonecrop | | | | | | |
| 22-1 | Whole plant | 1 | 25.21 | 62.22 | 167.00 | 86.74 | 104.40 |
| | | 10 | 42.68 | 31.69 | 299.54 | 188.65 | 248.60 |
| | | 100 | 74.41 | 29.28 | 354.94 | 492.63 | 257.00 |
| 22-2 | Roots | 1 | 20.18 | 75.52 | 140.40 | 107.45 | 118.15 |
| | | 10 | 48.98 | 37.28 | 259.33 | 195.04 | 177.34 |
| | | 100 | 84.35 | 26.28 | 369.34 | 347.79 | 346.72 |
| 22-3 | Stems | 1 | 21.45 | 83.59 | 166.08 | 84.24 | 142.09 |
| | | 10 | 34.76 | 32.10 | 238.71 | 156.73 | 163.77 |
| | | 100 | 80.01 | 28.93 | 368.37 | 426.92 | 200.63 |
| 22-4 | Leaves | 1 | 29.45 | 40.08 | 189.55 | 87.35 | 105.73 |
| | | 10 | 32.80 | 37.49 | 309.73 | 187.20 | 183.47 |
| | | 100 | 72.91 | 25.32 | 390.30 | 405.38 | 284.79 |
| 22-5 | Fruits | 1 | 27.84 | 76.43 | 114.11 | 90.01 | 131.19 |
| | | 10 | 34.82 | 30.34 | 332.74 | 183.49 | 238.74 |
| | | 100 | 80.14 | 24.84 | 383.33 | 320.53 | 208.09 |
| 22-6 | Flowers | 1 | 30.05 | 75.93 | 147.05 | 96.60 | 87.37 |
| | | 10 | 46.72 | 34.39 | 249.64 | 141.16 | 213.08 |
| | | 100 | 70.43 | 20.89 | 363.33 | 366.41 | 337.79 |

TABLE 46

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver heaith- | transcriptional |
| | | Conc. | secretion | absorption | activity | Cell viability | activity |
| No. | Sample | (ppm) | (% of control) | (% of control) | (% of control) | ( % of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 22 | Coastal | | | | | | |
| | moss-like | | | | | | |
| | stonecrop | | | | | | |
| 22-1 | Whole plant | 1 | 98.54 | 103.40 | 83.21 | 80.08 | 197.20 |
| | | 10 | 87.78 | 105.50 | 64.77 | 93.35 | 324.52 |
| | | 100 | 70.74 | 156.97 | 15.51 | 97.44 | 459.59 |
| 22-2 | Roots | 1 | 98.83 | 110.71 | 106.03 | 68.31 | 121.61 |
| | | 10 | 81.88 | 123.53 | 51.12 | 84.15 | 298.84 |
| | | 100 | 73.84 | 152.74 | 32.39 | 110.31 | 397.41 |
| 22-3 | Stems | 1 | 97.65 | 112.84 | 106.30 | 80.88 | 193.73 |
| | | 10 | 91.86 | 124.05 | 45.91 | 92.05 | 220.44 |
| | | 100 | 70.79 | 157.51 | 18.93 | 107.01 | 322.84 |
| 22-4 | Leaves | 1 | 101.09 | 97.26 | 88.43 | 72.17 | 132.80 |
| | | 10 | 82.69 | 125.98 | 67.17 | 89.31 | 256.88 |
| | | 100 | 73.06 | 160.47 | 42.40 | 91.49 | 345.45 |
| 22-5 | Fruits | 1 | 100.50 | 104.02 | 80.01 | 67.29 | 177.38 |
| | | 10 | 91.21 | 139.17 | 87.80 | 91.93 | 245.80 |
| | | 100 | 73.66 | 137.01 | 34.78 | 99.54 | 339.81 |
| 22-6 | Flowers | 1 | 97.14 | 99.15 | 100.84 | 83.04 | 102.39 |
| | | 10 | 84.86 | 123.84 | 76.49 | 84.44 | 315.57 |
| | | 100 | 74.89 | 146.17 | 11.60 | 108.10 | 308.76 |

### Example 23. Water Chickweed

TABLE 47

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 23 | Water | | | | | | |
| | chickweed | | | | | | |
| 23-1 | Whole plant | 1 | 20.41 | 82.51 | 184.54 | 89.68 | 103.43 |
| | | 10 | 35.74 | 38.41 | 344.63 | 148.85 | 137.16 |
| | | 100 | 80.94 | 26.38 | 483.97 | 447.13 | 290.31 |
| 23-2 | Roots | 1 | 26.35 | 87.30 | 114.88 | 92.57 | 107.53 |
| | | 10 | 37.57 | 39.96 | 273.74 | 163.61 | 190.79 |
| | | 100 | 82.95 | 25.65 | 496.34 | 363.70 | 235.58 |
| 23-3 | Stems | 1 | 22.35 | 83.63 | 121.17 | 83.55 | 108.20 |
| | | 10 | 40.77 | 31.34 | 303.69 | 194.04 | 110.89 |
| | | 100 | 71.02 | 21.20 | 497.95 | 314.39 | 309.84 |
| 23-4 | Leaves | 1 | 26.80 | 83.71 | 156.66 | 100.65 | 135.50 |
| | | 10 | 40.14 | 33.91 | 277.03 | 131.55 | 125.58 |
| | | 100 | 78.13 | 25.12 | 384.91 | 478.55 | 331.62 |
| 23-5 | Fruits | 1 | 26.20 | 70.92 | 146.48 | 100.97 | 110.77 |
| | | 10 | 39.68 | 32.92 | 345.90 | 110.58 | 216.23 |
| | | 100 | 85.32 | 29.61 | 475.24 | 431.75 | 272.78 |
| 23-6 | Flowers | 1 | 22.16 | 40.86 | 186.91 | 91.60 | 132.63 |
| | | 10 | 40.70 | 31.85 | 243.28 | 184.64 | 138.94 |
| | | 100 | 71.98 | 25.26 | 409.66 | 372.90 | 337.16 |

TABLE 48

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 23 | Water | | | | | | |
| | chickweed | | | | | | |
| 23-1 | Whole plant | 1 | 97.10 | 112.41 | 94.20 | 78.48 | 152.36 |
| | | 10 | 86.32 | 126.43 | 78.35 | 75.01 | 262.62 |
| | | 100 | 74.81 | 149.65 | 11.51 | 92.07 | 365.41 |
| 23-2 | Roots | 1 | 98.52 | 101.16 | 107.96 | 74.12 | 91.82 |
| | | 10 | 86.13 | 122.90 | 46.64 | 75.43 | 260.45 |
| | | 100 | 79.40 | 150.48 | 24.08 | 97.10 | 432.55 |
| 23-3 | Stems | 1 | 98.34 | 97.50 | 109.89 | 71.46 | 159.71 |
| | | 10 | 93.28 | 114.44 | 60.81 | 87.01 | 209.75 |
| | | 100 | 79.20 | 133.91 | 47.35 | 104.39 | 394.77 |
| 23-4 | Leaves | 1 | 103.71 | 113.48 | 88.38 | 85.98 | 171.20 |
| | | 10 | 89.99 | 115.05 | 61.84 | 93.58 | 256.89 |
| | | 100 | 76.40 | 140.30 | 47.27 | 95.44 | 435.74 |
| 23-5 | Fruits | 1 | 96.91 | 102.65 | 107.18 | 75.95 | 181.89 |
| | | 10 | 86.13 | 131.42 | 74.73 | 90.44 | 316.03 |
| | | 100 | 75.47 | 144.17 | 47.88 | 92.86 | 389.16 |
| 23-6 | Flowers | 1 | 96.43 | 111.24 | 81.25 | 68.77 | 174.16 |
| | | 10 | 95.38 | 119.81 | 51.11 | 95.19 | 201.33 |
| | | 100 | 74.40 | 153.78 | 27.27 | 100.54 | 491.64 |

### Example 24. Heartleaf Houttuynia

TABLE 49

| | | | Blood | Lipid- | Anti-obesity- | | Menopause- |
|---|---|---|---|---|---|---|---|
| | | | circulation- | choiesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 24 | Heartleaf | | | | | | |
| | Houttuynia | | | | | | |
| 24-1 | Whole plant | 1 | 30.52 | 49.33 | 183.17 | 95.75 | 81.45 |
| | | 10 | 36.03 | 30.03 | 240.49 | 146.21 | 249.46 |
| | | 100 | 83.13 | 20.07 | 361.24 | 388.58 | 219.07 |
| 24-2 | Roots | 1 | 30.47 | 54.36 | 173.03 | 84.59 | 85.72 |
| | | 10 | 41.98 | 38.62 | 319.33 | 154.35 | 208.52 |
| | | 100 | 71.12 | 29.58 | 365.90 | 435.45 | 249.65 |
| 24-3 | Stems | 1 | 24.18 | 47.05 | 138.07 | 80.51 | 87.11 |
| | | 10 | 37.36 | 40.04 | 231.28 | 134.14 | 237.51 |
| | | 100 | 72.30 | 28.09 | 446.96 | 334.10 | 241.65 |
| 24-4 | Leaves | 1 | 29.18 | 83.87 | 127.86 | 84.91 | 132.36 |
| | | 10 | 42.00 | 33.00 | 212.86 | 110.03 | 138.38 |
| | | 100 | 84.77 | 21.75 | 458.04 | 319.75 | 224.84 |
| 24-5 | Fruits | 1 | 20.44 | 63.46 | 142.31 | 102.57 | 121.13 |
| | | 10 | 38.75 | 36.84 | 309.47 | 165.11 | 121.40 |
| | | 100 | 82.93 | 24.46 | 410.88 | 443.36 | 243.57 |
| 24-6 | Flowers | 1 | 23.86 | 72.01 | 193.00 | 93.46 | 108.72 |
| | | 10 | 49.96 | 40.70 | 235.04 | 192.68 | 201.66 |
| | | 100 | 76.96 | 26.48 | 483.15 | 436.92 | 260.00 |

TABLE 50

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 24 | Heartleaf | | | | | | |
| | Houttuynia | | | | | | |
| 24-1 | Whole plant | 1 | 96.04 | 114.70 | 99.38 | 80.84 | 195.99 |
| | | 10 | 93.59 | 118.23 | 73.90 | 89.71 | 201.81 |
| | | 100 | 71.14 | 131.91 | 19.96 | 98.39 | 468.84 |
| 24-2 | Roots | 1 | 99.36 | 96.02 | 109.28 | 77.02 | 128.10 |
| | | 10 | 94.28 | 132.41 | 94.66 | 94.06 | 309.61 |
| | | 100 | 70.15 | 138.10 | 32.11 | 108.83 | 440.34 |
| 24-3 | Stems | 1 | 96.46 | 114.30 | 103.73 | 83.52 | 173.41 |
| | | 10 | 93.26 | 120.77 | 62.55 | 83.75 | 212.73 |
| | | 100 | 73.21 | 133.55 | 14.00 | 98.95 | 461.70 |
| 24-4 | Leaves | 1 | 101.71 | 101.85 | 92.24 | 66.56 | 146.13 |
| | | 10 | 82.13 | 136.82 | 65.37 | 83.41 | 279.05 |
| | | 100 | 75.39 | 130.86 | 8.71 | 109.80 | 443.02 |
| 24-5 | Fruits | 1 | 99.22 | 113.53 | 80.58 | 73.60 | 199.53 |
| | | 10 | 92.46 | 114.62 | 95.66 | 90.53 | 253.43 |
| | | 100 | 76.35 | 143.67 | 47.73 | 108.72 | 348.79 |
| 24-6 | Flowers | 1 | 101.04 | 110.11 | 105.58 | 73.53 | 162.01 |
| | | 10 | 92.76 | 136.46 | 70.37 | 79.16 | 188.79 |
| | | 100 | 78.27 | 132.87 | 18.20 | 108.68 | 311.45 |

### Example 25. Ulleungdo Liverleaf

TABLE 51

| | | | Mood | Lipid- | Anti-obesity- | | Menopause- |
|---|---|---|---|---|---|---|---|
| | | | circulation- | cholesterol | Glvcerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 25 | Ulleungdo | | | | | | |
| | liverleaf | | | | | | |
| 25-1 | Whole plant | 1 | 30.47 | 75.36 | 122.61 | 106.54 | 86.00 |
| | | 10 | 42.82 | 40.62 | 254.50 | 116.99 | 193.83 |
| | | 100 | 72.83 | 29.81 | 471.91 | 496.21 | 233.90 |
| 25-2 | Roots | 1 | 29.60 | 41.28 | 142.71 | 88.65 | 92.87 |
| | | 10 | 47.71 | 30.77 | 232.14 | 198.21 | 192.76 |
| | | 100 | 83.34 | 27.85 | 381.10 | 396.54 | 347.67 |
| 25-3 | Stems | 1 | 26.11 | 55.61 | 150.11 | 140.85 | 128.51 |
| | | 10 | 42.46 | 38.51 | 253.63 | 159.71 | 192.15 |
| | | 100 | 81.24 | 21.46 | 363.55 | 415.46 | 300.80 |
| 25-4 | Leaves | 1 | 25.78 | 60.43 | 134.80 | 80.57 | 150.69 |
| | | 10 | 39.90 | 36.83 | 322.74 | 169.16 | 217.23 |
| | | 100 | 79.47 | 28.88 | 392.72 | 448.63 | 266.78 |
| 25-5 | Fruits | 1 | 22.58 | 52.99 | 124.10 | 89.76 | 129.75 |
| | | 10 | 35.07 | 40.93 | 302.76 | 130.49 | 241.63 |
| | | 100 | 77.08 | 27.75 | 500.67 | 471.62 | 298.45 |
| 25-6 | Flowers | 1 | 26.44 | 75.70 | 132.55 | 102.71 | 130.36 |
| | | 10 | 46.47 | 39.67 | 297.71 | 174.37 | 220.72 |
| | | 100 | 82.56 | 24.32 | 496.81 | 452.55 | 349.22 |

TABLE 52

| | | | | Blood | Cognitive | | |
|---|---|---|---|---|---|---|---|
| | | | Anti- | glucose - | ability-β- | | Andropause-AR |
| | | | allergy-IgE | Glucose | secretase | Liver health- | transcriptional |
| | | Conc. | secretion | absorption | activity | Cell viability | activity |
| No. | Sampie | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 25 | Ulleungdo | | | | | | |
| | liverleaf | | | | | | |
| 25-1 | Whole plant | 1 | 96.89 | 98.22 | 107.75 | 83.39 | 162.62 |
| | | 10 | 93.59 | 106.80 | 71.73 | 94.08 | 327.07 |
| | | 100 | 71.25 | 146.17 | 33.92 | 96.85 | 486.60 |
| 25-2 | Roots | 1 | 97.90 | 107.31 | 83.89 | 84.43 | 96.32 |
| | | 10 | 82.04 | 116.80 | 66.28 | 94.52 | 206.10 |
| | | 100 | 79.40 | 132.12 | 41.21 | 100.73 | 351.05 |
| 25-3 | Stems | 1 | 96.23 | 106.48 | 103.25 | 82.81 | 203.54 |
| | | 10 | 84.28 | 134.25 | 46.50 | 84.37 | 253.84 |
| | | 100 | 79.17 | 144.93 | 30.21 | 110.40 | 427.32 |
| 25-4 | Leaves | 1 | 102.97 | 104.96 | 83.73 | 80.97 | 191.69 |
| | | 10 | 89.37 | 137.85 | 47.82 | 94.13 | 265.22 |
| | | 100 | 74.82 | 132.34 | 1.57 | 92.57 | 335.54 |
| 25-5 | Fruits | 1 | 96.20 | 108.36 | 94.12 | 85.65 | 155.93 |
| | | 10 | 84,28 | 107.66 | 70.53 | 90.25 | 202.70 |
| | | 100 | 70.57 | 145.92 | 36.62 | 95.16 | 455.43 |
| 25-6 | Flowers | 1 | 102.93 | 109.57 | 101.08 | 77.21 | 194.92 |
| | | 10 | 85.40 | 126.84 | 85.15 | 89.47 | 295.30 |
| | | 100 | 81.00 | 134.59 | 43.61 | 108.50 | 421.81 |

### Example 26. Chinese Buttercup

TABLE 53

| | | | Blood | Lipid - | Anti-obesity- | | Menopause- |
|---|---|---|---|---|---|---|---|
| | | | circulation- | cholesterol | Glyceroi | Immtunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 26 | Chinese | | | | | | |
| | buttercup | | | | | | |
| 26-1 | Whole plant | 1 | 23.27 | 79.23 | 168.22 | 107.31 | 130.08 |
| | | 10 | 33.98 | 35.91 | 330.81 | 132.45 | 112.45 |
| | | 100 | 78.05 | 29.87 | 405.42 | 404.42 | 296.96 |
| 26-2 | Roots | 1 | 25.65 | 59.50 | 114.14 | 82.91 | 125.31 |
| | | 10 | 47.95 | 40.64 | 290.70 | 166.93 | 155.85 |
| | | 100 | 85.08 | 28.33 | 404.93 | 352.94 | 349.74 |
| 26-3 | Stems | 1 | 24,83 | 65.39 | 111.37 | 92.47 | 140.57 |
| | | 10 | 46.30 | 36.77 | 239.84 | 167.81 | 102.53 |
| | | 100 | 83.34 | 30.09 | 383.27 | 338.74 | 268.16 |
| 26-4 | Leaves | 1 | 20.47 | 60.21 | 138.11 | 102.90 | 99.86 |
| | | 10 | 43.35 | 33.71 | 343.75 | 199.67 | 101.26 |
| | | 100 | 77.89 | 27.77 | 475.33 | 372.06 | 300.94 |
| 26-5 | Fruits | 1 | 26.24 | 72.22 | 152.94 | 84.75 | 115.37 |
| | | 10 | 32.40 | 36.20 | 283.74 | 114.35 | 246.42 |
| | | 100 | 73.63 | 25.10 | 357.14 | 301.45 | 338.29 |
| 26-6 | Flowers | 1 | 28.77 | 45.09 | 193.98 | 81.47 | 88.21 |
| | | 10 | 50.32 | 32.52 | 272.76 | 158.34 | 232.56 |
| | | 100 | 78.92 | 25.11 | 366.37 | 332.79 | 285.55 |

TABLE 54

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver heaith- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin 100 | (Dihydro- |
| | | | methasone | 1 mM) | Inhibitor IV | ppm) | testosterone |
| | | | 1 uM) | | 15 nM) | | 100 nM) |
| 26 | Chinese | | | | | | |
| | buttercup | | | | | | |
| 26-1 | Whole plant | 1 | 99.71 | 110.67 | 82.68 | 76.42 | 165.72 |
| | | 10 | 88.29 | 107.05 | 83.71 | 92.61 | 290.06 |
| | | 100 | 74.03 | 132.56 | 37.20 | 99.67 | 329.49 |
| 26-2 | Roots | 1 | 95.17 | 95.82 | 96.96 | 82.71 | 91.92 |
| | | 10 | 80.19 | 113.66 | 53.63 | 81.18 | 227.47 |
| | | 100 | 78.33 | 142.00 | 40.79 | 104.61 | 393.52 |
| 26-3 | Stems | 1 | 97.49 | 111.37 | 88.46 | 77.70 | 187.74 |
| | | 10 | 92.72 | 133.24 | 91.02 | 85.20 | 323.87 |
| | | 100 | 75.88 | 155.14 | 37.98 | 107.28 | 426.73 |
| 26-4 | Leaves | 1 | 101.65 | 114.55 | 99.04 | 70.40 | 137.41 |
| | | 10 | 89.58 | 127.05 | 80.47 | 92.16 | 313.33 |
| | | 100 | 76.30 | 135.90 | 37.36 | 91.40 | 494.32 |
| 26-5 | Fruits | 1 | 100.46 | 103.12 | 99.87 | 71.91 | 112.23 |
| | | 10 | 81.29 | 125.80 | 83.57 | 75.82 | 205.44 |
| | | 100 | 79.69 | 145.21 | 39.29 | 98.99 | 396.01 |
| 26-6 | Flowers | 1 | 100.18 | 103.16 | 82.10 | 84.05 | 102.54 |
| | | 10 | 82.40 | 130.41 | 62.18 | 86.59 | 172.48 |
| | | 100 | 75.68 | 155.28 | 28.62 | 96.09 | 396.44 |

### Example 27. Tricuspidate Falsenettle

TABLE 55

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 2.7 | Tricuspidate | | | | | | |
| | falsenettle | | | | | | |
| 27-1 | Whole plant | 1 | 25.80 | 42.00 | 117.89 | 86.58 | 84.06 |
| | | 10 | 45.77 | 36.21 | 329.45 | 120.83 | 185.61 |
| | | 100 | 71.52 | 23.65 | 479.74 | 325.99 | 295.28 |
| 27-2 | Roots | 1 | 27.02 | 45.49 | 116.09 | 91.07 | 121.79 |
| | | 10 | 34.81 | 35.08 | 252.80 | 122.02 | 164.00 |
| | | 100 | 85.64 | 29.10 | 495.73 | 467.30 | 326.76 |
| 27-3 | Stems | 1 | 24.04 | 75.72 | 165.67 | 102.06 | 126.06 |
| | | 10 | 45.24 | 32.24 | 244.08 | 169.59 | 191.17 |
| | | 100 | 85.12 | 21.42 | 401.96 | 429.04 | 313.74 |
| 27-4 | Leaves | 1 | 26.06 | 45.34 | 136.82 | 105.78 | 132.50 |
| | | 10 | 33.44 | 40.81 | 264.39 | 196.74 | 215.45 |
| | | 100 | 72.70 | 25.80 | 371.82 | 497.89 | 215.84 |
| 27-5 | Fruits | 1 | 24.71 | 47.82 | 176.38 | 109.17 | 102.99 |
| | | 10 | 33.10 | 33.83 | 232.47 | 197.93 | 216.13 |
| | | 100 | 73.04 | 23.52 | 458.04 | 484.80 | 310.16 |
| 27-6 | Flowers | 1 | 29.86 | 59.72 | 187.22 | 100.35 | 150.21 |
| | | 10 | 42.03 | 37.22 | 209.57 | 194.76 | 248.13 |
| | | 100 | 85.16 | 23.75 | 406.52 | 360.67 | 328.38 |

TABLE 56

| | | | | Blood | Cognitive | | |
|---|---|---|---|---|---|---|---|
| | | | Anti- | glucose- | ability-B- | | Andropause-AR |
| | | | allergy-IgE | Glucose | seeretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 27 | Tricuspidate | | | | | | |
| | falsenettle | | | | | | |
| 27-1 | Whole plant | 1 | 97.81 | 101.92 | 96.34 | 79.56 | 125.00 |
| | | 10 | 80.50 | 108.36 | 70.10 | 84.99 | 305.09 |
| | | 100 | 78.56 | 145.47 | 9.93 | 100.14 | 457.12 |
| 27-2 | Roots | 1 | 98.63 | 108.80 | 92.14 | 72.23 | 182.29 |
| | | 10 | 86.11 | 125.39 | 91.64 | 88.61 | 201.08 |
| | | 100 | 71.50 | 144.61 | 26.94 | 106.40 | 346.54 |
| 27-3 | Stems | 1 | 98.60 | 115.27 | 82.74 | 68.66 | 113.36 |
| | | 10 | 88.50 | 111.06 | 91.96 | 76.45 | 242.27 |
| | | 100 | 74.07 | 150.13 | 21.42 | 105.23 | 345.85 |
| 27-4 | Leaves | 1 | 97.57 | 107.35 | 101.50 | 82.18 | 138.71 |
| | | 10 | 82.99 | 100.32 | 55.70 | 95.25 | 305.13 |
| | | 100 | 78.40 | 105.04 | 30.73 | 90.47 | 407.08 |
| 27-5 | Fruits | 1 | 99.51 | 115.12 | 100.57 | 82.27 | 209.14 |
| | | 10 | 88.98 | 108.90 | 47.31 | 95.47 | 232.45 |
| | | 100 | 72.00 | 142.04 | 6.49 | 101.27 | 306.57 |
| 27-6 | Flowers | 1 | 97.29 | 95.11 | 97.19 | 84.45 | 136.36 |
| | | 10 | 91.11 | 129.90 | 78.81 | 77.90 | 223.66 |
| | | 100 | 80.00 | 157.47 | 8.64 | 96.63 | 328.81 |

### Example 28. Long-Calyx Pink

TABLE 57

| | | | Blood | Lipid- | Anti-obesity- | | Menopause- |
|---|---|---|---|---|---|---|---|
| | | | circulation | cholesterol | Glyeerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 28 | Long-calyx | | | | | | |
| | pink | | | | | | |
| 28-1 | Whole plant | 1 | 20.71 | 64.24 | 182.22 | 83.05 | 98.91 |
| | | 10 | 43.02 | 38.80 | 228.09 | 175.87 | 222.37 |
| | | 100 | 85.23 | 29.11 | 461.01 | 322.95 | 266.12 |
| 28-2 | Roots | 1 | 21.18 | 49.14 | 173.96 | 95.47 | 142.39 |
| | | 10 | 30.81 | 31.02 | 268.82 | 127.45 | 188.00 |
| | | 100 | 70.50 | 23.92 | 423.37 | 357.06 | 320.19 |
| 28-3 | Stems | 1 | 29.52 | 84.47 | 181.57 | 92.80 | 84.00 |
| | | 10 | 30.55 | 37.48 | 263.63 | 178.32 | 166.10 |
| | | 100 | 84.64 | 30.83 | 406.56 | 356.92 | 316.21 |
| 28-4 | Leaves | 1 | 21.26 | 78.43 | 163.22 | 80.01 | 93.73 |
| | | 10 | 34.66 | 36.32 | 234.27 | 121.65 | 125.66 |
| | | 100 | 76.93 | 23.81 | 418.39 | 438.06 | 277.37 |
| 28-5 | Fruits | 1 | 30.30 | 65.77 | 197.81 | 87.43 | 112.68 |
| | | 10 | 32.41 | 37.96 | 200.93 | 190.75 | 218.94 |
| | | 100 | 71.75 | 30.21 | 462.02 | 421.69 | 306.34 |
| 28-6 | Flowers | 1 | 23.85 | 70.66 | 135.80 | 101.54 | 127.05 |
| | | 10 | 39.65 | 35.18 | 274.02 | 160.51 | 152.18 |
| | | 100 | 72.39 | 23.01 | 384.55 | 393.10 | 204.08 |

TABLE 58

| | | | | Blood | Cognitive | | |
|---|---|---|---|---|---|---|---|
| | | | Anti- | glucose- | ability-B- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | ( % of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydro- |
| | | | 1 uM) | 1 mM) | Inhibitor IV | 100 ppm) | testosterone |
| | | | | | 15 nM) | | 100 nM) |
| 28 | Long-calyx | | | | | | |
| | pink | | | | | | |
| 28-1 | Whole plant | 1 | 95.56 | 96.84 | 103.75 | 79.78 | 102.85 |
| | | 10 | 90.55 | 128.87 | 88.07 | 85.05 | 298.03 |
| | | 100 | 80.36 | 133.99 | 47.92 | 107.84 | 408.58 |
| 28-2 | Roots | 1 | 100.40 | 101.96 | 96.79 | 66.10 | 210.01 |
| | | 10 | 95.24 | 122.14 | 95.96 | 80.37 | 305.32 |
| | | 100 | 80.40 | 146.25 | 26.01 | 108.66 | 387.56 |
| 28-3 | Stems | 1 | 102.96 | 109.59 | 84.09 | 65.91 | 158.68 |
| | | 10 | 93.93 | 107.19 | 71.53 | 83.36 | 229.26 |
| | | 100 | 71.67 | 142.73 | 2.79 | 103.92 | 337.57 |
| 28-4 | Leaves | 1 | 96.93 | 112.40 | 96.48 | 84.15 | 130.33 |
| | | 10 | 83.88 | 124.72 | 95.30 | 87.86 | 309.92 |
| | | 100 | 71.38 | 148.16 | 26.10 | 95.91 | 378.31 |
| 28-5 | Fruits | 1 | 97.98 | 114.94 | 90.21 | 78.25 | 185.58 |
| | | 10 | 80.13 | 121.16 | 54.04 | 77.74 | 281.23 |
| | | 100 | 73.94 | 158.84 | 30.92 | 109.75 | 330.65 |
| 28-6 | Flowers | 1 | 103.27 | 108.63 | 100.25 | 75.14 | 157.74 |
| | | 10 | 93.97 | 133.05 | 84.74 | 86.11 | 178.20 |
| | | 100 | 75.70 | 159.36 | 11.77 | 92.97 | 449.21 |

### Example 29. Curled Dock

TABLE 59

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 29 | Curled dock | | | | | | |
| 29-1 | Whole plant | 1 | 20.19 | 48.37 | 130.62 | 94.42 | 105.73 |
| | | 10 | 30.60 | 32.41 | 241.13 | 175.71 | 248.47 |
| | | 100 | 75.88 | 27.39 | 459.70 | 395.97 | 322.81 |
| 29-2 | Roots | 1 | 27.50 | 65.87 | 200.01 | 87.59 | 137.24 |
| | | 10 | 48.96 | 34.24 | 307.50 | 193.66 | 125.57 |
| | | 100 | 81.48 | 22.86 | 391.18 | 350.50 | 218.90 |
| 29-3 | Stems | 1 | 26.66 | 76.63 | 115.11 | 102.25 | 120.74 |
| | | 10 | 34.77 | 31.64 | 283.22 | 128.19 | 126.46 |
| | | 100 | 85.51 | 22.15 | 365.35 | 412.60 | 348.85 |
| 29-4 | Leaves | 1 | 22.21 | 71.49 | 158.12 | 91.79 | 122.77 |
| | | 10 | 32.90 | 31.32 | 138.05 | 170.17 | 193.23 |
| | | 100 | 70.56 | 21.63 | 166.43 | 94.14 | 344.32 |
| 29-5 | Fruits | 1 | 22.10 | 54.17 | 138.93 | 110.86 | 113.47 |
| | | 10 | 50.91 | 34.19 | 319.69 | 168.79 | 219.13 |
| | | 100 | 74,30 | 30.60 | 479.77 | 423.83 | 256.75 |
| 29-6 | Flowers | 1 | 24.44 | 43.56 | 192.41 | 90.95 | 148.84 |
| | | 10 | 47.22 | 32.47 | 234.58 | 189.68 | 228.95 |
| | | 100 | 83.79 | 21.76 | 363.93 | 331.38 | 241.18 |

TABLE 60

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 29 | Curled dock | | | | | | |
| 29-1 | Whole plant | 1 | 99.07 | 114.43 | 91.52 | 65.38 | 177.63 |
| | | 10 | 94.02 | 106.56 | 50.58 | 77.48 | 252.83 |
| | | 100 | 79.19 | 136.43 | 8.05 | 92.98 | 458.01 |
| 29-2 | Roots | 1 | 95.74 | 114.45 | 101.86 | 73.53 | 143.08 |
| | | 10 | 93.01 | 131.49 | 80.68 | 88.21 | 249.29 |
| | | 100 | 75.74 | 148.23 | 38.20 | 108.95 | 342.82 |
| 29-3 | Stems | 1 | 97.27 | 114.36 | 97.53 | 82.89 | 207.23 |
| | | 10 | 94.51 | 129.86 | 45.04 | 79.86 | 234.86 |
| | | 100 | 76.82 | 149.97 | 41.78 | 96.15 | 438.18 |
| 29-4 | Leaves | 1 | 98.90 | 103.25 | 107.07 | 80.34 | 161.22 |
| | | 10 | 92.77 | 80.67 | 107.93 | 82.68 | 308.70 |
| | | 100 | 103.98 | 86.82 | 118.28 | 102.50 | 368.77 |
| 29-5 | Fruits | 1 | 102.67 | 96.61 | 95.00 | 74.80 | 113.07 |
| | | 10 | 88.50 | 123.82 | 83.17 | 83.31 | 322.82 |
| | | 100 | 70.54 | 149.36 | 39.56 | 93.12 | 382.37 |
| 29-6 | Flowers | 1 | 96.88 | 101.47 | 106.30 | 68.34 | 145.86 |
| | | 10 | 94.70 | 120.80 | 74.96 | 78.10 | 332.68 |
| | | 100 | 76.27 | 159.57 | 42.03 | 98.99 | 405.49 |

### Example 30. Erect St. Johnswort

TABLE 61

| | | | Blood | Lipid- | Anti-obesity- | | Menopause- |
|---|---|---|---|---|---|---|---|
| | | | circulation- | cholesterol | Glycerol | Immnnity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 tiM) | 10 μM) | | 1 ng/ml) |
| 30 | Erect St. | | | | | | |
| | Johnswort | | | | | | |
| 30-1 | Whole plant | 1 | 25.69 | 42.46 | 179.27 | 89.02 | 137.66 |
| | | 10 | 35.99 | 40.64 | 312.62 | 181.94 | 225.78 |
| | | 100 | 83.38 | 29.42 | 464.29 | 390.20 | 350.74 |
| 30-2 | Roots | 1 | 30.58 | 51.68 | 153.01 | 110.99 | 135.52 |
| | | 10 | 31.58 | 36.67 | 221.57 | 140.21 | 145.99 |
| | | 100 | 81.33 | 30.59 | 460.65 | 394.96 | 290.14 |
| 30-3 | Stems | 1 | 21.21 | 41.39 | 139.87 | 100.42 | 113.69 |
| | | 10 | 34.33 | 33.88 | 249.44 | 158.27 | 203.90 |
| | | 100 | 72.82 | 30.27 | 486.98 | 337.84 | 283.38 |
| 30-4 | Leaves | 1 | 22.88 | 73.19 | 188.88 | 89.62 | 137.67 |
| | | 10 | 48.93 | 35.37 | 279.32 | 146.59 | 183.13 |
| | | 100 | 79.42 | 23.10 | 474.87 | 475.20 | 221.29 |
| 30-5 | Fruits | 1 | 26.58 | 49.21 | 125.68 | 89.65 | 119.31 |
| | | 10 | 49.64 | 34.82 | 200.37 | 190.60 | 225.28 |
| | | 100 | 82.43 | 23.06 | 400.76 | 421.64 | 290.91 |
| 30-6 | Flowers | 1 | 26.51 | 43.27 | 126.68 | 94.03 | 80.89 |
| | | 10 | 50.80 | 33.34 | 262.78 | 136.73 | 211.02 |
| | | 100 | 79.72 | 29.54 | 384.13 | 434.31 | 228.39 |

TABLE 62

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 30 | Erect St. | | | | | | |
| | Johnswort | | | | | | |
| 30-1 | Whole plant | 1 | 97.87 | 104.10 | 108.81 | 72.66 | 181.50 |
| | | 10 | 85.27 | 106.32 | 65.81 | 95.67 | 231.30 |
| | | 100 | 75.73 | 155.65 | 17.52 | 105.53 | 352.16 |
| 30-2 | Roots | 1 | 98.42 | 98.18 | 87.63 | 76.95 | 192.15 |
| | | 10 | 83.53 | 107.29 | 92.65 | 94.89 | 196.92 |
| | | 100 | 75.51 | 132.97 | 46.40 | 99.21 | 455.72 |
| 30-3 | Stems | 1 | 96.92 | 105.32 | 85.00 | 74.28 | 103.93 |
| | | 10 | 91.72 | 113.71 | 94.67 | 88.85 | 298.30 |
| | | 100 | 77.54 | 144.86 | 13.34 | 100.87 | 436.85 |
| 30-4 | Leaves | 1 | 97.20 | 101.95 | 100.00 | 78.75 | 110.24 |
| | | 10 | 94.09 | 104.77 | 95.43 | 75.51 | 288.65 |
| | | 100 | 70.44 | 80.72 | 11.17 | 77.46 | 436.59 |
| 30-5 | Fruits | 1 | 95.98 | 107.02 | 96.94 | 78.88 | 169.92 |
| | | 10 | 86.00 | 106.86 | 80.78 | 94.29 | 305.51 |
| | | 100 | 75.93 | 130.50 | 5.44 | 97.69 | 328.01 |
| 30-6 | Flowers | 1 | 96.49 | 98.27 | 90.73 | 79.15 | 125.63 |
| | | 10 | 95.19 | 123.00 | 78.37 | 91.17 | 250.98 |
| | | 100 | 75.96 | 153.56 | 33.12 | 95.17 | 329.81 |

### Example 31. Five-Leaf Gynostemma

TABLE 63

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (Ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/tnl) |
| 31 | Five-leaf | | | | | | |
| | gynostemma | | | | | | |
| 31-1 | Whole plant | 1 | 25.19 | 58.35 | 117.75 | 110.24 | 80.10 |
| | | 10 | 39.65 | 33.95 | 246.32 | 126.02 | 222.01 |
| | | 100 | 74.73 | 23.89 | 385.14 | 492.72 | 274.05 |
| 31-2 | Roots | 1 | 29.66 | 72.69 | 187.51 | 96.69 | 112.19 |
| | | 10 | 45.38 | 35.22 | 271.45 | 125.04 | 193.41 |
| | | 100 | 77.84 | 23.66 | 438.77 | 467.26 | 342.25 |
| 31-3 | Stems | 1 | 24.28 | 57.04 | 165.20 | 102.99 | 111.26 |
| | | 10 | 46.30 | 34.66 | 255.08 | 182.93 | 238.80 |
| | | 100 | 80.40 | 24.13 | 410.47 | 474.12 | 279.63 |
| 31-4 | Leaves | 1 | 30.09 | 87.41 | 104.84 | 90.00 | 90.00 |
| | | 10 | 37.78 | 86.33 | 128.54 | 94.73 | 193.10 |
| | | 100 | 85.01 | 96.63 | 96.86 | 96.96 | 276.26 |
| 31-5 | Fruits | 1 | 29.61 | 75.83 | 156.10 | 108.02 | 139.65 |
| | | 10 | 31.55 | 32.78 | 270.07 | 134.32 | 220.66 |
| | | 100 | 70.94 | 20.47 | 494.20 | 432.95 | 330.79 |
| 31-6 | Howers | 1 | 29.19 | 83.33 | 137.36 | 105.90 | 100.48 |
| | | 10 | 34.56 | 36.54 | 222.61 | 186.38 | 186.97 |
| | | 100 | 80.20 | 29.74 | 432.38 | 420.44 | 297.08 |

TABLE 64

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | ( % of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 31 | Five-leaf | | | | | | |
| | gynostemma | | | | | | |
| 31-1 | Whole plant | 1 | 101.30 | 98.13 | 101.28 | 66.28 | 139.05 |
| | | 10 | 82.91 | 117.51 | 67.15 | 75.07 | 244.20 |
| | | 100 | 80.56 | 149.50 | 46.82 | 109.65 | 464.96 |
| 31-2 | Roots | 1 | 103.43 | 105.84 | 102.87 | 80.96 | 145.23 |
| | | 10 | 82.55 | 127.11 | 67.13 | 84.64 | 290.40 |
| | | 100 | 78.64 | 142.00 | 33.56 | 95.44 | 446.84 |
| 31-3 | Stems | 1 | 101.80 | 109.79 | 86.35 | 65.41 | 207.31 |
| | | 10 | 94.13 | 116.95 | 55.37 | 85.78 | 287.65 |
| | | 100 | 72.58 | 144.86 | 7.24 | 97.79 | 426.53 |
| 31-4 | Leaves | 1 | 101.63 | 108.28 | 104.30 | 71.29 | 203.04 |
| | | 10 | 89.75 | 98.90 | 103.91 | 67.98 | 334.44 |
| | | 100 | 80.61 | 106.55 | 106.70 | 73.99 | 390.03 |
| 31-5 | Fruits | 1 | 103.54 | 106.04 | 87.96 | 82.89 | 118.08 |
| | | 10 | 82.01 | 114.94 | 86.39 | 85.49 | 207.30 |
| | | 100 | 72.83 | 151.32 | 20.36 | 100.39 | 421.54 |
| 31-6 | Flowers | 1 | 97.57 | 109.41 | 87.94 | 79.25 | 143.51 |
| | | 10 | 94.48 | 139.17 | 85.48 | 82.64 | 287.66 |
| | | 100 | 73.26 | 160.11 | 49.15 | 99.57 | 390.36 |

### Example 32. Bird's Egg Cucumber

TABLE 65

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 32 | Bird's egg | | | | | | |
| | cucumber | | | | | | |
| 32-1 | Whole plant | 1 | 27.28 | 62.76 | 179.15 | 96.41 | 117.53 |
| | | 10 | 48.03 | 40.61 | 299.24 | 115.82 | 136.63 |
| | | 100 | 76.86 | 24.22 | 368.96 | 325.76 | 238.54 |
| 32-2 | Roots | 1 | 22.04 | 87.49 | 191.18 | 105.61 | 93.81 |
| | | 10 | 30.59 | 31.97 | 229.00 | 124.85 | 196.19 |
| | | 100 | 84.29 | 25.80 | 424,17 | 367.94 | 261.41 |
| 32-3 | Stems | 1 | 26.50 | 38.81 | 163.56 | 93.99 | 119.80 |
| | | 10 | 46.14 | 32.52 | 248.73 | 110.93 | 242.72 |
| | | 100 | 73.83 | 21.36 | 464.75 | 419.28 | 278.35 |
| 32-4 | Leaves | 1 | 20.16 | 71.05 | 136.33 | 80.10 | 142.82 |
| | | 10 | 41.88 | 40.07 | 334.21 | 142.65 | 136.89 |
| | | 100 | 80.97 | 28.74 | 412.32 | 425.45 | 327.02 |
| 32-5 | Fruits | 1 | 22.44 | 75.15 | 134.98 | 98.62 | 147.73 |
| | | 10 | 50.02 | 38.09 | 200.24 | 112.40 | 178.94 |
| | | 100 | 72.22 | 28.14 | 417.65 | 335.98 | 215.26 |
| 32-6 | Flowers | 1 | 30.68 | 44.34 | 193.15 | 105.48 | 143.06 |
| | | 10 | 48.80 | 33.44 | 319.62 | 190.14 | 190.40 |
| | | 100 | 74,31 | 22.00 | 495.42 | 447.63 | 241.87 |

TABLE 66

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 32 | Bird's egg | | | | | | |
| | cucumber | | | | | | |
| 32-1 | Whole plant | 1 | 96.95 | 110.90 | 102.65 | 65.20 | 202.95 |
| | | 10 | 87.60 | 107.42 | 90.48 | 81.03 | 304.33 |
| | | 100 | 70.37 | 138.28 | 13.94 | 93.45 | 392.49 |
| 32-2 | Roots | 1 | 102.60 | 104.99 | 108.03 | 73.96 | 101.51 |
| | | 10 | 95.39 | 129.70 | 77.70 | 83,54 | 232.66 |
| | | 100 | 77.52 | 137.92 | 19.04 | 103.93 | 415.26 |
| 32-3 | Stems | 1 | 101.28 | 110.75 | 80.25 | 67.81 | 156.95 |
| | | 10 | 93.20 | 106.24 | 62.67 | 75.79 | 300.66 |
| | | 100 | 76.43 | 134,29 | 49.56 | 97.33 | 358.02 |
| 32-4 | Leaves | 1 | 99.15 | 111.58 | 90.92 | 72.31 | 113.81 |
| | | 10 | 80.54 | 124.06 | 72.35 | 95.94 | 179.37 |
| | | 100 | 76.47 | 156.80 | 2.13 | 103.52 | 437.65 |
| 32-5 | Fruits | 1 | 101.69 | 98.81 | 101.47 | 72.42 | 157.45 |
| | | 10 | 89.28 | 116.88 | 49.79 | 83.05 | 298.27 |
| | | 100 | 76.37 | 159.38 | 42.55 | 102.73 | 415.99 |
| 32-6 | Flowers | 1 | 103.14 | 99.63 | 85.01 | 83.44 | 131.86 |
| | | 10 | 88.10 | 125.33 | 53.65 | 82.56 | 235.49 |
| | | 100 | 74.44 | 158.86 | 16.18 | 98.33 | 367.64 |

### Example 33. Ulleungdo Rockcress

TABLE 67

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 33 | Ulleungdo | | | | | | |
| | rockcress | | | | | | |
| 33-1 | Whole plant | 1 | 22.17 | 72.77 | 142.95 | 84.85 | 147.32 |
| | | 10 | 40.55 | 35.54 | 205.32 | 199.45 | 104.71 |
| | | 100 | 85.34 | 26.52 | 440.25 | 393.53 | 331.80 |
| 33-2 | Roots | 1 | 28.84 | 48.35 | 193.15 | 90.00 | 138.91 |
| | | 10 | 42.82 | 34.11 | 287.93 | 200.61 | 206.76 |
| | | 100 | 77.65 | 30.35 | 353.05 | 325.53 | 345.32 |
| 33-3 | Stems | 1 | 28.26 | 77.43 | 157.34 | 109.36 | 107.85 |
| | | 10 | 42.00 | 34.09 | 224.48 | 172.91 | 121.91 |
| | | 100 | 79.26 | 30.23 | 490.88 | 300.23 | 257.54 |
| 33-4 | Leaves | 1 | 26.80 | 86.69 | 144.43 | 84.21 | 118.95 |
| | | 10 | 39.25 | 39.34 | 300.11 | 120.70 | 122.95 |
| | | 100 | 77.80 | 20.84 | 450.85 | 453.95 | 311.23 |
| 33-5 | Fruits | 1 | 22.52 | 42.61 | 146.57 | 88.86 | 121.06 |
| | | 10 | 41.52 | 35.90 | 334.00 | 132.50 | 209.11 |
| | | 100 | 72.64 | 23.73 | 383.05 | 465.85 | 209.95 |
| 33-6 | Flowers | 1 | 21.72 | 84.86 | 177.04 | 101.21 | 138.27 |
| | | 10 | 44.41 | 33.38 | 249.93 | 147.07 | 105.80 |
| | | 100 | 71.67 | 30.55 | 428.16 | 328.04 | 276.50 |

TABLE 68

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 33 | Ulleungdo | | | | | | |
| | rockcress | | | | | | |
| 33-1 | Whole plant | 1 | 98.25 | 102.54 | 85.44 | 65.25 | 98.16 |
| | | 10 | 85.61 | 112.62 | 64.90 | 93.99 | 192.89 |
| | | 100 | 78.87 | 130.85 | 2.38 | 94.78 | 333.39 |
| 33-2 | Roots | 1 | 96.93 | 97.92 | 110.85 | 68.23 | 113.74 |
| | | 10 | 92.25 | 125.59 | 62.91 | 80.16 | 206.32 |
| | | 100 | 74.42 | 138.26 | 16.13 | 103.43 | 345.12 |
| 33-3 | Stems | 1 | 103.22 | 96.93 | 99.29 | 67.67 | 129.62 |
| | | 10 | 91.69 | 128.48 | 55.65 | 86.05 | 229.08 |
| | | 100 | 80.94 | 160.97 | 47.27 | 97.89 | 474.12 |
| 33-4 | Leaves | 1 | 102.84 | 107.99 | 110.18 | 74.52 | 204.75 |
| | | 10 | 80.02 | 137.57 | 84.25 | 82.65 | 334.15 |
| | | 100 | 72.94 | 151.63 | 5.27 | 90.88 | 373.16 |
| 33-5 | Fruits | 1 | 96.27 | 102.65 | 107.34 | 72.13 | 129.16 |
| | | 10 | 83.03 | 108.26 | 62.98 | 79.18 | 194.74 |
| | | 100 | 71.72 | 131.77 | 46.78 | 108.19 | 398.02 |
| 33-6 | Flowers | 1 | 98.63 | 110.14 | 84.89 | 70.27 | 109.21 |
| | | 10 | 88.69 | 112.52 | 76.44 | 95.60 | 338.32 |
| | | 100 | 80.37 | 145.44 | 38.24 | 107.64 | 447.26 |

### Example 34. Hedge Mustard

TABLE 69

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 34 | Hedge | | | | | | |
| | Mustard | | | | | | |
| 34-1 | Whole plant | 1 | 27.67 | 51.33 | 119.09 | 89.81 | 123.40 |
| | | 10 | 42.62 | 35.69 | 214.37 | 170.04 | 247.55 |
| | | 100 | 70.17 | 28.98 | 417.17 | 410.09 | 280.76 |
| 34-2 | Roots | 1 | 21.55 | 83.18 | 177.00 | 86.78 | 108.68 |
| | | 10 | 50.48 | 39.21 | 275.83 | 196.30 | 173.16 |
| | | 100 | 71.32 | 28.90 | 393.12 | 324.69 | 222.12 |
| 34-3 | Stems | 1 | 21.49 | 78.46 | 121.32 | 106.89 | 127.89 |
| | | 10 | 36.22 | 30.53 | 302.90 | 162.71 | 234.01 |
| | | 100 | 83.07 | 23.74 | 361.12 | 410.47 | 215.85 |
| 34-4 | Leaves | 1 | 20.11 | 74.74 | 106.00 | 88.47 | 104.40 |
| | | 10 | 50.05 | 39.74 | 273.56 | 186.17 | 135.16 |
| | | 100 | 71.85 | 30.48 | 407.87 | 480.46 | 274.70 |
| 34-5 | Fruits | 1 | 28.15 | 80.54 | 179.17 | 88.60 | 105.03 |
| | | 10 | 46.07 | 33.45 | 294.26 | 112.57 | 209.76 |
| | | 100 | 74.47 | 24.30 | 469.16 | 399.32 | 305.74 |
| 34-6 | Flowers | 1 | 21.29 | 62.17 | 175.60 | 92.88 | 81.93 |
| | | 10 | 30.57 | 33.59 | 236.48 | 148.27 | 237.59 |
| | | 100 | 85.27 | 23.72 | 420.59 | 434.97 | 331.28 |

TABLE 70

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 34 | Hedge | | | | | | |
| | Mustard | | | | | | |
| 34-1 | Whole plant | 1 | 99.24 | 112.04 | 92.06 | 85.93 | 126.89 |
| | | 10 | 82.29 | 106.99 | 71.37 | 80.19 | 182.66 |
| | | 100 | 71.87 | 139.09 | 7.00 | 99.51 | 305.11 |
| 34-2 | Roots | 1 | 95.97 | 113.49 | 108.72 | 70.14 | 129.32 |
| | | 10 | 87.27 | 127.27 | 82.08 | 87.86 | 199.21 |
| | | 100 | 74.89 | 137.71 | 39.03 | 97.08 | 485.25 |
| 34-3 | Stems | 1 | 100.47 | 103.41 | 94.75 | 79.68 | 173.35 |
| | | 10 | 89.70 | 112.76 | 58.47 | 95.59 | 269.89 |
| | | 100 | 74.67 | 138.74 | 22.69 | 98.68 | 415.92 |
| 34-4 | Leaves | 1 | 99.91 | 107.82 | 93.48 | 65.02 | 134.28 |
| | | 10 | 84.70 | 130.88 | 81.44 | 93.67 | 185.62 |
| | | 100 | 71.66 | 134.34 | 10.69 | 100.17 | 301.15 |
| 34-5 | Fruits | 1 | 101.73 | 114.89 | 87.59 | 82.88 | 143.04 |
| | | 10 | 81.87 | 117.74 | 69.68 | 85.60 | 338.51 |
| | | 100 | 71.03 | 138.94 | 26.02 | 103.12 | 325.60 |
| 34-6 | Flowers | 1 | 102.08 | 105.99 | 84.56 | 70.79 | 156.28 |
| | | 10 | 85.60 | 113.32 | 63.54 | 77.48 | 278.30 |
| | | 100 | 73.12 | 130.92 | 19.86 | 101.03 | 483.71 |

### Example 35. Fauriei Rosebay

TABLE 71

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 35 | Fauriei | | | | | | |
| | rosebay | | | | | | |
| 35-1 | Whole plant | 1 | 22.77 | 75.13 | 178.98 | 96.55 | 104.17 |
| | | 10 | 46.98 | 33.80 | 262.28 | 178.50 | 190.25 |
| | | 100 | 70.89 | 28.23 | 358.89 | 334.34 | 253.76 |
| 35-2 | Roots | 1 | 27.26 | 85.51 | 167.02 | 88.58 | 112.59 |
| | | 10 | 39.07 | 35.59 | 263.20 | 129.13 | 232.47 |
| | | 100 | 77.75 | 28.66 | 361.77 | 403.50 | 228.71 |
| 35-3 | Stems | 1 | 25.83 | 52.50 | 148.85 | 83.92 | 125.27 |
| | | 10 | 35.51 | 31.83 | 248.41 | 148.78 | 195.86 |
| | | 100 | 74.54 | 27.93 | 407.09 | 459.16 | 233.89 |
| 35-4 | Leaves | 1 | 27.08 | 71.76 | 105.50 | 99.09 | 151.00 |
| | | 10 | 33.07 | 38.18 | 303.76 | 144.16 | 141.74 |
| | | 100 | 79.96 | 25.61 | 393.29 | 320.33 | 237.04 |
| 35-5 | Fruits | 1 | 24.64 | 56.12 | 129.79 | 83.83 | 114.13 |
| | | 10 | 34.76 | 37.86 | 228.86 | 146.53 | 202.49 |
| | | 100 | 75.14 | 30.55 | 362.55 | 309.63 | 243.20 |
| 35-6 | Flowers | 1 | 20.67 | 61.49 | 187.92 | 107.47 | 134.16 |
| | | 10 | 33.75 | 37.24 | 207.37 | 152.89 | 249.31 |
| | | 100 | 81.43 | 24.41 | 481.98 | 332.86 | 312.95 |

TABLE 72

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sampie | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 35 | Fauriei | | | | | | |
| | rosebay | | | | | | |
| 35-1 | Whole plant | 1 | 102.93 | 109.47 | 108.72 | 65.69 | 181.22 |
| | | 10 | 81.61 | 113.74 | 84.44 | 83.26 | 247.37 |
| | | 100 | 70.97 | 146.72 | 30.51 | 108.34 | 458.05 |
| 35-2 | Roots | 1 | 102.09 | 105.54 | 94.56 | 77.29 | 153.82 |
| | | 10 | 80.45 | 109.06 | 62.44 | 95.74 | 230.80 |
| | | 100 | 77 | 156.89 | 24.27 | 93.22 | 477.70 |
| 35-3 | Stems | 1 | 97.88 | 100.70 | 88.82 | 70.25 | 154.36 |
| | | 10 | 86.67 | 129.36 | 74.33 | 93.46 | 291.70 |
| | | 100 | 75.12 | 146.57 | 22.80 | 95.77 | 400.98 |
| 35-4 | Leaves | 1 | 98.91 | 100.86 | 81.49 | 83.85 | 129.15 |
| | | 10 | 88.67 | 106.85 | 61.02 | 76.31 | 221.08 |
| | | 100 | 74.67 | 132.85 | 6.37 | 107.13 | 389.53 |
| 35-5 | Fruits | 1 | 98.49 | 101.09 | 104.06 | 69.02 | 90.43 |
| | | 10 | 90.75 | 140.28 | 52.87 | 77.31 | 295.88 |
| | | 100 | 79.24 | 147.98 | 14.71 | 92.48 | 442.38 |
| 35-6 | Flowers | 1 | 101.81 | 107.83 | 102.72 | 72.85 | 106.13 |
| | | 10 | 91.51 | 122.49 | 75.48 | 80.09 | 182.19 |
| | | 100 | 70.12 | 159.29 | 34.04 | 106.01 | 338.98 |

### Example 36. Asian Prince's Pine

TABLE 73

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 36 | Asian | | | | | | |
| | prince's pine | | | | | | |
| 36-1 | Whole plant | 1 | 27.90 | 48.55 | 177.81 | 92.79 | 140.07 |
| | | 10 | 49.50 | 40.76 | 224.63 | 134.35 | 119.55 |
| | | 100 | 80.91 | 28.32 | 468.17 | 397.57 | 258.25 |
| 36-2 | Roots | 1 | 27.13 | 57.28 | 142.64 | 97.38 | 99.62 |
| | | 10 | 32.79 | 35.90 | 210.41 | 119.20 | 100.28 |
| | | 100 | 76.75 | 30.44 | 360.32 | 364.48 | 347.40 |
| 36-3 | Stems | 1 | 28.40 | 89.91 | 107.88 | 109.98 | 120.32 |
| | | 10 | 48.87 | 38.22 | 289.09 | 123.69 | 158.03 |
| | | 100 | 70.70 | 30.93 | 368.27 | 369.80 | 238.76 |
| 36-4 | Leaves | 1 | 28.64 | 67.49 | 182.11 | 103.74 | 123.20 |
| | | 10 | 35.16 | 36.79 | 265.70 | 139.45 | 114.40 |
| | | 100 | 77.82 | 24.13 | 378.46 | 495.12 | 266.18 |
| 36-5 | Fruits | 1 | 25.48 | 79.61 | 149.08 | 108.25 | 122.01 |
| | | 10 | 30.64 | 31.27 | 338.93 | 190.67 | 127.99 |
| | | 100 | 77.98 | 25.31 | 432.39 | 411.68 | 296.22 |
| 36-6 | Flowers | 1 | 22.56 | 47.28 | 130.71 | 82.33 | 84.22 |
| | | 10 | 43.09 | 38.24 | 254.54 | 178.40 | 164.18 |
| | | 100 | 85.79 | 28.28 | 476.16 | 370.70 | 280.95 |

TABLE 74

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 36 | Asian | | | | | | |
| | prince's pine | | | | | | |
| 36-1 | Whole plant | 1 | 95.19 | 114.80 | 109.17 | 75.41 | 136.75 |
| | | 10 | 93.45 | 122.94 | 47.83 | 77.18 | 177.50 |
| | | 100 | 73.99 | 132.32 | 45.81 | 108.19 | 419.46 |
| 36-2 | Roots | 1 | 97.77 | 96.96 | 86.03 | 66.70 | 141.05 |
| | | 10 | 91.19 | 125.30 | 53.40 | 90.32 | 321.14 |
| | | 100 | 72.00 | 155.27 | 32.04 | 92.66 | 353.42 |
| 36-3 | Stems | 1 | 97.54 | 98.50 | 88.71 | 81.81 | 93.05 |
| | | 10 | 88.75 | 114.71 | 61.94 | 79.16 | 331.11 |
| | | 100 | 80.53 | 144.86 | 22.12 | 110.67 | 323.80 |
| 36-4 | Leaves | 1 | 96.18 | 97.19 | 89.46 | 71.99 | 153.94 |
| | | 10 | 91.39 | 126.42 | 79.65 | 90.57 | 332.96 |
| | | 100 | 73.89 | 144.69 | 47.34 | 106.96 | 392.51 |
| 36-5 | Fruits | 1 | 103.10 | 99.43 | 109.09 | 81.56 | 122.13 |
| | | 10 | 88.74 | 129.70 | 92.99 | 87.42 | 205.57 |
| | | 100 | 77.71 | 147.57 | 23.68 | 110.09 | 342.67 |
| 36-6 | Flowers | 1 | 99.85 | 106.94 | 85.24 | 73.80 | 139.36 |
| | | 10 | 87.58 | 120.06 | 53.06 | 94.42 | 255.65 |
| | | 100 | 75.30 | 139.77 | 38.90 | 95.72 | 367.90 |

### Example 37. Marlberry

TABLE 75

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 37 | Marlberry | | | | | | |
| 37-1 | Whole plant | 1 | 25.48 | 43.89 | 192.00 | 104.78 | 94.82 |
| | | 10 | 48.38 | 34.40 | 202.03 | 161.37 | 140.90 |
| | | 100 | 84.79 | 30.35 | 363.65 | 309.20 | 344.15 |
| 37-2 | Roots | 1 | 29.50 | 86.12 | 172.40 | 82.89 | 105.01 |
| | | 10 | 35.69 | 30.08 | 293.69 | 162.51 | 198.10 |
| | | 100 | 85.77 | 26.31 | 381.71 | 311.58 | 310.77 |
| 37-3 | Stems | 1 | 28.87 | 57.75 | 173.25 | 90.90 | 124.46 |
| | | 10 | 44.64 | 39.13 | 240.87 | 148.25 | 113.83 |
| | | 100 | 79.57 | 22.04 | 355.77 | 490.49 | 326.08 |
| 37-4 | Leaves | 1 | 28.09 | 69.20 | 150.51 | 88.95 | 103.30 |
| | | 10 | 42.10 | 34.94 | 227.75 | 130.65 | 237.55 |
| | | 100 | 82.72 | 30.96 | 365.37 | 356.22 | 310.38 |
| 37-5 | Fruits | 1 | 26.22 | 63.25 | 163.54 | 109.34 | 115.92 |
| | | 10 | 39.77 | 30.74 | 286.94 | 138.50 | 118.01 |
| | | 100 | 76.84 | 24.65 | 471.76 | 349.77 | 289.17 |
| 37-6 | Flowers | 1 | 30.61 | 76.94 | 109.26 | 99.23 | 146.41 |
| | | 10 | 44.48 | 31.16 | 205.25 | 185.54 | 113.60 |
| | | 100 | 75.27 | 21.89 | 388.01 | 383.61 | 297.52 |

TABLE 76

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 37 | Marlberry | | | | | | |
| 37-1 | Whole plant | 1 | 97.30 | 113.23 | 95.56 | 81.32 | 134.17 |
| | | 10 | 92.35 | 120.40 | 47.13 | 94.18 | 319.82 |
| | | 100 | 79.38 | 147.11 | 18.96 | 109.56 | 340.39 |
| 37-2 | Roots | 1 | 103.09 | 108.59 | 100.02 | 74.88 | 199.15 |
| | | 10 | 82.54 | 119.73 | 63.63 | 75.31 | 333.80 |
| | | 100 | 77.50 | 147.83 | 31.65 | 91.77 | 433.85 |
| 37-3 | Stems | 1 | 103.86 | 99.56 | 104.00 | 72.43 | 97.61 |
| | | 10 | 89.47 | 135.42 | 87.08 | 80.68 | 293.14 |
| | | 100 | 78.25 | 140.97 | 46.28 | 99.95 | 372.45 |
| 37-4 | Leaves | 1 | 101.46 | 100.69 | 95.88 | 75.24 | 197.00 |
| | | 10 | 80.38 | 134.62 | 89.98 | 92.24 | 294.42 |
| | | 100 | 76.45 | 152.85 | 38.70 | 97.68 | 434.01 |
| 37-5 | Fruits | 1 | 99.62 | 96.96 | 110.68 | 65.27 | 186.58 |
| | | 10 | 93.29 | 108.95 | 47.25 | 89.87 | 317.58 |
| | | 100 | 76.45 | 131.20 | 15.56 | 92.46 | 382.38 |
| 37-6 | Flowers | 1 | 95.32 | 111.16 | 89.86 | 66.83 | 113.95 |
| | | 10 | 87.92 | 133.78 | 65.29 | 78.50 | 261.89 |
| | | 100 | 80.88 | 146.65 | 12.48 | 106.22 | 322.86 |

### Example 38. Spoon-Leaf Yellow Loosestrife

TABLE 77

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 38 | Spoon-leaf | | | | | | |
| | yellow | | | | | | |
| | loosestrife | | | | | | |
| 38-1 | Whole plant | 1 | 21.93 | 40.62 | 176.54 | 86.33 | 141.11 |
| | | 10 | 42.60 | 39.77 | 209.37 | 124.99 | 120.70 |
| | | 100 | 76.12 | 23.23 | 351.37 | 321.96 | 319.23 |
| 38-2 | Roots | 1 | 30.61 | 79.55 | 144.21 | 109.90 | 144.20 |
| | | 10 | 35.77 | 30.75 | 200.46 | 143.45 | 177.86 |
| | | 100 | 85.56 | 26.97 | 443.44 | 435.69 | 226.12 |
| 38-3 | Stems | 1 | 28.78 | 76.61 | 190.33 | 85.45 | 81.79 |
| | | 10 | 45.95 | 31.83 | 272.49 | 165.64 | 146.43 |
| | | 100 | 74.97 | 30.87 | 472.65 | 363.20 | 244.86 |
| 38-4 | Leaves | 1 | 20.68 | 45.31 | 180.02 | 83.59 | 120.40 |
| | | 10 | 38.20 | 30.26 | 215.41 | 113.07 | 192.47 |
| | | 100 | 77.28 | 30.21 | 494.81 | 303.82 | 341.88 |
| 38-5 | Fruits | 1 | 22.44 | 70.50 | 122.08 | 80.19 | 117.18 |
| | | 10 | 43.70 | 39.72 | 259.81 | 152.47 | 161.39 |
| | | 100 | 83.03 | 27.99 | 364.94 | 412.45 | 340.68 |
| 38-6 | Flowers | 1 | 23.84 | 77.40 | 104.31 | 98.48 | 80.20 |
| | | 10 | 41.88 | 38.82 | 246.80 | 190.12 | 244.13 |
| | | 100 | 81.40 | 24.85 | 372.01 | 472.86 | 220.91 |

TABLE 78

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 38 | Spoon-leaf | | | | | | |
| | yellow | | | | | | |
| | loosestrife | | | | | | |
| 38-1 | Whole plant | 1 | 95.01 | 104.97 | 97.99 | 82.87 | 168.08 |
| | | 10 | 83.57 | 112.71 | 58.12 | 75.90 | 190.95 |
| | | 100 | 76.88 | 156.20 | 40.67 | 103.86 | 341.75 |
| 38-2 | Roots | 1 | 99.78 | 97.13 | 84.62 | 78.75 | 135.56 |
| | | 10 | 95.65 | 138.70 | 47.70 | 89.49 | 232.98 |
| | | 100 | 74.53 | 138.89 | 17.97 | 107.81 | 397.93 |
| 38-3 | Stems | 1 | 98.27 | 105.66 | 93.31 | 82.40 | 127.99 |
| | | 10 | 90.83 | 111.75 | 70.70 | 79.35 | 210.19 |
| | | 100 | 76.45 | 136.94 | 23.43 | 96.01 | 341.41 |
| 38-4 | Leaves | 1 | 103.92 | 101.05 | 99.50 | 70.98 | 171.41 |
| | | 10 | 88.25 | 105.23 | 68.92 | 88.35 | 337.74 |
| | | 100 | 79.81 | 152.35 | 17.06 | 94.19 | 497.17 |
| 38-5 | Fruits | 1 | 97.60 | 112.67 | 84.63 | 83.05 | 107.68 |
| | | 10 | 87.23 | 129.42 | 85.64 | 90.84 | 279.90 |
| | | 100 | 79.78 | 143.03 | 31.43 | 100.41 | 357.56 |
| 38-6 | Flowers | 1 | 95.50 | 95.08 | 105.04 | 83.92 | 135.47 |
| | | 10 | 86.90 | 123.33 | 91.04 | 91.37 | 237.07 |
| | | 100 | 75.22 | 132.06 | 35.02 | 107.96 | 397.40 |

### Example 39. Climbing Hydrangea

TABLE 79

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 39 | Climbing | | | | | | |
| | hydrangea | | | | | | |
| 39-1 | Whole plant | 1 | 23.49 | 52.20 | 114.93 | 99.66 | 105.76 |
| | | 10 | 42.79 | 37.44 | 222.11 | 126.57 | 210.82 |
| | | 100 | 83.27 | 22.96 | 418.41 | 413.70 | 226.34 |
| 39-2 | Roots | 1 | 30.55 | 43.26 | 118.74 | 107.08 | 130.09 |
| | | 10 | 47.39 | 31.54 | 306.44 | 180.21 | 132.11 |
| | | 100 | 76.18 | 30.59 | 495.02 | 465.65 | 211.93 |
| 39-3 | Stems | 1 | 27.23 | 74.04 | 168.50 | 85.02 | 119.42 |
| | | 10 | 50.88 | 38.05 | 302.70 | 153.89 | 109.87 |
| | | 100 | 77.10 | 21.48 | 437.93 | 320.42 | 250.27 |
| 39-4 | Leaves | 1 | 24.52 | 43.88 | 182.34 | 93.17 | 143.85 |
| | | 10 | 39.06 | 30.36 | 272.60 | 119.64 | 114.82 |
| | | 100 | 81.86 | 21.61 | 451.10 | 466.82 | 259.97 |
| 39-5 | Fruits | 1 | 24.41 | 86.13 | 192.61 | 87.92 | 140.42 |
| | | 10 | 49.77 | 36.84 | 238.51 | 178.34 | 177.65 |
| | | 100 | 79.75 | 28.64 | 435.45 | 416.53 | 244.80 |
| 39-6 | Flowers | 1 | 23.08 | 50.25 | 157.43 | 98.22 | 137.65 |
| | | 10 | 33.78 | 39.36 | 309.87 | 112.11 | 165.61 |
| | | 100 | 80.04 | 24.31 | 400.59 | 465.56 | 226.90 |

TABLE 80

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 39 | Climbing | | | | | | |
| | hydrangea | | | | | | |
| 39-1 | Whole plant | 1 | 98.56 | 113.19 | 88.37 | 73.09 | 208.51 |
| | | 10 | 92.21 | 128.59 | 74.67 | 95.05 | 311.28 |
| | | 100 | 72.15 | 141.71 | 7.32 | 102.22 | 467.91 |
| 39-2 | Roots | 1 | 96.27 | 99.61 | 92.82 | 72.83 | 163.79 |
| | | 10 | 92.43 | 127.17 | 55.47 | 82.89 | 249.57 |
| | | 100 | 79.53 | 155.24 | 38.81 | 109.71 | 395.63 |
| 39-3 | Stems | 1 | 100.61 | 113.88 | 80.64 | 74.61 | 152.06 |
| | | 10 | 85.88 | 114.52 | 60.43 | 91.60 | 278.22 |
| | | 100 | 75.34 | 146.36 | 16.27 | 95.76 | 358.19 |
| 39-4 | Leaves | 1 | 99.50 | 114.59 | 95.18 | 72.84 | 160.29 |
| | | 10 | 95.09 | 119.28 | 67.33 | 79.81 | 210.55 |
| | | 100 | 71.29 | 150.95 | 20.94 | 103.58 | 342.35 |
| 39-5 | Fruits | 1 | 100.24 | 98.65 | 105.04 | 82.22 | 97.05 |
| | | 10 | 85.09 | 106.56 | 95.96 | 82.57 | 191.57 |
| | | 100 | 73.11 | 133.08 | 45.91 | 93.15 | 325.91 |
| 39-6 | Flowers | 1 | 99.01 | 99.50 | 98.75 | 83.37 | 209.05 |
| | | 10 | 81.33 | 108.85 | 67.69 | 81.96 | 245.22 |
| | | 100 | 76.25 | 130.73 | 24.59 | 106.04 | 326.35 |

### Example 40. Stringy Stonecrop

TABLE 81

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 40 | Stringy | | | | | | |
| | stonecrop | | | | | | |
| 40-1 | Whole plant | 1 | 28.89 | 89.06 | 153.83 | 82.81 | 98.93 |
| | | 10 | 36.22 | 31.06 | 312.08 | 183.08 | 144.33 |
| | | 100 | 81.05 | 29.44 | 415.95 | 409.20 | 254.92 |
| 40-2 | Roots | 1 | 30.22 | 71.37 | 135.51 | 86.44 | 101.44 |
| | | 10 | 45.92 | 30.91 | 315.26 | 164.26 | 120.17 |
| | | 100 | 81.59 | 27.24 | 413.86 | 490.39 | 325.20 |
| 40-3 | Stems | 1 | 28.55 | 85.67 | 185.30 | 83.86 | 118.13 |
| | | 10 | 38.49 | 39.27 | 225.84 | 175.10 | 208.48 |
| | | 100 | 72.73 | 30.54 | 409.30 | 438.12 | 262.92 |
| 40-4 | Leaves | 1 | 29.38 | 45.35 | 108.69 | 109.21 | 120.75 |
| | | 10 | 48.41 | 40.22 | 267.43 | 181.20 | 122.41 |
| | | 100 | 78.72 | 27.36 | 370.49 | 399.68 | 304.00 |
| 40-5 | Fruits | 1 | 20.34 | 76.48 | 190.26 | 81.89 | 111.79 |
| | | 10 | 42.97 | 38.42 | 213.05 | 161.22 | 127.70 |
| | | 100 | 82.13 | 30.42 | 351.92 | 413.42 | 335.28 |
| 40-6 | Flowers | 1 | 28.25 | 77.95 | 117.35 | 81.53 | 96.50 |
| | | 10 | 32.90 | 39.44 | 236.93 | 144.34 | 118.56 |
| | | 100 | 78.94 | 23.25 | 467.54 | 402.73 | 200.70 |

TABLE 82

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 40 | Stringy | | | | | | |
| | stonecrop | | | | | | |
| 40-1 | Whole plant | 1 | 97.96 | 111.45 | 100.23 | 73.92 | 119.76 |
| | | 10 | 87.84 | 129.87 | 54.93 | 81.09 | 239.29 |
| | | 100 | 75.76 | 148.17 | 12.48 | 95.96 | 467.09 |
| 40-2 | Roots | 1 | 95.05 | 104.37 | 83.99 | 79.20 | 153.44 |
| | | 10 | 83.48 | 133.54 | 52.42 | 77.99 | 184.80 |
| | | 100 | 76.73 | 144.94 | 23.45 | 99.11 | 327.17 |
| 40-3 | Stems | 1 | 103.63 | 100.90 | 80.61 | 76.29 | 199.89 |
| | | 10 | 88.72 | 127.76 | 88.10 | 80.79 | 178.12 |
| | | 100 | 80.66 | 140.56 | 45.67 | 91.70 | 392.41 |
| 40-4 | Leaves | 1 | 102.42 | 102.57 | 88.01 | 69.55 | 202.39 |
| | | 10 | 89.79 | 114.16 | 93.57 | 87.29 | 284.71 |
| | | 100 | 77.28 | 156.79 | 2.02 | 96.06 | 411.38 |
| 40-5 | Fruits | 1 | 99.05 | 97.99 | 102.91 | 85.89 | 201.92 |
| | | 10 | 80.84 | 106.67 | 53.59 | 85.06 | 223.90 |
| | | 100 | 77.64 | 156.82 | 17.81 | 109.08 | 389.77 |
| 40-6 | Flowers | 1 | 96.13 | 97.84 | 84.89 | 68.57 | 135.87 |
| | | 10 | 84.59 | 118.31 | 84.55 | 90.98 | 222.47 |
| | | 100 | 72.57 | 141.37 | 23.23 | 98.08 | 375.99 |

### Example 41. Ulleungdo Stonecrop

TABLE 83

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm ) | 70.4 nM) | 10 μM) | 1 ppm) | 1 ng/ml) |
| 41 | Ulleungdo | | | | | | |
| | stonecrop | | | | | | |
| 41-1 | Whole plant | 1 | 28.16 | 89.83 | 147.32 | 92.83 | 108.33 |
| | | 10 | 40.41 | 30.19 | 220.66 | 181.94 | 187.58 |
| | | 100 | 77.16 | 25.79 | 430.32 | 479.77 | 299.03 |
| 41-2 | Roots | 1 | 28.51 | 63.72 | 185.64 | 81.25 | 138.59 |
| | | 10 | 50.40 | 36.71 | 313.85 | 162.03 | 129.38 |
| | | 100 | 73.48 | 27.69 | 353.76 | 467.31 | 314.95 |
| 41-3 | Stems | 1 | 24.30 | 44.78 | 124.48 | 85.50 | 115.43 |
| | | 10 | 44.78 | 39.41 | 337.92 | 132.49 | 183.47 |
| | | 100 | 84.91 | 25.56 | 485.86 | 445.55 | 203.92 |
| 41-4 | Leaves | 1 | 29.75 | 56.47 | 175.28 | 105.90 | 145.94 |
| | | 10 | 43.61 | 32.70 | 206.01 | 130.75 | 217.32 |
| | | 100 | 79.16 | 23.85 | 403.01 | 341.54 | 223.70 |
| 41-5 | Fruits | 1 | 29.18 | 57.99 | 183.42 | 84.75 | 109.30 |
| | | 10 | 37.49 | 36.21 | 287.58 | 198.43 | 138.41 |
| | | 100 | 81.30 | 24.41 | 465.53 | 355.07 | 222.23 |
| 41-6 | Flowers | 1 | 21.35 | 59.77 | 125.26 | 85.29 | 109.24 |
| | | 10 | 39.13 | 38.57 | 270.38 | 193.86 | 144.05 |
| | | 100 | 82.25 | 24.11 | 434.83 | 481.25 | 250.47 |

TABLE 84

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 41 | Ulleungdo | | | | | | |
| | stonecrop | | | | | | |
| 41-1 | Whole plant | 1 | 98.04 | 102.49 | 102.32 | 76.43 | 207.72 |
| | | 10 | 84.46 | 133.50 | 70.45 | 88.13 | 323.03 |
| | | 100 | 78.94 | 160.45 | 15.37 | 109.58 | 437.31 |
| 41-2 | Roots | 1 | 96.28 | 105.92 | 90.56 | 85.02 | 136.13 |
| | | 10 | 91.93 | 117.75 | 85.51 | 84.29 | 315.64 |
| | | 100 | 71.02 | 135.01 | 15.84 | 101.01 | 374.18 |
| 41-3 | Stems | 1 | 98.59 | 108.13 | 102.61 | 83.72 | 199.00 |
| | | 10 | 80.26 | 137.89 | 80.03 | 92.64 | 291.40 |
| | | 100 | 75.44 | 159.62 | 30.14 | 101.83 | 316.32 |
| 41-4 | Leaves | 1 | 95.31 | 101.67 | 83.68 | 84.89 | 126.91 |
| | | 10 | 83.12 | 140.74 | 89.19 | 75.63 | 244.29 |
| | | 100 | 70.20 | 152.73 | 15.24 | 100.20 | 450.35 |
| 41-5 | Fruits | 1 | 102.06 | 97.04 | 104.25 | 71.66 | 203.64 |
| | | 10 | 91.19 | 114.80 | 48.70 | 95.86 | 274.38 |
| | | 100 | 75.86 | 136.31 | 15.91 | 102.48 | 322.61 |
| 41-6 | Flowers | 1 | 96.61 | 96.53 | 102.53 | 80.98 | 200.46 |
| | | 10 | 95.53 | 140.21 | 80.72 | 89.24 | 205.23 |
| | | 100 | 78.11 | 149.23 | 8.03 | 91.84 | 423.75 |

### Example 42. Foam Flower

TABLE 85

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 42 | Foam flower | | | | | | |
| 42-1 | Whole plant | 1 | 21.39 | 64.55 | 149.27 | 110.42 | 83.70 |
| | | 10 | 34.56 | 35.42 | 202.38 | 198.25 | 107.05 |
| | | 100 | 72.72 | 30.37 | 366.96 | 328.34 | 279.19 |
| 42-2 | Roots | 1 | 28.94 | 59.44 | 144.57 | 93.27 | 90.37 |
| | | 10 | 37.16 | 40.92 | 332.99 | 175.03 | 158.15 |
| | | 100 | 77.65 | 26.07 | 403.92 | 433.53 | 242.73 |
| 42-3 | Stems | 1 | 28.89 | 86.68 | 187.58 | 103.30 | 94.83 |
| | | 10 | 39.53 | 34.90 | 294.79 | 167.17 | 187.61 |
| | | 100 | 76.11 | 20.40 | 479.16 | 467.86 | 301.14 |
| 42-4 | Leaves | 1 | 23.37 | 76.29 | 193.95 | 101.32 | 83.36 |
| | | 10 | 33.73 | 30.40 | 308.88 | 150.91 | 211.48 |
| | | 100 | 72.57 | 28.02 | 437.35 | 400.24 | 246.90 |
| 42-5 | Fruits | 1 | 27.78 | 64.91 | 161.47 | 98.66 | 147.51 |
| | | 10 | 35.37 | 34.98 | 206.68 | 131.66 | 171.70 |
| | | 100 | 81.64 | 24.88 | 402.28 | 335.78 | 309.23 |
| 42-6 | Flowers | 1 | 22.16 | 77.99 | 131.79 | 80.53 | 132.96 |
| | | 10 | 31.92 | 33.00 | 278.14 | 196.46 | 217.97 |
| | | 100 | 71.93 | 30.79 | 410.85 | 415.16 | 298.71 |

TABLE 86

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 42 | Foam flower | | | | | | |
| 42-1 | Whole plant | 1 | 98.26 | 98.46 | 87.41 | 66.24 | 177.57 |
| | | 10 | 89.16 | 108.90 | 50.47 | 84.02 | 250.15 |
| | | 100 | 77.19 | 144.48 | 35.97 | 110.72 | 445.39 |
| 42-2 | Roots | 1 | 101.30 | 104.50 | 101.20 | 69.94 | 172.09 |
| | | 10 | 82.62 | 105.84 | 55.30 | 80.08 | 180.25 |
| | | 100 | 73.64 | 159.98 | 7.28 | 99.90 | 346.10 |
| 42-3 | Stems | 1 | 101.83 | 102.72 | 99.48 | 66.54 | 204.10 |
| | | 10 | 82.92 | 121.80 | 64.55 | 93.07 | 211.30 |
| | | 100 | 73.68 | 155.33 | 27.28 | 105.73 | 308.78 |
| 42-4 | Leaves | 1 | 101.49 | 113.23 | 99.49 | 71.93 | 153.62 |
| | | 10 | 92.71 | 124.49 | 76.86 | 92.13 | 175.74 |
| | | 100 | 72.56 | 147.20 | 1.45 | 109.19 | 325.54 |
| 42-5 | Fruits | 1 | 97.22 | 98.34 | 83.16 | 76.04 | 130.86 |
| | | 10 | 90.69 | 116.55 | 45.93 | 81.01 | 319.84 |
| | | 100 | 79.60 | 154.39 | 3.04 | 104.47 | 327.03 |
| 42-6 | Flowers | 1 | 101.18 | 102.72 | 101.85 | 80.17 | 164.12 |
| | | 10 | 81.39 | 127.45 | 84.42 | 76.45 | 333.74 |
| | | 100 | 79.32 | 156.46 | 35.04 | 93.96 | 380.37 |

### Example 43. East Asian Cinquefoil

TABLE 87

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 43 | East Asian | | | | | | |
| | cinquefoil | | | | | | |
| 43-1 | Whole plant | 1 | 22.36 | 84.93 | 135.28 | 82.53 | 89.45 |
| | | 10 | 40.28 | 33.97 | 200.96 | 141.23 | 121.04 |
| | | 100 | 75.44 | 24.56 | 411.25 | 318.15 | 337.59 |
| 43-2 | Roots | 1 | 29.69 | 84.99 | 124.21 | 82.59 | 146.38 |
| | | 10 | 36.96 | 36.19 | 296.12 | 150.96 | 106.16 |
| | | 100 | 73.33 | 23.06 | 468.56 | 494.12 | 222.29 |
| 43-3 | Stems | 1 | 23.76 | 51.44 | 134.98 | 83.80 | 101.89 |
| | | 10 | 32.17 | 31.12 | 305.70 | 141.76 | 118.72 |
| | | 100 | 78.34 | 24.30 | 393.05 | 335.62 | 213.53 |
| 43-4 | Leaves | 1 | 27.15 | 71.88 | 187.09 | 103.74 | 114.42 |
| | | 10 | 36.75 | 30.98 | 310.77 | 176.66 | 141.17 |
| | | 100 | 70.88 | 22.24 | 452.64 | 500.85 | 311.66 |
| 43-5 | Fruits | 1 | 25.34 | 45.38 | 155.72 | 81.44 | 146.85 |
| | | 10 | 47.08 | 37.90 | 233.24 | 115.41 | 211.80 |
| | | 100 | 85.95 | 25.40 | 356.98 | 462.24 | 315.93 |
| 43-6 | Flowers | 1 | 25.78 | 68.21 | 197.20 | 91.25 | 134.34 |
| | | 10 | 39.86 | 34.68 | 297.20 | 177.84 | 177.08 |
| | | 100 | 77.68 | 25.62 | 402.54 | 310.04 | 258.84 |

TABLE 88

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 43 | East Asian | | | | | | |
| | cinquefoil | | | | | | |
| 43-1 | Whole plant | 1 | 97.94 | 99.55 | 90.17 | 77.16 | 146.55 |
| | | 10 | 93.92 | 110.52 | 82.55 | 85.94 | 338.58 |
| | | 100 | 76.34 | 141.52 | 36.22 | 94.99 | 493.78 |
| 43-2 | Roots | 1 | 102.32 | 115.06 | 84.85 | 77.40 | 128.29 |
| | | 10 | 89.38 | 116.35 | 62.08 | 77.99 | 217.20 |
| | | 100 | 73.46 | 139.42 | 0.17 | 90.86 | 374.58 |
| 43-3 | Stems | 1 | 95.28 | 99.49 | 91.85 | 77.69 | 157.89 |
| | | 10 | 84.26 | 126.57 | 81.63 | 76.86 | 175.43 |
| | | 100 | 75.59 | 130.96 | 32.72 | 105.79 | 474.06 |
| 43-4 | Leaves | 1 | 95.77 | 106.23 | 82.58 | 65.16 | 125.35 |
| | | 10 | 88.07 | 137.22 | 94.45 | 79.10 | 195.95 |
| | | 100 | 79.98 | 139.70 | 45.19 | 94.17 | 435.94 |
| 43-5 | Fruits | 1 | 100.99 | 114.45 | 103.56 | 65.23 | 165.30 |
| | | 10 | 84.82 | 131.99 | 89.42 | 76.87 | 262.56 |
| | | 100 | 75.86 | 159.43 | 9.21 | 91.39 | 469.20 |
| 43-6 | Flowers | 1 | 97.27 | 109.30 | 89.32 | 78.83 | 91.46 |
| | | 10 | 87.81 | 115.16 | 80.31 | 95.57 | 324.82 |
| | | 100 | 77.50 | 131.17 | 10.72 | 93.57 | 433.56 |

### Example 44. Indigobush Amorpha

TABLE 89

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 44 | Indigobush | | | | | | |
| | Amorpha | | | | | | |
| 44-1 | Whole plant | 1 | 22.50 | 83.08 | 163.17 | 89.34 | 134.52 |
| | | 10 | 44.33 | 31.88 | 344.55 | 191.55 | 118.03 |
| | | 100 | 81.82 | 26.76 | 441.92 | 372.86 | 283.94 |
| 44-2 | Roots | 1 | 30.66 | 85.02 | 129.88 | 94.64 | 100.23 |
| | | 10 | 33.33 | 36.28 | 207.16 | 181.98 | 155.56 |
| | | 100 | 72.26 | 25.40 | 379.68 | 301.60 | 204.32 |
| 44-3 | Stems | 1 | 27.47 | 46.12 | 198.14 | 86.90 | 93.04 |
| | | 10 | 48.33 | 34.97 | 322.95 | 118.91 | 138.96 |
| | | 100 | 79.32 | 22.78 | 490.93 | 347.75 | 310.70 |
| 44-4 | Leaves | 1 | 23.82 | 79.95 | 118.10 | 101.48 | 139.99 |
| | | 10 | 38.03 | 36.31 | 268.46 | 177.94 | 129.96 |
| | | 100 | 72.67 | 23.45 | 460.95 | 307.45 | 211.55 |
| 44-5 | Fruits | 1 | 22.68 | 66.13 | 139.39 | 81.20 | 105.72 |
| | | 10 | 36.99 | 30.10 | 207.51 | 138.71 | 139.31 |
| | | 100 | 71.42 | 20.76 | 478.51 | 319.15 | 204.50 |
| 44-6 | Flowers | 1 | 23.29 | 49.42 | 149.44 | 80.94 | 120.65 |
| | | 10 | 30.29 | 35.92 | 277.66 | 153.66 | 148.69 |
| | | 100 | 72.99 | 20.02 | 473.50 | 384.05 | 280.25 |

TABLE 90

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 44 | Indigobush | | | | | | |
| | Amorpha | | | | | | |
| 44-1 | Whole plant | 1 | 102.24 | 113.84 | 108.51 | 69.03 | 151.24 |
| | | 10 | 81.71 | 128.79 | 61.41 | 85.48 | 298.81 |
| | | 100 | 72.62 | 138.30 | 21.80 | 91.26 | 472.07 |
| 44-2 | Roots | 1 | 103.78 | 114.78 | 110.26 | 85.29 | 116.26 |
| | | 10 | 95.58 | 114.02 | 70.56 | 76.47 | 340.51 |
| | | 100 | 79.22 | 131.08 | 42.31 | 91.89 | 394.81 |
| 44-3 | Stems | 1 | 95.59 | 96.36 | 91.44 | 78.53 | 163.94 |
| | | 10 | 91.47 | 128.22 | 50.30 | 90.82 | 305.55 |
| | | 100 | 74.50 | 142.97 | 42.21 | 107.27 | 406.77 |
| 44-4 | Leaves | 1 | 103.08 | 99.00 | 86.25 | 81.07 | 155.69 |
| | | 10 | 82.06 | 117.49 | 63.53 | 87.69 | 292.68 |
| | | 100 | 75.68 | 133.08 | 41.14 | 100.17 | 454.52 |
| 44-5 | Fruits | 1 | 96.07 | 109.89 | 80.90 | 72.11 | 194.92 |
| | | 10 | 86.33 | 139.91 | 72.47 | 92.54 | 333.77 |
| | | 100 | 74.89 | 150.11 | 11.12 | 90.39 | 441.15 |
| 44-6 | Flowers | 1 | 102.88 | 98.90 | 104.59 | 70.77 | 134.98 |
| | | 10 | 81.10 | 135.90 | 56.74 | 93.92 | 229.40 |
| | | 100 | 75.39 | 149.83 | 35.98 | 100.83 | 455.60 |

### Example 45. Sericea Lespedeza

TABLE 91

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 45 | Sericea | | | | | | |
| | lespedeza | | | | | | |
| 45-1 | Whole plant | 1 | 24.22 | 49.97 | 147.19 | 83.35 | 99.46 |
| | | 10 | 38.63 | 37.61 | 331.06 | 184.98 | 249.92 |
| | | 100 | 71.18 | 28.16 | 351.53 | 341.71 | 325.25 |
| 45-2 | Roots | 1 | 29.19 | 77.35 | 149.22 | 101.44 | 133.02 |
| | | 10 | 36.86 | 40.27 | 222.02 | 195.63 | 213.41 |
| | | 100 | 78.65 | 22.96 | 440.25 | 350.99 | 318.62 |
| 45-3 | Stems | 1 | 28.40 | 59.36 | 107.74 | 85.33 | 101.56 |
| | | 10 | 20.97 | 36.89 | 260.32 | 138.50 | 245.34 |
| | | 100 | 23.53 | 28.54 | 478.70 | 424.69 | 208.22 |
| 45-4 | Leaves | 1 | 28.47 | 53.42 | 157.89 | 97.98 | 112.80 |
| | | 10 | 30.94 | 34.61 | 346.84 | 113.67 | 191.41 |
| | | 100 | 12.63 | 24.29 | 385.51 | 451.30 | 231.36 |
| 45-5 | Fruits | 1 | 27.86 | 76.94 | 101.44 | 97.97 | 145.57 |
| | | 10 | 49.86 | 36.01 | 299.07 | 136.13 | 213.02 |
| | | 100 | 78.54 | 22.95 | 442.24 | 377.71 | 316.85 |
| 45-6 | Flowers | 1 | 23.50 | 60.78 | 112.70 | 96.49 | 137.50 |
| | | 10 | 33.58 | 40.77 | 319.45 | 166.78 | 203.78 |
| | | 100 | 76.55 | 23.67 | 407.47 | 395.12 | 267.08 |

TABLE 92

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | ( % of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 45 | Sericea | | | | | | |
| | lespedeza | | | | | | |
| 45-1 | Whole plant | 1 | 98.49 | 106.92 | 85.81 | 71.43 | 183.25 |
| | | 10 | 89.29 | 137.22 | 50.10 | 77.84 | 269.68 |
| | | 100 | 70.28 | 160.10 | 46.31 | 96.47 | 429.39 |
| 45-2 | Roots | 1 | 96.60 | 100.00 | 86.73 | 83.07 | 204.03 |
| | | 10 | 82.45 | 132.36 | 78.55 | 93.25 | 247.72 |
| | | 100 | 72.70 | 154.94 | 17.36 | 109.62 | 457.51 |
| 45-3 | Stems | 1 | 103.20 | 98.59 | 91.07 | 75.70 | 137.75 |
| | | 10 | 89.34 | 122.13 | 63.61 | 85.86 | 115.37 |
| | | 100 | 73.42 | 159.81 | 8.62 | 103.92 | 105.37 |
| 45-4 | Leaves | 1 | 96.28 | 109.34 | 102.08 | 83.13 | 91.52 |
| | | 10 | 92.48 | 133.39 | 78.74 | 89.90 | 125.85 |
| | | 100 | 71.15 | 155.68 | 49.73 | 92.51 | 103.90 |
| 45-5 | Fruits | 1 | 98.07 | 111.01 | 90.41 | 83.25 | 128.44 |
| | | 10 | 90.57 | 105.21 | 72.22 | 86.89 | 278.01 |
| | | 100 | 74.15 | 160.97 | 14.93 | 94.15 | 333.28 |
| 45-6 | Flowers | 1 | 97.90 | 101.28 | 102.16 | 66.22 | 160.38 |
| | | 10 | 95.10 | 120.75 | 73.30 | 92.05 | 207.15 |
| | | 100 | 70.26 | 143.46 | 12.03 | 109.99 | 494.40 |

### Example 46. Alfalfa

TABLE 93

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 46 | Alfalfa | | | | | | |
| 46-1 | Whole plant | 1 | 22.39 | 47.93 | 167.63 | 86.90 | 89.66 |
| | | 10 | 30.17 | 31.44 | 267.92 | 146.41 | 151.06 |
| | | 100 | 82.36 | 30.29 | 426.97 | 340.88 | 349.87 |
| 46-2 | Roots | 1 | 29.03 | 43.72 | 168.82 | 93.77 | 101.56 |
| | | 10 | 35.32 | 36.26 | 345.63 | 164.10 | 149.68 |
| | | 100 | 74.45 | 21.49 | 483.73 | 494.74 | 321.99 |
| 46-3 | Stems | 1 | 21.96 | 88.15 | 147.54 | 110.67 | 86.43 |
| | | 10 | 33.17 | 37.05 | 279.77 | 200.82 | 248.95 |
| | | 100 | 85.54 | 28.69 | 497.81 | 417.29 | 277.38 |
| 46-4 | Leaves | 1 | 24.96 | 76.20 | 128.02 | 85.24 | 98.94 |
| | | 10 | 41.36 | 40.57 | 266.30 | 165.84 | 176.65 |
| | | 100 | 74.63 | 28.58 | 484.46 | 416.08 | 330.90 |
| 46-5 | Fruits | 1 | 20.61 | 55.37 | 189.25 | 90.39 | 138.38 |
| | | 10 | 44.86 | 38.34 | 333.15 | 119.58 | 174.01 |
| | | 100 | 80.49 | 30.88 | 488.06 | 424.56 | 258.75 |
| 46-6 | Flowers | 1 | 27.18 | 53.72 | 119.30 | 94.38 | 142.02 |
| | | 10 | 33.02 | 34.24 | 205.24 | 119.77 | 242.58 |
| | | 100 | 76.22 | 27.78 | 477.26 | 488.81 | 255.05 |

TABLE 94

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 46 | Alfalfa | | | | | | |
| 46-1 | Whole plant | 1 | 97.87 | 101.68 | 85.25 | 79.86 | 181.52 |
| | | 10 | 82.60 | 138.93 | 70.85 | 89.09 | 333.19 |
| | | 100 | 76.21 | 150.61 | 3.25 | 109.90 | 445.39 |
| 46-2 | Roots | 1 | 101.99 | 112.60 | 88.29 | 83.27 | 119.38 |
| | | 10 | 89.62 | 125.49 | 78.36 | 81.22 | 335.79 |
| | | 100 | 70.99 | 136.77 | 35.45 | 94.00 | 492.69 |
| 46-3 | Stems | 1 | 100.41 | 106.28 | 107.12 | 81.82 | 197.30 |
| | | 10 | 84.49 | 131.14 | 91.99 | 83.36 | 321.30 |
| | | 100 | 72.30 | 152.30 | 25.79 | 92.71 | 431.71 |
| 46-4 | Leaves | 1 | 97.82 | 111.99 | 106.23 | 84.94 | 203.35 |
| | | 10 | 80.15 | 140.39 | 49.48 | 83.66 | 241.81 |
| | | 100 | 77.91 | 148.31 | 36.83 | 105.28 | 398.74 |
| 46-5 | Fruits | 1 | 100.05 | 102.85 | 106.43 | 84.80 | 148.30 |
| | | 10 | 86.25 | 114.40 | 91.70 | 77.50 | 250.97 |
| | | 100 | 79.23 | 160.85 | 13.57 | 92.64 | 351.20 |
| 46-6 | Flowers | 1 | 98.83 | 105.84 | 87.44 | 65.88 | 141.45 |
| | | 10 | 95.73 | 113.07 | 64.71 | 76.79 | 178.49 |
| | | 100 | 77.60 | 147.22 | 17.23 | 101.45 | 301.65 |

### Example 47. Amur Vetch

TABLE 95

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 47 | Amur vetch | | | | | | |
| 47-1 | Whole plant | 1 | 20.91 | 49.14 | 145.41 | 106.24 | 114.72 |
| | | 10 | 34.12 | 37.31 | 319.43 | 198.57 | 181.24 |
| | | 100 | 74.79 | 21.29 | 352.32 | 413.82 | 263.34 |
| 47-2 | Roots | 1 | 22.04 | 76.23 | 190.42 | 104.37 | 118.56 |
| | | 10 | 48.82 | 39.18 | 309.67 | 156.82 | 178.48 |
| | | 100 | 72.30 | 22.46 | 431.21 | 433.67 | 233.44 |
| 47-3 | Stems | 1 | 29.98 | 68.26 | 146.21 | 110.72 | 91.72 |
| | | 10 | 35.02 | 30.56 | 200.12 | 128.57 | 193.40 |
| | | 100 | 75.75 | 22.96 | 490.27 | 379.08 | 315.42 |
| 47-4 | Leaves | 1 | 26.00 | 48.69 | 127.30 | 88.64 | 112.72 |
| | | 10 | 41.28 | 31.97 | 329.03 | 185.23 | 153.66 |
| | | 100 | 83.92 | 21.75 | 407.85 | 476.14 | 350.67 |
| 47-5 | Fruits | 1 | 24.06 | 86.90 | 177.73 | 99.90 | 120.97 |
| | | 10 | 37.48 | 40.51 | 232.46 | 144.61 | 138.78 |
| | | 100 | 71.13 | 22.92 | 499.60 | 311.59 | 346.21 |
| 47-6 | Flowers | 1 | 29.55 | 44.07 | 163.80 | 95.50 | 105.30 |
| | | 10 | 32.19 | 32.65 | 255.28 | 200.21 | 189.00 |
| | | 100 | 84.31 | 28.92 | 470.54 | 309.48 | 306.12 |

TABLE 96

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 47 | Amur vetch | | | | | | |
| 47-1 | Whole plant | 1 | 102.52 | 101.23 | 108.85 | 65.78 | 165.88 |
| | | 10 | 84.04 | 121.90 | 69.76 | 91.82 | 209.71 |
| | | 100 | 80.65 | 148.55 | 44.09 | 105.32 | 313.27 |
| 47-2 | Roots | 1 | 102.09 | 109.24 | 101.37 | 76.04 | 106.11 |
| | | 10 | 87.80 | 115.21 | 83.84 | 92.19 | 331.35 |
| | | 100 | 80.89 | 151.16 | 5.76 | 106.03 | 461.19 |
| 47-3 | Stems | 1 | 101.58 | 111.61 | 101.25 | 77.27 | 135.78 |
| | | 10 | 92.93 | 111.23 | 67.79 | 78.13 | 282.13 |
| | | 100 | 73.54 | 160.28 | 20.77 | 94.30 | 396.52 |
| 47-4 | Leaves | 1 | 99.27 | 104.46 | 104.10 | 80.98 | 206.10 |
| | | 10 | 81.43 | 135.89 | 92.05 | 93.60 | 206.42 |
| | | 100 | 76.53 | 160.02 | 23.08 | 103.67 | 318.67 |
| 47-5 | Fruits | 1 | 99.83 | 98.85 | 106.50 | 75.27 | 180.27 |
| | | 10 | 83.08 | 138.61 | 92.94 | 76.33 | 260.61 |
| | | 100 | 78.96 | 148.05 | 36.08 | 92.70 | 354.67 |
| 47-6 | Flowers | 1 | 100.56 | 113.08 | 97.06 | 79.85 | 129.97 |
| | | 10 | 83.71 | 119.67 | 55.92 | 87.70 | 325.26 |
| | | 100 | 77.15 | 159.49 | 0.94 | 102.31 | 489.65 |

### Example 48. Hairy Purple Loosestrife

TABLE 97

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 48 | Hairy purple | | | | | | |
| | loosestrife | | | | | | |
| 48-1 | Whole plant | 1 | 27.14 | 71.84 | 109.74 | 93.38 | 83.21 |
| | | 10 | 32.91 | 38.02 | 335.18 | 147.55 | 177.26 |
| | | 100 | 85.20 | 25.25 | 388.77 | 302.74 | 284.23 |
| 48-2 | Roots | 1 | 23.06 | 59.35 | 115.45 | 109.66 | 138.38 |
| | | 10 | 46.96 | 35.57 | 345.40 | 177.21 | 231.62 |
| | | 100 | 74.99 | 22.57 | 389.25 | 319.45 | 255.17 |
| 48-3 | Stems | 1 | 28.55 | 52.05 | 146.67 | 100.10 | 85.51 |
| | | 10 | 30.32 | 31.67 | 227.75 | 129.68 | 155.82 |
| | | 100 | 85.72 | 26.48 | 391.96 | 315.79 | 269.78 |
| 48-4 | Leaves | 1 | 22.18 | 44.55 | 179.15 | 94.61 | 85.29 |
| | | 10 | 41.90 | 34.71 | 213.47 | 139.92 | 247.51 |
| | | 100 | 81.34 | 21.85 | 428.48 | 398.88 | 214.46 |
| 48-5 | Fruits | 1 | 24.04 | 70.76 | 112.74 | 108.39 | 121.78 |
| | | 10 | 33.23 | 35.53 | 247.23 | 113.66 | 243.79 |
| | | 100 | 79.44 | 21.21 | 426.71 | 486.89 | 256.28 |
| 48-6 | Flowers | 1 | 25.94 | 54.70 | 112.47 | 94.78 | 99.99 |
| | | 10 | 31.26 | 39.53 | 271.68 | 121.98 | 195.73 |
| | | 100 | 78.23 | 26.50 | 364.70 | 457.97 | 214.73 |
| 48-7 | Shoots | 1 | 29.05 | 62.80 | 104.22 | 88.91 | 119.78 |
| | | 10 | 43.52 | 36.46 | 77.05 | 124.05 | 137.22 |
| | | 100 | 83.27 | 23.64 | 116.59 | 402.38 | 206.76 |

TABLE 98

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 48 | Hairy purple | | | | | | |
| | loosestrife | | | | | | |
| 48-1 | Whole plant | 1 | 97.53 | 95.42 | 98.60 | 82.43 | 174.86 |
| | | 10 | 82.71 | 130.33 | 74.13 | 75.52 | 315.81 |
| | | 100 | 79.71 | 141.21 | 41.36 | 102.41 | 348.27 |
| 48-2 | Roots | 1 | 100.83 | 95.50 | 90.54 | 76.26 | 167.06 |
| | | 10 | 94.60 | 120.61 | 91.26 | 82.03 | 247.14 |
| | | 100 | 76.42 | 143.08 | 44.64 | 110.31 | 314.73 |
| 48-3 | Stems | 1 | 98.70 | 112.01 | 96.81 | 84.88 | 144.19 |
| | | 10 | 86.91 | 139.97 | 48.97 | 78.00 | 326.37 |
| | | 100 | 73.36 | 141.94 | 0.15 | 102.75 | 404.41 |
| 48-4 | Leaves | 1 | 103.10 | 105.40 | 101.64 | 80.28 | 159.17 |
| | | 10 | 93.05 | 114.75 | 61.08 | 84.33 | 287.01 |
| | | 100 | 70.18 | 140.34 | 4.70 | 90.46 | 305.15 |
| 48-5 | Fruits | 1 | 102.03 | 111.19 | 89.67 | 72.95 | 143.76 |
| | | 10 | 87.69 | 137.56 | 75.34 | 89.42 | 264.14 |
| | | 100 | 73.95 | 135.18 | 21.46 | 110.76 | 450.24 |
| 48-6 | Flowers | 1 | 103.10 | 100.74 | 96.71 | 68.18 | 135.54 |
| | | 10 | 94.09 | 127.60 | 82.66 | 88.97 | 174.03 |
| | | 100 | 80.92 | 157.81 | 40.08 | 102.99 | 471.02 |
| 48-7 | Shoots | 1 | 100.42 | 104.16 | 106.35 | 69.17 | 119.47 |
| | | 10 | 87.85 | 100.30 | 48.13 | 71.12 | 195.17 |
| | | 100 | 73.29 | 104.30 | 32.83 | 73.15 | 390.05 |

### Example 49. South Enchanter's Nightshade

TABLE 99

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 49 | South | | | | | | |
| | enchanter's | | | | | | |
| | nightshade | | | | | | |
| 49-1 | Whole plant | 1 | 20.17 | 69.34 | 152.04 | 97.40 | 127.90 |
| | | 10 | 35.98 | 39.89 | 289.24 | 118.54 | 196.50 |
| | | 100 | 76.96 | 29.00 | 363.54 | 354.05 | 271.84 |
| 49-2 | Roots | 1 | 28.35 | 42.72 | 129.87 | 82.48 | 102.74 |
| | | 10 | 39.23 | 34.28 | 279.29 | 142.79 | 198.70 |
| | | 100 | 79.75 | 24.76 | 493.70 | 332.00 | 298.14 |
| 49-3 | Stems | 1 | 28.63 | 58.17 | 137.70 | 84.34 | 86.96 |
| | | 10 | 43.16 | 38.68 | 261.14 | 135.66 | 242.02 |
| | | 100 | 80.74 | 27.88 | 487.29 | 367.76 | 207.79 |
| 49-4 | Leaves | 1 | 20.11 | 69.50 | 127.70 | 96.74 | 123.73 |
| | | 10 | 30.32 | 39.88 | 222.23 | 139.69 | 229.49 |
| | | 100 | 78.45 | 21.50 | 360.83 | 482.11 | 273.75 |
| 49-5 | Fruits | 1 | 25.62 | 89.00 | 112.58 | 94.23 | 130.50 |
| | | 10 | 34.71 | 36.43 | 319.01 | 125.49 | 246.25 |
| | | 100 | 82.45 | 27.58 | 389.99 | 313.45 | 266.75 |
| 49-6 | Flowers | 1 | 20.41 | 52.69 | 139.46 | 85.67 | 84.17 |
| | | 10 | 42.54 | 30.34 | 201.04 | 175.35 | 184.53 |
| | | 100 | 85.13 | 26.12 | 379.69 | 464.30 | 264.10 |

TABLE 100

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 49 | South | | | | | | |
| | enchanter's | | | | | | |
| | nightshade | | | | | | |
| 49-1 | Whole plant | 1 | 99.12 | 106.49 | 94.27 | 70.04 | 155.63 |
| | | 10 | 94.95 | 116.52 | 46.06 | 86.76 | 319.57 |
| | | 100 | 70.58 | 160.38 | 47.65 | 101.98 | 332.48 |
| 49-2 | Roots | 1 | 103.47 | 102.28 | 84.93 | 68.11 | 160.29 |
| | | 10 | 82.03 | 139.35 | 75.36 | 78.76 | 229.55 |
| | | 100 | 73.98 | 135.86 | 19.58 | 102.21 | 475.41 |
| 49-3 | Stems | 1 | 98.87 | 107.08 | 91.45 | 81.65 | 179.66 |
| | | 10 | 84.76 | 133.13 | 63.02 | 90.66 | 228.65 |
| | | 100 | 80.01 | 149.56 | 39.42 | 105.09 | 461.82 |
| 49-4 | Leaves | 1 | 98.04 | 107.38 | 109.99 | 82.50 | 183.97 |
| | | 10 | 87.65 | 124.92 | 46.20 | 80.09 | 339.12 |
| | | 100 | 74.05 | 138.48 | 47.42 | 103.64 | 439.51 |
| 49-5 | Fruits | 1 | 96.39 | 99.18 | 94.21 | 77.19 | 137.49 |
| | | 10 | 86.49 | 135.06 | 70.80 | 79.14 | 204.40 |
| | | 100 | 71.35 | 159.00 | 23.31 | 104.27 | 462.32 |
| 49-6 | Flowers | 1 | 100.30 | 97.88 | 81.81 | 81.39 | 166.92 |
| | | 10 | 93.49 | 121.90 | 56.21 | 80.86 | 307.93 |
| | | 100 | 72.27 | 137.95 | 32.84 | 92.55 | 320.36 |

### Example 50. Long-Seed Willowherb

TABLE 101

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity- |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 50 | Long-seed | | | | | | |
| | willowherb | | | | | | |
| 50-1 | Whole plant | 1 | 26.54 | 54.20 | 119.85 | 92.38 | 86.53 |
| | | 10 | 33.40 | 34.05 | 207.22 | 146.64 | 195.20 |
| | | 100 | 80.94 | 25.22 | 402.95 | 336.32 | 304.14 |
| 50-2 | Roots | 1 | 21.84 | 85.62 | 140.47 | 104.74 | 132.68 |
| | | 10 | 47.58 | 37.55 | 347.67 | 198.45 | 125.89 |
| | | 100 | 78.66 | 22.01 | 406.86 | 397.37 | 267.58 |
| 50-3 | Stems | 1 | 24.44 | 67.89 | 146.02 | 103.36 | 112.02 |
| | | 10 | 40.40 | 36.60 | 302.47 | 137.64 | 210.48 |
| | | 100 | 83.86 | 28.28 | 371.81 | 432.69 | 297.77 |
| 50-4 | Leaves | 1 | 22.75 | 62.92 | 178.41 | 96.74 | 94.35 |
| | | 10 | 34.29 | 38.96 | 323.24 | 131.73 | 197.85 |
| | | 100 | 82.36 | 22.11 | 475.49 | 487.50 | 328.90 |
| 50-5 | Fruits | 1 | 27.58 | 65.40 | 132.29 | 102.41 | 98.49 |
| | | 10 | 30.38 | 35.17 | 237.90 | 166.09 | 124.28 |
| | | 100 | 84.01 | 20.69 | 411.12 | 374.23 | 293.02 |
| 50-6 | Flowers | 1 | 24.70 | 88.90 | 135.13 | 108.14 | 100.03 |
| | | 10 | 38.25 | 30.78 | 232.21 | 172.82 | 102.07 |
| | | 100 | 82.19 | 24.71 | 388.08 | 336.27 | 343.14 |

TABLE 102

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM ) | | 100 nM) |
| 50 | Long-seed | | | | | | |
| | willowherb | | | | | | |
| 50-1 | Whole plant | 1 | 95.00 | 115.01 | 98.34 | 66.01 | 203.30 |
| | | 10 | 94.93 | 131.58 | 58.38 | 85.77 | 325.92 |
| | | 100 | 77.97 | 150.47 | 4.10 | 106.24 | 479.73 |
| 50-2 | Roots | 1 | 97.28 | 103.05 | 100.04 | 69.13 | 122.97 |
| | | 10 | 88.64 | 109.93 | 93.30 | 86.46 | 295.46 |
| | | 100 | 79.70 | 143.72 | 11.67 | 91.40 | 389.21 |
| 50-3 | Stems | 1 | 100.39 | 108.74 | 104.05 | 82.93 | 98.51 |
| | | 10 | 87.86 | 130.76 | 56.40 | 94.71 | 246.93 |
| | | 100 | 71.10 | 133.35 | 17.29 | 110.49 | 304.38 |
| 50-4 | Leaves | 1 | 95.43 | 109.49 | 104.95 | 81.64 | 197.51 |
| | | 10 | 91.62 | 116.34 | 56.78 | 87.06 | 338.70 |
| | | 100 | 71.07 | 159.50 | 49.17 | 105.04 | 347.05 |
| 50-5 | Fruits | 1 | 102.89 | 96.75 | 101.46 | 78.42 | 105.83 |
| | | 10 | 86.03 | 113.29 | 45.25 | 78.99 | 192.53 |
| | | 100 | 73.58 | 150.82 | 49.00 | 103.35 | 483.78 |
| 50-6 | Flowers | 1 | 97.52 | 112.25 | 87.07 | 74.16 | 117.64 |
| | | 10 | 82.37 | 108.36 | 90.35 | 88.86 | 230.92 |
| | | 100 | 78.21 | 142.30 | 30.03 | 98.27 | 477.12 |

### Example 51. Evening Primrose

TABLE 103

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 51 | Evening | | | | | | |
| | Primrose | | | | | | |
| 51-1 | Whole plant | 1 | 29.96 | 81.21 | 165.04 | 84.50 | 133.09 |
| | | 10 | 37.26 | 40.57 | 283.64 | 161.37 | 182.72 |
| | | 100 | 81.24 | 25.14 | 411.28 | 347.27 | 301.61 |
| 51-2 | Roots | 1 | 26.71 | 47.27 | 129.49 | 94.58 | 120.07 |
| | | 10 | 37.66 | 40.98 | 316.41 | 123.53 | 140.33 |
| | | 100 | 73.49 | 21.57 | 481.38 | 458.32 | 251.43 |
| 51-3 | Stems | 1 | 30.08 | 72.62 | 168.76 | 82.87 | 119.96 |
| | | 10 | 44.47 | 39.85 | 343.65 | 164.66 | 149.95 |
| | | 100 | 70.25 | 22.28 | 473.25 | 482.20 | 202.31 |
| 51-4 | Leaves | 1 | 30.93 | 88.11 | 179.01 | 103.04 | 80.07 |
| | | 10 | 43.80 | 36.44 | 208.96 | 116.44 | 114.56 |
| | | 100 | 72.73 | 30.65 | 363.78 | 333.91 | 302.22 |
| 51-5 | Fruits | 1 | 27.37 | 90.04 | 188.37 | 89.47 | 123.55 |
| | | 10 | 45.25 | 39.52 | 315.82 | 155.44 | 230.76 |
| | | 100 | 78.52 | 29.40 | 378.32 | 500.19 | 205.90 |
| 51-6 | Flowers | 1 | 30.50 | 49.22 | 199.32 | 86.60 | 130.15 |
| | | 10 | 40.05 | 32.61 | 209.43 | 141.04 | 203.42 |
| | | 100 | 74.56 | 22.64 | 455.01 | 460.81 | 336.34 |

TABLE 104

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 51 | Evening | | | | | | |
| | Primrose | | | | | | |
| 51-1 | Whole plant | 1 | 100.86 | 110.46 | 94.25 | 66.69 | 100.23 |
| | | 10 | 81.00 | 131.63 | 72.08 | 94.03 | 208.56 |
| | | 100 | 76.69 | 142.85 | 48.27 | 104.25 | 481.25 |
| 51-2 | Roots | 1 | 96.32 | 101.74 | 101.75 | 81.40 | 166.30 |
| | | 10 | 92.18 | 127.33 | 63.49 | 92.97 | 178.87 |
| | | 100 | 78.98 | 158.31 | 4.46 | 102.76 | 325.36 |
| 51-3 | Stems | 1 | 96.39 | 98.79 | 95.49 | 77.48 | 118.45 |
| | | 10 | 83.23 | 136.43 | 81.80 | 79.02 | 318.86 |
| | | 100 | 77.52 | 143.07 | 45.13 | 108.56 | 322.55 |
| 51-4 | Leaves | 1 | 101.37 | 104.89 | 88.48 | 68.28 | 175.65 |
| | | 10 | 92.51 | 124.78 | 89.46 | 84.23 | 185.70 |
| | | 100 | 78.12 | 137.59 | 6.60 | 109.41 | 369.02 |
| 51-5 | Fruits | 1 | 100.60 | 100.88 | 85.59 | 71.70 | 118.55 |
| | | 10 | 83.41 | 116.05 | 64.12 | 88.36 | 222.17 |
| | | 100 | 75.82 | 156.48 | 20.34 | 103.40 | 414.49 |
| 51-6 | Flowers | 1 | 99.38 | 107.58 | 83.05 | 80.45 | 194.90 |
| | | 10 | 87.74 | 118.42 | 56.01 | 75.62 | 228.44 |
| | | 100 | 80.20 | 147.46 | 9.84 | 101.99 | 368.98 |

### Example 52. Stolon Golden Saxifrage

TABLE 105

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 52 | Stolon | | | | | | |
| | golden | | | | | | |
| | saxifrage | | | | | | |
| 52-1 | Whole plant | 1 | 29.60 | 84.64 | 180.58 | 102.02 | 121.85 |
| | | 10 | 49.39 | 40.16 | 321.99 | 156.86 | 190.23 |
| | | 100 | 72.22 | 22.30 | 355.60 | 353.96 | 209.44 |
| 52-2 | Roots | 1 | 23.30 | 49.03 | 164.55 | 110.84 | 95.09 |
| | | 10 | 34.66 | 36.11 | 309.76 | 185.70 | 244.27 |
| | | 100 | 84.83 | 25.85 | 469.71 | 302.99 | 284.20 |
| 52-3 | Stems | 1 | 20.21 | 41.70 | 144.82 | 110.93 | 88.90 |
| | | 10 | 40.43 | 31.14 | 279.51 | 113.30 | 170.51 |
| | | 100 | 78.57 | 20.95 | 484.70 | 407.97 | 342.55 |
| 52-4 | Leaves | 1 | 24.08 | 42.52 | 152.67 | 96.57 | 111.79 |
| | | 10 | 33.29 | 36.46 | 208.70 | 163.68 | 182.16 |
| | | 100 | 78.88 | 22.70 | 441.15 | 448.41 | 332.99 |
| 52-5 | Fruits | 1 | 25.07 | 67.13 | 177.49 | 93.62 | 98.05 |
| | | 10 | 30.51 | 34.62 | 340.01 | 149.97 | 179.76 |
| | | 100 | 70.08 | 25.28 | 375.94 | 347.73 | 350.24 |
| 52-6 | Flowers | 1 | 20.62 | 48.26 | 141.67 | 94.84 | 102.89 |
| | | 10 | 43.47 | 37.37 | 321.06 | 171.32 | 212.34 |
| | | 100 | 81.44 | 24.99 | 449.05 | 380.48 | 231.34 |

TABLE 106

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 52 | Stolon | | | | | | |
| | golden | | | | | | |
| | saxifrage | | | | | | |
| 52-1 | Whole plant | 1 | 99.28 | 96.45 | 104.61 | 83.87 | 102.37 |
| | | 10 | 89.94 | 117.11 | 76.40 | 81.22 | 340.73 |
| | | 100 | 78.88 | 143.14 | 30.25 | 107.54 | 453.25 |
| 52-2 | Roots | 1 | 101.65 | 105.14 | 83.29 | 72.48 | 156.73 |
| | | 10 | 90.80 | 108.05 | 72.44 | 81.63 | 283.96 |
| | | 100 | 76.08 | 149.31 | 17.50 | 95.70 | 455.46 |
| 52-3 | Stems | 1 | 95.71 | 104.93 | 81.58 | 69.48 | 196.64 |
| | | 10 | 90.98 | 139.09 | 72.32 | 75.43 | 220.58 |
| | | 100 | 91.00 | 148.58 | 12.77 | 110.42 | 477.70 |
| 52-4 | Leaves | 1 | 101.26 | 107.69 | 94.72 | 78.56 | 139.28 |
| | | 10 | 94.52 | 128.11 | 80.84 | 84.57 | 279.56 |
| | | 100 | 108.26 | 158.52 | 0.07 | 106.22 | 442.47 |
| 52-5 | Fruits | 1 | 96.20 | 113.39 | 107.59 | 72.03 | 90.72 |
| | | 10 | 90.81 | 108.95 | 84.20 | 77.50 | 314.40 |
| | | 100 | 76.35 | 152.60 | 32.68 | 95.76 | 372.65 |
| 52-6 | Flowers | 1 | 97.06 | 105.23 | 80.48 | 76.50 | 118.86 |
| | | 10 | 81.33 | 124.27 | 52.68 | 84.23 | 196.41 |
| | | 100 | 76.50 | 143.02 | 20.32 | 95.18 | 348.09 |

### Example 53. Serrate-Petal Rockfoil

TABLE 107

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 53 | Serrate-petal | | | | | | |
| | rockfoil | | | | | | |
| 53-1 | Whole plant | 1 | 25.28 | 47.14 | 161.74 | 80.98 | 91.34 |
| | | 10 | 36.09 | 31.08 | 286.13 | 122.38 | 211.65 |
| | | 100 | 73.48 | 26.84 | 399.86 | 330.82 | 245.92 |
| 53-2 | Roots | 1 | 26.57 | 72.31 | 173.20 | 108.39 | 130.81 |
| | | 10 | 40.01 | 33.66 | 283.01 | 149.96 | 190.18 |
| | | 100 | 74.17 | 28.67 | 379.21 | 323.24 | 344.82 |
| 53-3 | Stems | 1 | 27.96 | 47.26 | 195.10 | 97.29 | 141.26 |
| | | 10 | 45.66 | 33.21 | 259.40 | 128.72 | 105.92 |
| | | 100 | 79.53 | 28.68 | 368.62 | 353.15 | 344.59 |
| 53-4 | Leaves | 1 | 21.08 | 76.06 | 126.21 | 93.93 | 123.81 |
| | | 10 | 48.40 | 31.53 | 331.08 | 165.69 | 130.58 |
| | | 100 | 80.26 | 20.48 | 428.49 | 316.62 | 200.56 |
| 53-5 | Fruits | 1 | 23.77 | 85.29 | 161.78 | 109.42 | 134.72 |
| | | 10 | 42.00 | 35.34 | 277.76 | 178.64 | 183.06 |
| | | 100 | 74.64 | 29.25 | 454.87 | 313.89 | 290.20 |
| 53-6 | Flowers | 1 | 29.41 | 55.02 | 114.51 | 98.77 | 121.51 |
| | | 10 | 45.00 | 30.96 | 298.27 | 187.16 | 183.14 |
| | | 100 | 85.28 | 24.59 | 446.87 | 344.66 | 249.97 |

TABLE 108

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 53 | Serrate-petal | | | | | | |
| | rockfoil | | | | | | |
| 53-1 | Whole plant | 1 | 102.20 | 95.57 | 83.96 | 80.33 | 184.40 |
| | | 10 | 86.49 | 134.97 | 48.72 | 87.92 | 230.72 |
| | | 100 | 77.66 | 136.34 | 20.03 | 102.56 | 458.63 |
| 53-2 | Roots | 1 | 100.27 | 98.83 | 94.89 | 78.18 | 148.47 |
| | | 10 | 92.02 | 135.36 | 51.96 | 93.53 | 250.00 |
| | | 100 | 77.58 | 131.29 | 7.03 | 97.36 | 378.75 |
| 53-3 | Stems | 1 | 97.50 | 104.62 | 91.45 | 79.36 | 141.34 |
| | | 10 | 94.01 | 130.91 | 57.56 | 87.44 | 301.40 |
| | | 100 | 78.17 | 134.42 | 0.61 | 90.90 | 489.22 |
| 53-4 | Leaves | 1 | 102.39 | 107.12 | 88.75 | 84.02 | 101.15 |
| | | 10 | 86.12 | 107.51 | 59.49 | 95.90 | 249.00 |
| | | 100 | 73.08 | 130.25 | 13.40 | 109.93 | 316.70 |
| 53-5 | Fruits | 1 | 101.90 | 104.25 | 99.38 | 68.52 | 93.59 |
| | | 10 | 91.68 | 114.69 | 90.93 | 79.17 | 213.41 |
| | | 100 | 77.66 | 159.90 | 41.79 | 102.06 | 439.25 |
| 53-6 | Flowers | 1 | 99.66 | 114.26 | 106.16 | 69.03 | 175.22 |
| | | 10 | 81.91 | 117.68 | 59.29 | 94.36 | 227.78 |
| | | 100 | 77.33 | 140.61 | 27.73 | 103.06 | 304.27 |

### Example 54. Ulleungdo Deadnettle

TABLE 109

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 54 | Ulleungdo | | | | | | |
| | deadnettle | | | | | | |
| 54-1 | Whole plant | 1 | 22.07 | 83.48 | 189.28 | 83.49 | 147.26 |
| | | 10 | 35.25 | 31.59 | 266.85 | 167.24 | 216.49 |
| | | 100 | 79.54 | 21.38 | 479.77 | 349.02 | 208.16 |
| 54-2 | Roots | 1 | 30.07 | 59.13 | 168.33 | 100.86 | 124.00 |
| | | 10 | 37.06 | 39.91 | 347.13 | 152.53 | 157.41 |
| | | 100 | 70.51 | 24.41 | 444.42 | 318.05 | 266.49 |
| 54-3 | Stems | 1 | 25.56 | 64.79 | 102.65 | 107.03 | 83.36 |
| | | 10 | 39.07 | 37.03 | 293.55 | 197.16 | 168.64 |
| | | 100 | 70.01 | 28.05 | 387.16 | 491.15 | 340.49 |
| 54-4 | Leaves | 1 | 26.10 | 40.38 | 149.89 | 109.33 | 102.71 |
| | | 10 | 41.60 | 38.95 | 295.56 | 158.57 | 118.24 |
| | | 100 | 81.71 | 20.51 | 422.18 | 412.33 | 255.96 |
| 54-5 | Fruits | 1 | 20.77 | 46.77 | 135.99 | 102.38 | 148.53 |
| | | 10 | 39.53 | 35.82 | 288.99 | 154.34 | 231.06 |
| | | 100 | 82.71 | 22.25 | 488.53 | 455.52 | 244.14 |
| 54-6 | Flowers | 1 | 24.05 | 76.89 | 112.11 | 96.56 | 141.24 |
| | | 10 | 50.61 | 36.68 | 287.97 | 136.32 | 162.90 |
| | | 100 | 72.54 | 25.30 | 474.48 | 447.15 | 248.97 |

TABLE 110

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 54 | Ulleungdo | | | | | | |
| | deadnettle | | | | | | |
| 54-1 | Whole plant | 1 | 96.82 | 101.64 | 86.85 | 85.08 | 105.69 |
| | | 10 | 92.74 | 114.69 | 81.83 | 77.45 | 215.32 |
| | | 100 | 77.25 | 130.10 | 35.82 | 91.45 | 384.03 |
| 54-2 | Roots | 1 | 103.99 | 103.05 | 109.33 | 66.52 | 142.10 |
| | | 10 | 81.26 | 120.63 | 78.72 | 77.63 | 204.87 |
| | | 100 | 80.88 | 154.91 | 39.49 | 93.10 | 352.27 |
| 54-3 | Stems | 1 | 102.21 | 104.81 | 107.88 | 80.60 | 127.33 |
| | | 10 | 81.10 | 116.46 | 91.29 | 83.49 | 235.25 |
| | | 100 | 80.71 | 142.51 | 7.00 | 102.76 | 438.09 |
| 54-4 | Leaves | 1 | 102.88 | 106.52 | 102.45 | 85.75 | 179.95 |
| | | 10 | 91.46 | 106.23 | 61.46 | 87.07 | 272.72 |
| | | 100 | 77.01 | 150.27 | 28.92 | 95.56 | 307.67 |
| 54-5 | Fruits | 1 | 103.67 | 115.15 | 80.93 | 85.58 | 119.16 |
| | | 10 | 85.55 | 127.34 | 73.61 | 75.54 | 181.82 |
| | | 100 | 77.01 | 135.85 | 21.78 | 98.00 | 386.54 |
| 54-6 | Flowers | 1 | 97.84 | 100.04 | 96.15 | 83.90 | 141.76 |
| | | 10 | 94.31 | 132.13 | 59.08 | 82.04 | 295.35 |
| | | 100 | 71.11 | 157.13 | 3.21 | 106.89 | 339.17 |

### Example 55. Island Ninebark

TABLE 111

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppnt) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 55 | Island | | | | | | |
| | ninebark | | | | | | |
| 55-1 | Whole plant | 1 | 27.22 | 54.24 | 118.80 | 103.35 | 145.05 |
| | | 10 | 43.05 | 32.89 | 240.84 | 112.51 | 107.60 |
| | | 100 | 79.77 | 29.58 | 446.43 | 315.92 | 340.60 |
| 55-2 | Roots | 1 | 27.54 | 42.54 | 178.59 | 86.37 | 126.82 |
| | | 10 | 43.29 | 38.30 | 254.43 | 160.57 | 100.02 |
| | | 100 | 71.74 | 27.06 | 451.60 | 351.85 | 272.98 |
| 55-3 | Stems | 1 | 21.51 | 60.34 | 190.30 | 81.93 | 144.67 |
| | | 10 | 34.78 | 36.53 | 249.18 | 144.42 | 124.86 |
| | | 100 | 71.17 | 28.67 | 395.08 | 344.82 | 317.30 |
| 55-4 | Leaves | 1 | 25.87 | 67.33 | 173.53 | 96.74 | 90.90 |
| | | 10 | 35.66 | 40.42 | 300.12 | 155.21 | 249.14 |
| | | 100 | 80.96 | 28.77 | 408.22 | 373.80 | 224.73 |
| 55-5 | Fruits | 1 | 24.58 | 42.98 | 124.68 | 88.66 | 136.18 |
| | | 10 | 40.67 | 33.94 | 212.03 | 158.10 | 179.72 |
| | | 100 | 85.92 | 25.63 | 481.38 | 397.09 | 349.31 |
| 55-6 | Flowers | 1 | 28.38 | 88.98 | 189.56 | 94.60 | 94.42 |
| | | 10 | 35.28 | 30.54 | 262.07 | 185.05 | 167.79 |
| | | 100 | 79.93 | 22.20 | 467.24 | 480.23 | 314.85 |

TABLE 112

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 55 | Island | | | | | | |
| | ninebark | | | | | | |
| 55-1 | Whole plant | 1 | 100.98 | 112.08 | 105.34 | 85.67 | 175.25 |
| | | 10 | 89.34 | 111.72 | 61.22 | 84.78 | 223.17 |
| | | 100 | 71.78 | 132.79 | 45.08 | 92.62 | 400.09 |
| 55-2 | Roots | 1 | 100.76 | 114.94 | 107.30 | 85.96 | 164.75 |
| | | 10 | 94.11 | 107.49 | 73.08 | 79.61 | 224.43 |
| | | 100 | 72.30 | 148.44 | 14.32 | 98.88 | 388.19 |
| 55-3 | Stems | 1 | 100.66 | 104.77 | 95.58 | 82.95 | 173.10 |
| | | 10 | 95.10 | 125.58 | 58.55 | 84.88 | 255.92 |
| | | 100 | 73.81 | 156.13 | 36.79 | 95.39 | 415.45 |
| 55-4 | Leaves | 1 | 102.51 | 111.64 | 103.89 | 65.02 | 142.49 |
| | | 10 | 85.37 | 111.95 | 89.39 | 90.34 | 242.08 |
| | | 100 | 77.86 | 134.22 | 16.21 | 108.62 | 322.77 |
| 55-5 | Fruits | 1 | 97.31 | 108.93 | 102.27 | 71.17 | 141.83 |
| | | 10 | 91.14 | 126.59 | 84.85 | 94.44 | 213.15 |
| | | 100 | 70.48 | 130.61 | 23.30 | 105.27 | 482.27 |
| 55-6 | Flowers | 1 | 103.24 | 102.64 | 83.10 | 78.17 | 164.80 |
| | | 10 | 91.44 | 139.34 | 80.87 | 95.10 | 314.78 |
| | | 100 | 70.63 | 154.31 | 9.91 | 97.74 | 423.52 |

### Example 56. Island Corydalis

TABLE 113

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 56 | Island | | | | | | |
| | corydalis | | | | | | |
| 56-1 | Whole plant | 1 | 21.96 | 74.21 | 173.73 | 81.22 | 94.13 |
| | | 10 | 43.31 | 30.56 | 319.32 | 159.07 | 183.63 |
| | | 100 | 80.29 | 23.25 | 416.14 | 364.84 | 332.71 |
| 56-2 | Roots | 1 | 20.55 | 77.26 | 133.36 | 86.38 | 136.55 |
| | | 10 | 33.22 | 40.82 | 264.19 | 168.90 | 110.08 |
| | | 100 | 76.39 | 21.48 | 383.03 | 455.00 | 299.03 |
| 56-3 | Stems | 1 | 30.46 | 66.21 | 108.48 | 97.76 | 119.70 |
| | | 10 | 49.06 | 40.86 | 222.72 | 121.52 | 160.71 |
| | | 100 | 78.34 | 23.27 | 420.45 | 307.59 | 318.34 |
| 56-4 | Leaves | 1 | 28.64 | 66.90 | 147.37 | 101.89 | 127.95 |
| | | 10 | 47.19 | 33.73 | 303.55 | 130.22 | 144.73 |
| | | 100 | 81.50 | 27.28 | 359.22 | 394.82 | 201.46 |
| 56-5 | Fruits | 1 | 23.12 | 84.63 | 168.47 | 100.98 | 121.75 |
| | | 10 | 32.28 | 36.26 | 327.07 | 163.87 | 121.14 |
| | | 100 | 80.74 | 27.11 | 435.26 | 438.09 | 225.37 |
| 56-6 | Flowers | 1 | 24.93 | 79.55 | 179.41 | 81.87 | 101.11 |
| | | 10 | 44.10 | 37.19 | 343.30 | 152.29 | 233.19 |
| | | 100 | 80.10 | 30.19 | 384.14 | 453.17 | 243.53 |

TABLE 114

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 56 | Island | | | | | | |
| | corydalis | | | | | | |
| 56-1 | Whole plant | 1 | 96.92 | 100.36 | 110.57 | 76.27 | 90.74 |
| | | 10 | 91.81 | 114.94 | 76.53 | 87.05 | 170.35 |
| | | 100 | 76.29 | 150.04 | 46.94 | 94.03 | 494.92 |
| 56-2 | Roots | 1 | 96.86 | 114.99 | 96.13 | 82.55 | 201.74 |
| | | 10 | 81.76 | 129.44 | 48.90 | 84.40 | 250.19 |
| | | 100 | 77.16 | 160.30 | 22.51 | 93.78 | 341.07 |
| 56-3 | Stems | 1 | 101.75 | 107.56 | 88.72 | 71.93 | 104.73 |
| | | 10 | 85.92 | 109.14 | 65.91 | 94.95 | 276.56 |
| | | 100 | 75.16 | 134.81 | 17.04 | 91.97 | 434.89 |
| 56-4 | Leaves | 1 | 101.86 | 112.42 | 80.90 | 83.84 | 121.80 |
| | | 10 | 93.16 | 132.83 | 50.48 | 84.78 | 312.45 |
| | | 100 | 76.11 | 159.03 | 37.83 | 108.00 | 338.18 |
| 56-5 | Fruits | 1 | 102.77 | 105.76 | 100.42 | 77.33 | 99.23 |
| | | 10 | 82.82 | 135.00 | 85.31 | 85.18 | 187.30 |
| | | 100 | 76.17 | 130.45 | 34.90 | 96.57 | 356.67 |
| 56-6 | Flowers | 1 | 99.77 | 97.63 | 84.49 | 81.15 | 155.50 |
| | | 10 | 87.72 | 120.24 | 83.86 | 75.65 | 329.92 |
| | | 100 | 76.86 | 145.61 | 37.05 | 102.88 | 410.07 |

### Example 57. Ulleungdo Raspberry

TABLE 115

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 57 | Ulleungdo | | | | | | |
| | raspberry | | | | | | |
| 57-1 | Whole plant | 1 | 23.09 | 66.05 | 194.56 | 87.48 | 95.82 |
| | | 10 | 39.52 | 40.52 | 266.25 | 178.26 | 108.92 |
| | | 100 | 84.27 | 28.73 | 377.13 | 348.56 | 314.56 |
| 57-2 | Roots | 1 | 29.09 | 65.15 | 129.77 | 99.47 | 89.47 |
| | | 10 | 36.47 | 40.57 | 275.24 | 116.58 | 171.77 |
| | | 100 | 78.91 | 25.66 | 430.86 | 338.30 | 314.41 |
| 57-3 | Stems | 1 | 27.63 | 44.31 | 130.02 | 109.32 | 142.59 |
| | | 10 | 45.77 | 35.49 | 345.15 | 110.14 | 177.76 |
| | | 100 | 83.47 | 23.59 | 359.15 | 316.89 | 203.45 |
| 57-4 | Leaves | 1 | 23.45 | 89.94 | 124.39 | 96.68 | 82.23 |
| | | 10 | 33.05 | 34.23 | 211.53 | 191.78 | 164.22 |
| | | 100 | 85.28 | 26.16 | 387.77 | 367.95 | 256.56 |
| 57-5 | Fruits | 1 | 22.87 | 85.20 | 157.22 | 81.46 | 126.71 |
| | | 10 | 34.88 | 36.93 | 224.18 | 132.30 | 231.15 |
| | | 100 | 77.86 | 20.23 | 356.90 | 402.75 | 209.50 |
| 57-6 | Flowers | 1 | 29.03 | 52.01 | 128.63 | 88.79 | 82.16 |
| | | 10 | 44.57 | 30.09 | 299.25 | 137.97 | 106.56 |
| | | 100 | 79.59 | 25.46 | 433.69 | 349.04 | 263.15 |

TABLE 116

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 57 | Ulleungdo | | | | | | |
| | raspberry | | | | | | |
| 57-1 | Whole plant | 1 | 103.53 | 99.32 | 82.13 | 79.31 | 200.98 |
| | | 10 | 81.34 | 137.21 | 57.39 | 88.58 | 297.94 |
| | | 100 | 73.22 | 136.66 | 22.22 | 96.26 | 447.58 |
| 57-2 | Roots | 1 | 96.16 | 95.17 | 99.84 | 73.84 | 183.87 |
| | | 10 | 93.25 | 114.23 | 82.14 | 87.96 | 220.78 |
| | | 100 | 77.06 | 157.08 | 2.65 | 90.99 | 318.45 |
| 57-3 | Stems | 1 | 98.65 | 110.02 | 80.56 | 70.37 | 132.18 |
| | | 10 | 85.68 | 108.74 | 51.70 | 80.70 | 252.46 |
| | | 100 | 74.38 | 137.04 | 34.95 | 94.05 | 348.59 |
| 57-4 | Leaves | 1 | 103.81 | 115.64 | 90.30 | 76.67 | 141.16 |
| | | 10 | 91.52 | 111.75 | 59.16 | 85.87 | 246.33 |
| | | 100 | 74.39 | 160.88 | 45.94 | 107.58 | 365.48 |
| 57-5 | Fruits | 1 | 99.35 | 96.52 | 91.19 | 82.10 | 195.52 |
| | | 10 | 86.63 | 109.22 | 79.41 | 75.55 | 283.42 |
| | | 100 | 80.34 | 145.44 | 11.56 | 103.80 | 309.05 |
| 57-6 | Flowers | 1 | 96.66 | 95.47 | 99.72 | 78.74 | 186.07 |
| | | 10 | 82.73 | 140.36 | 71.80 | 75.69 | 323.83 |
| | | 100 | 71.37 | 141.03 | 40.59 | 94.59 | 471.79 |

### Example 58. Korea Dystaenia

TABLE 117

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 58 | Korea | | | | | | |
| | Dystaenia | | | | | | |
| 58-1 | Whole plant | 1 | 23.91 | 57.57 | 113.49 | 91.31 | 129.48 |
| | | 10 | 45.92 | 37.53 | 329.22 | 188.32 | 209.77 |
| | | 100 | 79.08 | 25.71 | 371.21 | 375.33 | 234.69 |
| 58-2 | Roots | 1 | 25.89 | 65.17 | 193.60 | 104.15 | 100.57 |
| | | 10 | 47.17 | 34.17 | 349.18 | 148.61 | 117.05 |
| | | 100 | 76.33 | 25.50 | 350.15 | 480.22 | 246.63 |
| 58-3 | Stems | 1 | 30.07 | 84.40 | 163.26 | 86.11 | 130.69 |
| | | 10 | 40.15 | 38.86 | 311.05 | 168.28 | 234.32 |
| | | 100 | 80.85 | 27.45 | 462.19 | 374.55 | 216.59 |
| 58-4 | Leaves | 1 | 29.47 | 80.07 | 100.82 | 105.98 | 122.37 |
| | | 10 | 34.69 | 38.53 | 211.01 | 153.81 | 149.33 |
| | | 100 | 85.66 | 29.76 | 387.69 | 351.97 | 312.48 |
| 58-5 | Fruits | 1 | 23.28 | 47.92 | 106.79 | 83.18 | 99.17 |
| | | 10 | 48.08 | 37.89 | 331.32 | 112.97 | 106.60 |
| | | 100 | 80.45 | 30.11 | 399.44 | 313.54 | 326.36 |
| 58-6 | Flowers | 1 | 20.52 | 47.11 | 181.22 | 97.34 | 147.72 |
| | | 10 | 40.01 | 30.23 | 300.45 | 120.38 | 120.41 |
| | | 100 | 78.62 | 24.21 | 449.22 | 451.53 | 348.30 |

TABLE 118

| | | | | Blood | Cognitive | Liver | |
|---|---|---|---|---|---|---|---|
| | | | Anti- | glucose- | ability-β- | health- | Andropause-AR |
| | | | allergy-IgE | Glucose | secretase | Cell | transcriptional |
| | | Conc. | secretion | absorption | activity | viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexa- | (Metformin | (β-Secretase | (Silymarin | (Dihydro- |
| | | | methasone | 1 mM) | Inhibitor IV | 100 ppm) | testosterone |
| | | | 1 uM) | | 15 nM) | | 100 nM) |
| 58 | Korea | | | | | | |
| | Dystaenia | | | | | | |
| 58-1 | Whole plant | 1 | 99.76 | 110.21 | 89.46 | 74.55 | 182.75 |
| | | 10 | 84.83 | 134.20 | 64.49 | 80.51 | 277.48 |
| | | 100 | 72.91 | 152.84 | 14.59 | 103.69 | 444.93 |
| 58-2 | Roots | 1 | 97.63 | 99.66 | 85.10 | 76.02 | 178.41 |
| | | 10 | 90.12 | 132.97 | 56.88 | 94.70 | 286.18 |
| | | 100 | 77.50 | 156.46 | 5.30 | 101.33 | 406.45 |
| 58-3 | Stems | 1 | 95.34 | 96.83 | 82.02 | 85.44 | 206.51 |
| | | 10 | 82.27 | 130.40 | 64.46 | 81.09 | 229.56 |
| | | 100 | 77.43 | 159.56 | 49.05 | 95.07 | 400.02 |
| 58-4 | Leaves | 1 | 99.66 | 107.64 | 83.84 | 69.76 | 106.66 |
| | | 10 | 82.94 | 112.85 | 51.97 | 84.13 | 189.27 |
| | | 100 | 70.37 | 150.94 | 32.94 | 99.78 | 413.02 |
| 58-5 | Fruits | 1 | 101.81 | 103.01 | 104.32 | 81.33 | 128.72 |
| | | 10 | 86.78 | 112.64 | 81.52 | 84.00 | 196.83 |
| | | 100 | 79.58 | 154.89 | 44.78 | 106.35 | 493.44 |
| 58-6 | Flowers | 1 | 99.86 | 110.20 | 95.11 | 71.39 | 200.34 |
| | | 10 | 83.72 | 105.22 | 81.49 | 83.40 | 301.81 |
| | | 100 | 74.64 | 133.06 | 26.56 | 90.07 | 434.26 |

### Example 59. Ulleungdo Violet

TABLE 119

| | | | | | Anti- | | |
|---|---|---|---|---|---|---|---|
| | | | Blood | Lipid- | obesity- | Immunity- | Menopause- |
| | | | circulation- | cholesterol | Glycerol | IFNg | ER binding |
| | | Conc. | NO Conc. | synthesis | Conc. | secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 59 | Ulleungdo | | | | | | |
| | violet | | | | | | |
| 59-1 | Whole plant | 1 | 22.18 | 57.44 | 129.12 | 99.89 | 148.33 |
| | | 10 | 45.50 | 40.56 | 235.11 | 175.13 | 193.68 |
| | | 100 | 78.17 | 27.59 | 379.05 | 485.95 | 236.17 |
| 59-2 | Roots | 1 | 26.56 | 50.78 | 116.80 | 99.60 | 105.50 |
| | | 10 | 39.46 | 39.07 | 211.53 | 143.52 | 119.74 |
| | | 100 | 84.18 | 27.16 | 472.44 | 334.60 | 348.62 |
| 59-3 | Stems | 1 | 26.26 | 73.59 | 200.69 | 92.32 | 81.59 |
| | | 10 | 30.21 | 37.99 | 289.51 | 185.54 | 122.99 |
| | | 100 | 80.95 | 20.67 | 364.68 | 389.52 | 344.28 |
| 59-4 | Leaves | 1 | 23.26 | 56.33 | 100.65 | 107.67 | 150.75 |
| | | 10 | 39.52 | 34.91 | 248.55 | 119.61 | 108.84 |
| | | 100 | 82.87 | 30.69 | 372.97 | 363.74 | 269.48 |
| 59-5 | Fruits | 1 | 26.01 | 56.15 | 138.25 | 84.12 | 90.83 |
| | | 10 | 37.69 | 35.65 | 274.36 | 120.01 | 114.21 |
| | | 100 | 80.36 | 22.38 | 447.68 | 323.36 | 340.96 |
| 59-6 | Flowers | 1 | 21.89 | 42.04 | 194.97 | 94.62 | 94.24 |
| | | 10 | 50.74 | 39.02 | 316.91 | 183.75 | 128.00 |
| | | 100 | 77.56 | 22.43 | 381.86 | 313.43 | 298.94 |

TABLE 120

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 59 | Ulleungdo | | | | | | |
| | violet | | | | | | |
| 59-1 | Whole plant | 1 | 102.81 | 102.09 | 98.90 | 68.78 | 208.07 |
| | | 10 | 89.85 | 113.99 | 89.34 | 89.16 | 187.30 |
| | | 100 | 77.52 | 135.12 | 35.69 | 101.42 | 468.13 |
| 59-2 | Roots | 1 | 95.54 | 114.41 | 108.62 | 84.30 | 123.58 |
| | | 10 | 89.87 | 128.54 | 85.54 | 80.85 | 178.10 |
| | | 100 | 75.74 | 152.25 | 35.31 | 100.08 | 366.00 |
| 59-3 | Stems | 1 | 99.13 | 98.10 | 98.78 | 75.32 | 201.35 |
| | | 10 | 86.16 | 127.55 | 59.06 | 91.33 | 308.53 |
| | | 100 | 76.32 | 154.40 | 16.03 | 93.82 | 334.25 |
| 59-4 | Leaves | 1 | 96.72 | 105.92 | 104.96 | 69.08 | 93.82 |
| | | 10 | 83.05 | 131.82 | 88.96 | 91.90 | 270.80 |
| | | 100 | 76.63 | 136.20 | 29.43 | 91.07 | 377.06 |
| 59-5 | Fruits | 1 | 101.78 | 99.60 | 92.07 | 69.08 | 168.73 |
| | | 10 | 87.43 | 125.50 | 76.50 | 83.28 | 209.90 |
| | | 100 | 70.49 | 147.75 | 8.70 | 103.36 | 496.40 |
| 59-6 | Flowers | 1 | 99.94 | 98.65 | 101.97 | 82.54 | 99.28 |
| | | 10 | 83.89 | 108.95 | 63.13 | 78.96 | 287.27 |
| | | 100 | 75.37 | 131.57 | 33.87 | 99.06 | 349.33 |

### Example 60. Spindle Tree

TABLE 121

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 60 | Spindle Tree | | | | | | |
| 60-1 | Whole plant | 1 | 27.42 | 82.77 | 114.47 | 97.89 | 101.02 |
| | | 10 | 35.10 | 32.98 | 203.17 | 132.40 | 248.93 |
| | | 100 | 71.06 | 22.15 | 448.10 | 335.38 | 303.86 |
| 60-2 | Roots | 1 | 21.81 | 82.45 | 126.12 | 98.28 | 129.39 |
| | | 10 | 43.41 | 37.71 | 216.37 | 165.40 | 182.61 |
| | | 100 | 71.88 | 28.70 | 458.32 | 336.66 | 299.39 |
| 60-3 | Stems | 1 | 23.57 | 82.39 | 196.03 | 87.90 | 83.06 |
| | | 10 | 45.22 | 36.09 | 235.12 | 138.83 | 172.26 |
| | | 100 | 82.22 | 25.92 | 367.98 | 471.23 | 215.60 |
| 60-4 | Leaves | 1 | 27.91 | 90.95 | 119.91 | 107.49 | 84.01 |
| | | 10 | 33.33 | 33.79 | 330.26 | 187.37 | 247.30 |
| | | 100 | 81.14 | 20.47 | 359.02 | 376.65 | 349.63 |
| 60-5 | Fruits | 1 | 21.67 | 45.29 | 125.74 | 86.52 | 95.63 |
| | | 10 | 38.83 | 37.79 | 208.85 | 130.08 | 119.47 |
| | | 100 | 73.15 | 23.47 | 363.53 | 416.13 | 248.14 |
| 60-6 | Flowers | 1 | 22.29 | 79.04 | 184.86 | 90.32 | 91.57 |
| | | 10 | 33.85 | 37.12 | 311.55 | 183.45 | 172.06 |
| | | 100 | 70.59 | 22.29 | 423.95 | 461.66 | 234.69 |

TABLE 122

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 60 | Spindle Tree | | | | | | |
| 60-1 | Whole plant | 1 | 99.38 | 101.12 | 84.49 | 82.57 | 133.44 |
| | | 10 | 83.53 | 134.21 | 59.67 | 91.44 | 198.27 |
| | | 100 | 79.65 | 135.26 | 50.65 | 90.49 | 446.24 |
| 60-2 | Roots | 1 | 97.98 | 95.43 | 98.00 | 75.00 | 90.82 |
| | | 10 | 95.29 | 130.84 | 64.72 | 91.70 | 192.65 |
| | | 100 | 79.35 | 140.28 | 41.25 | 107.68 | 481.00 |
| 60-3 | Stems | 1 | 103.30 | 98.35 | 97.45 | 73.35 | 146.04 |
| | | 10 | 90.45 | 113.47 | 49.27 | 88.10 | 306.81 |
| | | 100 | 70.96 | 147.58 | 3.99 | 101.64 | 418.84 |
| 60-4 | Leaves | 1 | 97.15 | 102.85 | 92.04 | 69.60 | 193.65 |
| | | 10 | 84.65 | 140.51 | 50.71 | 79.77 | 320.56 |
| | | 100 | 79.69 | 155.03 | 43.99 | 98.55 | 478.37 |
| 60-5 | Fruits | 1 | 97.60 | 102.73 | 82.21 | 85.13 | 168.79 |
| | | 10 | 85.79 | 106.26 | 47.31 | 91.87 | 285.95 |
| | | 100 | 80.71 | 135.72 | 44.50 | 110.22 | 500.78 |
| 60-6 | Flowers | 1 | 100.48 | 103.94 | 102.50 | 71.71 | 167.82 |
| | | 10 | 83.71 | 123.47 | 70.04 | 92.88 | 318.99 |
| | | 100 | 75.38 | 160.89 | 43.42 | 92.12 | 323.29 |

### Example 61. Crimson Grapevine

TABLE 123

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 61 | Crimson | | | | | | |
| | grapevine | | | | | | |
| 61-1 | Whole plant | 1 | 23.21 | 53.53 | 134.89 | 94.24 | 81.38 |
| | | 10 | 45.73 | 39.27 | 256.01 | 122.54 | 247.61 |
| | | 100 | 70.59 | 22.30 | 443.25 | 311.59 | 255.11 |
| 61-2 | Roots | 1 | 27.37 | 80.57 | 155.02 | 93.91 | 82.84 |
| | | 10 | 39.34 | 35.72 | 301.68 | 188.15 | 197.42 |
| | | 100 | 82.89 | 29.22 | 488.32 | 489.45 | 333.64 |
| 61-3 | Stems | 1 | 21.26 | 48.54 | 107.78 | 95.94 | 141.19 |
| | | 10 | 43.43 | 34.98 | 331.02 | 122.09 | 107.99 |
| | | 100 | 84.88 | 21.61 | 373.15 | 375.35 | 280.78 |
| 61-4 | Leaves | 1 | 23.08 | 83.79 | 113.09 | 90.81 | 111.77 |
| | | 10 | 50.06 | 34.42 | 218.85 | 176.80 | 198.54 |
| | | 100 | 76.76 | 27.24 | 410.66 | 389.52 | 263.83 |
| 61-5 | Fruits | 1 | 20.94 | 71.89 | 93.67 | 80.28 | 120.14 |
| | | 10 | 45.86 | 37.10 | 97.92 | 83.93 | 205.03 |
| | | 100 | 84.97 | 30.20 | 103.81 | 86.52 | 312.16 |
| 61-6 | Flowers | 1 | 24.88 | 41.42 | 127.20 | 83.03 | 133.52 |
| | | 10 | 31.96 | 37.70 | 281.72 | 168.40 | 103.73 |
| | | 100 | 71.30 | 30.93 | 460.50 | 493.25 | 285.93 |

TABLE 124

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 61 | Crimson | | | | | | |
| | grapevine | | | | | | |
| 61-1 | Whole plant | 1 | 100.17 | 100.35 | 97.47 | 71.85 | 92.48 |
| | | 10 | 89.17 | 136.41 | 74.45 | 92.90 | 272.15 |
| | | 100 | 78.09 | 143.13 | 8.78 | 91.92 | 357.95 |
| 61-2 | Roots | 1 | 99.03 | 99.97 | 84.86 | 78.01 | 146.29 |
| | | 10 | 92.56 | 127.10 | 82.76 | 84.96 | 249.34 |
| | | 100 | 75.36 | 156.18 | 29.68 | 98.60 | 393.51 |
| 61-3 | Stems | 1 | 96.18 | 100.30 | 87.13 | 73.51 | 131.38 |
| | | 10 | 81.33 | 113.22 | 68.14 | 83.72 | 184.18 |
| | | 100 | 73.19 | 159.00 | 24.33 | 97.01 | 447.49 |
| 61-4 | Leaves | 1 | 102.62 | 105.62 | 85.38 | 17.64 | 187.51 |
| | | 10 | 80.43 | 109.92 | 90.71 | 22.00 | 220.19 |
| | | 100 | 73.49 | 153.37 | 20.10 | 30.39 | 500.46 |
| 61-5 | Fruits | 1 | 101.57 | 90.68 | 82.08 | 75.71 | 186.92 |
| | | 10 | 90.05 | 94.57 | 53.35 | 90.44 | 173.45 |
| | | 100 | 72.24 | 102.88 | 36.06 | 93.62 | 322.63 |
| 61-6 | Flowers | 1 | 103.67 | 113.92 | 102.99 | 67.01 | 164.08 |
| | | 10 | 91.42 | 136.85 | 51.96 | 80.90 | 243.45 |
| | | 100 | 76.08 | 153.72 | 40.98 | 109.53 | 417.15 |

### Example 62. Aralia Continentalis

TABLE 125

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 62 | Aralia | | | | | | |
| | continentalis | | | | | | |
| 62-1 | Whole plant | 1 | 29.85 | 86.58 | 138.53 | 81.33 | 81.18 |
| | | 10 | 48.84 | 38.44 | 204.52 | 134.25 | 152.56 |
| | | 100 | 71.63 | 20.11 | 470.62 | 399.69 | 241.55 |
| 62-2 | Roots | 1 | 25.43 | 41.10 | 83.19 | 91.97 | 103.27 |
| | | 10 | 35.77 | 38.05 | 91.00 | 170.85 | 206.16 |
| | | 100 | 75.59 | 25.35 | 95.73 | 452.83 | 254.57 |
| 62-3 | Stems | 1 | 21.87 | 74.30 | 181.07 | 95.49 | 135.58 |
| | | 10 | 43.16 | 40.38 | 203.34 | 185.93 | 149.57 |
| | | 100 | 78.35 | 22.43 | 399.17 | 368.53 | 318.98 |
| 62-4 | Leaves | 1 | 25.94 | 88.87 | 156.86 | 55.65 | 91.63 |
| | | 10 | 39.26 | 37.00 | 200.73 | 60.41 | 211.80 |
| | | 100 | 85.39 | 22.60 | 445.06 | 63.72 | 286.36 |
| 62-5 | Fruits | 1 | 20.50 | 65.28 | 167.17 | 109.21 | 144.72 |
| | | 10 | 41.45 | 32.61 | 343.32 | 169.28 | 129.18 |
| | | 100 | 80.59 | 30.49 | 401.04 | 459.96 | 209.30 |
| 62-5 | Flowers | 1 | 20.31 | 75.25 | 187.31 | 95.80 | 144.21 |
| | | 10 | 46.75 | 33.53 | 270.07 | 136.81 | 176.41 |
| | | 100 | 85.08 | 29.13 | 441.10 | 399.33 | 349.43 |

TABLE 126

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 62 | Aralia | | | | | | |
| | continentalis | | | | | | |
| 62-1 | Whole plant | 1 | 103.23 | 106.28 | 82.22 | 77.63 | 186.31 |
| | | 10 | 91.00 | 112.19 | 55.75 | 88.42 | 179.68 |
| | | 100 | 72.95 | 135.33 | 4.32 | 101.92 | 492.04 |
| 62-2 | Roots | 1 | 105.12 | 99.41 | 94.55 | 75.46 | 99.46 |
| | | 10 | 100.64 | 105.27 | 92.39 | 86.60 | 102.15 |
| | | 100 | 98.15 | 156.49 | 47.14 | 106.96 | 103.30 |
| 62-3 | Stems | 1 | 101.53 | 105.18 | 87.55 | 79.86 | 140.30 |
| | | 10 | 84.80 | 130.56 | 54.63 | 91.11 | 303.89 |
| | | 100 | 80.68 | 140.04 | 34.67 | 106.21 | 429.95 |
| 62-4 | Leaves | 1 | 99.81 | 106.65 | 102.79 | 67.24 | 197.12 |
| | | 10 | 82.49 | 124.06 | 73.10 | 84.28 | 273.62 |
| | | 100 | 76.56 | 151.01 | 47.10 | 108.25 | 308.89 |
| 62-5 | Fruits | 1 | 102.46 | 111.34 | 85.95 | 84.55 | 94.71 |
| | | 10 | 84.10 | 106.53 | 77.97 | 79.74 | 298.79 |
| | | 100 | 75.97 | 154.79 | 18.50 | 106.60 | 314.77 |
| 62-5 | Flowers | 1 | 97.86 | 103.84 | 86.24 | 68.14 | 129.10 |
| | | 10 | 84.78 | 107.47 | 81.45 | 86.90 | 190.63 |
| | | 100 | 78.96 | 132.19 | 37.96 | 106.10 | 403.14 |

### Example 63. Japanese Angelica

TABLE 127

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 63 | Japanese | | | | | | |
| | Angelica | | | | | | |
| 63-1 | Whole plant | 1 | 22.08 | 54.88 | 138.14 | 100.18 | 101.39 |
| | | 10 | 42.28 | 30.66 | 337.81 | 172.35 | 185.68 |
| | | 100 | 85.64 | 27.70 | 467.48 | 335.23 | 207.41 |
| 63-2 | Roots | 1 | 22.62 | 48.31 | 193.35 | 104.35 | 144.83 |
| | | 10 | 30.84 | 36.64 | 319.09 | 115.52 | 101.24 |
| | | 100 | 73.03 | 24.10 | 373.49 | 432.97 | 289.21 |
| 63-3 | Stems | 1 | 24.34 | 70.48 | 179.86 | 100.32 | 145.95 |
| | | 10 | 35.99 | 33.25 | 350.86 | 150.53 | 206.39 |
| | | 100 | 79.28 | 22.79 | 366.14 | 412.54 | 233.48 |
| 63-4 | Leaves | 1 | 5.30 | 94.89 | 97.84 | 82.00 | 99.79 |
| | | 10 | 7.80 | 85.37 | 104.34 | 90.32 | 147.90 |
| | | 100 | 10.17 | 81.94 | 108.58 | 102.00 | 264.88 |
| 63-5 | Fruits | 1 | 21.56 | 59.68 | 122.18 | 82.88 | 124.86 |
| | | 10 | 43.07 | 37.48 | 277.30 | 180.39 | 195.33 |
| | | 100 | 76.82 | 21.71 | 360.78 | 387.87 | 316.57 |
| 63-6 | Flowers | 1 | 29.54 | 54.66 | 145.81 | 99.95 | 119.50 |
| | | 10 | 34.94 | 31.01 | 326.57 | 152.39 | 168.77 |
| | | 100 | 81.54 | 21.00 | 386.54 | 322.65 | 283.80 |

TABLE 128

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 63 | Japanese | | | | | | |
| | Angelica | | | | | | |
| 63-1 | Whole plant | 1 | 96.27 | 108.55 | 87.81 | 69.90 | 197.98 |
| | | 10 | 91.77 | 105.65 | 89.72 | 79.07 | 170.87 |
| | | 100 | 70.25 | 158.68 | 12.69 | 92.56 | 470.57 |
| 63-2 | Roots | 1 | 101.62 | 89.57 | 108.13 | 78.17 | 148.65 |
| | | 10 | 90.59 | 95.83 | 83.40 | 92.47 | 234.47 |
| | | 100 | 77.12 | 100.04 | 20.61 | 106.37 | 349.17 |
| 63-3 | Stems | 1 | 100.33 | 88.47 | 87.41 | 75.61 | 115.25 |
| | | 10 | 91.98 | 94.53 | 45.50 | 79.01 | 185.39 |
| | | 100 | 71.55 | 105.05 | 28.99 | 105.41 | 404.96 |
| 63-4 | Leaves | 1 | 97.84 | 105.13 | 96.94 | 28.38 | 115.64 |
| | | 10 | 87.12 | 124.17 | 73.46 | 35.49 | 276.24 |
| | | 100 | 73.77 | 148.35 | 21.34 | 47.78 | 395.26 |
| 63-5 | Fruits | 1 | 96.78 | 108.56 | 84.53 | 71.57 | 169.24 |
| | | 10 | 88.61 | 140.46 | 80.79 | 90.92 | 317.24 |
| | | 100 | 73.18 | 158.04 | 43.41 | 109.48 | 388.82 |
| 63-6 | Flowers | 1 | 102.47 | 103.13 | 106.24 | 66.41 | 178.74 |
| | | 10 | 81.65 | 105.97 | 46.97 | 88.23 | 239.60 |
| | | 100 | 72.84 | 133.92 | 42.97 | 92.19 | 418.24 |

### Example 64. Glossy-Leaf Paper Plant

TABLE 129

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 64 | Glossy-leaf | | | | | | |
| | paper plant | | | | | | |
| 64-1 | Whole plant | 1 | 22.90 | 52.80 | 130.59 | 91.39 | 145.63 |
| | | 10 | 33.90 | 35.70 | 202.39 | 190.51 | 181.57 |
| | | 100 | 72.85 | 22.83 | 485.46 | 353.10 | 203.23 |
| 64-2 | Roots | 1 | 27.52 | 81.67 | 166.88 | 101.16 | 100.29 |
| | | 10 | 48.00 | 40.06 | 307.91 | 192.95 | 203.68 |
| | | 100 | 71.89 | 25.52 | 359.00 | 349.09 | 338.06 |
| 64-3 | Stems | 1 | 26.84 | 70.11 | 187.57 | 83.58 | 115.24 |
| | | 10 | 41.56 | 40.13 | 305.58 | 136.34 | 166.42 |
| | | 100 | 72.05 | 21.20 | 381.15 | 308.80 | 275.18 |
| 64-4 | Leaves | 1 | 20.11 | 45.12 | 148.13 | 82.14 | 88.52 |
| | | 10 | 46.34 | 30.64 | 341.34 | 154.41 | 189.62 |
| | | 100 | 80.80 | 24.66 | 500.44 | 490.30 | 280.48 |
| 64-5 | Fruits | 1 | 25.79 | 74.34 | 130.13 | 87.98 | 91.07 |
| | | 10 | 45.12 | 37.48 | 211.15 | 162.04 | 134.89 |
| | | 100 | 77.15 | 28.92 | 400.67 | 312.50 | 271.13 |
| 64-6 | Flowers | 1 | 29.20 | 51.96 | 105.70 | 94.68 | 110.21 |
| | | 10 | 45.54 | 37.84 | 246.52 | 147.91 | 103.53 |
| | | 100 | 72.91 | 24.42 | 369.69 | 397.78 | 313.08 |

TABLE 130

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 64 | Glossy-leaf | | | | | | |
| | paper plant | | | | | | |
| 64-1 | Whole plant | 1 | 100.96 | 96.27 | 110.90 | 80.70 | 198.48 |
| | | 10 | 89.49 | 125.73 | 65.05 | 81.27 | 339.50 |
| | | 100 | 73.16 | 143.21 | 37.89 | 91.04 | 391.13 |
| 64-2 | Roots | 1 | 101.65 | 108.79 | 87.91 | 78.96 | 167.75 |
| | | 10 | 80.35 | 127.83 | 64.63 | 89.99 | 323.35 |
| | | 100 | 77.55 | 140.39 | 47.29 | 109.80 | 301.16 |
| 64-3 | Stems | 1 | 101.11 | 115.57 | 110.85 | 84.05 | 135.71 |
| | | 10 | 92.18 | 127.57 | 67.09 | 92.65 | 245.61 |
| | | 100 | 80.28 | 147.46 | 1.45 | 96.82 | 420.52 |
| 64-4 | Leaves | 1 | 95.63 | 114.12 | 84.18 | 75.74 | 91.44 |
| | | 10 | 83.55 | 108.98 | 57.77 | 87.33 | 300.03 |
| | | 100 | 77.53 | 147.47 | 16.54 | 103.01 | 422.42 |
| 64-5 | Fruits | 1 | 99.71 | 96.18 | 108.64 | 69.77 | 156.37 |
| | | 10 | 91.76 | 107.74 | 78.96 | 91.39 | 183.73 |
| | | 100 | 78.12 | 136.44 | 23.22 | 101.15 | 489.29 |
| 64-6 | Flowers | 1 | 99.54 | 99.27 | 82.22 | 65.37 | 110.75 |
| | | 10 | 92.94 | 109.86 | 46.43 | 84.96 | 238.30 |
| | | 100 | 74.96 | 146.88 | 16.08 | 91.54 | 441.98 |

### Example 65. Three-Leaf Clematis

TABLE 131

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 65 | Three-leaf | | | | | | |
| | clematis | | | | | | |
| 65-1 | Whole plant | 1 | 30.47 | 75.52 | 125.67 | 102.64 | 83.12 |
| | | 10 | 30.19 | 35.70 | 200.83 | 162.48 | 156.88 |
| | | 100 | 75.12 | 30.60 | 445.55 | 337.00 | 233.69 |
| 65-2 | Roots | 1 | 30.57 | 87.47 | 113.44 | 100.48 | 97.83 |
| | | 10 | 49.62 | 35.77 | 252.30 | 190.51 | 181.16 |
| | | 100 | 73.12 | 25.93 | 438.67 | 395.41 | 294.50 |
| 65-3 | Stems | 1 | 30.82 | 70.20 | 144.91 | 98.03 | 142.05 |
| | | 10 | 46.28 | 35.40 | 306.56 | 199.74 | 215.49 |
| | | 100 | 84.31 | 29.06 | 467.79 | 424.76 | 312.96 |
| 65-4 | Leaves | 1 | 30.82 | 51.89 | 101.28 | 89.73 | 126.08 |
| | | 10 | 31.59 | 35.20 | 215.40 | 187.32 | 190.55 |
| | | 100 | 84.45 | 28.54 | 376.82 | 321.08 | 230.24 |
| 65-5 | Fruits | 1 | 25.21 | 68.14 | 196.98 | 85.55 | 148.35 |
| | | 10 | 38.04 | 40.30 | 343.89 | 128.45 | 137.28 |
| | | 100 | 70.16 | 20.14 | 433.68 | 450.35 | 300.63 |
| 65-5 | Flowers | 1 | 23.14 | 68.10 | 105.21 | 100.68 | 133.12 |
| | | 10 | 31.38 | 40.65 | 279.91 | 113.73 | 168.94 |
| | | 100 | 78.14 | 26.64 | 496.21 | 483.73 | 294.67 |

TABLE 132

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 65 | Three-leaf | | | | | | |
| | clematis | | | | | | |
| 65-1 | Whole plant | 1 | 97.90 | 103.64 | 105.62 | 83.78 | 114.47 |
| | | 10 | 82.52 | 135.21 | 75.43 | 95.83 | 293.20 |
| | | 100 | 80.42 | 145.36 | 8.30 | 106.56 | 442.91 |
| 65-2 | Roots | 1 | 98.79 | 114.87 | 94.33 | 74.39 | 210.46 |
| | | 10 | 84.66 | 121.34 | 60.43 | 82.46 | 205.93 |
| | | 100 | 80.03 | 151.82 | 8.70 | 96.77 | 373.97 |
| 65-3 | Stems | 1 | 98.49 | 113.68 | 102.85 | 81.95 | 123.40 |
| | | 10 | 81.88 | 111.28 | 76.94 | 84.76 | 180.71 |
| | | 100 | 75.56 | 152.62 | 9.00 | 94.92 | 437.85 |
| 65-4 | Leaves | 1 | 96.82 | 100.65 | 87.24 | 84.17 | 205.24 |
| | | 10 | 92.32 | 137.91 | 75.69 | 75.56 | 219.88 |
| | | 100 | 76.75 | 145.06 | 11.91 | 99.18 | 379.39 |
| 65-5 | Fruits | 1 | 97.95 | 108.20 | 92.20 | 70.11 | 198.05 |
| | | 10 | 88.08 | 114.38 | 55.82 | 80.63 | 282.22 |
| | | 100 | 80.29 | 154.56 | 6.18 | 101.22 | 376.05 |
| 65-5 | Flowers | 1 | 100.48 | 112.13 | 90.17 | 82.73 | 170.13 |
| | | 10 | 89.08 | 111.47 | 75.83 | 91.37 | 340.83 |
| | | 100 | 79.76 | 138.36 | 5.61 | 99.99 | 421.33 |

### Example 66. Lyre-Leaf Nightshade

TABLE 133

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 66 | Lyre-leaf | | | | | | |
| | nightshade | | | | | | |
| 66-1 | Whole plant | 1 | 20.09 | 80.86 | 100.61 | 92.41 | 90.21 |
| | | 10 | 46.15 | 33.66 | 286.03 | 159.47 | 215.16 |
| | | 100 | 78.21 | 24.34 | 498.77 | 427.43 | 241.81 |
| 66-2 | Roots | 1 | 24.16 | 82.38 | 153.04 | 106.55 | 102.70 |
| | | 10 | 35.14 | 40.36 | 255.07 | 169.65 | 225.26 |
| | | 100 | 79.40 | 23.63 | 393.49 | 452.45 | 295.57 |
| 66-3 | Stems | 1 | 30.52 | 72.74 | 131.03 | 97.05 | 126.61 |
| | | 10 | 50.02 | 30.14 | 247.38 | 114.38 | 227.91 |
| | | 100 | 85.51 | 25.13 | 468.26 | 462.58 | 350.75 |
| 66-4 | Leaves | 1 | 30.72 | 63.80 | 183.29 | 81.54 | 139.81 |
| | | 10 | 41.21 | 34.49 | 247.13 | 173.78 | 148.27 |
| | | 100 | 76.40 | 28.14 | 448.44 | 420.36 | 229.91 |
| 66-5 | Fruits | 1 | 28.03 | 48.09 | 189.62 | 99.07 | 99.67 |
| | | 10 | 36.17 | 38.76 | 266.05 | 189.32 | 110.80 |
| | | 100 | 74.81 | 29.21 | 429.43 | 341.81 | 244.75 |
| 66-6 | Flowers | 1 | 21.93 | 63.12 | 190.69 | 108.25 | 82.25 |
| | | 10 | 30.51 | 35.00 | 277.19 | 135.50 | 203.17 |
| | | 100 | 78.30 | 28.38 | 374.49 | 455.64 | 268.19 |

TABLE 134

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 66 | Lyre-leaf | | | | | | |
| | nightshade | | | | | | |
| 66-1 | Whole plant | 1 | 99.33 | 96.10 | 105.23 | 84.79 | 199.65 |
| | | 10 | 83.40 | 132.12 | 52.57 | 89.86 | 187.41 |
| | | 100 | 71.49 | 135.83 | 13.87 | 90.31 | 369.70 |
| 66-2 | Roots | 1 | 103.36 | 101.45 | 88.51 | 66.32 | 192.00 |
| | | 10 | 91.18 | 127.90 | 93.92 | 82.91 | 316.75 |
| | | 100 | 80.46 | 133.17 | 39.30 | 105.36 | 331.08 |
| 66-3 | Stems | 1 | 103.48 | 98.47 | 106.36 | 81.22 | 192.80 |
| | | 10 | 92.36 | 112.17 | 55.46 | 85.51 | 229.23 |
| | | 100 | 78.65 | 149.66 | 38.77 | 99.08 | 384.31 |
| 66-4 | Leaves | 1 | 103.25 | 115.16 | 82.50 | 65.80 | 168.64 |
| | | 10 | 92.89 | 111.62 | 63.51 | 78.73 | 290.34 |
| | | 100 | 77.08 | 130.32 | 33.68 | 99.85 | 460.66 |
| 66-5 | Fruits | 1 | 96.58 | 109.42 | 82.13 | 77.95 | 147.96 |
| | | 10 | 90.71 | 129.58 | 64.14 | 92.81 | 291.99 |
| | | 100 | 73.47 | 158.52 | 0.10 | 104.25 | 334.57 |
| 66-6 | Flowers | 1 | 100.43 | 108.33 | 99.95 | 67.78 | 116.57 |
| | | 10 | 88.46 | 140.40 | 88.18 | 86.84 | 333.53 |
| | | 100 | 75.06 | 153.41 | 30.10 | 109.29 | 371.63 |

### Example 67. Ivy Morning Glory

TABLE 135

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 67 | Ivy morning | | | | | | |
| | glory | | | | | | |
| 67-1 | Whole plant | 1 | 27.24 | 63.18 | 125.10 | 92.24 | 105.20 |
| | | 10 | 37.57 | 34.99 | 319.79 | 113.07 | 114.74 |
| | | 100 | 84.79 | 20.56 | 454.73 | 428.44 | 345.20 |
| 67-2 | Roots | 1 | 28.29 | 69.42 | 115.60 | 99.23 | 134.07 |
| | | 10 | 35.70 | 32.92 | 219.06 | 159.13 | 179.57 |
| | | 100 | 70.28 | 30.33 | 423.14 | 446.33 | 229.52 |
| 67-3 | Stems | 1 | 20.56 | 46.61 | 114.38 | 108.95 | 133.23 |
| | | 10 | 49.64 | 30.10 | 288.10 | 154.70 | 146.89 |
| | | 100 | 75.05 | 29.78 | 365.74 | 417.91 | 261.23 |
| 67-4 | Leaves | 1 | 28.73 | 77.83 | 169.50 | 93.43 | 138.32 |
| | | 10 | 42.12 | 31.28 | 277.46 | 177.99 | 178.50 |
| | | 100 | 76.96 | 27.39 | 459.73 | 367.15 | 288.54 |
| 67-5 | Fruits | 1 | 23.55 | 73.52 | 196.02 | 107.79 | 87.46 |
| | | 10 | 45.56 | 33.61 | 309.88 | 148.00 | 144.26 |
| | | 100 | 72.53 | 21.88 | 450.61 | 380.73 | 280.89 |
| 67-6 | Flowers | 1 | 22.50 | 48.49 | 134.88 | 85.07 | 145.39 |
| | | 10 | 50.99 | 37.37 | 312.57 | 156.14 | 157.40 |
| | | 100 | 75.02 | 30.80 | 428.83 | 496.66 | 278.65 |

TABLE 136

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 67 | Ivy morning | | | | | | |
| | glory | | | | | | |
| 67-1 | Whole plant | 1 | 98.56 | 108.76 | 95.38 | 66.16 | 98.55 |
| | | 10 | 86.29 | 121.47 | 78.66 | 90.68 | 210.97 |
| | | 100 | 78.04 | 136.06 | 41.44 | 102.58 | 419.41 |
| 67-2 | Roots | 1 | 98.58 | 108.08 | 81.18 | 82.77 | 93.87 |
| | | 10 | 81.02 | 126.65 | 75.94 | 89.53 | 174.92 |
| | | 100 | 72.61 | 145.42 | 25.29 | 102.40 | 446.34 |
| 67-3 | Stems | 1 | 98.50 | 101.89 | 91.52 | 68.99 | 144.54 |
| | | 10 | 88.04 | 140.55 | 58.15 | 94.71 | 230.62 |
| | | 100 | 73.09 | 134.29 | 50.72 | 91.35 | 302.24 |
| 67-4 | Leaves | 1 | 97.63 | 114.85 | 88.16 | 81.98 | 147.44 |
| | | 10 | 88.73 | 118.97 | 79.21 | 81.38 | 170.67 |
| | | 100 | 73.84 | 149.96 | 0.00 | 94.72 | 425.66 |
| 67-5 | Fruits | 1 | 102.97 | 108.70 | 97.44 | 71.51 | 109.01 |
| | | 10 | 93.43 | 138.16 | 77.88 | 75.90 | 218.65 |
| | | 100 | 78.81 | 159.90 | 3.38 | 92.25 | 337.11 |
| 67-6 | Flowers | 1 | 95.18 | 106.00 | 108.75 | 72.31 | 137.51 |
| | | 10 | 83.78 | 138.44 | 84.18 | 81.05 | 218.65 |
| | | 100 | 70.80 | 147.46 | 5.51 | 104.17 | 429.95 |

### Example 68. Beach Morning Glory

TABLE 137

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 68 | Beach | | | | | | |
| | morning | | | | | | |
| | glory | | | | | | |
| 68-1 | Whole plant | 1 | 25.20 | 58.25 | 199.86 | 83.60 | 137.68 |
| | | 10 | 37.02 | 38.32 | 204.71 | 137.98 | 110.32 |
| | | 100 | 83.03 | 22.87 | 351.65 | 403.74 | 260.17 |
| 68-2 | Roots | 1 | 20.09 | 76.78 | 109.85 | 81.67 | 94.19 |
| | | 10 | 49.32 | 31.90 | 224.60 | 135.74 | 108.27 |
| | | 100 | 80.49 | 23.14 | 453.01 | 414.82 | 303.54 |
| 68-3 | Stems | 1 | 30.66 | 62.64 | 190.71 | 81.11 | 96.05 |
| | | 10 | 50.56 | 36.49 | 232.35 | 187.84 | 104.58 |
| | | 100 | 84.07 | 28.75 | 440.89 | 410.48 | 343.73 |
| 68-4 | Leaves | 1 | 24.00 | 76.28 | 117.61 | 104.25 | 85.78 |
| | | 10 | 41.48 | 38.21 | 248.95 | 163.01 | 133.00 |
| | | 100 | 81.53 | 25.46 | 417.39 | 332.98 | 340.05 |
| 68-5 | Fruits | 1 | 28.07 | 54.83 | 154.85 | 100.31 | 114.23 |
| | | 10 | 32.54 | 30.78 | 234.05 | 146.63 | 242.78 |
| | | 100 | 71.86 | 30.04 | 469.10 | 417.59 | 247.91 |
| 68-6 | Flowers | 1 | 26.28 | 77.33 | 147.61 | 92.10 | 83.09 |
| | | 10 | 50.21 | 34.27 | 211.34 | 185.07 | 225.37 |
| | | 100 | 79.72 | 20.43 | 422.86 | 371.94 | 273.10 |
| 68-7 | Underground | 1 | 28.07 | 54.83 | 154.85 | 100.31 | 114.23 |
| | stems | 10 | 32.54 | 30.78 | 234.05 | 146.63 | 242.78 |
| | | 100 | 71.86 | 30.04 | 469.10 | 417.59 | 247.91 |
| 68-8 | Shoots | 1 | 26.28 | 77.33 | 147.61 | 92.10 | 83.09 |
| | | 10 | 50.21 | 34.27 | 211.34 | 185.07 | 225.37 |
| | | 100 | 79.72 | 20.43 | 422.86 | 371.94 | 273.10 |

TABLE 138

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 68 | Beach morning | | | | | | |
| | glory | | | | | | |
| 68-1 | Whole plant | 1 | 98.41 | 110.05 | 105.04 | 79.71 | 136.67 |
| | | 10 | 81.32 | 140.39 | 63.46 | 78.42 | 338.10 |
| | | 100 | 74.93 | 149.48 | 35.08 | 104.25 | 310.68 |
| 68-2 | Roots | 1 | 100.49 | 102.75 | 83.76 | 80.57 | 106.92 |
| | | 10 | 80.13 | 132.60 | 95.95 | 87.67 | 275.28 |
| | | 100 | 78.01 | 146.06 | 49.58 | 90.52 | 445.73 |
| 68-3 | Stems | 1 | 103.31 | 110.75 | 83.39 | 83.89 | 91.95 |
| | | 10 | 83.64 | 134.96 | 81.62 | 82.94 | 187.47 |
| | | 100 | 72.72 | 150.72 | 38.25 | 106.39 | 334.04 |
| 68-4 | Leaves | 1 | 103.74 | 102.95 | 108.56 | 83.07 | 140.03 |
| | | 10 | 80.90 | 127.33 | 61.43 | 86.09 | 204.76 |
| | | 100 | 77.94 | 147.03 | 47.03 | 106.20 | 455.88 |
| 68-5 | Fruits | 1 | 98.62 | 112.65 | 109.59 | 80.07 | 199.06 |
| | | 10 | 81.04 | 119.98 | 93.06 | 85.18 | 226.19 |
| | | 100 | 75.03 | 133.34 | 11.20 | 106.99 | 402.38 |
| 68-6 | Flowers | 1 | 17.53 | 95.10 | 93.69 | 65.42 | 110.02 |
| | | 10 | 21.84 | 112.31 | 62.41 | 80.12 | 242.79 |
| | | 100 | 28.81 | 160.65 | 3.19 | 104.36 | 373.35 |
| 68-7 | Underground | 1 | 98.62 | 112.65 | 107.82 | 80.07 | 199.06 |
| | stems | 10 | 81.04 | 119.98 | 62.54 | 85.18 | 226.19 |
| | | 100 | 75.03 | 133.34 | 42.89 | 106.99 | 402.38 |
| 68-8 | Shoots | 1 | 97.53 | 95.10 | 97.03 | 65.42 | 110.02 |
| | | 10 | 91.84 | 112.31 | 62.54 | 80.12 | 242.79 |
| | | 100 | 78.81 | 160.65 | 48.14 | 104.36 | 373.35 |

### Example 69. East Asian Beautyberry

TABLE 139

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 69 | East Asian | | | | | | |
| | beautyberry | | | | | | |
| 69-1 | Whole plant | 1 | 25.89 | 87.30 | 154.43 | 94.61 | 98.41 |
| | | 10 | 45.27 | 32.70 | 346.54 | 122.02 | 210.33 |
| | | 100 | 79.39 | 25.56 | 409.06 | 317.17 | 312.36 |
| 69-2 | Roots | 1 | 22.60 | 58.79 | 115.96 | 102.96 | 129.38 |
| | | 10 | 43.90 | 35.44 | 246.06 | 162.22 | 132.22 |
| | | 100 | 82.23 | 20.70 | 492.30 | 300.40 | 325.95 |
| 69-3 | Stems | 1 | 26.29 | 59.45 | 159.05 | 91.40 | 101.99 |
| | | 10 | 30.61 | 32.01 | 290.11 | 150.71 | 106.43 |
| | | 100 | 72.43 | 27.35 | 405.27 | 431.82 | 204.95 |
| 69-4 | Leaves | 1 | 23.16 | 52.84 | 100.67 | 84.12 | 134.17 |
| | | 10 | 47.92 | 39.57 | 241.04 | 160.33 | 186.79 |
| | | 100 | 81.89 | 27.69 | 385.10 | 471.29 | 230.33 |
| 69-5 | Fruits | 1 | 21.11 | 67.74 | 126.76 | 89.78 | 124.47 |
| | | 10 | 37.10 | 39.81 | 242.28 | 113.55 | 213.85 |
| | | 100 | 80.26 | 28.18 | 386.93 | 493.00 | 323.31 |
| 69-6 | Flowers | 1 | 26.72 | 40.31 | 151.61 | 108.88 | 102.25 |
| | | 10 | 44.90 | 35.31 | 301.99 | 192.68 | 246.61 |
| | | 100 | 77.76 | 28.27 | 470.36 | 317.55 | 258.90 |

TABLE 140

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 69 | East Asian | | | | | | |
| | beautyberry | | | | | | |
| 69-1 | Whole plant | 1 | 95.00 | 109.13 | 108.33 | 66.17 | 178.36 |
| | | 10 | 83.50 | 110.30 | 82.89 | 88.50 | 265.82 |
| | | 100 | 71.66 | 154.22 | 1.44 | 107.97 | 335.49 |
| 69-2 | Roots | 1 | 97.19 | 103.67 | 96.48 | 75.64 | 144.96 |
| | | 10 | 88.92 | 134.92 | 61.09 | 77.44 | 209.64 |
| | | 100 | 73.63 | 137.62 | 3.00 | 103.91 | 332.32 |
| 69-3 | Stems | 1 | 101.09 | 100.34 | 82.56 | 78.55 | 112.95 |
| | | 10 | 87.67 | 132.08 | 95.82 | 94.32 | 281.58 |
| | | 100 | 78.04 | 148.73 | 40.92 | 109.50 | 328.89 |
| 69-4 | Leaves | 1 | 103.91 | 112.16 | 109.28 | 84.26 | 103.48 |
| | | 10 | 90.78 | 134.28 | 92.31 | 86.28 | 273.94 |
| | | 100 | 74.12 | 156.68 | 25.05 | 101.06 | 356.46 |
| 69-5 | Fruits | 1 | 97.59 | 111.35 | 108.78 | 83.01 | 168.36 |
| | | 10 | 92.29 | 105.56 | 87.57 | 78.61 | 299.17 |
| | | 100 | 78.20 | 155.34 | 38.48 | 108.68 | 324.90 |
| 69-6 | Flowers | 1 | 102.17 | 107.39 | 103.92 | 83.47 | 195.15 |
| | | 10 | 83.68 | 131.00 | 72.35 | 87.83 | 215.60 |
| | | 100 | 79.52 | 155.43 | 40.45 | 94.38 | 407.37 |

### Example 70. Korean Mint

TABLE 141

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 70 | Korean mint | | | | | | |
| 70-1 | Whole plant | 1 | 3.55 | 88.22 | 86.63 | 72.30 | 104.65 |
| | | 10 | 11.75 | 81.47 | 91.09 | 93.81 | 186.79 |
| | | 100 | 25.93 | 76.23 | 105.47 | 106.82 | 237.86 |
| 70-2 | Roots | 1 | 27.88 | 79.10 | 188.11 | 105.25 | 133.04 |
| | | 10 | 37.19 | 31.44 | 225.96 | 161.06 | 193.59 |
| | | 100 | 71.06 | 23.66 | 420.86 | 401.51 | 340.94 |
| 70-3 | Stems | 1 | 21.00 | 70.50 | 165.00 | 92.30 | 92.77 |
| | | 10 | 50.38 | 36.28 | 261.77 | 158.70 | 223.08 |
| | | 100 | 78.26 | 30.53 | 372.27 | 419.34 | 256.64 |
| 70-4 | Leaves | 1 | 20.57 | 59.74 | 140.38 | 85.22 | 120.16 |
| | | 10 | 48.58 | 34.24 | 260.80 | 165.32 | 244.95 |
| | | 100 | 81.68 | 20.13 | 483.36 | 345.93 | 274.10 |
| 70-5 | Fruits | 1 | 22.38 | 53.05 | 173.07 | 109.69 | 144.62 |
| | | 10 | 30.28 | 36.77 | 241.90 | 139.69 | 233.56 |
| | | 100 | 83.14 | 21.78 | 390.77 | 309.70 | 336.08 |
| 70-6 | Flowers | 1 | 28.87 | 58.53 | 148.90 | 88.26 | 125.92 |
| | | 10 | 48.54 | 39.29 | 263.01 | 146.74 | 146.10 |
| | | 100 | 75.53 | 25.63 | 436.15 | 340.62 | 237.75 |

TABLE 142

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 70 | Korean mini | | | | | | |
| 70-1 | Whole plant | 1 | 95.74 | 98.59 | 106.46 | 56.29 | 149.71 |
| | | 10 | 88.05 | 124.18 | 88.25 | 61.34 | 280.89 |
| | | 100 | 77.51 | 146.45 | 43.30 | 65.65 | 314.15 |
| 70-2 | Roots | 1 | 95.70 | 107.86 | 110.71 | 80.28 | 207.80 |
| | | 10 | 90.76 | 136.06 | 74.08 | 94.25 | 311.17 |
| | | 100 | 75.92 | 137.72 | 10.33 | 104.64 | 438.65 |
| 70-3 | Stems | 1 | 100.11 | 109.47 | 91.28 | 76.67 | 203.21 |
| | | 10 | 84.16 | 108.39 | 88.44 | 93.65 | 321.89 |
| | | 100 | 79.27 | 136.24 | 20.10 | 105.31 | 338.24 |
| 70-4 | Leaves | 1 | 98.91 | 109.38 | 99.60 | 68.20 | 116.77 |
| | | 10 | 86.99 | 120.01 | 64.99 | 88.21 | 224.24 |
| | | 100 | 79.74 | 157.92 | 13.71 | 110.18 | 391.11 |
| 70-5 | Fruits | 1 | 100.05 | 115.23 | 84.93 | 82.58 | 91.76 |
| | | 10 | 90.30 | 129.37 | 83.94 | 88.64 | 296.13 |
| | | 100 | 73.88 | 149.67 | 40.76 | 98.81 | 310.49 |
| 70-6 | Flowers | 1 | 96.75 | 100.63 | 89.09 | 84.69 | 185.19 |
| | | 10 | 94.81 | 114.67 | 89.34 | 92.62 | 319.39 |
| | | 100 | 80.69 | 138.29 | 15.40 | 109.92 | 391.56 |

### Example 71. Small-Flower Asian Calamint

TABLE 143

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 71 | Small-flower | | | | | | |
| | Asian calamint | | | | | | |
| 71-1 | Whole plant | 1 | 21.12 | 86.77 | 173.65 | 87.19 | 134.61 |
| | | 10 | 32.10 | 40.93 | 216.54 | 145.43 | 236.78 |
| | | 100 | 72.52 | 25.83 | 451.78 | 484.70 | 271.14 |
| 71-2 | Roots | 1 | 24.23 | 40.63 | 169.48 | 84.25 | 141.67 |
| | | 10 | 38.13 | 35.86 | 435.12 | 155.42 | 131.02 |
| | | 100 | 75.50 | 21.45 | 423.16 | 333.01 | 299.80 |
| 71-3 | Stems | 1 | 26.14 | 75.93 | 101.60 | 92.53 | 117.81 |
| | | 10 | 38.98 | 39.72 | 246.11 | 171.06 | 246.93 |
| | | 100 | 70.40 | 27.89 | 476.62 | 422.79 | 315.59 |
| 71-4 | Leaves | 1 | 16.54 | 79.31 | 153.81 | 86.64 | 147.08 |
| | | 10 | 23.28 | 30.86 | 246.95 | 193.78 | 205.72 |
| | | 100 | 35.75 | 29.68 | 419.69 | 404.12 | 348.38 |
| 71-5 | Fruits | 1 | 25.06 | 47.61 | 132.89 | 84.21 | 147.89 |
| | | 10 | 45.04 | 38.52 | 217.23 | 192.19 | 122.33 |
| | | 100 | 72.99 | 26.74 | 396.29 | 322.48 | 336.14 |
| 71-6 | Flowers | 1 | 20.53 | 43.81 | 188.20 | 108.45 | 111.60 |
| | | 10 | 35.78 | 40.39 | 318.18 | 127.07 | 153.61 |
| | | 100 | 71.31 | 26.15 | 426.87 | 470.98 | 260.71 |

TABLE 144

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 71 | Small-flower | | | | | | |
| | Asian calamint | | | | | | |
| 71-1 | Whole plant | 1 | 101.78 | 115.09 | 85.35 | 78.48 | 127.63 |
| | | 10 | 84.24 | 132.48 | 69.74 | 90.63 | 216.32 |
| | | 100 | 72.28 | 148.56 | 10.40 | 98.06 | 480.05 |
| 71-2 | Roots | 1 | 96.47 | 98.61 | 93.38 | 84.18 | 117.79 |
| | | 10 | 80.73 | 116.41 | 87.64 | 93.27 | 323.15 |
| | | 100 | 79.98 | 142.49 | 7.98 | 110.52 | 457.70 |
| 71-3 | Stems | 1 | 95.43 | 95.69 | 110.31 | 77.78 | 136.51 |
| | | 10 | 91.54 | 122.47 | 82.40 | 79.21 | 175.32 |
| | | 100 | 73.82 | 132.83 | 12.49 | 103.29 | 499.49 |
| 71-4 | Leaves | 1 | 98.79 | 93.53 | 82.43 | 72.02 | 158.28 |
| | | 10 | 84.13 | 98.72 | 81.80 | 92.31 | 223.33 |
| | | 100 | 78.32 | 107.24 | 7.22 | 90.41 | 346.30 |
| 71-5 | Fruits | 1 | 100.76 | 108.77 | 97.24 | 72.11 | 105.78 |
| | | 10 | 91.04 | 117.47 | 90.28 | 77.30 | 240.99 |
| | | 100 | 77.03 | 135.47 | 15.93 | 92.96 | 491.34 |
| 71-6 | Flowers | 1 | 101.20 | 103.31 | 100.37 | 73.58 | 203.45 |
| | | 10 | 88.39 | 128.41 | 70.03 | 92.47 | 309.47 |
| | | 100 | 75.14 | 138.72 | 24.17 | 102.46 | 427.46 |

### Example 72. Henbit Deadnettle

TABLE 145

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 72 | Henbit | | | | | | |
| | deadnettle | | | | | | |
| 72-1 | Whole plant | 1 | 27.78 | 74.14 | 89.49 | 80.69 | 112.25 |
| | | 10 | 48.44 | 34.45 | 96.75 | 121.77 | 204.46 |
| | | 100 | 82.22 | 23.33 | 100.45 | 431.28 | 349.19 |
| 72-2 | Roots | 1 | 28.75 | 75.81 | 111.56 | 97.87 | 90.31 |
| | | 10 | 49.06 | 31.30 | 331.61 | 127.54 | 185.51 |
| | | 100 | 81.07 | 23.38 | 361.82 | 414.12 | 276.75 |
| 72-3 | Stems | 1 | 23.01 | 88.76 | 146.41 | 82.72 | 93.02 |
| | | 10 | 49.92 | 38.49 | 299.44 | 155.62 | 248.57 |
| | | 100 | 83.70 | 22.12 | 499.07 | 421.06 | 210.12 |
| 72-4 | Leaves | 1 | 29.38 | 49.16 | 102.23 | 92.67 | 142.91 |
| | | 10 | 39.30 | 40.54 | 309.43 | 136.16 | 249.02 |
| | | 100 | 70.88 | 23.08 | 364.50 | 407.21 | 253.63 |
| 72-5 | Fruits | 1 | 25.45 | 41.55 | 163.12 | 93.91 | 119.23 |
| | | 10 | 48.27 | 34.62 | 340.68 | 120.56 | 212.14 |
| | | 100 | 75.75 | 22.78 | 369.10 | 409.89 | 204.30 |
| 72-6 | Flowers | 1 | 22.61 | 69.14 | 104.35 | 92.94 | 146.30 |
| | | 10 | 44.95 | 31.37 | 242.94 | 187.78 | 237.54 |
| | | 100 | 71.43 | 28.86 | 374.31 | 406.22 | 227.16 |

TABLE 146

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 72 | Henbit | | | | | | |
| | deadnettle | | | | | | |
| 72-1 | Whole plant | 1 | 102.84 | 98.08 | 98.96 | 77.61 | 168.89 |
| | | 10 | 86.81 | 112.06 | 91.14 | 94.16 | 278.55 |
| | | 100 | 77.68 | 141.21 | 40.54 | 103.76 | 470.76 |
| 72-2 | Roots | 1 | 99.83 | 96.73 | 91.23 | 79.94 | 190.43 |
| | | 10 | 94.64 | 129.06 | 75.59 | 86.30 | 278.41 |
| | | 100 | 71.08 | 131.85 | 31.90 | 91.46 | 392.26 |
| 72-3 | Stems | 1 | 95.28 | 114.24 | 85.74 | 69.35 | 157.33 |
| | | 10 | 93.94 | 111.34 | 75.23 | 95.33 | 329.71 |
| | | 100 | 70.43 | 136.51 | 42.95 | 105.83 | 398.67 |
| 72-4 | Leaves | 1 | 97.37 | 106.29 | 95.35 | 77.48 | 174.26 |
| | | 10 | 84.83 | 130.20 | 74.26 | 95.50 | 222.73 |
| | | 100 | 77.90 | 134.04 | 7.03 | 110.06 | 405.01 |
| 72-5 | Fruits | 1 | 95.27 | 95.61 | 102.52 | 66.66 | 163.66 |
| | | 10 | 80.94 | 119.17 | 53.78 | 79.26 | 314.91 |
| | | 100 | 75.70 | 151.62 | 36.05 | 99.69 | 433.91 |
| 72-6 | Flowers | 1 | 96.97 | 99.55 | 106.57 | 80.36 | 180.27 |
| | | 10 | 90.96 | 110.36 | 71.30 | 87.67 | 273.33 |
| | | 100 | 72.99 | 136.41 | 34.32 | 103.62 | 493.08 |

### Example 73. Oriental Motherwort

TABLE 147

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 73 | Oriental | | | | | | |
| | motherwort | | | | | | |
| 73-1 | Whole plant | 1 | 18.84 | 84.71 | 106.10 | 87.74 | 121.26 |
| | | 10 | 21.11 | 80.05 | 115.78 | 92.16 | 224.29 |
| | | 100 | 23.67 | 77.70 | 124.56 | 100.56 | 290.41 |
| 73-2 | Roots | 1 | 28.88 | 80.07 | 159.44 | 105.43 | 136.52 |
| | | 10 | 37.23 | 36.46 | 242.33 | 166.12 | 243.07 |
| | | 100 | 77.28 | 22.95 | 478.03 | 455.14 | 257.33 |
| 73-3 | Stems | 1 | 22.66 | 65.99 | 157.74 | 103.46 | 109.89 |
| | | 10 | 46.18 | 30.53 | 223.94 | 155.46 | 176.90 |
| | | 100 | 78.71 | 29.38 | 446.53 | 394.11 | 347.55 |
| 73-4 | Leaves | 1 | 24.66 | 40.87 | 199.03 | 82.63 | 93.37 |
| | | 10 | 38.39 | 32.74 | 233.58 | 191.79 | 97.93 |
| | | 100 | 73.21 | 27.07 | 459.22 | 382.55 | 107.88 |
| 73-5 | Fruits | 1 | 20.06 | 67.19 | 141.41 | 110.86 | 116.92 |
| | | 10 | 39.76 | 30.04 | 215.86 | 149.91 | 212.74 |
| | | 100 | 70.17 | 20.91 | 423.77 | 406.79 | 209.35 |
| 73-6 | Flowers | 1 | 25.69 | 78.37 | 143.72 | 100.68 | 93.75 |
| | | 10 | 46.96 | 30.31 | 283.84 | 138.33 | 210.37 |
| | | 100 | 74.02 | 22.38 | 424.34 | 362.89 | 329.00 |

TABLE 148

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 73 | Oriental | | | | | | |
| | motherwort | | | | | | |
| 73-1 | Whole plant | 1 | 99.38 | 109.75 | 91.48 | 39.60 | 130.18 |
| | | 10 | 94.98 | 129.67 | 82.23 | 53.31 | 324.09 |
| | | 100 | 79.27 | 140.15 | 26.80 | 62.26 | 417.18 |
| 73-2 | Roots | 1 | 96.40 | 114.77 | 104.37 | 75.54 | 140.12 |
| | | 10 | 81.18 | 140.93 | 81.26 | 75.80 | 205.91 |
| | | 100 | 78.55 | 160.73 | 33.45 | 92.65 | 358.63 |
| 73-3 | Stems | 1 | 100.40 | 106.42 | 92.01 | 73.52 | 166.57 |
| | | 10 | 83.54 | 126.79 | 85.63 | 95.16 | 266.24 |
| | | 100 | 80.33 | 146.22 | 34.93 | 105.83 | 354.33 |
| 73-4 | Leaves | 1 | 103.39 | 112.51 | 92.85 | 68.53 | 160.83 |
| | | 10 | 94.30 | 108.42 | 88.10 | 77.24 | 268.07 |
| | | 100 | 71.76 | 150.43 | 15.52 | 100.34 | 490.38 |
| 73-5 | Fruits | 1 | 99.08 | 107.15 | 83.86 | 71.18 | 148.00 |
| | | 10 | 91.42 | 130.33 | 70.41 | 87.62 | 218.96 |
| | | 100 | 70.25 | 160.48 | 39.75 | 108.21 | 356.29 |
| 73-6 | Flowers | 1 | 95.26 | 99.35 | 88.81 | 85.89 | 161.39 |
| | | 10 | 83.04 | 125.87 | 92.13 | 78.91 | 291.61 |
| | | 100 | 80.04 | 132.11 | 12.33 | 104.13 | 428.62 |

### Example 74. Long-Stalk Low Meadow-Rue

TABLE 149

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 74 | Long-stalk | | | | | | |
| | low | | | | | | |
| | meadow-rue | | | | | | |
| 74-1 | Whole plant | 1 | 21.03 | 40.29 | 185.47 | 102.60 | 127.38 |
| | | 10 | 47.71 | 40.02 | 210.69 | 170.42 | 227.24 |
| | | 100 | 75.84 | 26.77 | 383.61 | 397.39 | 227.06 |
| 74-2 | Roots | 1 | 27.02 | 61.98 | 111.15 | 96.54 | 130.40 |
| | | 10 | 44.67 | 34.57 | 221.56 | 147.49 | 108.15 |
| | | 100 | 75.93 | 23.75 | 445.14 | 408.03 | 224.73 |
| 74-3 | Stems | 1 | 20.01 | 42.16 | 147.47 | 103.75 | 116.23 |
| | | 10 | 43.20 | 37.66 | 285.40 | 111.37 | 229.07 |
| | | 100 | 72.10 | 20.85 | 474.23 | 435.03 | 240.46 |
| 74-4 | Leaves | 1 | 26.87 | 75.15 | 151.45 | 91.16 | 113.69 |
| | | 10 | 46.82 | 34.52 | 307.06 | 143.14 | 244.14 |
| | | 100 | 76.66 | 27.98 | 486.05 | 336.00 | 290.25 |
| 74-5 | Fruits | 1 | 20.44 | 76.59 | 173.65 | 107.39 | 141.13 |
| | | 10 | 49.74 | 32.77 | 261.59 | 164.44 | 140.25 |
| | | 100 | 80.08 | 20.33 | 484.01 | 436.18 | 260.24 |
| 74-6 | Flowers | 1 | 26.18 | 71.51 | 134.20 | 88.29 | 99.48 |
| | | 10 | 37.98 | 38.87 | 308.81 | 194.54 | 134.54 |
| | | 100 | 82.17 | 30.96 | 398.00 | 429.68 | 200.26 |

TABLE 150

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 74 | Long-stalk | | | | | | |
| | low | | | | | | |
| | meadow-rue | | | | | | |
| 74-1 | Whole plant | 1 | 98.96 | 111.05 | 84.64 | 80.95 | 112.81 |
| | | 10 | 90.58 | 135.21 | 75.43 | 95.05 | 207.44 |
| | | 100 | 75.50 | 157.68 | 48.91 | 107.00 | 412.42 |
| 74-2 | Roots | 1 | 96.94 | 114.90 | 109.82 | 74.51 | 132.98 |
| | | 10 | 81.60 | 109.10 | 83.01 | 85.13 | 222.52 |
| | | 100 | 72.38 | 133.33 | 26.83 | 96.70 | 307.47 |
| 74-3 | Stems | 1 | 98.35 | 114.54 | 93.29 | 79.12 | 176.39 |
| | | 10 | 84.29 | 124.16 | 45.12 | 83.50 | 174.23 |
| | | 100 | 71.90 | 139.11 | 38.19 | 108.95 | 347.37 |
| 74-4 | Leaves | 1 | 99.76 | 104.66 | 94.52 | 77.60 | 144.61 |
| | | 10 | 88.24 | 112.25 | 59.79 | 91.06 | 296.99 |
| | | 100 | 76.04 | 131.27 | 10.27 | 107.61 | 436.02 |
| 74-5 | Fruits | 1 | 100.11 | 95.54 | 89.42 | 74.81 | 170.11 |
| | | 10 | 86.79 | 124.25 | 56.95 | 85.86 | 319.42 |
| | | 100 | 75.11 | 131.98 | 42.63 | 107.08 | 339.92 |
| 74-6 | Flowers | 1 | 98.81 | 108.41 | 87.99 | 69.74 | 175.87 |
| | | 10 | 89.05 | 117.52 | 65.01 | 88.10 | 250.36 |
| | | 100 | 80.26 | 157.82 | 14.79 | 101.56 | 324.76 |

### Example 75. Korean Spice Viburnum

TABLE 151

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 75 | Korean spice | | | | | | |
| | viburnum | | | | | | |
| 75-1 | Whole plant | 1 | 27.07 | 58.11 | 141.83 | 105.61 | 120.31 |
| | | 10 | 49.44 | 30.47 | 339.79 | 158.87 | 198.51 |
| | | 100 | 84.57 | 29.14 | 381.49 | 337.60 | 325.59 |
| 75-2 | Roots | 1 | 29.05 | 81.13 | 193.58 | 104.34 | 107.95 |
| | | 10 | 37.40 | 38.73 | 310.42 | 136.15 | 112.07 |
| | | 100 | 73.04 | 23.21 | 470.01 | 467.81 | 205.48 |
| 75-3 | Stems | 1 | 26.45 | 88.82 | 162.54 | 103.60 | 144.44 |
| | | 10 | 43.61 | 40.16 | 342.32 | 184.21 | 160.00 |
| | | 100 | 78.72 | 28.02 | 441.37 | 337.37 | 310.76 |
| 75-4 | Leaves | 1 | 21.38 | 60.05 | 164.03 | 95.03 | 97.26 |
| | | 10 | 48.10 | 30.84 | 335.00 | 137.82 | 230.85 |
| | | 100 | 79.72 | 20.20 | 397.88 | 487.58 | 244.87 |
| 75-5 | Fruits | 1 | 24.23 | 67.60 | 176.08 | 94.16 | 117.54 |
| | | 10 | 36.63 | 34.83 | 339.57 | 115.43 | 217.98 |
| | | 100 | 74.11 | 27.73 | 500.66 | 495.42 | 232.87 |
| 75-6 | Flowers | 1 | 27.33 | 53.78 | 140.41 | 104.00 | 103.85 |
| | | 10 | 33.54 | 38.18 | 274.37 | 138.97 | 130.83 |
| | | 100 | 74.63 | 27.39 | 458.54 | 417.41 | 328.30 |

TABLE 152

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 75 | Korean spice | | | | | | |
| | viburnum | | | | | | |
| 75-1 | Whole plant | 1 | 100.49 | 109.76 | 109.66 | 70.92 | 129.31 |
| | | 10 | 90.52 | 128.16 | 52.69 | 82.10 | 232.57 |
| | | 100 | 77.87 | 133.53 | 4.92 | 110.88 | 323.63 |
| 75-2 | Roots | 1 | 95.38 | 111.62 | 84.73 | 84.31 | 160.81 |
| | | 10 | 92.75 | 116.89 | 55.81 | 86.39 | 211.16 |
| | | 100 | 78.79 | 142.04 | 30.27 | 90.17 | 387.04 |
| 75-3 | Stems | 1 | 95.28 | 95.15 | 107.36 | 72.92 | 159.93 |
| | | 10 | 90.21 | 137.38 | 89.83 | 80.27 | 305.84 |
| | | 100 | 78.69 | 153.12 | 30.99 | 98.99 | 321.60 |
| 75-4 | Leaves | 1 | 100.32 | 101.78 | 102.14 | 66.41 | 96.83 |
| | | 10 | 86.84 | 109.75 | 67.01 | 75.09 | 294.44 |
| | | 100 | 79.11 | 131.77 | 37.92 | 93.83 | 340.39 |
| 75-5 | Fruits | 1 | 101.90 | 109.84 | 93.97 | 81.11 | 136.08 |
| | | 10 | 91.57 | 127.16 | 47.89 | 77.21 | 333.58 |
| | | 100 | 76.52 | 159.26 | 8.25 | 93.93 | 380.33 |
| 75-6 | Flowers | 1 | 96.50 | 96.87 | 96.25 | 72.21 | 117.28 |
| | | 10 | 93.48 | 123.72 | 76.25 | 77.69 | 268.61 |
| | | 100 | 77.64 | 151.31 | 34.37 | 103.14 | 338.34 |

### Example 76. Asian Greater Celandine

TABLE 153

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 76 | Asian greater | | | | | | |
| | celandine | | | | | | |
| 76-1 | Whole plant | 1 | 28.22 | 71.80 | 147.64 | 101.14 | 137.22 |
| | | 10 | 37.21 | 34.50 | 238.66 | 151.84 | 221.80 |
| | | 100 | 72.35 | 26.03 | 384.00 | 386.09 | 232.47 |
| 76-2 | Roots | 1 | 25.28 | 46.78 | 184.98 | 94.90 | 84.63 |
| | | 10 | 34.22 | 37.86 | 255.52 | 127.05 | 172.31 |
| | | 100 | 70.82 | 21.21 | 443.57 | 319.98 | 260.09 |
| 76-3 | Stems | 1 | 29.75 | 46.29 | 119.93 | 91.53 | 150.00 |
| | | 10 | 37.78 | 32.86 | 256.93 | 154.89 | 194.42 |
| | | 100 | 72.85 | 28.02 | 417.58 | 461.69 | 320.80 |
| 76-4 | Leaves | 1 | 24.94 | 74.88 | 198.13 | 109.22 | 94.08 |
| | | 10 | 46.27 | 34.70 | 260.92 | 124.79 | 220.13 |
| | | 100 | 81.07 | 29.79 | 414.21 | 318.39 | 311.42 |
| 76-5 | Fruits | 1 | 24.46 | 47.41 | 102.64 | 101.36 | 89.79 |
| | | 10 | 34.06 | 35.46 | 321.60 | 162.74 | 111.50 |
| | | 100 | 85.50 | 24.81 | 360.78 | 416.47 | 217.89 |
| 76-6 | Flowers | 1 | 21.07 | 42.34 | 104.47 | 104.91 | 121.47 |
| | | 10 | 49.18 | 33.84 | 266.75 | 139.26 | 135.17 |
| | | 100 | 76.91 | 26.78 | 357.48 | 311.65 | 343.55 |

TABLE 154

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 76 | Asian greater | | | | | | |
| | celandine | | | | | | |
| 76-1 | Whole plant | 1 | 99.20 | 109.47 | 101.83 | 65.98 | 199.05 |
| | | 10 | 90.07 | 127.64 | 48.33 | 94.30 | 284.45 |
| | | 100 | 77.20 | 133.47 | 50.12 | 91.46 | 444.41 |
| 76-2 | Roots | 1 | 99.07 | 98.83 | 80.10 | 77.79 | 120.89 |
| | | 10 | 91.94 | 131.77 | 89.35 | 80.39 | 282.63 |
| | | 100 | 77.40 | 138.19 | 18.41 | 99.73 | 313.91 |
| 76-3 | Stems | 1 | 97.70 | 105.80 | 87.03 | 77.11 | 209.09 |
| | | 10 | 89.40 | 118.37 | 53.57 | 75.69 | 201.29 |
| | | 100 | 79.94 | 135.22 | 29.61 | 98.98 | 489.42 |
| 76-4 | Leaves | 1 | 100.99 | 115.52 | 110.83 | 79.93 | 129.98 |
| | | 10 | 85.36 | 140.58 | 71.00 | 91.46 | 286.83 |
| | | 100 | 80.17 | 145.10 | 5.51 | 99.88 | 383.99 |
| 76-5 | Fruits | 1 | 98.39 | 95.51 | 80.09 | 66.88 | 128.58 |
| | | 10 | 91.61 | 138.68 | 48.95 | 91.49 | 236.54 |
| | | 100 | 70.01 | 130.65 | 25.49 | 104.23 | 394.70 |
| 76-6 | Flowers | 1 | 102.20 | 108.83 | 80.61 | 83.34 | 142.64 |
| | | 10 | 81.21 | 106.12 | 66.38 | 81.14 | 293.28 |
| | | 100 | 77.06 | 158.88 | 11.40 | 102.70 | 380.58 |

### Example 77. Seashore Spatulate Aster

TABLE 155

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| | Seashore | | | | | | |
| | spatulate | | | | | | |
| | aster | | | | | | |
| 77-1 | Whole plant | 1 | 20.56 | 98.38 | 99.30 | 83.67 | 105.75 |
| | | 10 | 47.88 | 90.47 | 105.61 | 184.09 | 124.01 |
| | | 100 | 70.37 | 85.34 | 111.22 | 464.94 | 331.27 |
| 77-2 | Roots | 1 | 28.60 | 85.61 | 186.03 | 80.05 | 123.71 |
| | | 10 | 48.57 | 38.33 | 252.94 | 118.05 | 192.15 |
| | | 100 | 70.18 | 28.79 | 480.73 | 374.44 | 346.75 |
| 77-3 | Stems | 1 | 24.37 | 85.36 | 187.27 | 107.25 | 119.10 |
| | | 10 | 33.07 | 32.71 | 331.29 | 121.28 | 176.76 |
| | | 100 | 77.49 | 23.88 | 411.93 | 477.50 | 249.73 |
| 77-4 | Leaves | 1 | 22.94 | 87.23 | 183.56 | 95.49 | 148.23 |
| | | 10 | 42.58 | 32.12 | 347.70 | 158.78 | 138.65 |
| | | 100 | 70.24 | 26.62 | 358.35 | 473.30 | 204.53 |
| 77-5 | Fruits | 1 | 30.85 | 74.80 | 194.81 | 98.93 | 127.51 |
| | | 10 | 39.22 | 40.26 | 345.36 | 149.35 | 130.19 |
| | | 100 | 70.84 | 27.93 | 423.57 | 368.24 | 344.33 |
| 77-6 | Flowers | 1 | 22.46 | 83.46 | 198.11 | 105.94 | 92.36 |
| | | 10 | 32.55 | 36.56 | 236.95 | 128.39 | 198.10 |
| | | 100 | 73.15 | 23.59 | 467.96 | 338.62 | 215.67 |

TABLE 156

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 77 | Seashore | | | | | | |
| | spatulate | | | | | | |
| | aster | | | | | | |
| 77-1 | Whole plant | 1 | 98.28 | 96.22 | 85.12 | 81.05 | 165.23 |
| | | 10 | 93.90 | 103.72 | 84.89 | 82.60 | 302.28 |
| | | 100 | 72.39 | 109.27 | 13.48 | 104.67 | 388.72 |
| 77-2 | Roots | 1 | 98.46 | 105.20 | 87.24 | 74.87 | 169.12 |
| | | 10 | 94.06 | 135.75 | 63.32 | 77.09 | 298.40 |
| | | 100 | 77.45 | 140.55 | 28.85 | 100.32 | 352.92 |
| 77-3 | Stems | 1 | 101.11 | 103.53 | 104.86 | 69.75 | 140.44 |
| | | 10 | 92.03 | 114.51 | 94.84 | 83.12 | 313.51 |
| | | 100 | 72.17 | 138.10 | 7.80 | 105.25 | 469.07 |
| 77-4 | Leaves | 1 | 98.66 | 113.49 | 95.47 | 70.71 | 201.72 |
| | | 10 | 89.08 | 113.34 | 57.15 | 77.74 | 243.90 |
| | | 100 | 74.33 | 135.05 | 22.64 | 106.37 | 309.90 |
| 77-5 | Fruits | 1 | 102.90 | 110.81 | 81.25 | 85.65 | 115.33 |
| | | 10 | 90.19 | 111.71 | 53.40 | 85.59 | 185.47 |
| | | 100 | 78.44 | 158.40 | 2.10 | 98.30 | 327.95 |
| 77-6 | Flowers | 1 | 98.27 | 114.55 | 109.40 | 69.09 | 168.56 |
| | | 10 | 92.70 | 107.11 | 59.79 | 76.96 | 250.07 |
| | | 100 | 74.86 | 153.83 | 28.85 | 90.78 | 461.47 |

### Example 78. Leopard Plant

TABLE 157

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 78 | Leopard | | | | | | |
| | plant | | | | | | |
| 78-1 | Whole plant | 1 | 21.35 | 53.04 | 102.94 | 107.69 | 89.07 |
| | | 10 | 31.30 | 37.14 | 303.97 | 161.78 | 127.73 |
| | | 100 | 72.64 | 29.47 | 487.84 | 411.07 | 207.56 |
| 78-2 | Roots | 1 | 20.87 | 45.44 | 171.83 | 81.09 | 146.49 |
| | | 10 | 41.77 | 38.16 | 327.19 | 163.88 | 119.56 |
| | | 100 | 72.86 | 24.98 | 427.69 | 366.83 | 233.14 |
| 78-3 | Stems | 1 | 21.68 | 70.60 | 193.14 | 99.52 | 128.55 |
| | | 10 | 37.24 | 35.29 | 209.36 | 137.15 | 157.97 |
| | | 100 | 78.08 | 27.07 | 376.53 | 346.10 | 205.19 |
| 78-4 | Leaves | 1 | 25.93 | 58.59 | 193.40 | 107.47 | 123.96 |
| | | 10 | 45.17 | 33.07 | 328.19 | 135.10 | 247.16 |
| | | 100 | 85.69 | 30.54 | 394.03 | 457.13 | 341.04 |
| 78-5 | Fruits | 1 | 24.64 | 72.86 | 155.77 | 91.68 | 119.02 |
| | | 10 | 41.86 | 35.75 | 200.68 | 135.44 | 182.87 |
| | | 100 | 80.78 | 30.98 | 412.13 | 442.19 | 336.63 |
| 78-6 | Flowers | 1 | 30.59 | 52.44 | 118.09 | 107.19 | 126.76 |
| | | 10 | 35.37 | 30.69 | 303.00 | 199.18 | 246.26 |
| | | 100 | 73.59 | 30.08 | 365.29 | 446.16 | 261.52 |

TABLE 158

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 78 | Leopard | | | | | | |
| | plant | | | | | | |
| 78-1 | Whole plant | 1 | 95.31 | 111.80 | 85.28 | 85.17 | 94.05 |
| | | 10 | 94.10 | 138.58 | 93.74 | 79.79 | 230.24 |
| | | 100 | 79.53 | 141.24 | 23.14 | 96.69 | 340.32 |
| 78-2 | Roots | 1 | 103.08 | 102.75 | 105.07 | 72.98 | 163.56 |
| | | 10 | 90.07 | 133.67 | 47.14 | 92.18 | 299.97 |
| | | 100 | 79.81 | 160.70 | 9.15 | 108.94 | 359.08 |
| 78-3 | Stems | 1 | 98.78 | 103.53 | 105.34 | 65.43 | 154.69 |
| | | 10 | 86.85 | 125.74 | 59.42 | 89.71 | 311.65 |
| | | 100 | 70.89 | 139.66 | 48.16 | 92.56 | 426.20 |
| 78-4 | Leaves | 1 | 96.55 | 109.86 | 105.01 | 71.20 | 112.71 |
| | | 10 | 81.00 | 117.46 | 88.77 | 80.53 | 258.98 |
| | | 100 | 70.69 | 160.23 | 26.25 | 91.93 | 385.55 |
| 78-5 | Fruits | 1 | 96.44 | 100.32 | 96.21 | 81.63 | 121.31 |
| | | 10 | 92.07 | 138.87 | 47.34 | 84.85 | 195.70 |
| | | 100 | 70.55 | 157.17 | 49.16 | 102.21 | 309.36 |
| 78-6 | Flowers | 1 | 98.54 | 110.23 | 86.87 | 76.05 | 147.36 |
| | | 10 | 85.25 | 117.19 | 48.33 | 88.55 | 174.63 |
| | | 100 | 74.95 | 160.45 | 46.78 | 102.85 | 499.88 |

### Example 79. Oriental Yellowhead

TABLE 159

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 79 | Oriental | | | | | | |
| | yellowhead | | | | | | |
| 79-1 | Whole plant | 1 | 25.42 | 86.33 | 138.02 | 105.69 | 108.52 |
| | | 10 | 41.15 | 40.57 | 297.29 | 124.87 | 223.93 |
| | | 100 | 84.22 | 26.36 | 460.48 | 313.50 | 345.59 |
| 79-2 | Roots | 1 | 21.81 | 56.25 | 125.06 | 100.48 | 134.49 |
| | | 10 | 39.71 | 30.76 | 225.54 | 194.02 | 182.15 |
| | | 100 | 84.64 | 22.62 | 418.91 | 337.76 | 306.14 |
| 79-3 | Stems | 1 | 21.86 | 47.62 | 194.48 | 100.53 | 111.43 |
| | | 10 | 50.33 | 33.98 | 279.20 | 145.14 | 235.30 |
| | | 100 | 76.62 | 29.89 | 443.19 | 361.36 | 346.34 |
| 79-4 | Leaves | 1 | 29.27 | 82.60 | 164.12 | 88.04 | 121.16 |
| | | 10 | 41.67 | 39.38 | 293.48 | 122.18 | 242.76 |
| | | 100 | 70.50 | 26.04 | 407.88 | 303.55 | 207.73 |
| 79-5 | Fruits | 1 | 30.07 | 63.93 | 119.03 | 86.13 | 112.41 |
| | | 10 | 47.23 | 39.77 | 262.82 | 148.42 | 206.52 |
| | | 100 | 74.51 | 29.45 | 382.27 | 471.65 | 318.76 |
| 79-6 | Flowers | 1 | 26.91 | 67.90 | 197.25 | 99.12 | 91.91 |
| | | 10 | 37.20 | 35.32 | 319.94 | 124.71 | 194.00 |
| | | 100 | 74.56 | 23.53 | 446.62 | 461.59 | 248.71 |

TABLE 160

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 79 | Oriental | | | | | | |
| | yellowhead | | | | | | |
| 79-1 | Whole plant | 1 | 100.39 | 114.43 | 90.22 | 70.48 | 152.43 |
| | | 10 | 95.36 | 124.85 | 87.95 | 80.06 | 254.72 |
| | | 100 | 75.71 | 157.86 | 10.11 | 106.17 | 324.44 |
| 79-2 | Roots | 1 | 102.84 | 102.19 | 102.84 | 72.34 | 111.87 |
| | | 10 | 90.40 | 106.13 | 84.37 | 92.58 | 245.77 |
| | | 100 | 77.83 | 133.16 | 37.68 | 103.49 | 399.77 |
| 79-3 | Stems | 1 | 103.18 | 96.84 | 88.34 | 73.93 | 171.62 |
| | | 10 | 89.95 | 127.86 | 55.95 | 76.77 | 261.11 |
| | | 100 | 78.90 | 137.38 | 2.56 | 100.53 | 305.73 |
| 79-4 | Leaves | 1 | 102.54 | 114.12 | 110.98 | 83.32 | 182.23 |
| | | 10 | 91.47 | 135.69 | 51.89 | 89.15 | 196.30 |
| | | 100 | 73.99 | 144.30 | 27.50 | 108.40 | 348.96 |
| 79-5 | Fruits | 1 | 97.31 | 114.00 | 106.23 | 65.52 | 190.01 |
| | | 10 | 88.71 | 120.60 | 73.34 | 94.89 | 218.07 |
| | | 100 | 74.83 | 139.91 | 7.31 | 92.68 | 318.52 |
| 79-6 | Flowers | 1 | 103.42 | 107.82 | 103.48 | 80.91 | 169.98 |
| | | 10 | 97.77 | 128.97 | 90.57 | 81.02 | 313.58 |
| | | 100 | 88.49 | 137.69 | 4.32 | 91.69 | 493.08 |

### Example 80. Giant Butterbur

TABLE 161

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 80 | Giant | | | | | | |
| | butterbur | | | | | | |
| 80-1 | Whole plant | 1 | 25.41 | 85.99 | 175.17 | 101.67 | 96.44 |
| | | 10 | 45.55 | 34.25 | 205.07 | 111.76 | 122.47 |
| | | 100 | 80.00 | 26.13 | 419.99 | 497.50 | 321.88 |
| 80-1 | Roots | 1 | 20.14 | 69.98 | 138.57 | 83.88 | 111.50 |
| | | 10 | 48.75 | 38.30 | 272.96 | 147.72 | 181.24 |
| | | 100 | 75.96 | 22.24 | 421.47 | 427.33 | 227.38 |
| 80-2 | Stems | 1 | 29.29 | 40.66 | 161.01 | 103.29 | 120.30 |
| | | 10 | 43.46 | 31.76 | 267.96 | 140.49 | 243.54 |
| | | 100 | 73.56 | 21.27 | 475.63 | 476.68 | 282.52 |
| 80-3 | Leaves | 1 | 27.19 | 80.48 | 147.94 | 110.27 | 134.30 |
| | | 10 | 36.11 | 35.81 | 223.53 | 187.77 | 244.91 |
| | | 100 | 71.48 | 25.56 | 433.20 | 404.78 | 346.58 |
| 80-4 | Fruits | 1 | 25.38 | 82.56 | 146.65 | 80.59 | 94.61 |
| | | 10 | 47.34 | 38.62 | 276.32 | 135.22 | 239.54 |
| | | 100 | 76.63 | 20.46 | 421.71 | 479.10 | 286.23 |
| 80-5 | Flowers | 1 | 28.78 | 53.03 | 142.47 | 83.71 | 110.32 |
| | | 10 | 42.28 | 37.82 | 316.03 | 167.78 | 225.11 |
| | | 100 | 77.31 | 25.11 | 379.89 | 385.73 | 214.97 |

TABLE 162

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 80 | Giant | | | | | | |
| | butterbur | | | | | | |
| 80-1 | Whole plant | 1 | 97.56 | 115.81 | 83.86 | 77.49 | 129.15 |
| | | 10 | 82.27 | 133.76 | 80.97 | 94.54 | 332.00 |
| | | 100 | 78.28 | 131.91 | 46.41 | 102.51 | 403.59 |
| 80-1 | Roots | 1 | 99.29 | 111.67 | 85.49 | 76.23 | 124.01 |
| | | 10 | 84.99 | 137.45 | 69.59 | 93.21 | 201.65 |
| | | 100 | 74.23 | 133.76 | 33.01 | 93.21 | 310.27 |
| 80-2 | Stems | 1 | 97.55 | 113.60 | 103.65 | 71.23 | 120.57 |
| | | 10 | 91.87 | 128.69 | 101.94 | 76.74 | 246.78 |
| | | 100 | 70.35 | 159.87 | 84.91 | 107.03 | 334.72 |
| 80-3 | Leaves | 1 | 101.11 | 115.39 | 109.68 | 69.36 | 141.68 |
| | | 10 | 96.53 | 116.81 | 110.70 | 88.10 | 226.02 |
| | | 100 | 88.86 | 136.51 | 133.79 | 99.55 | 448.24 |
| 80-4 | Fruits | 1 | 101.64 | 104.46 | 109.52 | 76.99 | 113.80 |
| | | 10 | 87.63 | 124.76 | 66.36 | 75.51 | 197.19 |
| | | 100 | 78.20 | 135.18 | 47.94 | 101.47 | 303.93 |
| 80-5 | Flowers | 1 | 103.21 | 99.66 | 110.55 | 70.73 | 185.59 |
| | | 10 | 85.04 | 112.09 | 85.44 | 77.20 | 257.58 |
| | | 100 | 71.77 | 160.83 | 12.28 | 110.98 | 338.16 |

### Example 81. Ciliated-Fruit Sedge

TABLE 163

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS | (17β-estradiol |
| | | | 0.05 ppm) | 70.4 nM) | 10 μM) | 1 ppm) | 1 ng/ml) |
| 81 | Ciliated-fruit | | | | | | |
| | sedge | | | | | | |
| 81-1 | Whole plant | 1 | 20.11 | 89.97 | 150.31 | 100.48 | 88.51 |
| | | 10 | 47.78 | 34.64 | 306.00 | 145.52 | 200.12 |
| | | 100 | 72.60 | 30.54 | 471.92 | 376.59 | 324.81 |
| 81-2 | Roots | 1 | 30.47 | 54.68 | 190.47 | 109.61 | 133.59 |
| | | 10 | 33.42 | 33.70 | 324.22 | 133.41 | 116.67 |
| | | 100 | 71.75 | 23.10 | 481.37 | 395.57 | 308.39 |
| 81-3 | Stems | 1 | 22.18 | 51.64 | 163.71 | 101.33 | 88.42 |
| | | 10 | 48.62 | 34.65 | 258.62 | 133.22 | 202.59 |
| | | 100 | 73.06 | 22.15 | 424.99 | 485.38 | 324.30 |
| 81-4 | Leaves | 1 | 30.05 | 51.44 | 170.33 | 82.91 | 141.26 |
| | | 10 | 42.00 | 31.91 | 237.20 | 117.11 | 229.23 |
| | | 100 | 74.10 | 24.39 | 438.97 | 455.68 | 348.23 |
| 81-5 | Fruits | 1 | 26.26 | 65.88 | 117.59 | 88.81 | 146.59 |
| | | 10 | 45.87 | 35.38 | 305.21 | 122.66 | 180.69 |
| | | 100 | 74.49 | 30.06 | 418.93 | 306.15 | 252.48 |
| 81-6 | Flowers | 1 | 27.01 | 58.59 | 188.56 | 93.31 | 148.19 |
| | | 10 | 36.42 | 36.55 | 324.01 | 185.27 | 182.05 |
| | | 100 | 77.69 | 28.67 | 445.53 | 461.59 | 230.33 |

TABLE 164

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 81 | Ciliated-fruit | | | | | | |
| | sedge | | | | | | |
| 81-1 | Whole plant | 1 | 98.16 | 100.71 | 107.12 | 72.04 | 180.98 |
| | | 10 | 86.45 | 119.53 | 74.92 | 82.74 | 294.66 |
| | | 100 | 79.57 | 133.59 | 32.46 | 105.80 | 480.84 |
| 81-2 | Roots | 1 | 103.64 | 97.37 | 82.83 | 67.22 | 104.96 |
| | | 10 | 81.61 | 111.08 | 53.02 | 78.81 | 185.88 |
| | | 100 | 79.90 | 149.12 | 48.91 | 91.93 | 445.37 |
| 81-3 | Stems | 1 | 102.00 | 103.26 | 96.60 | 67.17 | 209.73 |
| | | 10 | 85.77 | 109.19 | 58.92 | 93.12 | 244.29 |
| | | 100 | 79.30 | 136.30 | 15.56 | 96.35 | 446.88 |
| 81-4 | Leaves | 1 | 102.12 | 106.97 | 95.65 | 68.70 | 125.77 |
| | | 10 | 95.39 | 113.55 | 58.29 | 75.81 | 276.00 |
| | | 100 | 78.18 | 153.77 | 33.10 | 109.23 | 359.68 |
| 81-5 | Fruits | 1 | 96.35 | 104.06 | 92.71 | 79.28 | 189.51 |
| | | 10 | 83.44 | 113.31 | 86.71 | 87.08 | 255.14 |
| | | 100 | 80.23 | 134.04 | 42.48 | 95.51 | 319.84 |
| 81-6 | Flowers | 1 | 95.39 | 111.09 | 93.44 | 85.70 | 113.46 |
| | | 10 | 95.50 | 127.11 | 71.32 | 80.35 | 271.35 |
| | | 100 | 76.29 | 154.43 | 7.93 | 93.53 | 416.39 |

### Example 82. Short-Stem Sedge

TABLE 165

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 82 | Short-stem | | | | | | |
| | sedge | | | | | | |
| 82-1 | Whole plant | 1 | 22.27 | 57.16 | 159.29 | 102.01 | 89.22 |
| | | 10 | 34.88 | 39.16 | 212.39 | 175.53 | 127.90 |
| | | 100 | 84.06 | 25.69 | 423.86 | 432.15 | 225.23 |
| 82-2 | Roots | 1 | 26.19 | 51.94 | 191.86 | 81.61 | 119.58 |
| | | 10 | 46.75 | 36.03 | 220.66 | 125.56 | 215.21 |
| | | 100 | 74.83 | 29.62 | 408.27 | 401.20 | 312.19 |
| 82-3 | Stems | 1 | 25.01 | 90.65 | 104.66 | 93.30 | 106.67 |
| | | 10 | 49.76 | 34.96 | 275.43 | 155.92 | 147.42 |
| | | 100 | 80.43 | 30.61 | 350.83 | 457.09 | 252.90 |
| 82-4 | Leaves | 1 | 24.18 | 86.58 | 200.30 | 92.56 | 105.42 |
| | | 10 | 36.18 | 32.41 | 292.97 | 177.73 | 244.90 |
| | | 100 | 77.16 | 27.83 | 370.06 | 333.68 | 219.75 |
| 82-5 | Fruits | 1 | 28.05 | 91.00 | 131.77 | 87.90 | 137.78 |
| | | 10 | 43.34 | 34.98 | 271.51 | 196.32 | 242.34 |
| | | 100 | 81.05 | 23.91 | 474.79 | 323.59 | 299.81 |
| 82-6 | Flowers | 1 | 29.99 | 40.08 | 182.34 | 86.06 | 139.41 |
| | | 10 | 41.05 | 39.59 | 217.92 | 117.32 | 134.16 |
| | | 100 | 81.07 | 29.34 | 431.32 | 419.96 | 241.72 |

TABLE 166

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 82 | Short-stem | | | | | | |
| | sedge | | | | | | |
| 82-1 | Whole plant | 1 | 97.27 | 107.25 | 98.93 | 75.99 | 182.33 |
| | | 10 | 83.32 | 108.89 | 58.88 | 87.57 | 203.55 |
| | | 100 | 80.03 | 141.87 | 10.73 | 106.14 | 314.29 |
| 82-2 | Roots | 1 | 99.13 | 106.77 | 81.79 | 75.64 | 109.22 |
| | | 10 | 86.03 | 135.94 | 94.44 | 78.58 | 326.48 |
| | | 100 | 75.77 | 142.46 | 47.86 | 99.80 | 375.60 |
| 82-3 | Stems | 1 | 97.96 | 112.04 | 95.48 | 80.00 | 158.77 |
| | | 10 | 82.25 | 106.69 | 48.80 | 80.96 | 294.13 |
| | | 100 | 73.64 | 142.10 | 4.08 | 110.62 | 444.80 |
| 82-4 | Leaves | 1 | 98.81 | 106.44 | 103.68 | 80.14 | 169.34 |
| | | 10 | 86.18 | 132.64 | 60.23 | 89.98 | 331.66 |
| | | 100 | 76.23 | 144.24 | 0.03 | 103.51 | 482.83 |
| 82-5 | Fruits | 1 | 101.22 | 113.61 | 87.74 | 84.18 | 162.07 |
| | | 10 | 92.15 | 135.91 | 76.16 | 92.24 | 218.43 |
| | | 100 | 72.78 | 139.86 | 40.01 | 90.59 | 446.06 |
| 82-6 | Flowers | 1 | 102.50 | 100.41 | 98.36 | 66.95 | 117.13 |
| | | 10 | 91.92 | 140.29 | 49.67 | 95.58 | 247.98 |
| | | 100 | 76.18 | 132.30 | 39.81 | 93.79 | 370.13 |

### Example 83. Purple Maiden Silvergrass

TABLE 167

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 83 | Purple | | | | | | |
| | maiden | | | | | | |
| | silvergrass | | | | | | |
| 83-1 | Whole plant | 1 | 27.04 | 45.94 | 136.41 | 103.55 | 103.58 |
| | | 10 | 46.87 | 31.26 | 324.06 | 197.64 | 218.01 |
| | | 100 | 85.57 | 26.18 | 389.20 | 491.88 | 227.10 |
| 83-2 | Roots | 1 | 23.44 | 87.96 | 177.56 | 104.44 | 99.80 |
| | | 10 | 47.71 | 34.00 | 236.03 | 158.33 | 240.22 |
| | | 100 | 77.63 | 23.66 | 362.53 | 333.38 | 329.26 |
| 83-3 | Stems | 1 | 29.95 | 41.90 | 157.25 | 100.17 | 122.27 |
| | | 10 | 46.74 | 38.47 | 267.37 | 176.77 | 178.53 |
| | | 100 | 75.81 | 22.61 | 390.19 | 425.17 | 289.01 |
| 83-4 | Leaves | 1 | 24.43 | 69.05 | 132.40 | 95.67 | 87.76 |
| | | 10 | 49.36 | 34.07 | 309.69 | 117.41 | 110.93 |
| | | 100 | 81.33 | 23.78 | 422.26 | 316.23 | 346.49 |
| 83-5 | Fruits | 1 | 29.13 | 71.09 | 112.47 | 108.83 | 105.20 |
| | | 10 | 42.41 | 36.84 | 248.13 | 118.71 | 136.40 |
| | | 100 | 76.62 | 24.77 | 486.63 | 352.16 | 297.02 |
| 83-6 | Flowers | 1 | 23.12 | 41.48 | 123.55 | 102.70 | 106.26 |
| | | 10 | 38.68 | 31.77 | 280.86 | 131.24 | 119.93 |
| | | 100 | 85.83 | 30.25 | 479.54 | 357.92 | 273.90 |
| 83-7 | Shoots | 1 | 28.71 | 84.86 | 156.18 | 101.69 | 109.10 |
| | | 10 | 41.58 | 40.44 | 307.55 | 110.85 | 197.78 |
| | | 100 | 77.11 | 29.59 | 449.03 | 358.75 | 327.93 |

TABLE 168

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 83 | Purple | | | | | | |
| | maiden | | | | | | |
| | silvergrass | | | | | | |
| 83-1 | Whole plant | 1 | 95.22 | 104.51 | 84.21 | 83.67 | 108.80 |
| | | 10 | 90.08 | 106.33 | 81.04 | 93.69 | 251.07 |
| | | 100 | 72.54 | 139.71 | 40.69 | 90.94 | 468.21 |
| 83-2 | Roots | 1 | 100.35 | 113.41 | 100.60 | 71.95 | 91.06 |
| | | 10 | 91.24 | 125.27 | 69.07 | 77.20 | 177.27 |
| | | 100 | 75.07 | 159.83 | 34.27 | 93.44 | 357.84 |
| 83-3 | Stems | 1 | 96.67 | 115.25 | 81.58 | 73.94 | 119.29 |
| | | 10 | 94.13 | 116.46 | 74.11 | 81.08 | 212.02 |
| | | 100 | 70.42 | 157.79 | 42.90 | 104.63 | 411.48 |
| 83-4 | Leaves | 1 | 95.83 | 106.30 | 83.16 | 83.67 | 91.71 |
| | | 10 | 90.20 | 112.42 | 91.50 | 80.79 | 217.46 |
| | | 100 | 79.70 | 140.21 | 17.01 | 106.91 | 485.34 |
| 83-5 | Fruits | 1 | 103.63 | 100.39 | 98.62 | 76.29 | 127.28 |
| | | 10 | 90.62 | 116.98 | 88.72 | 95.86 | 322.93 |
| | | 100 | 80.60 | 145.28 | 29.39 | 109.81 | 431.57 |
| 83-6 | Flowers | 1 | 96.26 | 103.34 | 100.81 | 65.46 | 113.66 |
| | | 10 | 89.83 | 130.58 | 73.19 | 84.12 | 216.66 |
| | | 100 | 80.73 | 133.41 | 9.50 | 100.08 | 301.79 |
| 83-7 | Shoots | 1 | 110.50 | 114.29 | 92.90 | 65.57 | 109.92 |
| | | 10 | 104.20 | 120.67 | 83.91 | 83.27 | 308.34 |
| | | 100 | 98.10 | 134.66 | 47.49 | 90.73 | 310.24 |

### Example 84. Foxtail Fountaingrass

TABLE 169

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 uM) | | 1 ng/ml) |
| 84 | Foxtail | | | | | | |
| | fountaingrass | | | | | | |
| 84-1 | Whole plant | 1 | 20.57 | 40.59 | 109.12 | 98.29 | 114.33 |
| | | 10 | 31.87 | 30.17 | 302.75 | 139.60 | 157.69 |
| | | 100 | 84.66 | 30.59 | 495.71 | 301.99 | 268.97 |
| 84-2. | Roots | 1 | 20.85 | 74.34 | 194.50 | 110.29 | 82.02 |
| | | 10 | 38.47 | 30.37 | 250.04 | 129.07 | 202.92 |
| | | 100 | 72.42 | 26.73 | 408.51 | 467.93 | 271.28 |
| 84-3 | Stems | 1 | 23.85 | 54.20 | 157.96 | 85.19 | 113.24 |
| | | 10 | 40.91 | 34.19 | 329.26 | 197.18 | 209.63 |
| | | 100 | 82.20 | 23.57 | 384.02 | 363.32 | 314.17 |
| 84-4 | Leaves | 1 | 27.14 | 89.64 | 154.98 | 86.92 | 98.39 |
| | | 10 | 47.86 | 32.20 | 223.48 | 126.78 | 110.11 |
| | | 100 | 70.50 | 28.04 | 366.32 | 379.39 | 336.24 |
| 84-5 | Fruits | 1 | 21.50 | 43.17 | 164.88 | 81.86 | 139.40 |
| | | 10 | 40.84 | 33.10 | 267.01 | 169.08 | 185.59 |
| | | 100 | 78.61 | 21.26 | 402.80 | 481.49 | 268.56 |
| 84-6 | Flowers | 1 | 28.57 | 65.63 | 134.04 | 103.23 | 124.19 |
| | | 10 | 44.36 | 40.45 | 286.00 | 134.29 | 160.67 |
| | | 100 | 75.40 | 27.58 | 444.52 | 487.33 | 244.24 |

TABLE 170

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 84 | Foxtail | | | | | | |
| | fountaingrass | | | | | | |
| 84-1 | Whole plant | 1 | 98.02 | 102.10 | 89.01 | 79.07 | 162.05 |
| | | 10 | 90.72 | 129.07 | 69.91 | 84.15 | 187.13 |
| | | 100 | 77.28 | 154.97 | 31.21 | 95.56 | 406.11 |
| 84-2 | Roots | 1 | 103.14 | 104.98 | 109.54 | 73.25 | 159.52 |
| | | 10 | 93.53 | 111.81 | 73.39 | 92.01 | 276.16 |
| | | 100 | 74.91 | 142.94 | 5.58 | 98.59 | 332.69 |
| 84-3 | Stems | 1 | 100.35 | 96.01 | 103.16 | 65.77 | 91.58 |
| | | 10 | 88.35 | 112.30 | 93.22 | 76.95 | 298.58 |
| | | 100 | 70.13 | 143.15 | 2.33 | 109.79 | 311.82 |
| 84-4 | Leaves | 1 | 102.84 | 106.91 | 96.07 | 81.72 | 169.32 |
| | | 10 | 85.31 | 121.36 | 89.77 | 80.41 | 186.85 |
| | | 100 | 77.52 | 137.27 | 48.08 | 102.21 | 480.53 |
| 84-5 | Fruits | 1 | 99.28 | 100.51 | 85.32 | 75.10 | 202.90 |
| | | 10 | 92.23 | 127.32 | 85.84 | 81.78 | 186.49 |
| | | 100 | 75.24 | 153.58 | 47.77 | 107.40 | 500.32 |
| 84-6 | Flowers | 1 | 99.79 | 102.64 | 80.75 | 79.02 | 110.45 |
| | | 10 | 92.90 | 123.85 | 56.21 | 81.35 | 260.32 |
| | | 100 | 71.04 | 145.13 | 43.27 | 103.67 | 387.32 |

### Example 85. Arrow Bamboo

TABLE 171

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 85 | Arrow | | | | | | |
| | bamboo | | | | | | |
| 85-1 | Whole plant | 1 | 25.56 | 50.43 | 107.51 | 98.37 | 105.84 |
| | | 10 | 30.35 | 39.85 | 266.45 | 160.10 | 225.50 |
| | | 100 | 81.42 | 29.20 | 440.01 | 359.42 | 317.15 |
| 85-2 | Roots | 1 | 29.70 | 77.99 | 138.83 | 99.93 | 107.85 |
| | | 10 | 35.14 | 40.78 | 225.35 | 176.68 | 142.64 |
| | | 100 | 85.00 | 20.24 | 451.07 | 390.49 | 306.08 |
| 85-3 | Stems | 1 | 23.89 | 41.75 | 132.67 | 89.45 | 136.86 |
| | | 10 | 34.89 | 35.15 | 334.23 | 123.68 | 109.69 |
| | | 100 | 70.44 | 25.72 | 408.18 | 391.00 | 222.09 |
| 85-4 | Leaves | 1 | 22.08 | 99.69 | 101.23 | 86.86 | 142.84 |
| | | 10 | 35.45 | 86.69 | 235.91 | 133.31 | 247.04 |
| | | 100 | 84.87 | 76.75 | 355.97 | 457.67 | 270.27 |
| 85-5 | Fruits | 1 | 26.37 | 63.61 | 130.70 | 98.53 | 137.47 |
| | | 10 | 43.89 | 37.58 | 205.23 | 144.21 | 212.25 |
| | | 100 | 76.02 | 25.83 | 434.79 | 330.89 | 245.08 |
| 85-6 | Flowers | 1 | 26.75 | 83.08 | 115.35 | 83.53 | 141.63 |
| | | 10 | 46.30 | 38.52 | 243.28 | 110.55 | 103.87 |
| | | 100 | 82.12 | 24.20 | 417.87 | 383.00 | 324.20 |

TABLE 172

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 85 | Arrow | | | | | | |
| | bamboo | | | | | | |
| 85-1 | Whole plant | 1 | 98.73 | 98.84 | 101.89 | 65.23 | 104.12 |
| | | 10 | 89.45 | 119.25 | 64.78 | 93.64 | 336.94 |
| | | 100 | 73.78 | 135.64 | 29.42 | 97.15 | 383.10 |
| 85-2 | Roots | 1 | 97.72 | 113.07 | 98.50 | 79.28 | 172.87 |
| | | 10 | 90.69 | 115.52 | 75.83 | 93.66 | 206.54 |
| | | 100 | 78.37 | 140.97 | 19.33 | 97.32 | 336.59 |
| 85-3 | Stems | 1 | 103.12 | 125.25 | 92.76 | 65.00 | 180.45 |
| | | 10 | 88.87 | 111.91 | 54.15 | 75.70 | 193.25 |
| | | 100 | 73.94 | 146.00 | 46.48 | 103.44 | 417.03 |
| 85-4 | Leaves | 1 | 100.48 | 108.17 | 86.38 | 85.19 | 158.00 |
| | | 10 | 86.92 | 111.60 | 73.18 | 75.02 | 326.35 |
| | | 100 | 77.75 | 138.78 | 10.29 | 103.79 | 405.52 |
| 85-5 | Fruits | 1 | 102.31 | 113.94 | 82.51 | 81.44 | 192.11 |
| | | 10 | 85.64 | 106.89 | 75.72 | 92.41 | 322.31 |
| | | 100 | 78.61 | 155.40 | 11.85 | 93.76 | 353.33 |
| 85-6 | Flowers | 1 | 102.47 | 113.41 | 80.50 | 73.19 | 125.46 |
| | | 10 | 89.14 | 138.18 | 59.52 | 80.16 | 195.71 |
| | | 100 | 76.98 | 132.82 | 8.74 | 94.47 | 304.77 |

### Example 86. Kuril Bamboo

TABLE 173

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 86 | Kuril | | | | | | |
| | bamboo | | | | | | |
| 86-1 | Whole plant | 1 | 26.29 | 53.16 | 183.53 | 109.78 | 123.17 |
| | | 10 | 41.27 | 30.60 | 278.16 | 149.87 | 150.57 |
| | | 100 | 83.41 | 27.77 | 439.21 | 498.58 | 304.71 |
| 86-2 | Roots | 1 | 22.54 | 55.76 | 179.91 | 98.52 | 127.22 |
| | | 10 | 36.86 | 31.55 | 281.87 | 136.96 | 198.05 |
| | | 100 | 75.48 | 27.69 | 393.78 | 426.69 | 335.25 |
| 86-3 | Stems | 1 | 28.41 | 86.07 | 182.83 | 99.70 | 88.52 |
| | | 10 | 41.79 | 39.79 | 214.45 | 178.46 | 249.64 |
| | | 100 | 77.56 | 29.21 | 450.97 | 416.80 | 223.51 |
| 86-4 | Leaves | 1 | 29.60 | 82.04 | 107.03 | 109.08 | 146.39 |
| | | 10 | 43.21 | 32.99 | 222.17 | 124.59 | 249.35 |
| | | 100 | 70.58 | 24.69 | 396.62 | 352.67 | 282.33 |
| 86-5 | Fruits | 1 | 27.15 | 56.37 | 189.98 | 90.55 | 89.60 |
| | | 10 | 31.36 | 34.89 | 332.52 | 159.94 | 109.18 |
| | | 100 | 74,54 | 27.39 | 404,66 | 453.15 | 260.39 |
| 86-6 | Flowers | 1 | 26.84 | 47.14 | 120.26 | 97.75 | 100.41 |
| | | 10 | 41.15 | 37.09 | 289.63 | 190.76 | 167.80 |
| | | 100 | 82.51 | 27.27 | 402.10 | 426.87 | 219.92 |

TABLE 174

| | | | | Blood | Cognitive | | Andropanse- |
|---|---|---|---|---|---|---|---|
| | | | Anti- | glucose - | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 86 | Kuril | | | | | | |
| | bamboo | | | | | | |
| 86-1 | Whole plant | 1 | 96.85 | 110.17 | 85.36 | 76.31 | 166.97 |
| | | 10 | 80.60 | 113.84 | 80.70 | 91.17 | 244.01 |
| | | 100 | 74.26 | 142.48 | 12.65 | 104.37 | 432.17 |
| 86-2 | Roots | 1 | 103.60 | 110.80 | 96.13 | 72.23 | 117.37 |
| | | 10 | 83.00 | 128.98 | 52.48 | 93.83 | 319.95 |
| | | 100 | 78.53 | 141.62 | 30.95 | 108.13 | 369.36 |
| 86-3 | Stems | 1 | 101.66 | 96.87 | 105.56 | 71.11 | 157.02 |
| | | 10 | 82.11 | 111.74 | 74.17 | 89.00 | 340.17 |
| | | 100 | 71.42 | 136.02 | 30.72 | 90.31 | 420.68 |
| 86-4 | Leaves | 1 | 100.21 | 99.62 | 92.66 | 78.37 | 179.60 |
| | | 10 | 85.22 | 137.24 | 55.18 | 85.57 | 188.45 |
| | | 100 | 77.06 | 141.96 | 21.70 | 93.23 | 393.88 |
| 86-5 | Fruits | 1 | 96.75 | 110.46 | 109.76 | 81.22 | 103.40 |
| | | 10 | 94.93 | 123.11 | 51.94 | 84.55 | 200.10 |
| | | 100 | 79.45 | 137.25 | 12.63 | 90.28 | 311.16 |
| 86-6 | Flowers | 1 | 101.51 | 101.56 | 104.19 | 69.35 | 210.52 |
| | | 10 | 80.59 | 133.10 | 47.65 | 76.90 | 254.76 |
| | | 100 | 75.02 | 158.88 | 49.62 | 91.02 | 423.21 |

### Example 87. Shrubby Sophora

TABLE 175

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 uM) | | 1 ng/ml) |
| 87 | Shrubby | | | | | | |
| | sophora | | | | | | |
| 87-1 | Whole plant | 1 | 24.18 | 56.72 | 139.00 | 98.26 | 109.03 |
| | | 10 | 39.07 | 36.32 | 273.83 | 196.35 | 124.00 |
| | | 100 | 77.93 | 27.34 | 359.49 | 376.18 | 216.46 |
| 87-2 | Roots | 1 | 28.24 | 61.20 | 197.18 | 108.18 | 104.06 |
| | | 10 | 33.80 | 38.24 | 310.95 | 124.17 | 190.54 |
| | | 100 | 76.35 | 20.87 | 389.31 | 424.92 | 339.93 |
| 87-3 | Stems | 1 | 29.89 | 90.60 | 174.08 | 97.13 | 127.06 |
| | | 10 | 42.47 | 37.60 | 290.67 | 152.21 | 162.29 |
| | | 100 | 79.72 | 25.31 | 407.27 | 481.52 | 226.76 |
| 87-4 | Leaves | 1 | 27.54 | 84.99 | 139.83 | 89.36 | 83.71 |
| | | 10 | 38.05 | 39.55 | 210.80 | 144.29 | 118.97 |
| | | 100 | 73.04 | 23.11 | 435.30 | 333.78 | 290.75 |
| 87-5 | Fruits | 1 | 30.09 | 83.67 | 113.60 | 100.13 | 124.41 |
| | | 10 | 39.83 | 33.47 | 267.32 | 166.96 | 248.74 |
| | | 100 | 81.56 | 24.28 | 385.64 | 394.22 | 221.79 |
| 87-6 | Flowers | 1 | 25.57 | 78.56 | 106.52 | 90.66 | 114.19 |
| | | 10 | 45.77 | 37.78 | 282.31 | 139.69 | 189.72 |
| | | 100 | 71.58 | 28.70 | 400.82 | 304.80 | 300.74 |

TABLE 176

| | | | | Blood | Cognitive | | Andropaase- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 87 | Shrubby | | | | | | |
| | sophora | | | | | | |
| 87-1 | Whole plant | 1 | 102.49 | 106.90 | 105.66 | 84.61 | 117.00 |
| | | 10 | 90.40 | 136.49 | 85.47 | 95.74 | 178.73 |
| | | 100 | 74.05 | 143.82 | 14.86 | 95.36 | 353.75 |
| 87-2 | Roots | 1 | 98.02 | 99.88 | 99.21 | 85.26 | 147.82 |
| | | 10 | 91.87 | 131.77 | 57.16 | 83.89 | 206.65 |
| | | 100 | 71.92 | 158.32 | 9.49 | 95.75 | 385.75 |
| 87-3 | Stems | 1 | 103.28 | 110.01 | 90.23 | 81.78 | 182.41 |
| | | 10 | 92.84 | 128.10 | 58.38 | 84.42 | 279.16 |
| | | 100 | 79.30 | 143.33 | 0.18 | 107.21 | 477.15 |
| 87-4 | Leaves | 1 | 96.95 | 99.00 | 81.93 | 65.62 | 137.01 |
| | | 10 | 89.17 | 109.97 | 86.32 | 82.28 | 312.11 |
| | | 100 | 72.56 | 138.85 | 22.10 | 101.73 | 457.89 |
| 87-5 | Fruits | 1 | 100.64 | 101.03 | 88.44 | 67.85 | 170.21 |
| | | 10 | 91.56 | 115.07 | 77.48 | 90.93 | 318.22 |
| | | 100 | 75.76 | 141.41 | 33.15 | 100.92 | 387.15 |
| 87-6 | Flowers | 1 | 103.09 | 98.65 | 100.26 | 65.02 | 134.25 |
| | | 10 | 83.12 | 136.22 | 85.58 | 84.59 | 186.78 |
| | | 100 | 75.86 | 153.91 | 31.40 | 99.28 | 454.34 |

### Example 88. Orange Daylily

TABLE 177

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 88 | Orange | | | | | | |
| | Daylily | | | | | | |
| 88-1 | Whole plant | 1 | 22.48 | 41.05 | 149.07 | 90.54 | 113.18 |
| | | 10 | 42.16 | 37.75 | 316.61 | 167.54 | 159.10 |
| | | 100 | 77.27 | 28.56 | 363.41 | 399.12 | 266.65 |
| 88-2 | Roots | 1 | 29.41 | 65.77 | 121.92 | 108.04 | 139.63 |
| | | 10 | 42.65 | 40.51 | 262.50 | 162.79 | 237.78 |
| | | 100 | 75.52 | 25.49 | 357.65 | 470.94 | 332.42 |
| 88-3 | Stems | 1 | 23.51 | 42.36 | 200.00 | 108.58 | 150.31 |
| | | 10 | 45.35 | 30.33 | 276.58 | 198.13 | 210.17 |
| | | 100 | 80.00 | 22.64 | 455.58 | 336.91 | 216.38 |
| 88-4 | Leaves | 1 | 22.00 | 51.69 | 113.27 | 24.29 | 146.68 |
| | | 10 | 47.33 | 30.07 | 246.44 | 69.60 | 150.55 |
| | | 100 | 73.93 | 30.61 | 419.37 | 79.30 | 271.26 |
| 88-5 | Fruits | 1 | 23.05 | 58.04 | 152.73 | 88.81 | 120.96 |
| | | 10 | 37.74 | 39.51 | 315.21 | 187.24 | 186.94 |
| | | 100 | 70.52 | 20.77 | 393.49 | 328.98 | 328.51 |
| 88-6 | Flowers | 1 | 20.17 | 48.90 | 95.81 | 24.74 | 93.88 |
| | | 10 | 36.98 | 33.02 | 108.45 | 79.32 | 204.15 |
| | | 100 | 81.24 | 30.19 | 188.17 | 82.36 | 283.02 |

TABLE 178

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 88 | Orange | | | | | | |
| | Daylily | | | | | | |
| 88-1 | Whole plant | 1 | 100.87 | 110.26 | 94.03 | 74.33 | 166.60 |
| | | 10 | 85.09 | 127.01 | 88.95 | 85.22 | 253.39 |
| | | 100 | 77.70 | 134.61 | 43.12 | 95.44 | 400.43 |
| 88-2 | Roots | 1 | 95.92 | 115.93 | 87.80 | 73.87 | 153.61 |
| | | 10 | 86.61 | 118.07 | 94.12 | 83.72 | 334.18 |
| | | 100 | 73.13 | 153.33 | 1.50 | 104.04 | 330.00 |
| 88-3 | Stems | 1 | 98.29 | 110.15 | 81.39 | 69.38 | 153.78 |
| | | 10 | 91.74 | 130.82 | 88.51 | 82.85 | 192.02 |
| | | 100 | 77.89 | 156.71 | 36.52 | 108.76 | 320.48 |
| 88-4 | Leaves | 1 | 99.98 | 105.32 | 101.10 | 73.45 | 102.03 |
| | | 10 | 91.09 | 127.40 | 64.83 | 83.13 | 205.24 |
| | | 100 | 80.66 | 148.14 | 6.66 | 96.26 | 496.62 |
| 88-5 | Fruits | 1 | 95.45 | 102.65 | 87.60 | 12.36 | 130.46 |
| | | 10 | 87.11 | 139.87 | 66.38 | 25.64 | 339.56 |
| | | 100 | 70.56 | 152.34 | 48.95 | 30.18 | 438.44 |
| 88-6 | Flowers | 1 | 96.86 | 112.31 | 89.41 | 70.92 | 139.95 |
| | | 10 | 83.26 | 139.66 | 64.63 | 76.58 | 340.61 |
| | | 100 | 75.28 | 132.15 | 39.18 | 93.42 | 495.75 |

### Example 89. Tiger Lily

TABLE 179

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 89 | Tiger lily | | | | | | |
| 89-1 | Whole plant | 1 | 29.50 | 76.23 | 183.47 | 90.85 | 110.05 |
| | | 10 | 42.28 | 33.83 | 288.32 | 189.15 | 177.82 |
| | | 100 | 77.17 | 26.22 | 384.42 | 478.14 | 322.70 |
| 89-2 | Roots | 1 | 30.75 | 81.23 | 184.66 | 26.28 | 22.61 |
| | | 10 | 45.80 | 35.43 | 323.97 | 32.67 | 36.90 |
| | | 100 | 76.94 | 23.86 | 355.11 | 48.24 | 46.87 |
| 89-3 | Stems | 1 | 22.69 | 81.30 | 133.91 | 35.45 | 128.19 |
| | | 10 | 41.24 | 31.68 | 284.79 | 44.13 | 151.67 |
| | | 100 | 76.74 | 28.81 | 428.43 | 53.44 | 322.30 |
| 89-4 | Leaves | 1 | 20.66 | 81.95 | 185.51 | 93.38 | 114.85 |
| | | 10 | 40.94 | 40.98 | 345.56 | 114.32 | 123.96 |
| | | 100 | 71.98 | 23.82 | 495.14 | 493.63 | 261.50 |
| 89-5 | Fruits | 1 | 25.24 | 53.94 | 119.90 | 87.99 | 142.18 |
| | | 10 | 32.98 | 30.51 | 286.75 | 183.35 | 163.08 |
| | | 100 | 72.65 | 26.38 | 386.08 | 485.34 | 252.43 |
| 89-6 | Flowers | 1 | 29.89 | 61.99 | 103.69 | 29.52 | 80.55 |
| | | 10 | 50.18 | 37.57 | 323.73 | 33.67 | 159.97 |
| | | 100 | 80.90 | 25.50 | 405.59 | 40.67 | 303.35 |

TABLE 180

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 89 | Tiger lily | | | | | | |
| 89-1 | Whole plant | 1 | 100.04 | 105.62 | 99.38 | 81.22 | 182.99 |
| | | 10 | 83.38 | 114.87 | 94.51 | 90.38 | 323.61 |
| | | 100 | 71.73 | 141.02 | 33.75 | 92.63 | 414.42 |
| 89-2 | Roots | 1 | 103.75 | 95.61 | 95.69 | 68.77 | 133.09 |
| | | 10 | 84.70 | 118.28 | 80.45 | 86.42 | 254.48 |
| | | 100 | 73.64 | 150.54 | 36.45 | 101.44 | 427.84 |
| 89-3 | Stems | 1 | 116.62 | 106.90 | 91.65 | 76.13 | 91.80 |
| | | 10 | 110.36 | 127.88 | 55.61 | 93.79 | 313.69 |
| | | 100 | 104.83 | 149.44 | 40.73 | 108.16 | 407.27 |
| 89-4 | Leaves | 1 | 100.44 | 100.39 | 100.45 | 74.96 | 107.70 |
| | | 10 | 80.34 | 127.59 | 53.30 | 81.90 | 170.48 |
| | | 100 | 73.55 | 140.64 | 20.51 | 107.23 | 337.47 |
| 89-5 | Fruits | 1 | 96.16 | 111.28 | 108.51 | 72.04 | 186.19 |
| | | 10 | 87.29 | 126.13 | 67.81 | 81.99 | 231.41 |
| | | 100 | 80.06 | 152.78 | 19.41 | 102.12 | 313.58 |
| 89-6 | Flowers | 1 | 95.74 | 99.54 | 87.75 | 76.10 | 129.08 |
| | | 10 | 85.45 | 109.19 | 80.90 | 83.05 | 238.48 |
| | | 100 | 79.34 | 147.05 | 19.99 | 110.95 | 399.59 |

### Example 90. Big Blue Lilyturf

TABLE 181

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 90 | Big blue | | | | | | |
| | lilyturf | | | | | | |
| 90-1 | Whole plant | 1 | 21.64 | 47.47 | 183.72 | 94.23 | 112.28 |
| | | 10 | 35.83 | 30.38 | 231.30 | 125.78 | 177.67 |
| | | 100 | 82.39 | 23.55 | 450.19 | 314.82 | 306.06 |
| 90-2 | Roots | 1 | 0.00 | 92.20 | 29.29 | 13.81 | 92.62 |
| | | 10 | 3.27 | 90.34 | 55.71 | 18.07 | 187.71 |
| | | 100 | 8.16 | 85.70 | 66.07 | 26.77 | 258.04 |
| 90-3 | Stems | 1 | 25.78 | 55.21 | 154.43 | 90.60 | 146.12 |
| | | 10 | 44.33 | 40.30 | 241.41 | 158.35 | 101.09 |
| | | 100 | 84.14 | 23.77 | 381.85 | 365.53 | 288.27 |
| 90-4 | Leaves | 1 | 23.66 | 87.96 | 190.51 | 83.54 | 106.31 |
| | | 10 | 31.04 | 40.85 | 303.50 | 138.99 | 202.53 |
| | | 100 | 77.84 | 29.22 | 433.73 | 453.53 | 283.77 |
| 90-5 | Fruits | 1 | 23.34 | 72.36 | 182.91 | 83.90 | 148.82 |
| | | 10 | 32.56 | 38.27 | 323.82 | 131.13 | 162.03 |
| | | 100 | 72.78 | 29.61 | 495.11 | 447.03 | 309.06 |
| 90-6 | Flowers | 1 | 26.11 | 48.46 | 130.61 | 102.80 | 92.82 |
| | | 10 | 49.55 | 33.17 | 346.46 | 132.84 | 172.87 |
| | | 100 | 84.48 | 28.88 | 379.46 | 304.73 | 262.96 |

TABLE 182

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 90 | Big blue | | | | | | |
| | lilyturf | | | | | | |
| 90-1 | Whole plant | 1 | 95.10 | 113.00 | 97.10 | 73.50 | 174.17 |
| | | 10 | 92.64 | 108.11 | 91.55 | 78.48 | 293.62 |
| | | 100 | 80.57 | 134.88 | 4.63 | 98.70 | 347.25 |
| 90-2 | Roots | 1 | 106.87 | 19.03 | 83.07 | 7.37 | 110.99 |
| | | 10 | 100.48 | 25.42 | 93.58 | 8.18 | 307.51 |
| | | 100 | 96.26 | 46.47 | 85.85 | 10.70 | 455.86 |
| 90-3 | Stems | 1 | 97.58 | 105.25 | 92.11 | 79.60 | 129.00 |
| | | 10 | 92.93 | 124.27 | 49.90 | 82.68 | 300.02 |
| | | 100 | 74.91 | 150.93 | 39.31 | 95.61 | 428.21 |
| 90-4 | Leaves | 1 | 100.43 | 122.02 | 96.26 | 77.51 | 98.46 |
| | | 10 | 84.64 | 118.60 | 89.89 | 89.51 | 307.35 |
| | | 100 | 75.77 | 145.67 | 22.19 | 97.32 | 410.38 |
| 90-5 | Fruits | 1 | 97.34 | 110.21 | 101.32 | 80.06 | 136.26 |
| | | 10 | 93.67 | 137.32 | 58.29 | 78.63 | 287.47 |
| | | 100 | 72.40 | 141.48 | 8.06 | 90.34 | 396.43 |
| 90-6 | Flowers | 1 | 98.51 | 115.66 | 97.60 | 71.56 | 203.18 |
| | | 10 | 90.42 | 115.85 | 92.56 | 94.78 | 238.84 |
| | | 100 | 76.63 | 158.70 | 11.45 | 104.29 | 422.77 |

### Example 91. False Lily of the Valley

TABLE 183

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 pM) | | 1 ng/ml) |
| 91 | False lily of | | | | | | |
| | the valley | | | | | | |
| 91-1 | Whole plant | 1 | 25.93 | 45.30 | 107.39 | 98.41 | 106.95 |
| | | 10 | 47.19 | 39.42 | 312.23 | 116.47 | 212.70 |
| | | 100 | 74.18 | 23.49 | 401.59 | 444.27 | 306.43 |
| 91-2 | Roots | 1 | 25.62 | 51.79 | 142.81 | 106.97 | 124.21 |
| | | 10 | 43.64 | 31.55 | 333.33 | 152.52 | 163.41 |
| | | 100 | 70.19 | 23.79 | 484.03 | 388.51 | 271.71 |
| 91-3 | Stems | 1 | 26.44 | 70.73 | 133.96 | 90.80 | 125.34 |
| | | 10 | 40.59 | 31.23 | 324.77 | 183.45 | 173.28 |
| | | 100 | 79.17 | 30.86 | 429.92 | 347.73 | 292.69 |
| 91-4 | Leaves | 1 | 22.94 | 77.71 | 141.03 | 99.22 | 114.70 |
| | | 10 | 36.73 | 38.67 | 301.35 | 143.17 | 130.58 |
| | | 100 | 71.90 | 24.17 | 476.31 | 409.13 | 332.84 |
| 91-5 | Fruits | 1 | 29.88 | 68.89 | 116.89 | 108.98 | 109.78 |
| | | 10 | 48.00 | 34.16 | 304.38 | 175.95 | 125.87 |
| | | 100 | 72.84 | 21.77 | 464.16 | 458.95 | 224.46 |
| 91-6 | Flowers | 1 | 26.30 | 83.35 | 126.48 | 87.36 | 140.14 |
| | | 10 | 49.95 | 40.73 | 310.78 | 195.20 | 168.43 |
| | | 100 | 70.85 | 29.61 | 408.43 | 402.07 | 305.62 |

TABLE 184

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 91 | False lily of | | | | | | |
| | the valley | | | | | | |
| 91-1 | Whole plant | 1 | 99.40 | 97.96 | 103.00 | 87.47 | 181.66 |
| | | 10 | 91.78 | 109.24 | 74.64 | 83.10 | 269.80 |
| | | 100 | 77.33 | 148.75 | 29.98 | 103.53 | 453.82 |
| 91-2 | Roots | 1 | 97.91 | 109.20 | 85.76 | 70.89 | 155.04 |
| | | 10 | 91.32 | 115.80 | 89.43 | 82.21 | 238.69 |
| | | 100 | 73.85 | 146.98 | 24.94 | 101.67 | 450.60 |
| 91-3 | Stems | 1 | 101.38 | 104.12 | 80.15 | 83.57 | 109.11 |
| | | 10 | 86.42 | 122.51 | 70.14 | 89.71 | 330.69 |
| | | 100 | 78.03 | 143.55 | 48.97 | 104.70 | 381.96 |
| 91-4 | Leaves | 1 | 101.74 | 102.17 | 80.17 | 85.53 | 108.94 |
| | | 10 | 90.34 | 129.27 | 82.52 | 77.58 | 210.02 |
| | | 100 | 74.00 | 153.61 | 31.29 | 91.80 | 338.66 |
| 91-5 | Fruits | 1 | 103.26 | 105.00 | 107.62 | 81.41 | 123.36 |
| | | 10 | 82.69 | 136.73 | 75.93 | 91.18 | 190.44 |
| | | 100 | 76.71 | 152.69 | 46.04 | 94.82 | 330.98 |
| 91-6 | Flowers | 1 | 98.59 | 103.72 | 109.06 | 80.53 | 180.60 |
| | | 10 | 87.25 | 114.15 | 79.98 | 86.95 | 316.29 |
| | | 100 | 75.35 | 152.59 | 31.12 | 101.85 | 431.32 |

### Example 92. Japanese Cedar

TABLE 185

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 pM) | | 1 ng/ml) |
| 92 | Japanese | | | | | | |
| | Cedar | | | | | | |
| 92-1 | Whole plant | 1 | 25.54 | 84.94 | 179.98 | 91.02 | 146.76 |
| | | 10 | 42.07 | 33.93 | 204.69 | 117.95 | 110.06 |
| | | 100 | 83.66 | 28.16 | 374.13 | 481.44 | 205.12 |
| 92-2 | Roots | 1 | 20.11 | 55.46 | 123.13 | 110.86 | 81.50 |
| | | 10 | 46.72 | 31.24 | 345.06 | 181.96 | 149.14 |
| | | 100 | 84.73 | 28.94 | 374.36 | 458.45 | 262.84 |
| 92-3 | Stems | 1 | 26.87 | 57.01 | 153.76 | 99.42 | 85.41 |
| | | 10 | 31.48 | 38.00 | 311.80 | 163.31 | 184.75 |
| | | 100 | 83.98 | 30.35 | 439.75 | 305.79 | 234.70 |
| 92-4 | Leaves | 1 | 21.36 | 75.38 | 164.09 | 108.08 | 108.38 |
| | | 10 | 47.98 | 33.88 | 268.57 | 157.07 | 218.22 |
| | | 100 | 72.61 | 25.42 | 360.34 | 494.22 | 266.99 |
| 92-5 | Fruits | 1 | 23.11 | 52.32 | 106.23 | 83.62 | 90.60 |
| | | 10 | 44.77 | 33.78 | 239.69 | 146.54 | 116.13 |
| | | 100 | 83.93 | 21.51 | 370.83 | 377.91 | 297.60 |
| 92-6 | Flowers | 1 | 24.52 | 64.14 | 197.28 | 83.45 | 106.14 |
| | | 10 | 48.56 | 35.54 | 254.97 | 120.30 | 211.50 |
| | | 100 | 80.95 | 28.99 | 373.91 | 353.89 | 213.44 |

TABLE 186

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 92 | Japanese | | | | | | |
| | Cedar | | | | | | |
| 92-1 | Whole plant | 1 | 100.43 | 106.43 | 90.86 | 78.67 | 209.08 |
| | | 10 | 87.56 | 129.37 | 47.89 | 91.89 | 324.72 |
| | | 100 | 78.91 | 150.25 | 29.76 | 91.46 | 333.79 |
| 92-2 | Roots | 1 | 103.89 | 106.34 | 109.80 | 67.57 | 159.51 |
| | | 10 | 91.40 | 110.74 | 82.24 | 82.11 | 312.55 |
| | | 100 | 80.14 | 160.20 | 14.11 | 97.32 | 324.08 |
| 92-3 | Stems | 1 | 98.86 | 111.52 | 81.63 | 78.10 | 153.47 |
| | | 10 | 95.24 | 106.49 | 81.78 | 75.59 | 183.95 |
| | | 100 | 71.70 | 131.15 | 12.60 | 105.86 | 480.91 |
| 92-4 | Leaves | 1 | 103.67 | 97.76 | 109.31 | 77.35 | 110.29 |
| | | 10 | 80.44 | 128.56 | 48.51 | 77.91 | 272.96 |
| | | 100 | 70.03 | 146.98 | 27.74 | 95.56 | 476.55 |
| 92-5 | Fruits | 1 | 97.53 | 98.23 | 89.68 | 69.44 | 104.20 |
| | | 10 | 86.83 | 127.91 | 77.12 | 85.74 | 289.16 |
| | | 100 | 71.72 | 154.41 | 35.54 | 95.78 | 320.53 |
| 92-6 | Flowers | 1 | 102.86 | 109.04 | 88.03 | 72.61 | 122.23 |
| | | 10 | 81.95 | 116.05 | 54.54 | 83.99 | 237.72 |
| | | 100 | 79.39 | 133.58 | 7.49 | 108.57 | 448.42 |

### Example 93. Thunberg's Bay-Tree

TABLE 187

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 93 | Thunberg's | | | | | | |
| | bay-tree | | | | | | |
| 93-1 | Whole plant | 1 | 27.00 | 73.91 | 171.98 | 103.04 | 141.18 |
| | | 10 | 48.38 | 33.68 | 210.73 | 130.61 | 242.46 |
| | | 100 | 85.90 | 28.56 | 443.12 | 364.19 | 345.45 |
| 93-2 | Roots | 1 | 20.93 | 58.04 | 144.81 | 106.51 | 80.06 |
| | | 10 | 40.54 | 36.02 | 330.91 | 183.32 | 208.83 |
| | | 100 | 83.55 | 26.25 | 425.81 | 341.76 | 262.54 |
| 93-3 | Stems | 1 | 20.11 | 69.80 | 179.33 | 87.91 | 139.19 |
| | | 10 | 50.88 | 34.51 | 249.69 | 135.47 | 140.10 |
| | | 100 | 84.43 | 30.43 | 487.77 | 301.61 | 348.17 |
| 93-4 | Leaves | 1 | 20.71 | 42.89 | 168.02 | 102.84 | 108.05 |
| | | 10 | 41.47 | 33.60 | 340.12 | 166.27 | 109.06 |
| | | 100 | 77.25 | 28.37 | 370.13 | 438.88 | 300.82 |
| 93-5 | Fruits | 1 | 24.85 | 86.90 | 146.92 | 93.73 | 80.99 |
| | | 10 | 42.78 | 37.17 | 275.85 | 129.94 | 131.81 |
| | | 100 | 75.83 | 30.26 | 481.61 | 448.29 | 229.03 |
| 93-6 | Flowers | 1 | 21.43 | 49.74 | 144.33 | 91.80 | 109.04 |
| | | 10 | 44.97 | 30.02 | 210.57 | 149.76 | 163.34 |
| | | 100 | 84.55 | 24.36 | 454.66 | 473.11 | 310.81 |

TABLE 188

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 93 | Thunberg's | | | | | | |
| | bay-tree | | | | | | |
| 93-1 | Whole plant | 1 | 98.32 | 104.49 | 106.06 | 84.99 | 101.22 |
| | | 10 | 89.53 | 115.47 | 94.48 | 86.18 | 182.16 |
| | | 100 | 79.70 | 133.64 | 37.37 | 95.80 | 501.00 |
| 93-2 | Roots | 1 | 102.50 | 102.26 | 83.39 | 67.87 | 115.96 |
| | | 10 | 84.79 | 109.79 | 47.03 | 84.17 | 217.86 |
| | | 100 | 73.47 | 136.33 | 46.74 | 106.97 | 491.54 |
| 93-3 | Stems | 1 | 98.01 | 97.10 | 96.61 | 69.37 | 209.30 |
| | | 10 | 92.45 | 131.89 | 76.72 | 94.11 | 242.55 |
| | | 100 | 77.11 | 149.60 | 10.07 | 110.82 | 425.54 |
| 93-4 | Leaves | 1 | 100.10 | 96.57 | 98.67 | 82.07 | 102.14 |
| | | 10 | 88.26 | 115.05 | 51.13 | 81.85 | 244.14 |
| | | 100 | 79.93 | 133.09 | 12.40 | 99.95 | 396.72 |
| 93-5 | Fruits | 1 | 100.26 | 114.74 | 101.29 | 73.62 | 184.46 |
| | | 10 | 82.04 | 110.35 | 92.31 | 77.02 | 311.63 |
| | | 100 | 76.09 | 153.87 | 23.78 | 105.34 | 434.60 |
| 93-6 | Flowers | 1 | 102.89 | 102.16 | 81.55 | 67.56 | 163.42 |
| | | 10 | 90.42 | 126.29 | 79.88 | 90.91 | 224.56 |
| | | 100 | 71.58 | 135.19 | 46.73 | 92.55 | 433.95 |

### Example 94. Ulleungdo White Pine

TABLE 189

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 94 | Ulleungdo | | | | | | |
| | white pine | | | | | | |
| 94-1 | Whole plant | 1 | 25.70 | 45.38 | 181.92 | 80.72 | 87.40 |
| | | 10 | 47.44 | 38.01 | 239.08 | 171.91 | 139.69 |
| | | 100 | 76.72 | 20.31 | 373.94 | 386.78 | 275.00 |
| 94-2 | Roots | 1 | 27.55 | 59.16 | 127.16 | 110.05 | 121.87 |
| | | 10 | 35.62 | 33.64 | 282.12 | 143.05 | 234.96 |
| | | 100 | 80.93 | 28.35 | 361.80 | 480.54 | 214.18 |
| 94-3 | Stems | 1 | 29.43 | 61.36 | 128.66 | 103.74 | 127.79 |
| | | 10 | 32.95 | 39.29 | 244.07 | 113.84 | 209.84 |
| | | 100 | 79.84 | 26.94 | 462.48 | 436.35 | 333.28 |
| 94-4 | Leaves | 1 | 26.83 | 89.13 | 130.52 | 105.02 | 81.28 |
| | | 10 | 36.00 | 37.35 | 339.22 | 180.40 | 146.84 |
| | | 100 | 72.78 | 26.16 | 367.23 | 439.31 | 325.05 |
| 94-5 | Fruits | 1 | 25.40 | 86.88 | 171.57 | 104.86 | 113.13 |
| | | 10 | 50.62 | 35.13 | 301.77 | 196.57 | 210.05 |
| | | 100 | 83.07 | 28.51 | 396.18 | 483.59 | 314.32 |
| 94-6 | Flowers | 1 | 30.43 | 75.50 | 143.90 | 88.28 | 129.59 |
| | | 10 | 45.05 | 38.06 | 201.55 | 166.78 | 235.39 |
| | | 100 | 71.67 | 27.25 | 473.42 | 342.46 | 317.43 |

TABLE 190

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 94 | Ulleungdo | | | | | | |
| | white pine | | | | | | |
| 94-1 | Whole plant | 1 | 98.98 | 102.69 | 107.62 | 85.22 | 118.44 |
| | | 10 | 95.11 | 115.21 | 49.37 | 79.54 | 235.24 |
| | | 100 | 73.75 | 154.72 | 36.60 | 100.22 | 339.25 |
| 94-2 | Roots | 1 | 103.39 | 109.96 | 86.36 | 69.49 | 165.17 |
| | | 10 | 84.47 | 107.40 | 95.28 | 75.58 | 208.92 |
| | | 100 | 70.42 | 160.47 | 6.97 | 97.10 | 467.82 |
| 94-3 | Stems | 1 | 95.52 | 113.08 | 80.13 | 82.84 | 122.90 |
| | | 10 | 89.41 | 133.21 | 88.27 | 78.46 | 234.39 |
| | | 100 | 71.74 | 139.45 | 26.85 | 96.63 | 343.61 |
| 94-4 | Leaves | 1 | 99.53 | 108.94 | 107.95 | 75.14 | 108.64 |
| | | 10 | 86.47 | 132.49 | 89.17 | 85.25 | 282.34 |
| | | 100 | 78.95 | 153.98 | 30.46 | 95.58 | 439.46 |
| 94-5 | Fruits | 1 | 97.51 | 108.19 | 96.66 | 85.94 | 194.75 |
| | | 10 | 83.39 | 132.15 | 95.19 | 80.80 | 288.35 |
| | | 100 | 72.41 | 142.70 | 41.10 | 109.80 | 490.50 |
| 94-6 | Flowers | 1 | 103.36 | 109.67 | 107.16 | 80.27 | 175.54 |
| | | 10 | 87.60 | 137.75 | 54.47 | 79.75 | 253.36 |
| | | 100 | 70.09 | 132.79 | 25.20 | 108.53 | 307.49 |

### Example 95. Ulleungdo Hemlock

TABLE 191

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 95 | Ulleungdo | | | | | | |
| | hemlock | | | | | | |
| 95-1 | Whole plant | 1 | 29.93 | 41.27 | 188.05 | 109.74 | 140.13 |
| | | 10 | 39.72 | 39.17 | 282.05 | 198.61 | 243.55 |
| | | 100 | 75.28 | 29.45 | 443.55 | 389.95 | 226.52 |
| 95-2 | Roots | 1 | 30.62 | 62.62 | 179.54 | 84.20 | 88.73 |
| | | 10 | 40.39 | 39.95 | 201.57 | 125.44 | 145.10 |
| | | 100 | 81.10 | 26.02 | 413.57 | 387.76 | 253.17 |
| 95-3 | Stems | 1 | 26.92 | 50.29 | 195.55 | 95.35 | 145.59 |
| | | 10 | 38.58 | 36.63 | 276.91 | 176.05 | 184.35 |
| | | 100 | 82.56 | 27.44 | 468.72 | 424.85 | 330.06 |
| 95-4 | Leaves | 1 | 20.07 | 51.76 | 154.50 | 88.52 | 144.99 |
| | | 10 | 32.11 | 35.58 | 340.21 | 111.66 | 144.51 |
| | | 100 | 78.86 | 24.85 | 499.66 | 320.30 | 312.51 |
| 95-5 | Fruits | 1 | 24.26 | 43.33 | 155.17 | 99.28 | 89.27 |
| | | 10 | 40.36 | 38.18 | 219.96 | 176.44 | 112.45 |
| | | 100 | 76.49 | 28.88 | 411.87 | 419.25 | 336.11 |
| 95-6 | Flowers | 1 | 25.80 | 77.57 | 114.93 | 83.32 | 100.32 |
| | | 10 | 44.39 | 40.02 | 230.60 | 192.94 | 220.03 |
| | | 100 | 77.44 | 23.66 | 427.89 | 444.64 | 304.41 |

TABLE 192

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 95 | Ulleungdo | | | | | | |
| | hemlock | | | | | | |
| 95-1 | Whole plant | 1 | 100.67 | 105.49 | 80.41 | 67.11 | 105.81 |
| | | 10 | 88.62 | 111.47 | 71.64 | 81.43 | 185.10 |
| | | 100 | 75.90 | 143.57 | 7.01 | 107.83 | 343.85 |
| 95-2 | Roots | 1 | 98.03 | 115.87 | 86.36 | 68.82 | 110.33 |
| | | 10 | 92.42 | 118.31 | 88.96 | 80.36 | 212.68 |
| | | 100 | 75.47 | 134.94 | 18.56 | 103.19 | 478.79 |
| 95-3 | Stems | 1 | 97.93 | 108.88 | 95.25 | 73.84 | 144.72 |
| | | 10 | 83.27 | 130.66 | 83.75 | 91.82 | 190.14 |
| | | 100 | 80.45 | 135.29 | 42.51 | 104.96 | 417.60 |
| 95-4 | Leaves | 1 | 101.15 | 104.73 | 99.60 | 73.86 | 153.10 |
| | | 10 | 80.77 | 116.41 | 53.90 | 87.69 | 323.75 |
| | | 100 | 73.77 | 138.13 | 35.01 | 105.21 | 475.27 |
| 95-5 | Fruits | 1 | 102.37 | 101.47 | 82.74 | 82.02 | 161.96 |
| | | 10 | 93.68 | 113.83 | 52.79 | 82.51 | 271.39 |
| | | 100 | 80.02 | 140.02 | 20.51 | 103.41 | 356.39 |
| 95-6 | Flowers | 1 | 103.46 | 105.14 | 92.92 | 66.00 | 129.93 |
| | | 10 | 95.35 | 137.29 | 45.19 | 88.56 | 186.22 |
| | | 100 | 73.05 | 151.34 | 39.72 | 94.83 | 420.46 |

### Example 96. Sericeous Newlitsea

TABLE 193

| | | | Blood | Lipid- | Anti-obesity- | | Menopause- |
|---|---|---|---|---|---|---|---|
| | | | circulation- | cholesterol | Glycerol | Immunity- | ER binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 | — | — | — | — |
| | control | | (oxLDL | | | | |
| | | | 200 ppm) | | | | |
| | Positive | | 98.29 | 50.00 | 872.36 | 964.24 | 182.11 |
| | control | | (Rosuvastatin | (Atorvastatin | (Isoproterenol | (LPS 1 ppm) | (17β-estradiol |
| | | | 0.05 ppm) | 70.4 nM) | 10 μM) | | 1 ng/ml) |
| 96 | Sericeous | | | | | | |
| | newlitsea | | | | | | |
| 96-1 | Whole plant | 1 | 29.57 | 88.77 | 188.28 | 81.06 | 137.07 |
| | | 10 | 44.30 | 32.91 | 248.76 | 147.21 | 116.21 |
| | | 100 | 71.30 | 29.18 | 425.88 | 405.98 | 239.72 |
| 96-2 | Roots | 1 | 26.40 | 72.28 | 117.82 | 100.79 | 117.02 |
| | | 10 | 39.18 | 40.70 | 347.13 | 125.05 | 108.60 |
| | | 100 | 76.02 | 22.90 | 350.85 | 389.94 | 216.97 |
| 96-3 | Stems | 1 | 27.32 | 41.36 | 120.68 | 90.42 | 138.27 |
| | | 10 | 35.77 | 30.60 | 201.74 | 199.86 | 243.81 |
| | | 100 | 76.30 | 21.98 | 382.13 | 310.23 | 240.91 |
| 96-4 | Leaves | 1 | 25.88 | 64.64 | 162.66 | 94.96 | 82.66 |
| | | 10 | 42.16 | 30.69 | 212.70 | 121.06 | 186.88 |
| | | 100 | 78.66 | 25.65 | 421.93 | 402.72 | 348.79 |
| 96-5 | Fruits | 1 | 30.40 | 53.19 | 156.56 | 93.29 | 94.45 |
| | | 10 | 30.60 | 32.82 | 320.97 | 171.60 | 153.71 |
| | | 100 | 71.57 | 22.13 | 410.86 | 419.97 | 225.06 |
| 96-6 | Flowers | 1 | 21.27 | 56.79 | 188.73 | 90.15 | 148.08 |
| | | 10 | 44.24 | 30.44 | 232.26 | 112.56 | 127.14 |
| | | 100 | 79.42 | 28.24 | 459.86 | 428.69 | 350.25 |

TABLE 194

| | | | | Blood | Cognitive | | Andropause- |
|---|---|---|---|---|---|---|---|
| | | | Anti- | glucose- | ability-β- | | AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 | — |
| | control | | | | | (Ethanol | |
| | | | | | | 100 nm) | |
| | Positive | | 67.27 | 183.77 | 50.00 | 88.28 | 753.39 |
| | control | | (Dexamethasone | (Metformin | (β-Secretase | (Silymarin | (Dihydrotestosterone |
| | | | 1 uM) | 1 mM) | Inhibitor IV | 100 ppm) | 100 nM) |
| | | | | | 15 nM) | | |
| 96 | Sericeous | | | | | | |
| | newlitsea | | | | | | |
| 96-1 | Whole plant | 1 | 100.67 | 108.87 | 98.79 | 67.47 | 129.14 |
| | | 10 | 87.32 | 124.66 | 75.67 | 85.41 | 317.84 |
| | | 100 | 74.87 | 147.87 | 50.96 | 98.93 | 342.89 |
| 96-2 | Roots | 1 | 103.50 | 99.64 | 92.33 | 76.26 | 191.34 |
| | | 10 | 93.89 | 139.90 | 95.07 | 87.03 | 271.33 |
| | | 100 | 71.39 | 137.09 | 14.04 | 90.38 | 337.28 |
| 96-3 | Stems | 1 | 102.01 | 104.14 | 100.39 | 66.88 | 187.41 |
| | | 10 | 89.74 | 128.54 | 62.54 | 82.73 | 316.46 |
| | | 100 | 80.17 | 160.88 | 46.46 | 91.08 | 381.31 |
| 96-4 | Leaves | 1 | 101.24 | 113.10 | 100.07 | 79.41 | 162.58 |
| | | 10 | 91.39 | 112.88 | 95.53 | 83.49 | 293.03 |
| | | 100 | 74.59 | 154.16 | 10.41 | 110.70 | 308.99 |
| 96-5 | Fruits | 1 | 97.49 | 109.79 | 97.92 | 84.00 | 184.36 |
| | | 10 | 86.93 | 118.29 | 91.98 | 92.00 | 190.52 |
| | | 100 | 70.51 | 157.47 | 19.96 | 96.47 | 443.36 |
| 96-6 | Flowers | 1 | 95.14 | 95.26 | 95.60 | 71.70 | 96.27 |
| | | 10 | 94.89 | 132.94 | 49.36 | 77.19 | 310.72 |
| | | 100 | 72.98 | 150.29 | 7.18 | 98.77 | 300.35 |

### Example 97. Macropodous Daphniphyllum

TABLE 195

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 97 | Macropodous | | | | | | |
| | daphniphyllum | | | | | | |
| 97-1 | Whole plant | 1 | 29.30 | 59.68 | 106.19 | 94.86 | 116.02 |
| | | 10 | 43.86 | 37.88 | 220.92 | 158.58 | 194.16 |
| | | 100 | 79.81 | 30.78 | 472.14 | 483.88 | 319.24 |
| 97-2 | Roots | 1 | 23.56 | 50.35 | 190.69 | 104.92 | 111.94 |
| | | 10 | 30.10 | 30.64 | 242.70 | 140.40 | 135.73 |
| | | 100 | 71.28 | 29.33 | 364.93 | 486.82 | 319.07 |
| 97-3 | Stems | 1 | 24.65 | 83.21 | 164.83 | 104.75 | 136.71 |
| | | 10 | 35.97 | 31.22 | 226.09 | 150.94 | 191.11 |
| | | 100 | 78.09 | 22.75 | 402.31 | 420.92 | 252.81 |
| 97-4 | Leaves | 1 | 25.41 | 59.67 | 149.98 | 94.74 | 112.03 |
| | | 10 | 40.54 | 32.55 | 315.58 | 182.62 | 244.22 |
| | | 100 | 78.47 | 24.29 | 451.73 | 357.34 | 260.48 |
| 97-5 | Fruits | 1 | 25.00 | 82.34 | 107.89 | 90.92 | 129.63 |
| | | 10 | 35.41 | 35.20 | 200.46 | 140.51 | 178.41 |
| | | 100 | 75.37 | 20.38 | 435.54 | 364.45 | 254.41 |
| 97-6 | Flowers | 1 | 29.91 | 53.72 | 153.65 | 91.68 | 116.88 |
| | | 10 | 48.99 | 33.26 | 275.15 | 177.91 | 208.00 |
| | | 100 | 74.42 | 22.02 | 490.60 | 379.87 | 336.88 |

TABLE 196

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 97 | Macropodous | | | | | | |
| | daphniphyllum | | | | | | |
| 97-1 | Whole plant | 1 | 102.96 | 96.60 | 92.79 | 78.33 | 148.33 |
| | | 10 | 86.43 | 135.35 | 77.79 | 84.17 | 210.16 |
| | | 100 | 73.29 | 149.84 | 40.54 | 109.02 | 483.63 |
| 97-2 | Roots | 1 | 99.44 | 113.82 | 97.92 | 83.83 | 90.56 |
| | | 10 | 95.29 | 129.27 | 92.94 | 92.24 | 187.17 |
| | | 100 | 71.64 | 138.13 | 4.84 | 99.69 | 344.36 |
| 97-3 | Stems | 1 | 99.18 | 112.02 | 80.99 | 67.23 | 169.48 |
| | | 10 | 89.97 | 136.84 | 52.15 | 94.79 | 243.21 |
| | | 100 | 76.59 | 130.83 | 14.09 | 104.35 | 485.05 |
| 97-4 | Leaves | 1 | 101.87 | 110.30 | 86.22 | 78.41 | 132.50 |
| | | 10 | 90.62 | 130.76 | 94.27 | 94.20 | 315.09 |
| | | 100 | 78.59 | 137.97 | 24.89 | 92.35 | 381.63 |
| 97-5 | Fruits | 1 | 95.67 | 99.74 | 104.32 | 73.45 | 115.38 |
| | | 10 | 86.53 | 111.89 | 49.58 | 83.61 | 262.87 |
| | | 100 | 74.09 | 133.36 | 23.34 | 108.10 | 325.49 |
| 97-6 | Flowers | 1 | 103.79 | 105.47 | 110.46 | 68.36 | 188.14 |
| | | 10 | 84.05 | 114.32 | 51.27 | 80.80 | 217.61 |
| | | 100 | 73.99 | 157.88 | 47.85 | 99.64 | 300.57 |

### Example 98. Scabrous Aphananthe

TABLE 197

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 98 | Scabrous | | | | | | |
| | aphananthe | | | | | | |
| 98-1 | Whole plant | 1 | 29.03 | 79.88 | 155.65 | 105.27 | 84.80 |
| | | 10 | 36.31 | 32.32 | 328.00 | 141.70 | 186.42 |
| | | 100 | 83.56 | 21.54 | 363.38 | 477.26 | 316.44 |
| 98-2 | Roots | 1 | 26.34 | 75.81 | 138.56 | 109.93 | 126.82 |
| | | 10 | 49.04 | 31.45 | 275.09 | 156.96 | 217.77 |
| | | 100 | 82.77 | 25.05 | 394.42 | 462.13 | 305.78 |
| 98-3 | Stems | 1 | 23.27 | 83.22 | 127.80 | 80.99 | 137.35 |
| | | 10 | 38.33 | 37.29 | 338.61 | 194.08 | 233.85 |
| | | 100 | 83.01 | 20.21 | 414.77 | 352.21 | 230.02 |
| 98-4 | Leaves | 1 | 26.96 | 57.27 | 161.75 | 95.27 | 82.18 |
| | | 10 | 31.89 | 37.09 | 256.72 | 178.40 | 139.95 |
| | | 100 | 76.33 | 26.57 | 500.74 | 360.04 | 336.63 |
| 98-5 | Fruits | 1 | 22.38 | 59.21 | 129.16 | 91.04 | 80.55 |
| | | 10 | 49.33 | 35.66 | 262.49 | 166.67 | 212.58 |
| | | 100 | 70.59 | 23.80 | 435.62 | 344.39 | 243.35 |
| 98-6 | Flowers | 1 | 26.18 | 83.99 | 140.37 | 97.07 | 127.08 |
| | | 10 | 39.85 | 33.84 | 289.83 | 193.30 | 239.55 |
| | | 100 | 79.14 | 25.48 | 363.95 | 473.68 | 287.08 |

TABLE 198

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | ( % of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 98 | Scabrous | | | | | | |
| | aphananthe | | | | | | |
| 98-1 | Whole plant | 1 | 99.40 | 96.18 | 103.36 | 80.06 | 144.79 |
| | | 10 | 80.88 | 128.54 | 92.25 | 85.43 | 222.47 |
| | | 100 | 77.94 | 130.56 | 3.87 | 90.68 | 404.67 |
| 98-2 | Roots | 1 | 99.44 | 100.18 | 99.10 | 73.29 | 154.39 |
| | | 10 | 92.34 | 139.32 | 57.35 | 87.95 | 204.62 |
| | | 100 | 71.65 | 145.20 | 30.58 | 108.13 | 475.19 |
| 98-3 | Stems | 1 | 102.70 | 103.99 | 93.36 | 72.66 | 184.30 |
| | | 10 | 88.16 | 132.80 | 80.07 | 81.87 | 235.63 |
| | | 100 | 74.76 | 140.45 | 43.79 | 105.23 | 408.34 |
| 98-4 | Leaves | 1 | 97.79 | 98.70 | 107.30 | 65.02 | 103.81 |
| | | 10 | 91.20 | 107.02 | 75.69 | 82.57 | 309.29 |
| | | 100 | 73.81 | 130.78 | 22.80 | 109.56 | 414.55 |
| 98-5 | Fruits | 1 | 102.56 | 95.15 | 101.46 | 74.17 | 145.33 |
| | | 10 | 93.30 | 132.20 | 95.84 | 92.54 | 196.11 |
| | | 100 | 78.43 | 135.56 | 29.31 | 101.55 | 423.89 |
| 98-6 | Flowers | 1 | 95.26 | 97.87 | 110.31 | 78.32 | 148.97 |
| | | 10 | 86.69 | 124.93 | 59.98 | 76.09 | 260.30 |
| | | 100 | 76.74 | 135.58 | 27.70 | 100.50 | 320.70 |

### Example 99. Caudate-Leaf Hackberry

TABLE 199

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 99 | Caudate-leaf | | | | | | |
| | hackberry | | | | | | |
| 99-1 | Whole plant | 1 | 24.12 | 58.38 | 185.39 | 96.94 | 124.44 |
| | | 10 | 37.37 | 40.07 | 254.80 | 113.47 | 201.88 |
| | | 100 | 83.47 | 30.16 | 445.84 | 438.15 | 346.24 |
| 99-2 | Roots | 1 | 27.26 | 42.32 | 127.26 | 88.40 | 83.91 |
| | | 10 | 50.89 | 36.39 | 284.59 | 114.72 | 250.95 |
| | | 100 | 78.63 | 24.33 | 351.66 | 307.29 | 338.33 |
| 99-3 | Stems | 1 | 22.67 | 83.41 | 157.92 | 106.00 | 139.19 |
| | | 10 | 46.83 | 33.93 | 331.33 | 142.34 | 218.47 |
| | | 100 | 74.98 | 29.78 | 356.67 | 413.37 | 239.11 |
| 99-4 | Leaves | 1 | 28.92 | 90.21 | 173.10 | 105.49 | 107.73 |
| | | 10 | 46.53 | 33.10 | 306.66 | 116.15 | 242.38 |
| | | 100 | 72.70 | 21.79 | 464.32 | 308.10 | 328.11 |
| 99-5 | Fruits | 1 | 23.83 | 64.67 | 108.57 | 88.22 | 84.28 |
| | | 10 | 38.19 | 30.01 | 299.88 | 140.74 | 108.56 |
| | | 100 | 70.62 | 30.40 | 456.92 | 495.46 | 309.89 |
| 99-6 | Flowers | 1 | 27.90 | 86.54 | 148.46 | 82.91 | 82.86 |
| | | 10 | 49.07 | 40.95 | 223.90 | 142.03 | 106.22 |
| | | 100 | 83.50 | 26.18 | 470.35 | 306.23 | 246.80 |

TABLE 200

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 99 | Caudate-leaf | | | | | | |
| | hackberry | | | | | | |
| 99-1 | Whole plant | 1 | 100.91 | 104.89 | 106.05 | 84.40 | 210.61 |
| | | 10 | 93.23 | 130.09 | 49.95 | 80.69 | 214.18 |
| | | 100 | 79.18 | 149.04 | 9.43 | 107.73 | 312.13 |
| 99-2 | Roots | 1 | 99.29 | 107.33 | 105.68 | 85.37 | 135.82 |
| | | 10 | 84.62 | 124.28 | 80.41 | 77.99 | 299.31 |
| | | 100 | 78.81 | 155.37 | 48.30 | 96.54 | 309.79 |
| 99-3 | Stems | 1 | 100.52 | 113.66 | 106.55 | 84.16 | 142.91 |
| | | 10 | 94.74 | 114.16 | 45.21 | 81.25 | 197.66 |
| | | 100 | 73.28 | 139.99 | 28.60 | 90.88 | 358.74 |
| 99-4 | Leaves | 1 | 97.40 | 106.53 | 84.24 | 70.72 | 95.24 |
| | | 10 | 85.61 | 114.49 | 83.62 | 90.40 | 294.32 |
| | | 100 | 72.34 | 147.88 | 3.66 | 110.31 | 359.77 |
| 99-5 | Fruits | 1 | 101.18 | 108.06 | 103.12 | 73.34 | 205.04 |
| | | 10 | 86.39 | 135.81 | 71.97 | 81.50 | 262.50 |
| | | 100 | 75.04 | 158.27 | 21.13 | 91.02 | 325.19 |
| 99-6 | Flowers | 1 | 101.90 | 95.32 | 80.54 | 80.87 | 163.16 |
| | | 10 | 95.59 | 112.00 | 80.46 | 79.34 | 336.34 |
| | | 100 | 78.39 | 156.42 | 38.64 | 97.55 | 370.44 |

### Example 100. Manchurian Elm

TABLE 201

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 100 | Manchurian | | | | | | |
| | Elm | | | | | | |
| 100-1 | Whole plant | 1 | 20.79 | 60.31 | 126.79 | 109.62 | 127.03 |
| | | 10 | 42.05 | 39.11 | 303.28 | 140.43 | 171.92 |
| | | 100 | 82.34 | 20.07 | 408.67 | 463.03 | 302.19 |
| 100-2 | Roots | 1 | 24.61 | 58.39 | 181.77 | 107.61 | 92.35 |
| | | 10 | 42.05 | 34.58 | 290.82 | 170.69 | 188.16 |
| | | 100 | 78.73 | 27.24 | 408.47 | 388.27 | 291.54 |
| 100-3 | Stems | 1 | 20.90 | 75.10 | 130.87 | 88.61 | 132.05 |
| | | 10 | 30.04 | 39.66 | 258.46 | 192.67 | 100.86 |
| | | 100 | 82.25 | 29.42 | 350.45 | 494.14 | 338.56 |
| 100-4 | Leaves | 1 | 20.31 | 68.48 | 154.51 | 91.88 | 88.99 |
| | | 10 | 49.89 | 30.44 | 320.85 | 165.23 | 195.55 |
| | | 100 | 72.19 | 22.61 | 422.98 | 409.94 | 275.61 |
| 100-5 | Fruits | 1 | 25.23 | 80.47 | 130.70 | 84.05 | 102.64 |
| | | 10 | 48.85 | 34.54 | 319.28 | 173.13 | 242.73 |
| | | 100 | 72.25 | 26.36 | 432.04 | 472.28 | 205.67 |
| 100-6 | Flowers | 1 | 25.39 | 71.94 | 108.44 | 87.02 | 115.82 |
| | | 10 | 50.50 | 40.54 | 207.75 | 162.44 | 117.23 |
| | | 100 | 71.33 | 25.78 | 442.90 | 375.57 | 337.96 |

TABLE 202

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 100 | Manchurian | | | | | | |
| | Elm | | | | | | |
| 100-1 | Whole plant | 1 | 101.61 | 110.44 | 81.47 | 83.13 | 138.36 |
| | | 10 | 81.13 | 120.32 | 50.69 | 80.14 | 250.83 |
| | | 100 | 77.19 | 132.27 | 26.04 | 110.40 | 389.70 |
| 100-2 | Roots | 1 | 102.17 | 110.90 | 106.07 | 77.13 | 185.24 |
| | | 10 | 91.67 | 129.71 | 66.92 | 90.56 | 299.38 |
| | | 100 | 72.83 | 149.35 | 43.52 | 104.76 | 387.05 |
| 100-3 | Stems | 1 | 96.41 | 107.14 | 95.03 | 84.63 | 186.52 |
| | | 10 | 87.44 | 118.92 | 60.50 | 93.12 | 308.63 |
| | | 100 | 74.31 | 158.25 | 21.86 | 95.55 | 480.07 |
| 100-4 | Leaves | 1 | 97.03 | 100.92 | 105.61 | 84.24 | 101.22 |
| | | 10 | 86.55 | 106.41 | 64.72 | 93.59 | 216.20 |
| | | 100 | 74.83 | 145.29 | 30.54 | 95.04 | 445.04 |
| 100-5 | Fruits | 1 | 101.25 | 103.23 | 95.77 | 82.79 | 105.80 |
| | | 10 | 95.54 | 123.94 | 60.87 | 91.26 | 284.26 |
| | | 100 | 78.50 | 158.72 | 39.89 | 100.21 | 444.73 |
| 100-6 | Flowers | 1 | 103.90 | 109.61 | 101.67 | 67.55 | 148.55 |
| | | 10 | 80.02 | 114.93 | 86.91 | 94.65 | 294.17 |
| | | 100 | 77.43 | 131.53 | 12.66 | 109.19 | 465.18 |

### Example 101. Montane Alder

TABLE 203

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | ( % of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 101 | Montane | | | | | | |
| | alder | | | | | | |
| 101-1 | Whole plant | 1 | 28.43 | 67.24 | 151.87 | 101.77 | 85.65 |
| | | 10 | 42.43 | 34.65 | 294.03 | 139.37 | 230.16 |
| | | 100 | 79.23 | 23.33 | 401.31 | 436.59 | 218.11 |
| 101-2 | Roots | 1 | 25.47 | 90.82 | 147.41 | 85.30 | 80.39 |
| | | 10 | 32.05 | 37.03 | 207.94 | 119.77 | 139.36 |
| | | 100 | 78.10 | 25.84 | 483.93 | 405.43 | 327.91 |
| 101-3 | Stems | 1 | 20.30 | 54.04 | 179.83 | 102.96 | 134.24 |
| | | 10 | 38.09 | 36.77 | 217.68 | 194.29 | 108.40 |
| | | 100 | 70.05 | 30.41 | 379.77 | 349.69 | 217.08 |
| 101-4 | Leaves | 1 | 24.91 | 68.28 | 153.67 | 81.71 | 123.42 |
| | | 10 | 37.47 | 33.72 | 230.77 | 126.02 | 125.32 |
| | | 100 | 70.51 | 27.89 | 441.02 | 341.45 | 262.85 |
| 101-5 | Fruits | 1 | 25.25 | 49.38 | 102.18 | 82.55 | 96.21 |
| | | 10 | 32.22 | 35.69 | 254.61 | 146.21 | 156.43 |
| | | 100 | 72.68 | 25.73 | 457.67 | 410.04 | 318.99 |
| 101-6 | Flowers | 1 | 25.49 | 76.85 | 195.32 | 84.65 | 128.56 |
| | | 10 | 36.79 | 36.06 | 330.49 | 149.35 | 249.47 |
| | | 100 | 79.00 | 21.22 | 368.75 | 410.51 | 294.40 |

TABLE 204

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 101 | Montane | | | | | | |
| | alder | | | | | | |
| 101-1 | Whole plant | 1 | 96.15 | 97.99 | 88.38 | 77.84 | 161.93 |
| | | 10 | 92.82 | 127.06 | 55.76 | 92.78 | 336.57 |
| | | 100 | 73.41 | 149.11 | 22.87 | 109.93 | 318.77 |
| 101-2 | Roots | 1 | 96.96 | 102.18 | 84.21 | 67.11 | 207.05 |
| | | 10 | 80.21 | 127.18 | 65.64 | 76.71 | 247.26 |
| | | 100 | 73.98 | 143.80 | 15.63 | 99.35 | 454.16 |
| 101-3 | Stems | 1 | 101.34 | 95.14 | 108.32 | 65.17 | 176.35 |
| | | 10 | 85.68 | 119.20 | 47.76 | 79.09 | 283.01 |
| | | 100 | 75.50 | 136.34 | 28.28 | 105.72 | 395.65 |
| 101-4 | Leaves | 1 | 95.86 | 107.97 | 87.74 | 74.47 | 198.91 |
| | | 10 | 85.94 | 138.57 | 68.47 | 93.58 | 261.89 |
| | | 100 | 70.26 | 135.38 | 45.28 | 100.06 | 337.32 |
| 101-5 | Fruits | 1 | 95.98 | 100.41 | 110.84 | 84.12 | 115.02 |
| | | 10 | 85.06 | 124.53 | 94.75 | 76.01 | 175.19 |
| | | 100 | 80.48 | 132.90 | 0.40 | 98.73 | 403.80 |
| 101-6 | Flowers | 1 | 96.22 | 101.75 | 102.67 | 79.39 | 110.83 |
| | | 10 | 83.22 | 132.81 | 47.03 | 81.91 | 318.80 |
| | | 100 | 77.72 | 154.63 | 30.19 | 94.30 | 401.20 |

### Example 102. Ulleungdo Linden

TABLE 205

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 102 | Ulleungdo | | | | | | |
| | linden | | | | | | |
| 102-1 | Whole plant | 1 | 28.71 | 85.25 | 182.16 | 89.70 | 114.13 |
| | | 10 | 44.00 | 32.97 | 322.79 | 110.01 | 182.63 |
| | | 100 | 80.49 | 29.90 | 402.14 | 419.69 | 274.17 |
| 102-2 | Roots | 1 | 22.48 | 71.21 | 143.43 | 101.54 | 142.61 |
| | | 10 | 42.27 | 33.81 | 327.19 | 140.05 | 192.67 |
| | | 100 | 85.63 | 22.20 | 454.09 | 482.16 | 252.44 |
| 102-3 | Stems | 1 | 27.98 | 55.30 | 122.16 | 82.11 | 87.47 |
| | | 10 | 33.82 | 39.20 | 294.19 | 161.85 | 239.65 |
| | | 100 | 83.15 | 30.48 | 484.01 | 429.12 | 285.33 |
| 102-4 | Leaves | 1 | 22.15 | 87.12 | 113.73 | 93.19 | 127.49 |
| | | 10 | 40.55 | 40.44 | 316.35 | 110.79 | 145.02 |
| | | 100 | 75.56 | 29.33 | 401.87 | 409.18 | 343.18 |
| 102-5 | Fruits | 1 | 30.18 | 78.38 | 102.97 | 82.46 | 126.58 |
| | | 10 | 45.12 | 35.39 | 219.30 | 156.39 | 118.53 |
| | | 100 | 72.10 | 22.65 | 446.64 | 452.17 | 290.16 |
| 102-6 | Flowers | 1 | 26.15 | 89.75 | 172.95 | 102.36 | 132.33 |
| | | 10 | 43.43 | 34.18 | 252.43 | 122.33 | 214.47 |
| | | 100 | 78.34 | 21.55 | 471.91 | 485.63 | 261.18 |

TABLE 206

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 102 | Ulleungdo | | | | | | |
| | linden | | | | | | |
| 102-1 | Whole plant | | 100.75 | 102.31 | 99.21 | 80.80 | 189.98 |
| | | 10 | 92.19 | 114.49 | 87.25 | 95.50 | 285.17 |
| | | 100 | 72.74 | 140.83 | 16.74 | 90.19 | 397.87 |
| 102-2 | Roots | 1 | 102.59 | 96.65 | 85.51 | 70.81 | 110.73 |
| | | 10 | 93.84 | 128.24 | 87.44 | 94.31 | 205.24 |
| | | 100 | 80.59 | 150.52 | 17.85 | 103.59 | 473.62 |
| 102-3 | Stems | 1 | 101.65 | 97.51 | 95.13 | 74.75 | 210.57 |
| | | 10 | 92.38 | 124.21 | 73.60 | 83.43 | 331.30 |
| | | 100 | 71.50 | 142.27 | 17.20 | 90.79 | 435.62 |
| 102-4 | Leaves | 1 | 96.57 | 114.76 | 105.10 | 68.68 | 127.99 |
| | | 10 | 89.70 | 107.31 | 83.40 | 76.65 | 200.46 |
| | | 100 | 70.28 | 144.88 | 41.62 | 108.87 | 387.90 |
| 102-5 | Fruits | 1 | 97.21 | 95.08 | 84.07 | 76.89 | 121.86 |
| | | 10 | 84.29 | 110.78 | 67.99 | 77.36 | 269.52 |
| | | 100 | 71.51 | 134.15 | 1.68 | 101.52 | 473.99 |
| 102-6 | Flowers | 1 | 96.70 | 97.61 | 93.34 | 79.52 | 210.41 |
| | | 10 | 80.16 | 110.64 | 89.98 | 90.15 | 245.26 |
| | | 100 | 70.27 | 152.01 | 1.18 | 92.65 | 415.59 |

### Example 103. Fragrant Snowbell

TABLE 207

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 103 | Fragrant | | | | | | |
| | snowbell | | | | | | |
| 103-1 | Whole plant | 1 | 24.96 | 64.08 | 181.96 | 81.96 | 119.93 |
| | | 10 | 47.98 | 40.88 | 244.05 | 122.32 | 160.76 |
| | | 100 | 76.76 | 29.46 | 411.53 | 436.35 | 205.19 |
| 103-2 | Roots | 1 | 25.75 | 58.56 | 126.82 | 109.27 | 133.74 |
| | | 10 | 48.78 | 40.29 | 271.25 | 199.84 | 186.01 |
| | | 100 | 82.39 | 27.88 | 409.55 | 389.57 | 238.23 |
| 103-3 | Stems | 1 | 26.73 | 57.77 | 146.09 | 101.17 | 133.47 |
| | | 10 | 39.17 | 30.91 | 265.40 | 179.43 | 242.51 |
| | | 100 | 72.14 | 28.78 | 399.76 | 461.05 | 202.63 |
| 103-4 | Leaves | 1 | 25.93 | 68.12 | 184.19 | 92.36 | 101.47 |
| | | 10 | 46.70 | 31.92 | 210.87 | 121.76 | 103.76 |
| | | 100 | 70.29 | 30.25 | 439.60 | 395.18 | 299.88 |
| 103-5 | Fruits | 1 | 25.47 | 75.84 | 153.08 | 99.77 | 106.18 |
| | | 10 | 48.84 | 34.77 | 292.69 | 150.92 | 156.55 |
| | | 100 | 77.82 | 30.60 | 484.01 | 392.31 | 336.95 |
| 103-6 | Flowers | 1 | 30.88 | 60.93 | 158.43 | 81.92 | 129.50 |
| | | 10 | 38.51 | 36.32 | 217.62 | 151.73 | 116.85 |
| | | 100 | 72.82 | 26.91 | 443.31 | 450.32 | 221.66 |

TABLE 208

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 103 | Fragrant | | | | | | |
| | snowbell | | | | | | |
| 103-1 | Whole plant | 1 | 102.61 | 97.45 | 91.09 | 72.77 | 197.60 |
| | | 10 | 82.38 | 140.76 | 53.10 | 78.98 | 253.15 |
| | | 100 | 75.15 | 155.18 | 48.63 | 101.60 | 303.75 |
| 103-2 | Roots | 1 | 96.57 | 102.37 | 89.27 | 72.14 | 185.78 |
| | | 10 | 82.11 | 109.35 | 47.17 | 90.20 | 185.81 |
| | | 100 | 80.42 | 133.58 | 26.91 | 105.16 | 407.08 |
| 103-3 | Stems | 1 | 101.90 | 106.73 | 102.19 | 70.93 | 188.00 |
| | | 10 | 91.52 | 136.85 | 59.70 | 75.56 | 171.96 |
| | | 100 | 73.03 | 140.34 | 25.77 | 90.69 | 437.02 |
| 103-4 | Leaves | 1 | 95.79 | 109.64 | 106.78 | 85.77 | 194.33 |
| | | 10 | 81.98 | 119.82 | 46.89 | 91.10 | 206.42 |
| | | 100 | 70.30 | 141.07 | 13.19 | 105.87 | 313.41 |
| 103-5 | Fruits | 1 | 97.08 | 112.32 | 85.12 | 85.16 | 108.76 |
| | | 10 | 91.14 | 139.78 | 59.95 | 95.18 | 298.99 |
| | | 100 | 76.65 | 131.78 | 9.88 | 92.75 | 458.73 |
| 103-6 | Flowers | 1 | 96.94 | 103.71 | 100.80 | 77.17 | 175.25 |
| | | 10 | 83.85 | 112.99 | 78.89 | 89.69 | 287.43 |
| | | 100 | 74.31 | 155.86 | 6.25 | 90.23 | 449.53 |

### Example 104. Ulleungdo Flowering Cherry

TABLE 209

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 104 | Ulleungdo | | | | | | |
| | flowering | | | | | | |
| | cherry | | | | | | |
| 104-1 | Whole plant | 1 | 27.61 | 48.40 | 129.82 | 95.58 | 113.44 |
| | | 10 | 40.03 | 32.75 | 293.50 | 147.11 | 229.08 |
| | | 100 | 83.84 | 29.37 | 355.64 | 369.59 | 296.67 |
| 104-2 | Roots | 1 | 21.62 | 88.01 | 101.46 | 86.79 | 112.63 |
| | | 10 | 35.01 | 39.64 | 282.61 | 186.54 | 147.13 |
| | | 100 | 72.66 | 23.74 | 451.58 | 450.71 | 336.64 |
| 104-3 | Stems | 1 | 24.82 | 53.55 | 155.26 | 100.24 | 110.06 |
| | | 10 | 48.80 | 39.34 | 266.21 | 165.17 | 114.78 |
| | | 100 | 73.26 | 20.99 | 449.99 | 306.40 | 221.55 |
| 104-4 | Leaves | 1 | 22.72 | 47.78 | 153.14 | 83.32 | 93.76 |
| | | 10 | 45.06 | 37.88 | 279.88 | 132.33 | 209.87 |
| | | 100 | 75.75 | 24.00 | 457.59 | 385.98 | 281.65 |
| 104-5 | Fruits | 1 | 22.66 | 54.64 | 107.60 | 105.11 | 110.54 |
| | | 10 | 38.71 | 33.95 | 225.03 | 143.83 | 134.56 |
| | | 100 | 73.87 | 22.55 | 368.06 | 372.60 | 307.40 |
| 104-6 | Flowers | 1 | 28.72 | 85.71 | 179.12 | 92.87 | 81.85 |
| | | 10 | 48.19 | 40.18 | 291.40 | 151.30 | 175.93 |
| | | 100 | 74.60 | 29.91 | 395.33 | 483.40 | 337.22 |

TABLE 210

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 104 | Ulleungdo | | | | | | |
| | flowering | | | | | | |
| | cherry | | | | | | |
| 104-1 | Whole plant | 1 | 102.82 | 113.93 | 82.73 | 81.67 | 198.35 |
| | | 10 | 85.17 | 108.86 | 83.34 | 91.84 | 201.38 |
| | | 100 | 77.73 | 148.23 | 24.68 | 110.84 | 442.67 |
| 104-2 | Roots | 1 | 103.26 | 109.71 | 103.43 | 68.46 | 123.59 |
| | | 10 | 85.31 | 121.05 | 93.20 | 83.28 | 185.56 |
| | | 100 | 74.96 | 159.10 | 28.23 | 101.26 | 466.66 |
| 104-3 | Stems | 1 | 96.67 | 115.59 | 90.43 | 85.12 | 165.78 |
| | | 10 | 81.03 | 139.38 | 72.19 | 94.12 | 312.39 |
| | | 100 | 77.31 | 152.84 | 47.43 | 105.85 | 486.14 |
| 104-4 | Leaves | 1 | 99.90 | 100.82 | 94.11 | 74.51 | 144.57 |
| | | 10 | 92.07 | 120.56 | 59.81 | 94.25 | 249.69 |
| | | 100 | 70.77 | 134.25 | 13.89 | 103.01 | 436.50 |
| 104-5 | Fruits | 1 | 95.13 | 106.71 | 93.52 | 71.30 | 124.09 |
| | | 10 | 90.80 | 134.21 | 90.20 | 83.09 | 247.25 |
| | | 100 | 72.85 | 133.65 | 35.32 | 93.03 | 488.95 |
| 104-6 | Flowers | 1 | 96.14 | 100.71 | 83.99 | 79.31 | 201.94 |
| | | 10 | 81.05 | 105.48 | 74.11 | 83.13 | 229.14 |
| | | 100 | 70.14 | 143.76 | 45.74 | 95.44 | 382.52 |

### Example 105. Spotted Laurel

TABLE 211

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 105 | Spotted | | | | | | |
| | laurel | | | | | | |
| 105-1 | Whole plant | 1 | 24.37 | 85.29 | 126.14 | 107.71 | 89.47 |
| | | 10 | 30.49 | 38.25 | 268.28 | 171.00 | 162.81 |
| | | 100 | 80.28 | 21.36 | 496.55 | 497.66 | 248.32 |
| 105-2 | Roots | 1 | 20.79 | 78.27 | 130.45 | 104.02 | 97.69 |
| | | 10 | 50.25 | 36.04 | 215.06 | 112.65 | 133.60 |
| | | 100 | 80.89 | 23.80 | 487.86 | 334.37 | 328.19 |
| 105-3 | Stems | 1 | 21.92 | 77.97 | 126.03 | 86.80 | 120.72 |
| | | 10 | 49.08 | 36.89 | 236.02 | 124.21 | 243.41 |
| | | 100 | 72.76 | 22.43 | 456.15 | 437.61 | 333.34 |
| 105-4 | Leaves | 1 | 27.15 | 67.65 | 108.34 | 107.06 | 113.24 |
| | | 10 | 47.78 | 36.24 | 262.63 | 196.49 | 165.04 |
| | | 100 | 77.94 | 27.82 | 372.15 | 393.74 | 238.44 |
| 105-5 | Fruits | 1 | 26.42 | 73.94 | 104.41 | 99.10 | 111.14 |
| | | 10 | 35.05 | 33.08 | 211.38 | 179.06 | 235.51 |
| | | 100 | 79.31 | 28.28 | 449.96 | 327.37 | 227.25 |
| 105-6 | Flowers | 1 | 28.14 | 82.77 | 141.35 | 97.66 | 110.80 |
| | | 10 | 40.30 | 33.90 | 292.90 | 190.11 | 167.36 |
| | | 100 | 79.70 | 28.31 | 445.21 | 374.71 | 299.96 |

TABLE 212

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 105 | Spotted | | | | | | |
| | laurel | | | | | | |
| 105-1 | Whole plant | 1 | 98.20 | 97.46 | 84.95 | 70.35 | 137.05 |
| | | 10 | 82.61 | 136.86 | 91.00 | 81.28 | 335.47 |
| | | 100 | 75.40 | 143.05 | 40.88 | 90.40 | 413.41 |
| 105-2 | Roots | 1 | 100.20 | 97.78 | 108.96 | 72.66 | 106.21 |
| | | 10 | 94.97 | 116.32 | 76.46 | 87.84 | 301.04 |
| | | 100 | 70.37 | 135.38 | 43.66 | 108.47 | 481.91 |
| 105-3 | Stems | 1 | 96.22 | 99.64 | 94.02 | 84.45 | 185.01 |
| | | 10 | 89.88 | 109.22 | 68.91 | 82.55 | 241.98 |
| | | 100 | 76.53 | 155.90 | 43.42 | 101.86 | 344.16 |
| 105-4 | Leaves | 1 | 98.26 | 104.38 | 102.34 | 66.53 | 173.26 |
| | | 10 | 95.41 | 120.56 | 82.05 | 87.38 | 317.93 |
| | | 100 | 76.26 | 132.93 | 45.35 | 92.74 | 434.56 |
| 105-5 | Fruits | 1 | 96.14 | 109.27 | 94.27 | 83.79 | 168.76 |
| | | 10 | 90.55 | 117.86 | 82.73 | 78.48 | 279.85 |
| | | 100 | 77.45 | 152.83 | 35.33 | 107.96 | 382.10 |
| 105-6 | Flowers | 1 | 100.03 | 110.55 | 107.62 | 72.26 | 136.98 |
| | | 10 | 91.13 | 133.55 | 65.27 | 77.83 | 263.76 |
| | | 100 | 80.60 | 145.71 | 45.06 | 104.67 | 350.58 |

### Example 106. Ulleungdo Maple

TABLE 213

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 106 | Ulleungdo | | | | | | |
| | maple | | | | | | |
| 106-1 | Whole plant | 1 | 25.75 | 78.64 | 175.60 | 82.09 | 81.76 |
| | | 10 | 38.17 | 34.80 | 331.78 | 159.55 | 224.85 |
| | | 100 | 82.93 | 23.57 | 441.05 | 497.31 | 336.69 |
| 106-2 | Roots | 1 | 25.77 | 82.87 | 130.38 | 105.71 | 89.78 |
| | | 10 | 43.69 | 34.58 | 306.23 | 156.28 | 129.47 |
| | | 100 | 80.88 | 20.09 | 424.73 | 420.32 | 282.04 |
| 106-3 | Stems | 1 | 25.59 | 67.74 | 125.87 | 88.39 | 113.57 |
| | | 10 | 36.48 | 38.75 | 205.40 | 138.59 | 130.43 |
| | | 100 | 75.94 | 28.85 | 388.87 | 388.72 | 266.82 |
| 106-4 | Leaves | 1 | 24.02 | 50.26 | 107.80 | 89.57 | 97.50 |
| | | 10 | 30.06 | 32.94 | 222.73 | 121.52 | 175.12 |
| | | 100 | 85.12 | 21.10 | 465.61 | 330.84 | 314.84 |
| 106-5 | Fruits | 1 | 22.47 | 68.15 | 166.62 | 89.07 | 80.51 |
| | | 10 | 45.30 | 33.69 | 268.72 | 192.19 | 127.10 |
| | | 100 | 84.68 | 24.91 | 473.86 | 304.68 | 233.10 |
| 106-6 | Flowers | 1 | 26.22 | 70.04 | 182.15 | 80.59 | 143.43 |
| | | 10 | 43.95 | 40.57 | 304.49 | 116.78 | 116.13 |
| | | 100 | 81.52 | 26.05 | 472.45 | 461.87 | 335.24 |

TABLE 214

| | | | | Blood | Cognitive | | |
|---|---|---|---|---|---|---|---|
| | | | Anti- | glocose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 106 | Ulleungdo | | | | | | |
| | maple | | | | | | |
| 106-1 | Whole plant | 1 | 103.43 | 104.85 | 88.44 | 67.43 | 122.60 |
| | | 10 | 82.62 | 140.83 | 78.70 | 75.37 | 227.88 |
| | | 100 | 79.84 | 155.15 | 0.94 | 108.04 | 362.51 |
| 106-2 | Roots | 1 | 99.70 | 102.97 | 91.68 | 73.47 | 178.34 |
| | | 10 | 81.55 | 129.18 | 65.01 | 89.20 | 288.54 |
| | | 100 | 75.24 | 153.27 | 36.83 | 94.25 | 379.50 |
| 106-3 | Stems | 1 | 98.40 | 101.32 | 108.72 | 82.80 | 96.75 |
| | | 10 | 80.54 | 122.96 | 75.45 | 86.86 | 325.02 |
| | | 100 | 78.05 | 130.75 | 33.96 | 101.70 | 384.12 |
| 106-4 | Leaves | 1 | 101.63 | 115.63 | 88.79 | 72.14 | 134.95 |
| | | 10 | 86.49 | 125.97 | 93.48 | 80.76 | 196.24 |
| | | 100 | 70.60 | 151.28 | 19.78 | 110.91 | 335.59 |
| 106-5 | Fruits | 1 | 95.91 | 105.04 | 89.37 | 67.40 | 132.88 |
| | | 10 | 82.04 | 138.74 | 48.56 | 91.03 | 267.65 |
| | | 100 | 73.93 | 131.98 | 32.75 | 92.56 | 411.42 |
| 106-6 | Flowers | 1 | 98.51 | 97.39 | 99.14 | 85.24 | 102.23 |
| | | 10 | 89.58 | 109.73 | 76.94 | 77.36 | 291.47 |
| | | 100 | 79.64 | 134.53 | 28.65 | 98.20 | 347.20 |

### Example 107. Ulleungdo Amur Corktree

TABLE 215

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 107 | Ulleungdo amur | | | | | | |
| | corktree | | | | | | |
| 107-1 | Whole plant | 1 | 28.66 | 86.01 | 183.09 | 85.60 | 108.41 |
| | | 10 | 49.34 | 37.16 | 207.76 | 168.26 | 215.62 |
| | | 100 | 72.69 | 26.62 | 439.63 | 449.73 | 210.29 |
| 107-2 | Roots | 1 | 26.01 | 82.24 | 101.41 | 92.46 | 102.97 |
| | | 10 | 38.65 | 38.56 | 299.07 | 141.90 | 231.67 |
| | | 100 | 85.08 | 24.36 | 400.45 | 325.46 | 223.21 |
| 107-3 | Stems | 1 | 25.01 | 44.05 | 170.81 | 102.21 | 142.16 |
| | | 10 | 39.65 | 40.69 | 240.02 | 141.53 | 106.21 |
| | | 100 | 78.65 | 30.86 | 400.87 | 428.62 | 315.64 |
| 107-4 | Leaves | 1 | 20.25 | 81.99 | 152.10 | 87.72 | 129.72 |
| | | 10 | 35.01 | 33.48 | 350.83 | 187.60 | 232.37 |
| | | 100 | 77.70 | 24.79 | 498.64 | 332.17 | 234.90 |
| 107-5 | Fruits | 1 | 24.02 | 74.74 | 148.67 | 97.92 | 100.87 |
| | | 10 | 40.29 | 31.70 | 209.35 | 156.47 | 145.77 |
| | | 100 | 81.07 | 24.09 | 446.48 | 430.94 | 337.91 |
| 107-6 | Flowers | 1 | 26.50 | 46.44 | 156.85 | 87.25 | 123.10 |
| | | 10 | 41.65 | 37.80 | 247.50 | 188.45 | 149.04 |
| | | 100 | 76.30 | 28.24 | 431.26 | 480.38 | 312.60 |

TABLE 216

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 107 | Ulleungdo | | | | | | |
| | amur corktree | | | | | | |
| 107-1 | Whole plant | | 98.00 | 108.65 | 85.29 | 77.74 | 94.78 |
| | | 10 | 88.42 | 131.81 | 52.16 | 81.57 | 268.07 |
| | | 100 | 77.64 | 142.60 | 31.26 | 106.38 | 401.86 |
| 107-2 | Roots | 1 | 100.00 | 102.95 | 110.09 | 72.00 | 171.86 |
| | | 10 | 88.61 | 123.24 | 77.39 | 79.51 | 314.01 |
| | | 100 | 75.92 | 153.32 | 16.95 | 109.14 | 443.30 |
| 107-3 | Stems | 1 | 97.04 | 111.58 | 101.78 | 74.95 | 117.86 |
| | | 10 | 89.18 | 138.14 | 79.71 | 81.10 | 173.96 |
| | | 100 | 73.14 | 137.51 | 40.94 | 95.40 | 335.23 |
| 107-4 | Leaves | 1 | 103.33 | 108.08 | 99.94 | 74.45 | 113.57 |
| | | 10 | 94.19 | 112.75 | 69.29 | 94.63 | 329.02 |
| | | 100 | 74.26 | 153.60 | 7.61 | 103.58 | 479.79 |
| 107-5 | Fruits | 1 | 100.35 | 105.10 | 104.61 | 77.95 | 176.95 |
| | | 10 | 94.73 | 130.06 | 46.92 | 85.15 | 299.90 |
| | | 100 | 71.63 | 137.56 | 21.46 | 99.17 | 372.40 |
| 107-6 | Flowers | 1 | 100.21 | 113.86 | 105.74 | 79.35 | 150.70 |
| | | 10 | 95.16 | 127.42 | 49.13 | 88.80 | 295.89 |
| | | 100 | 72.55 | 131.99 | 38.92 | 96.75 | 380.35 |

### Example 108. Alianthus-Like Prickly-Ash

TABLE 217

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 108 | Alianthus- | | | | | | |
| | like prickly- | | | | | | |
| | ash | | | | | | |
| 108-1 | Whole plant | 1 | 30.36 | 64.60 | 132.61 | 86.75 | 105.15 |
| | | 10 | 35.62 | 33.73 | 285.44 | 152.15 | 193.73 |
| | | 100 | 83.83 | 24.38 | 451.55 | 480.42 | 235.53 |
| 108-2 | Roots | 1 | 29.94 | 58.72 | 125.27 | 107.19 | 142.65 |
| | | 10 | 38.90 | 34.60 | 266.75 | 167.15 | 119.55 |
| | | 100 | 71.25 | 30.97 | 468.24 | 499.23 | 292.94 |
| 108-3 | Stems | 1 | 26.80 | 52.01 | 168.10 | 106.09 | 138.51 |
| | | 10 | 44.10 | 38.40 | 338.17 | 144.12 | 175.89 |
| | | 100 | 74.10 | 24.29 | 429.93 | 434.72 | 262.81 |
| 108-4 | Leaves | 1 | 21.28 | 83.50 | 169.45 | 82.14 | 110.35 |
| | | 10 | 34.65 | 31.03 | 324.75 | 115.05 | 215.01 |
| | | 100 | 71.38 | 21.60 | 445.59 | 480.43 | 303.00 |
| 108-5 | Fruits | 1 | 27.88 | 76.25 | 107.18 | 104.06 | 119.68 |
| | | 10 | 33.38 | 35.75 | 270.26 | 116.54 | 155.25 |
| | | 100 | 71.93 | 29.51 | 377.01 | 367.46 | 271.67 |
| 108-6 | Flowers | 1 | 30.69 | 50.49 | 102.71 | 104.95 | 143.25 |
| | | 10 | 40.56 | 37.92 | 346.75 | 148.31 | 177.90 |
| | | 100 | 79.00 | 25.20 | 454.54 | 336.93 | 297.44 |

TABLE 218

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 108 | Alianthus- | | | | | | |
| | like prickly- | | | | | | |
| | ash | | | | | | |
| 108-1 | Whole plant | 1 | 95.33 | 114.05 | 88.21 | 70.55 | 121.58 |
| | | 10 | 86.11 | 127.39 | 47.59 | 75.86 | 176.50 |
| | | 100 | 75.01 | 150.28 | 43.89 | 109.55 | 484.30 |
| 108-2 | Roots | 1 | 102.98 | 95.10 | 80.02 | 67.14 | 192.41 |
| | | 10 | 95.87 | 127.84 | 92.52 | 82.85 | 339.64 |
| | | 100 | 71.05 | 135.86 | 30.03 | 95.65 | 313.17 |
| 108-3 | Stems | 1 | 95.90 | 109.48 | 92.72 | 70.62 | 145.05 |
| | | 10 | 91.17 | 113.26 | 83.39 | 93.76 | 219.00 |
| | | 100 | 74.36 | 134.29 | 33.90 | 97.71 | 457.33 |
| 108-4 | Leaves | 1 | 103.84 | 106.44 | 95.80 | 80.77 | 162.97 |
| | | 10 | 92.98 | 120.78 | 52.40 | 79.25 | 176.00 |
| | | 100 | 70.87 | 160.66 | 29.44 | 96.68 | 367.51 |
| 108-5 | Fruits | 1 | 98.93 | 108.73 | 86.01 | 71.27 | 113.46 |
| | | 10 | 85.47 | 113.36 | 46.94 | 88.07 | 207.90 |
| | | 100 | 78.17 | 148.70 | 37.44 | 109.99 | 480.56 |
| 108-6 | Flowers | 1 | 100.46 | 112.49 | 86.25 | 71.74 | 108.75 |
| | | 10 | 85.78 | 129.98 | 94.75 | 77.24 | 292.43 |
| | | 100 | 71.27 | 144.99 | 16.11 | 104.36 | 467.46 |

### Example 109. Wax-Leaf Privet

TABLE 219

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 109 | Wax-leaf | | | | | | |
| | privet | | | | | | |
| 109-1 | Whole plant | 1 | 21.11 | 75.54 | 166.60 | 85.00 | 92.03 |
| | | 10 | 45.93 | 38.64 | 222.50 | 199.00 | 240.28 |
| | | 100 | 73.35 | 24.52 | 498.03 | 491.74 | 324.50 |
| 109-2 | Roots | 1 | 26.52 | 64.90 | 173.92 | 87.75 | 90.88 |
| | | 10 | 49.81 | 37.26 | 251.53 | 142.62 | 151.61 |
| | | 100 | 80.78 | 29.79 | 389.32 | 380.24 | 293.79 |
| 109-3 | Stems | 1 | 27.34 | 71.86 | 118.03 | 84.17 | 150.13 |
| | | 10 | 31.22 | 34.17 | 303.25 | 158.81 | 107.52 |
| | | 100 | 78.06 | 24.98 | 362.60 | 376.98 | 210.19 |
| 109-4 | Leaves | 1 | 20.56 | 73.79 | 107.15 | 87.04 | 135.76 |
| | | 10 | 38.74 | 38.71 | 248.87 | 171.41 | 178.41 |
| | | 100 | 70.01 | 27.50 | 459.77 | 423.45 | 271.12 |
| 109-5 | Fruits | 1 | 25.55 | 58.61 | 106.86 | 109.70 | 142.87 |
| | | 10 | 50.78 | 40.73 | 225.37 | 177.82 | 109.32 |
| | | 100 | 81.39 | 20.95 | 382.93 | 339.23 | 339.51 |
| 109-6 | Flowers | 1 | 28.08 | 41.71 | 181.84 | 80.20 | 95.37 |
| | | 10 | 39.61 | 39.74 | 324.64 | 177.21 | 219.00 |
| | | 100 | 83.08 | 24.46 | 368.50 | 470.28 | 301.07 |

TABLE 220

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 109 | Wax-leaf | | | | | | |
| | privet | | | | | | |
| 109-1 | Whole plant | 1 | 101.94 | 96.20 | 92.78 | 83.17 | 143.51 |
| | | 10 | 87.19 | 119.40 | 55.79 | 78.54 | 262.38 |
| | | 100 | 70.77 | 151.31 | 11.79 | 98.35 | 341.07 |
| 109-2 | Roots | 1 | 102.23 | 101.86 | 96.40 | 75.23 | 155.35 |
| | | 10 | 95.55 | 126.19 | 63.52 | 93.99 | 272.19 |
| | | 100 | 72.04 | 154.83 | 45.10 | 106.40 | 385.31 |
| 109-3 | Stems | 1 | 98.06 | 112.67 | 102.83 | 76.50 | 154.97 |
| | | 10 | 87.38 | 128.15 | 55.48 | 81.88 | 183.82 |
| | | 100 | 73.37 | 147.85 | 18.98 | 92.39 | 405.04 |
| 109-4 | Leaves | 1 | 103.70 | 104.49 | 107.80 | 74.30 | 177.68 |
| | | 10 | 84.37 | 106.01 | 82.67 | 78.98 | 173.40 |
| | | 100 | 76.44 | 160.43 | 7.94 | 108.95 | 489.25 |
| 109-5 | Fruits | 1 | 98.40 | 108.64 | 81.73 | 78.79 | 91.32 |
| | | 10 | 81.99 | 115.44 | 47.07 | 84.02 | 210.98 |
| | | 100 | 80.02 | 158.53 | 12.52 | 107.94 | 324.60 |
| 109-6 | Flowers | 1 | 96.54 | 101.94 | 91.79 | 80.12 | 99.38 |
| | | 10 | 93.67 | 127.83 | 63.70 | 92.80 | 252.72 |
| | | 100 | 80.63 | 143.42 | 22.45 | 91.22 | 409.63 |

### Example 110. Ulleungdo Honeysuckle

TABLE 221

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 110 | Ulleungdo | | | | | | |
| | honeysuckle | | | | | | |
| 110-1 | Whole plant | 1 | 23.02 | 79.14 | 139.75 | 103.79 | 87.47 |
| | | 10 | 31.50 | 33.68 | 254.63 | 146.94 | 246.34 |
| | | 100 | 81.11 | 24.95 | 491.07 | 411.52 | 289.77 |
| 110-2 | Roots | 1 | 23.09 | 85.55 | 185.31 | 98.55 | 87.54 |
| | | 10 | 31.47 | 40.66 | 258.18 | 190.07 | 149.21 |
| | | 100 | 76.37 | 24.45 | 373.40 | 432.66 | 249.73 |
| 110-3 | Stems | 1 | 23.85 | 67.20 | 182.43 | 110.94 | 146.57 |
| | | 10 | 40.51 | 40.60 | 307.23 | 159.46 | 154.84 |
| | | 100 | 78.53 | 20.48 | 460.80 | 330.02 | 205.36 |
| 110-4 | Leaves | 1 | 30.14 | 85.80 | 105.49 | 105.21 | 117.60 |
| | | 10 | 30.13 | 33.64 | 290.50 | 118.11 | 172.38 |
| | | 100 | 82.65 | 24.11 | 361.00 | 497.38 | 339.40 |
| 110-5 | Fruits | 1 | 21.11 | 72.34 | 101.73 | 96.34 | 93.00 |
| | | 10 | 48.99 | 35.33 | 257.06 | 148.59 | 121.97 |
| | | 100 | 72.80 | 21.95 | 394.02 | 382.56 | 284.11 |
| 110-6 | Flowers | 1 | 24.16 | 76.75 | 163.70 | 93.19 | 113.62 |
| | | 10 | 36.19 | 34.49 | 263.81 | 112.21 | 142.36 |
| | | 100 | 82.45 | 25.81 | 439.78 | 439.49 | 203.15 |

TABLE 222

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestoserone |
| | control | | 1 uM) | 1 mM) | Inhibitor IV 15 nM) | 100 ppm) | 100 nM) |
| 110 | Ulleungdo | | | | | | |
| | honeysuckle | | | | | | |
| 110-1 | Whole plant | 1 | 96.25 | 109.80 | 86.19 | 84.55 | 167.17 |
| | | 10 | 80.67 | 120.13 | 93.39 | 84.48 | 284.57 |
| | | 100 | 73.79 | 150.34 | 34.69 | 109.20 | 358.41 |
| 110-2 | Roots | 1 | 101.41 | 98.28 | 83.99 | 74.80 | 139.24 |
| | | 10 | 94.43 | 116.81 | 58.38 | 76.39 | 176.85 |
| | | 100 | 74.48 | 135.06 | 18.64 | 107.96 | 328.06 |
| 110-3 | Stems | 1 | 98.66 | 105.71 | 100.22 | 80.48 | 119.66 |
| | | 10 | 81.76 | 121.20 | 92.35 | 80.70 | 174.40 |
| | | 100 | 77.94 | 143.10 | 28.85 | 96.82 | 466.15 |
| 110-4 | Leaves | 1 | 99.89 | 104.72 | 81.07 | 69.32 | 181.38 |
| | | 10 | 85.32 | 126.68 | 85.70 | 82.54 | 232.24 |
| | | 100 | 74.33 | 133.97 | 20.38 | 92.15 | 444.56 |
| 110-5 | Fruits | 1 | 103.03 | 102.45 | 86.51 | 73.16 | 98.65 |
| | | 10 | 83.60 | 121.01 | 59.56 | 78.05 | 276.17 |
| | | 100 | 79.03 | 138.97 | 24.30 | 105.14 | 318.62 |
| 110-6 | Flowers | 1 | 97.56 | 96.06 | 81.18 | 69.25 | 204.76 |
| | | 10 | 93.09 | 112.25 | 91.12 | 86.51 | 288.86 |
| | | 100 | 72.83 | 141.89 | 24.77 | 107.01 | 396.23 |

### Example 111. Mountain Hydrangea

TABLE 223

| | | | Blood | Lipid- | Anti-obesity- | | Menopause-ER |
|---|---|---|---|---|---|---|---|
| | | | circulation- | cholesterol | Glycerol | Immunity- | binding |
| | | Conc. | NO Conc. | synthesis | Conc. | IFNg secretion | capacity |
| No. | Sample | (ppm) | (% of control) | (% of control) | (% of control) | (% of control) | (% of control) |
| | Negative | | 21.32 (oxLDL | — | — | — | — |
| | control | | 200 ppm) | | | | |
| | Positive | | 98.29 (Rosuvastatin | 50.00 (Atorvastatin | 872.36 (Isoproterenol | 964.24 (LPS | 182.11 (17β-estradiol |
| | control | | 0.05 ppm) | 70.4 nM) | 10 μM) | 1 ppm) | 1 ng/ml) |
| 111 | Mountain | | | | | | |
| | hydrangea | | | | | | |
| 111- | Whole plant | 1 | 20.36 | 46.11 | 153.34 | 81.66 | 81.50 |
| | | 10 | 49.76 | 38.43 | 300.52 | 156.06 | 138.97 |
| | | 100 | 80.02 | 30.53 | 445.00 | 337.09 | 269.18 |
| 111-2 | Roots | 1 | 21.46 | 64.37 | 108.32 | 87.03 | 143.89 |
| | | 10 | 49.25 | 34.28 | 318.53 | 180.43 | 163.73 |
| | | 100 | 82.41 | 26.49 | 489.52 | 301.02 | 262.92 |
| 111-3 | Stems | 1 | 21.96 | 74.55 | 130.95 | 98.18 | 87.71 |
| | | 10 | 44.93 | 40.14 | 322.35 | 122.05 | 234.56 |
| | | 100 | 72.54 | 29.74 | 471.40 | 359.11 | 295.20 |
| 111-4 | Leaves | 1 | 24.49 | 97.26 | 129.74 | 105.22 | 146.62 |
| | | 10 | 42.62 | 92.27 | 242.48 | 177.02 | 246.97 |
| | | 100 | 85.02 | 90.18 | 387.09 | 372.03 | 327.62 |
| 111-5 | Fruits | 1 | 29.43 | 71.92 | 151.42 | 94.54 | 104.15 |
| | | 10 | 37.29 | 30.56 | 318.49 | 165.48 | 231.31 |
| | | 100 | 78.95 | 23.49 | 410.62 | 389.12 | 263.45 |
| 111-6 | Flowers | 1 | 29.40 | 58.05 | 155.84 | 110.65 | 103.25 |
| | | 10 | 50.36 | 39.70 | 272.25 | 147.53 | 231.76 |
| | | 100 | 85.00 | 23.12 | 371.53 | 386.31 | 303.49 |

TABLE 224

| | | | | Blood | Cognitive | | |
|---|---|---|---|---|---|---|---|
| | | | Anti- | glucose- | ability-β- | | Andropause-AR |
| | | | allergy-IgE | Glucose | secretase | Liver health- | transcriptional |
| | | Conc. | secretion | absorption | activity | Cell viability | activity |
| No. | Sample | (ppm) | (% of control) | (% of control) | (% of control) | (% of control) | (% of control) |
| | Negative | | — | — | — | 68.25 (Ethanol | — |
| | control | | | | | 100 nm) | |
| | Positive | | 67.27 (Dexamethasone | 183.77 (Metformin | 50.00 (β-Secretase | 88.28 (Silymarin | 753.39 (Dihydrotestosterone |
| | control | | 1 uM) | 1 mM) | Inhibitor IV 15 nM) | 100 ppm) | 100 nM) |
| 111 | Mountain | | | | | | |
| | hydrangea | | | | | | |
| 111- | Whole plant | 1 | 97.09 | 102.11 | 93.11 | 74.77 | 185.88 |
| | | 10 | 95.96 | 124.29 | 45.96 | 80.85 | 320.89 |
| | | 100 | 72.38 | 153.06 | 35.19 | 105.93 | 454.43 |
| 111-2 | Roots | 1 | 97.23 | 110.50 | 89.81 | 65.47 | 147.00 |
| | | 10 | 92.27 | 121.48 | 45.26 | 83.47 | 244.22 |
| | | 100 | 75.17 | 141.34 | 44.45 | 104.57 | 335.77 |
| 111-3 | Stems | 1 | 103.03 | 101.63 | 105.52 | 79.69 | 124.22 |
| | | 10 | 90.61 | 113.64 | 95.05 | 89.80 | 250.23 |
| | | 100 | 77.46 | 143.46 | 40.46 | 105.31 | 325.85 |
| 111-4 | Leaves | 1 | 97.33 | 88.92 | 97.09 | 81.33 | 142.59 |
| | | 10 | 81.60 | 89.23 | 88.87 | 78.68 | 266.60 |
| | | 100 | 76.57 | 94.67 | 24.78 | 109.94 | 302.77 |
| 111-5 | Fruits | 1 | 101.34 | 98.03 | 87.11 | 68.19 | 162.24 |
| | | 10 | 90.12 | 110.72 | 82.01 | 83.36 | 173.42 |
| | | 100 | 71.57 | 130.05 | 40.38 | 99.94 | 411.26 |
| 111-6 | Flowers | 1 | 96.01 | 111.97 | 106.13 | 75.17 | 131.18 |
| | | 10 | 93.97 | 106.52 | 61.46 | 93.89 | 221.26 |
| | | 100 | 78.76 | 134.25 | 3.56 | 97.19 | 332.17 |

### Formulation Example 1: Preparation of Pharmaceutical Preparations

1. Preparation of powder

0.001 g of plant extracts

1 g of lactose

The above ingredients were mixed, and a sealable bag was filled with the mixed ingredients to prepare powder.

2. Preparation of Tablets

0.2 mg of plant extracts

100 mg of corn starch

100 mg of lactose

2 mg of magnesium stearate

After mixing the above ingredients, tableting was performed according to a conventional tablet preparation method to prepare tablets.

3. Preparation of Capsules

0.2 mg of plant extracts

100 mg of corn starch

100 mg of lactose

2 mg of magnesium stearate

After mixing the above ingredients, gelatin capsules were filled with the mixed ingredients according to a conventional capsule preparation method to prepare capsules.

4. Preparation of Pills

0.003 g of plant extracts

1.5 g of lactose

1 g of glycerin

0.5 g of xylitol

After mixing the above ingredients, according to a conventional method, pills were prepared so that the weight of one pill was 4 g.

5. Preparation of Granules

2 mg of plant extracts

50 mg of soybean extracts

200 mg of glucose

600 mg of starch

After mixing the above ingredients, 100 mg of 30% ethanol was added thereto, and drying was performed at 60° C. to form granules. Then, a capsule was filled with the granules.

### Formulation Example 2: Manufacture of Food

Foods containing the plant extracts of the present invention were prepared as follows.

1. Manufacture of Flour Food

0.05 to 1.0 part by weight of the plant extracts was added to flour to prepare a mixture. This mixture was used to manufacture health foods such as breads, cakes, cookies, crackers, and noodles.

2. Manufacture of Dairy Products

0.2 parts by weight of the plant extracts was added to milk, and various dairy products such as butter and ice cream were manufactured using the milk.

3. Manufacture of Mixed Grain Powder

Brown rice, barley, glutinous rice, and adlay were dried through pregelatinization by a known method, and then the dried grains were roasted and pulverized using a grinder to obtain powder having a particle size of 60 mesh. In addition, black beans, black sesame, and perilla were steamed and dried according to a known method. Then, the dried grains were roasted and pulverized using a grinder to obtain powder having a particle size of 60 mesh. The plant extracts were concentrated under reduced pressure using a vacuum concentrator and dried through spraying and hot air drying to obtain a dried product. The dried product was pulverized using a grinder to obtain powder having a particle size of 60 mesh.

Based on 100 parts by weight of the mixed powder, the dry powder of grains, seeds, and plant extracts prepared above was blended in the following ratios.

Grains (30 parts by weight of brown rice, 15 parts by weight of adlay, 20 parts by weight of barley),

Seeds (7 parts by weight of perilla, 8 parts by weight of black beans, 7 parts by weight of black sesame),

0.1 parts by weight of plant extracts,

0.5 parts by weight of lingzhi mushroom,

0.5 parts by weight of adhesive rehmannia

### Formulation Example 3: Manufacture of Beverages

1. Manufacture of Health Drinks

0.1 mg of plant extracts

1,000 mg of citric acid

100 g of oligosaccharide

2 g of plum concentrate

1 g of taurine

Adjusting the total volume to 900 mL with water

After mixing the above ingredients according to a conventional health drink manufacturing method, the mixture was stirred and heated at 85° C. for about 1 hour to obtain a solution. The solution was placed in a filtered and sterilized 2 L container, sealed, and sterilized. Thereafter, the filtered solution was refrigerated. Then, the solution was used in preparation of the health drink composition of the present invention.

As a preferred embodiment, the composition ratio is determined based on ingredients suitable for favorite drinks. However, the composition ratio may be arbitrarily changed according to regional and ethnic preferences such as demand class, demand country, and use purpose.

2. Manufacture of Vegetable Juice

1 g of the plant extracts of the present invention was added to 1,000 mL of tomato or carrot juice to prepare vegetable juice for health promotion.

3. Manufacture of Fruit Juice

1 g of the plant extracts of the present invention was added to 1,000 mL of apple or grape juice to prepare fruit juice for health promotion.

1. A method of improving blood circulation, improving cholesterol, suppressing obesity, improving immune function, improving female menopause, suppressing allergies, controlling blood glucose, improving cognitive function, improving liver health, or improving andropause, the method comprising administering a composition comprising one or more plant extracts selected from the group consisting of Island thistle (scientific name: Cirsium nipponicum (Maxim.) Makino), Deodeok (scientific name: Codonopsis lanceolata), and Ulleungdo aster (scientific name: Aster glehnii) as active ingredients to a subject in need thereof.

2. The method according to claim 1, wherein the plant extracts are substances extracted from plants using water, an organic solvent, or a mixture thereof.

3. The method according to claim 2, wherein the organic solvent is a low-grade alcohol having 1 to 6 carbon atoms, a polyhydric alcohol, a hydrocarbon-based solvent, or a mixture thereof.

4. The method according to claim 1, wherein, based on 100 parts by weight of the composition, the plant extracts are comprised in an amount of 0.0001 to 10 parts by weight.

5.-9. (canceled)

10. The method according to claim 1, the composition is a food composition, or a pharmaceutical composition.

11. The method according to claim 10, wherein the food composition is a health functional food.
